(19) United States
(12) Patent Application Publication
(10) Pub. No.: US 2026/0022421 A1
(43) Pub. Date: Jan. 22, 2026

(54) DIRECTIONAL ALTERATION OF AN INDIVIDUAL'S MICROBIOME

(71) Applicant: Zoe Limited, London (GB)

(72) Inventors: Francesca Giordano, London (GB); Francesco Asnicar, Trento (IT); Jonathan Thomas Wolf, London (GB); Richard James Davies, London (GB); Nicola Segata, Trento (IT)

(73) Assignee: Zoe Limited, London (GB)

(21) Appl. No.: 19/342,389

(22) Filed: Sep. 26, 2025

Related U.S. Application Data
(63) Continuation-in-part of application No. PCT/EP2024/058262, filed on Mar. 27, 2024.
(63) Continuation-in-part of application No. PCT/EP2024/058286, filed on Mar. 27, 2024.
(63) Continuation of application No. PCT/EP2024/058290, filed on Mar. 27, 2024.
(60) Provisional application No. 63/492,718, filed on Mar. 28, 2023.
(60) Provisional application No. 63/492,708, filed on Mar. 28, 2023.
(60) Provisional application No. 63/492,718, filed on Mar. 28, 2023.
(60) Provisional application No. 63/492,708, filed on Mar. 28, 2023.
(60) Provisional application No. 63/492,718, filed on Mar. 28, 2023.
(60) Provisional application No. 63/492,708, filed on Mar. 28, 2023.

(51) Int. Cl. C12Q 1/6883; C12Q 1/689; G16B 40/00; G16H 50/30

(52) U.S. Cl. CPC C12Q 1/6883; C12Q 1/689; G16B 40/00; G16H 50/30; C12Q 2600/118; C12Q 2600/156

(57) ABSTRACT

Using deep metagenomic sequencing of individual gut microbiomes, coupled with techniques such as detailed long-term diet, fasting, and same-meal postprandial cardiometabolic blood markers analyses, intestinal microbes (defined by clusters of genomes referred to as species-level genome bins (SGBs)) have been identified as associated with different conditions and/or habits. Techniques are disclosed for using food guidance to intentionally alter the composition of the microbiome of an individual. Such alteration may include increasing the presence, abundance, or relative abundance of pro-health indicator microbe(s) and/or decreasing the presence, abundance, or relative abundance of poor health indicator microbe(s).

### CROSS-REFERENCE TO RELATED APPLICATION

This is a continuation of International Patent Application No. PCT/EP2024/058290 filed Mar. 27, 2024; a continuation-in-part of PCT/EP2024/058262 filed Mar. 27, 2024; and a continuation-in-part of PCT/EP2024/058286 filed Mar. 27, 2024. Each of PCT/EP2024/058290, PCT/EP2024/058262, and PCT/EP2024/058286 claim priority to and the benefit of the earlier filing of U.S. Provisional Application No. 63/492,718, filed on Mar. 28, 2023 and U.S. Provisional Application No. 63/492,708, filed on Mar. 28, 2023. Each of these applications is incorporated by reference herein in its entirety.

### FIELD OF THE DISCLOSURE

The present disclosure relates generally to microbiome analyses, as well as methods of modifying the microbiome of an individual, and methods of diagnosing, maintaining, ameliorating, and treating health conditions, based on such analyses.

### BACKGROUND OF THE DISCLOSURE

Growing evidence implicates the gut microbiome as a factor in the development of a number of disease processes including inflammatory bowel diseases, atherosclerosis, obesity, diabetes, and colon cancer. The association of disease processes with an altered microbial community structure suggests that interventions that restore or ameliorate the normal resilient gut microbial community might be an innovative intervention, as well as a way to influence overall health and wellness.

The gut microbiome is made up of trillions of microorganisms, such as bacteria, archaea or archaebacteria, viruses, and microeukaryotes, all of which may be helpful and potentially harmful. Gut microorganisms appear to play an important role in digesting food, assisting with absorption and synthesis of nutrients, regulating metabolism, body weight, and immune function, as well as contributing to regulating brain functions and mood. However, the specific details of the microbiome's role in health (both poor health and good health) have proven difficult to reproducibly define.

Microbiomes of different individuals vary greatly. For instance, it is estimated that only ten to thirty percent of the bacterial species in a microbiome is common across different individuals. Much of this diversity of microbiomes remains unexplained, though diet, environment, and host genetics each appear to play a part. Home medical tests allow individuals to obtain an analysis of their microbiome by mailing a sample to a company for analysis. Determining how to utilize the results of the microbiome analysis can be challenging, as species identification provides little insight into the quality of the microbiome and/or actions that may be taken to improve an individual microbiome.

Microbiomes may be altered by illness, disease, medication, stress, injury, and changes in diet. Current guidance about what to eat and how to change a diet can be very difficult and confusing for the consumer. Not only do individuals have a large variety of food choices, but food that is healthy for one individual may not be healthy for another individual. For example, while low carbohydrate food or low-fat food may be beneficial for one individual, that same low carbohydrate or low-fat food choice may be deleterious for another individual.

### SUMMARY OF THE DISCLOSURE

The current disclosure provides methods of using a group of microbes found in the gut microbiome to determine a health condition in a human subject.

In embodiments of the invention, the methods comprise: obtaining a biological sample from the human subject; and analyzing the biological sample to determine the presence, absence, or relative abundance of indicator microbes.

In embodiments of the invention, the methods comprise: obtaining a biological sample from the human subject; identifying in the biological sample at least 10, at least 20, at least 30, at least 40, at least 50, at least 60, at least 70, at least 80, at least 90, at least 100, at least 125, at least 150, at least 175, at least 200, or more than 200 different microbes in the biological sample; and determining the health condition of the human subject based on presence, absence, and/or absolute or relative abundance of the identified microbes in the biological sample.

In embodiments of the invention, the health condition comprises at least one of: overall good health, overall poor health, obesity, BMI, diabetes risk, cardiometabolic risk, cardiovascular disease risk, or postprandial response to food intake.

In embodiments of the invention, the methods comprise detecting the presence, absence, or relative abundance of at least one of the microbes in a microbiome sample from the human subject. In this context, the absence of a microbe may be detected by analyzing a sample for the microbe and determining that the microbe is absent.

In embodiments of the invention, the detecting comprises one or more of: sequencing one or more nucleic acids of an indicator microbe, hybridizing a nucleic acid probe to a nucleic acid of an indicator microbe, detecting one or more proteins from an indicator microbe, or measuring activity of one or more proteins of an indicator microbe.

In embodiments of the invention, the detecting comprises shotgun metagenomics.

In embodiments of the invention, the biological sample comprises a stool sample.

Another aspect provides methods of predicting a health condition in a subject, the method including: determining the presence, absence, or relative abundance of at least three indicator microbes in a microbiome of the subject; and predicting the health condition of the subject, based on the presence, absence, or relative abundance of the indicator microbes in the microbiome of the subject; wherein at least one of the indicator microbes is selected from Table 1A.

In embodiments of the invention, the health condition comprises at least one of obesity, increased cardiometabolic risk, diabetes risk, or overall poor health; and the health condition is predicted by the presence and/or relative abundance of indicator microbes; and/or the health condition comprises at least one of overall good health or absence of obesity, reduced cardiometabolic risk, or reduced diabetes risk; and the health condition is predicted by the presence and/or relative abundance indicator microbes.

Also provided are methods to predict overall good or poor general health in a non-diseased human subject, which methods include: obtaining a microbiome sample from the human subject; isolating a nucleic acid fraction from the microbiome sample; detecting, within the nucleic acid fraction, presence, absence, or relative abundance of at least one unique marker sequence indicative of at least one indicator microbe of Table 1A. In some aspects, methods to predict good or poor general health in non-disease human subject by obtaining a microbiome sample from the human subject; isolating a nucleic acid fraction from the microbiome sample detecting, within the nucleic acid fraction, presence, absence, or relative abundance of a pro-health indicator microbe selected from the group GOOD BUGS (as listed in Table 1B, or another subset of pro-health/good microbes described herein); or a poor health indicator microbes selected from the group including BAD BUGS (as listed in Table 1C, or another subset of poor-health/bad microbes described herein); and at least one of predicting the human subject has overall good general health if the pro-health indicator microbes outnumber or are relatively more abundant than the poor-health indicator microbes; or predicting the human subject has overall poor general health if the poor health indicator microbes outnumber or are relatively more abundant than the pro-health indicator microbes.

In embodiments of the invention, the methods comprise providing to the human subject a dietary recommendation based on the presence, absence, or relative abundance of one or more indicator microbes.

This disclosure further provides an assay, which includes: subjecting nucleic acid extracted from a test sample of a human subject to a genotyping assay that detects at least one indicator microbe from Table 1A, the test sample including microbiota from a gut of the subject; determining a relative abundance of the at least one indicator microbe that is below a predetermined abundance; and selecting, when the relative abundance is below the predetermined abundance, a treatment regimen that includes at least one of: (i) modifying microbiota of the gut of the subject using at least one of a prebiotic, probiotic, or pharmaceutical, or (ii) altering the diet of the human subject.

Another aspect is an assay, which includes: subjecting nucleic acid extracted from a test sample of a human subject to a genotyping assay that detects at least one indicator microbe of table 1A, the test sample including microbiota from a gut of the subject; determining a relative abundance of the at least one indicator microbe that is above a predetermined abundance; and selecting, when the relative abundance is above the predetermined abundance, a treatment regimen that includes at least one of: (i) modifying microbiota of the gut of the subject using at least one of a prebiotic, probiotic, or pharmaceutical, or (ii) altering the diet of the human subject.

Also described herein are microbial signatures (fingerprints) for good health, which include the presence or relative abundance of at least 10 ranked indicator microbes selected from Table 1A over a threshold rank.

This disclosure also describes microbial signatures (fingerprints) for poor health, including the absence or relatively low abundance of ranked indicator microbes selected from Table 1A over a threshold rank and the presence or relatively high abundance of indicator microbes below a threshold rank.

An aspect of the disclosure further relates to use of microbial signatures of the disclosure to guide treatment decisions for a human subject.

In some embodiments, the treatment decision comprises selecting one or more of: modifying overall diet, increasing intake of at least one specified food or supplement, decreasing intake of at least one specified food or supplement, administration of a probiotic composition, administration of a prebiotic composition, or administration of an antibiotic compound.

Another aspect provides methods for targeting a microbiome of a human subject to promote health, which methods include: (A) detecting in a microbiome sample from the human subject one or more ranked indicator microbes of Table 1A above a threshold rank; and administering to the human a composition that increases growth or survival of the indicator microbe(s) above the threshold rank; and/or (B) detecting in a microbiome sample from the human subject one or more ranked indicator microbes of Table 1A below a threshold rank and administering to the human a composition that decreases growth or survival of the ranked indicator microbe(s) below the threshold rank.

Also described are probiotic compositions for ingestion by a human subject, which include at least one of the ranked indicator microbes of Table 1A which are above a threshold rank. Also provided are methods of altering abundance of one or more microbes in gut microflora of a subject, which includes administering such a probiotic composition to the subject.

In some embodiments, the probiotic compositions comprise at least three, at least five, at least seven, at least 9, at least 10, at least 12, at least 14, or all of the listed microbes above a threshold rank.

Another aspect relates to methods of altering the abundance of one or more microbes in gut microflora of a subject, comprising administering the probiotic composition of the disclosure to the subject.

Yet another embodiment is a system to assay a biological condition in a subject, which system includes: a nucleic acid sample isolation device, which is adapted to isolate a nucleic acid sample from the subject; a sequencing device, which is connected to the nucleic acid sample isolation device and adapted to sequence the nucleic acid sample, thereby obtaining a sequencing result; and an alignment device, which is connected to the sequencing device and adapted to align the sequencing result against sequence from one or more of microbes in order to determine the presence or absence of the microbe(s) based on the alignment result, wherein the microbes include one or more of ranked indicator microbes of Table 1A.

Methods, assays, microbial signatures, use of microbial signatures, probiotic compositions, and/or systems of the disclosure may for example permit improved, more efficient, easier, cheaper, or more rapid diagnosis, prediction, or determination of health conditions. Further, health of human subjects may be improved.

Provided herein are methods and systems for altering the microbiome composition of a subject, such as a human subject, through diet modification.

Disclosed herein is the first demonstration the inventors are aware of whereby dietary guidance is shown to provide a material (and positive) change to the microbiome of subjects when the dietary guidance is adhered to.

In a described embodiment, correlations between food consumed and microbiome members (“bugs”) for thousands of people were used to identify foods that are to be promoted (e.g., those that tend to support a healthy condition in the subject, or to move a subject more toward a healthy state) and foods that are to be demoted (e.g., those that do not support a healthy condition, or that tend to support an unhealthy condition in the subject, or to move a subject more toward a less healthy state). When a subject/individual is advised (based on these correlations) which foods to promote and which to demote in their diet, as part of a food guidance or diet program, and the subject then adheres to this food guidance program over a period of time, the subject's/individual's gut health (and metabolic health) is improved.

This improvement in gut health may include an increase in the presence or abundance or relative abundance of at least one ranked indicator microbe above a threshold rank in the subject's/individual's microbiome, or a decrease in the presence or abundance or relative abundance of at least one ranked indicator microbe below a threshold rank in the subject's/individual's microbiome, or some combination of both.

This disclosure provides methods of altering the presence, absence, and/or relative or absolute quantity of member(s) of a gut microbiome in a subject, by altering the diet (e.g., pattern and/or content of food and beverages consumed) of the subject. Broadly speaking, the subject's diet is altered by providing the subject with a diet or food guidance program that is different from their current intake regimen, which program is then adhered to by the subject for a period of time. The content of the subject's microbiome is assessed at least once, for instance, before the diet or food guidance program is implemented; by measuring before, the microbiome fingerprint of the subject can be used as factor(s) that influence the program itself. Optionally, the microbiome is assayed more than once, for instance a second time that is some period after initiation of adherence to the food guidance program. This latter microbiome fingerprint can be used to assess the success (in improving gut health) of the food guidance program, for instance.

Also provided is a reiterative method, which allows for continued improvement over time. In these reiterative embodiments, measurement of change in a microbiome fingerprint from one sample timepoint to the next is used in order to improve generalized or personalized food guidance to that individual. In examples of such methods, how many “good” and/or “bad” bugs have changed between the two time points (and other changes in the fingerprint or microbiome score like diversity, etc.) is measured, and this is with correlate food guidance provided to the individual over time. Such correlations can be improved, for instance, by tracking what the individual consumes-including as an aspect of tracking their adherence to the food guidance program more generally. Machine learning can be used to compare the data gathered from a single subject/individual with similar data gathered from a databank of other people (for instance, thousands of other people), also following particular food guidance and for whom measurements in changes in microbiome diversity and content are available at at least two time points. The comparison is then used to revise and improve the dietary guidance (such as personalized dietary guidance) that is given to the individual, and optionally to other individuals, so as to improve on the levels of decrease in bad bugs (and increase in good bugs). It is also contemplated that in some embodiments, the individual's updated/reiterated diet program is influenced by the current and prior record of their own results and changes, rather than relying on comparison to a database of results from other individuals.

In the methods, systems, and uses described herein, the food guidance program that is conveyed to a single individual (also referred to herein as a subject) is prepared at least in part based on or in reference to a database of biomarkers and other information related to, for instance, general health, gut health, microbiome content (such as the presence, absence, abundance, or relative abundance of specific microbes, and/or overall diversity, and other recognized measures of microbiome health), nutritional information, circadian rhythm, gathered from a plurality of individuals. Methods to prepare such an aggregated database of information are known, including methods developed by Zoe Limited. Additional guidance regarding representative methods to generate a database useful in preparing food guidance for a group or an individual (e.g., a personalized food guidance) may be found for instance in: WO 2019/155436 “Generating Predicted Values of Biomarkers For Scoring Food”; WO 2019/155437 “Generating Personalized Nutritional Recommendations Using Predicted Values Of Biomarkers”; WO 2019/224308 “Improving the Accuracy of Measuring Nutritional Responses in a Non-Clinical Setting”; WO 2020/043702 “Generating Personalized Food Recommendations from Different Food Sources”; WO 2020/043705 “Improving The Accuracy of Test Data Outside the Clinic”; WO 2020/043706 “Using at Home Measures to Predict Clinical State and Improving the Accuracy of At Home Measurements/Predictions Data Associated with Circadian Rhythm and Meal Timing”; WO 2021/038530 “Generalized Personalized Food Guidance Using Predicted Food Responses”; US 2021-0065873 A1 “Generating Personalized Food Guidance Using Predicted Food Responses”; and WO 2021/186047 A1 “Microbiome Fingerprints, Dietary Fingerprints, and Microbiome Ancestry, and Methods of their Use”.

The current disclosure provides in exemplar embodiments use of a diet program (which is optionally a personalized diet program, personalized for the individual subject, or personalized to a group to which the subject belongs) to improve the gut microbiome of a subject, where the improvement to the gut microbiome includes: increasing presence or relative abundance of at least one ranked indicator microbe above a threshold rank in the microbiome of the subject; decreasing presence or relative abundance of at least one ranked indicator microbe below a threshold rank in the microbiome of the subject; or both. By way of example, the diet program may be prepared at least in part based on: the presence, absence, or relative abundance of at least one ranked indicator microbe above a threshold rank in the microbiome of the subject; and/or presence, absence, or relative abundance of at least ranked indicator microbe below a threshold rank in the microbiome of the subject.

Also provided are methods that include: preparing a food guidance program (which is optionally a personalized food guidance program, personalized for the individual subject, or personalized to a group to which the subject belongs) for a subject; communicating the food guidance program to the subject; wherein, when the subject follows the food guidance program: presence or relative abundance of at least ranked indicator microbe above a threshold rank in the microbiome of the subject is increased; presence or relative abundance of at least one ranked indicator microbe below a threshold rank in the microbiome of the subject is decreased; or both.

Yet another embodiment is a method that includes: detecting in a microbiome sample from the human subject one or more ranked indicator microbes of Table 1A above a threshold rank; and modifying the diet of the human using a food guidance program (which is optionally a personalized food guidance program, personalized for the individual subject, or personalized to a group to which the subject belongs) that increases growth or survival of the indicator microbe(s) above a threshold rank; and/or detecting in a microbiome sample from the human subject one or more health indicator microbe ranked indicator microbe below a threshold rank; and modifying the diet of the human using a food guidance program (which is optionally a personalized food guidance program, personalized for the individual subject, or personalized to a group to which the subject belongs) that decreases growth or survival of the poor health indicator microbe(s).

Also provided are methods of altering abundance of at least one microbial species in a gut microbiome of a subject, including: providing the subject with a personalized food guidance program, which personalized food guidance program is developed based at least in part on a food score personalized for the subject, and modifying food intake of the subject based on the personalized food guidance plan, thereby altering the abundance of at least one microbial species in the gut microbiome of the subject. For instance, in some examples of this method embodiment, altering abundance includes: increasing presence or relative abundance of at least one pro-health indicator microbe in the microbiome of the subject; decreasing presence or relative abundance of at least one poor health indicator microbe in the microbiome of the subject; or both.

Yet another embodiment is a method of improving gut microbiome balance/health of a subject, including developing a personalized food guidance program for the subject, communicating the personalized food guidance program to the subject, modifying food intake of the subject, in accordance with the personalized food guidance program, thereby improving gut microbiome balance/health of the subject.

Also described are methods of improving a gut microbiome profile of a subject, which methods include: assaying the gut microbiome of the subject, thereby producing a first microbiome signature of the subject; developing a personalized food guidance program for the subject, which personalized food guidance program will improve the gut microbiome of the subject when the subject follows the program; communicating the personalized food guidance program to the subject; assaying the gut microbiome of the subject at a time the after the subject begins to follow the customized diet guidance plan, thereby producing a second microbiome signature of the subject; comparing the first microbiome signature of the subject to the second microbiome signature of the subject, wherein the first and second microbiome signatures include the presence, absence, or relative abundance of one or more ranked indicator microbes above a threshold rank and/or the presence, absence, or relative abundance of one or more ranked indicator microbe below a threshold rank; and wherein an increase in the presence or relative abundance of the one or more ranked indicator microbe above a threshold rank and/or a decrease in the presence or relative abundance of the one or more ranked indicator microbe below a threshold rank from the first signature to the second signature constitutes an improved gut microbiome profile of the subject.

In any of the described embodiments, the personalized food guidance program is based at least based in part on one or more of: a non-microbial biomarker of the subject; a nutritional response of the subject; medical history of the subject; a health condition of the subject; predicted hunger of the subject; predicted response to food consumption of the subject; glucose response of the subject; fat response of the subject; microbiome data of the subject; data about the subject's overall health; and/or potential health risks for the subject.

For instance, in some specific uses and methods, the indicator microbe(s) includes a specific subset of GOOD BUG listed in Table 1B (for instance, a subset of pro-health/good microbes described herein including ranked microbes above a threshold rank shown in Table 1A). Alternatively, or in addition, in some uses and methods the indicator microbe(s) includes one or more of BAD BUGS listed in Table 1C (for instance, a subset of poor-health/bad microbes described herein including ranked microbes below a threshold rank shown in Table 1A).

In any of the use and method embodiments, the food guidance program may be developed based at least in part based on or in reference to a database including one or more of: correlations between food consumed and microbiome members for thousands of individual subjects; and/or a plurality of foods designated as “to be promoted in a diet”, wherein this designation indicates the food tends to support a healthy condition in the subject, or tends to move a subject incorporating the food in their diet more toward a healthy state; and/or a plurality of foods designated as “to be demoted in a diet”, wherein this designation indicates the food tends to not support a healthy condition, or tend to support an unhealthy condition in the subject, or tends to move a subject incorporating the food in their diet more toward a less healthy state.

Also provided in another embodiment is use of a diet program, such as a personalized diet program, to improve the gut microbiome of a subject or to alter the abundance of at least one microbial species in a gut microbiome of a subject, essentially as disclosed herein.

In embodiments of the invention, the methods include: receiving first test data from a remote device, the test data representing quantities of microbes present in a microbiome associated with an individual at a first time; accessing a ranked list of indicator microbes; determining, based at least in part on the first test data and the list of indicator microbes, a first microbiome score representing a quality of the microbiome associated with the individual at the first time; and sending the microbiome score to a storage location accessible by a device associated with the individual.

In embodiments of the invention, the methods include: using one or more weighted values associated with types of microbes in determining the microbiome score.

In embodiments of the invention, the methods include: receiving first diet data from a remote device, the diet data representing the diet of the individual at the first time; and using the first diet data at least in part in determining the microbiome score.

In embodiments of the invention, the methods include: generating, based at least in part on the first microbiome score and the first test data, a first recommended action, the first recommended action to increase a relative quantity of pro-health associated indicator microbes or decrease a relative quantity of poor health associated indicator microbes in the microbiome associated with the individual.

In embodiments of the invention, the first recommended action is a change in diet of the individual.

In embodiments of the invention, the first recommended action is a consumption of prebiotics or probiotics by the individual.

In embodiments of the invention, the methods include: receiving second test data from the remote device, the second test data representing quantities of microbes present in the microbiome associated with the individual at a second time, the second time subsequent to the first time; determining, based at least in part on the second test data and the list of indicator microbes, a second microbiome score representing a quality of the microbiome associated with the individual at the second time; and sending the second microbiome score to the storage location accessible by the device associated with the individual.

In embodiments of the invention, the methods include: receiving tracking data associated with the first recommended action from the device associated with the individual; and wherein the second microbiome score's improvement compared against the first microbiome score is correlated to determining, based at least in part on the tracking data, that the individual has followed the recommended action for a predetermined period for time or until a target associated with the first recommended action is achieved.

In embodiments of the invention, the methods include: determining the one or more weighted values based at least in part on health and/or diet data and microbiome data associated with a plurality of individuals.

In embodiments of the invention, the methods include: determining the one or more weighted values based at least in part on microbiome data associated with a plurality of individuals at two or more times per individual.

In embodiments of the invention, the methods include: determining the one or more weighted values further includes: training one or more machine learned models using microbiome data associated with a plurality of individuals; and receiving, from the one or more machine learned models, at least one weighted value associated with a type of microbe.

In embodiments of the invention, the methods include: determining the first microbiome score further includes: inputting the first test data into one or more machine learned models trained using microbiome data associated with a plurality of individuals; and receiving, as an output from the one or more machine learned models, the first microbiome score.

In embodiments of the invention, the methods include: generating a graphical representation of changes in the microbiome associated with the individual over a period of time including the first time and the second time; and causing the graphical representation to be presented on a display of the device associated with the individual.

In embodiments of the invention, the quantity of microbes is expressed as a percentage of the microbiome.

In embodiments of the invention, the methods include: at least ten of the indicator microbes (identified by its species-level genome bin (SGB) designation) selected from: SGB15249, SGB6340, SGB4964, SGB14252, SGB15229, SGB6174_group, SGB15317, SGB14179, SGB15225, SGB4894, SGB4643, SGB4963, SGB79840, SGB4893, SGB6276, SGB3952, SGB4638, SGB15236, SGB4191, SGB15053_group, SGB15368, SGB4782, SGB14042, SGB4706, SGB4644, SGB49188, SGB4781, SGB4777, SGB14921, SGB15234, SGB8601, SGB5087, SGB14311, SGB4953, SGB7258, SGB4882, SGB6367, SGB15106, SGB4778, SGB15131, SGB4198_group, SGB15031, SGB13981, SGB15123, SGB54300, SGB4665, SGB13979, SGB15410, SGB2290, SGB14954, SGB14306, SGB4805, SGB14899, SGB4803, SGB13982, SGB15265_group, SGB14114, SGB47656, SGB6749, SGB14253, SGB15346, SGB4810, SGB4770, SGB14043, SGB4886, SGB25497, SGB4815_group, SGB25416, SGB4957, SGB4654, SGB15373, SGB15254, SGB6571, SGB15323, SGB71759, SGB4648, SGB15180, SGB15413, SGB49168, SGB14960, SGB4133, SGB15051, SGB4993, SGB15395, SGB15145, SGB5111, SGB6317, SGB4966, SGB4780, SGB14198, SGB63101, SGB4779, SGB15233, SGB4769, SGB2295, SGB72336, SGB4658, SGB14770, SGB6148, SGB25493, SGB4831_group, SGB14965, SGB15224, SGB4938, SGB15402, SGB15291, SGB9333, SGB4664, SGB4906, SGB4711, SGB15065, SGB714_group, SGB4772, SGB3958, SGB4629, SGB14048, SGB15052, SGB14861, SGB9205, SGB4280, SGB4829, SGB4816, SGB2317, SGB15411, SGB5117, SGB14250, SGB14924, SGB4767, SGB6376, SGB4714, SGB4691, SGB14341, SGB15244, SGB5082_group, SGB4910, SGB4914, SGB8599, SGB4936, SGB15374, SGB72916, SGB4909, SGB15390, SGB15164, SGB15093, SGB13983, SGB5042, SGB4771, SGB15356, SGB72479, SGB4557, SGB3988, SGB15041, SGB14128, SGB15385, SGB6750, SGB4184, SGB3573, SGB66170, SGB15201, SGB15203, SGB79798, SGB15382, SGB4652, SGB9346, SGB14969, SGB4262, SGB4394, SGB61601, SGB15216, SGB14027, SGB4674, SGB14937, SGB15090, SGB9391, SGB15383, SGB29347, SGB14991, SGB14940, SGB4809, SGB6141, SGB4687, SGB63163, SGB14177, SGB4832, SGB15160, SGB48024, SGB6179, SGB4768, SGB5090_group, SGB29302, SGB9712_group, SGB3813, SGB79833, SGB4659, SGB4328, SGB4776, SGB1790, SGB14313, SGB5043, SGB15127, SGB15049, SGB42321, SGB15403, SGB15115, SGB4905, SGB14838, SGB15012, SGB9202, SGB80143, SGB3992, SGB7259, SGB4546, SGB14974, SGB13976, SGB15342, SGB2296, SGB14941, SGB3996, SGB53497, SGB15470, SGB14020, SGB1858, SGB14851, SGB6305, SGB14932, SGB15089, SGB1862, SGB15401, SGB4027, SGB15140, SGB2325, SGB14317, SGB4628, SGB4669, SGB15299, SGB6478, SGB14262, SGB63342, SGB4960, SGB63333, SGB15316_group, SGB4651, SGB1965, SGB15081, SGB59819, SGB2326, SGB14912, SGB14322_group, SGB3940, SGB4029, SGB2301, SGB63167, SGB14797_group, SGB5200, SGB17347, SGB4868, SGB15067, SGB53515, SGB15075, SGB4421, SGB5121, SGB9226, SGB2318, SGB14894, SGB4817, SGB14966, SGB3989, SGB15370, SGB14975, SGB4436, SGB14839, SGB14993_group, SGB15322, SGB9387, SGB3959, SGB6362, SGB4063, SGB14773_group, SGB29334, SGB14151, SGB15087, SGB14022, SGB14972, SGB15045, SGB4712, SGB15389, SGB82503, SGB15318_group, SGB1857, SGB4828_group, SGB4788_group, SGB79822, SGB4269, SGB14923, SGB2328, SGB1784, SGB14137, SGB15204, SGB7256, SGB15295_group, SGB14182, SGB29342, SGB4438, SGB14824_group, SGB2303, SGB9262, SGB14952, SGB14951, SGB15459, SGB6358, SGB14929, SGB15286, SGB5060, SGB15332_group, SGB15126, SGB72433_group, SGB4045, SGB1626, SGB5180, SGB4867, SGB4825, SGB4925, SGB53517, SGB1844, SGB14844, SGB4871_group, SGB14050, SGB25547, SGB1815, SGB3957, SGB54347, SGB6140, SGB14127, SGB29339, SGB1832, SGB9342_group, SGB15278, SGB9203, SGB1962, SGB5076, SGB4808, SGB15119, SGB3962, SGB14892, SGB4775, SGB4166, SGB7144, SGB4959, SGB63353, SGB14953, SGB6139, SGB16971, SGB15300, SGB3574, SGB47850, SGB63327, SGB4181, SGB15506, SGB5190, SGB14181, SGB1846, SGB15068, SGB4537, SGB14906, SGB9347, SGB1786, SGB7265, SGB4571, SGB29321, SGB4531, SGB15073, SGB14933, SGB15154, SGB6747, SGB15273, SGB4367, SGB15091, SGB3965, SGB63369, SGB9224, SGB3964, SGB6952, SGB4765, SGB29305, SGB4285_group, SGB5089, SGB4581, SGB59869, SGB4727, SGB25431, SGB2299, SGB1829, SGB4990, SGB1891_group, SGB2286, SGB17278, SGB1949, SGB63343, SGB5045, SGB5075_group, SGB8007_group, SGB29375, SGB6847, SGB1798, SGB4670, SGB4582_group, SGB4290, SGB14891, SGB1785, SGB15209, SGB14150, SGB33551, SGB14958, SGB14807, SGB4820, SGB1812, SGB4716, SGB4425_group, SGB9340, SGB14779, SGB14125, SGB14259, SGB4784, SGB15260, SGB14741, SGB4577_group, SGB71281, SGB14307, SGB5803, SGB58519, SGB5077, SGB15125, SGB15143, SGB4116, SGB4677, SGB15467_group, SGB7202, SGB6178, SGB6796_group, SGB6783_group, SGB1860, SGB4059, SGB63326, SGB9272_group, SGB15350, SGB14334, SGB1941, SGB16986, SGB14909, SGB1963, SGB4774, SGB14143, SGB15272, SGB29380, SGB4811_group, SGB4595, SGB4834, SGB4030, SGB8071, SGB2311, SGB3991, SGB4722, SGB17244, SGB3993, SGB4553, SGB6956, SGB1699, SGB6962_group, SGB4705, SGB1957, SGB4532, SGB4327_group, SGB59559, SGB4594, SGB5765_group, SGB17169, SGB15120, SGB1948, SGB14853, SGB14898, SGB48424, SGB5792, SGB6754, SGB1934, SGB14854, SGB6768, SGB15156_group, SGB4750, SGB14142, SGB17153_group, SGB4552_group, SGB4951, SGB4303, SGB9286, SGB5051, SGB17237, SGB4940, SGB4597, SGB4563_group, SGB1867, SGB47515, SGB59562, SGB8059_group, SGB4991, SGB4540_group, SGB14862, SGB4422, SGB15124, SGB14987, SGB7263, SGB9283, SGB4348, SGB14895, SGB17154, SGB17167, SGB4626, SGB5843, SGB4575, SGB4613, SGB15076, SGB17130, SGB15904, SGB4080, SGB6846, SGB5825_group, SGB14808, SGB4874, SGB9228, SGB6320, SGB9260, SGB8002, SGB6936, SGB14962, SGB8053, SGB4701, SGB5197, SGB4036, SGB4744, SGB1877, SGB29313, SGB14845, SGB14963, SGB8056, SGB6939, SGB17256, SGB4749, SGB3961, SGB7142, SGB14999, SGB4747, SGB15149, SGB4987, SGB6767, SGB17248, SGB4725, SGB59576, SGB1903_group, SGB5183, SGB49059, SGB4121, SGB25538, SGB3970, SGB3969, SGB14890, SGB1830_group, SGB7967, SGB17168, SGB8047, SGB4046, SGB1855_group, SGB3922, SGB14995, SGB7253, SGB48013, SGB4741, SGB4671, SGB15878, SGB6769, SGB14180, SGB29433, SGB1871, SGB5182, SGB5736, SGB6771, SGB4721, SGB14837, SGB4044, SGB8095, SGB17152, SGB1861, SGB66069, SGB4031, SGB6153, SGB7264, SGB15121, SGB25437, SGB14546_group, SGB4933_group, SGB4763, SGB5193, SGB7985, SGB4699, SGB19850_group, SGB17137, SGB4785, SGB15078, SGB53821, SGB15452, SGB15271, SGB4724, SGB8255_group, SGB79823, SGB8028_group, SGB4573_group, SGB14874, SGB4988, SGB5184, SGB4786, SGB4826_group, SGB4041, SGB7984, SGB4761, SGB4447, SGB6744, SGB1836_group, SGB1814, SGB4630_group, SGB8163, SGB4617, SGB4588_group, SGB4742, SGB4572, SGB10115, SGB15158, SGB29328, SGB4791, SGB4688, SGB10068, SGB71883, SGB4760, SGB4037_group, SGB4529, SGB4837_group, SGB79883, SGB4762, SGB4797, SGB14809, SGB4758_group, SGB4703, SGB4606, SGB4584, SGB15132, SGB4746, SGB4862, SGB4798, SGB4861, SGB4608, SGB4035, SGB4794_group, SGB4753, SGB4583.

In embodiments of the invention, the microbes are ranked based at least in part on a diet rank or health rank.

In embodiments of the invention, at least ten of the selected microbes are ranked in the same order relative to each other as per their diet ranks as reflected in Table 1A. In some aspects, the order is sequential but not necessarily contiguous.

In embodiments of the invention, at least ten of the selected microbes are ranked in the same order relative to each other as per their health ranks as reflected in Table 1A. In some aspects, the order is sequential but not necessarily contiguous.

In embodiments of the invention, a system includes one or more processors; and one or more non-transitory computer readable media storing instructions executable by the one or more processors, wherein the instruction, when executed, causes the one or more processors to perform operations including: receiving one or more weighted values from one or more first machine learned models trained on microbiome data associated with a plurality of individuals, each of the one or more weighted values representing at least in part a pro-health or poor health impact of a microbe or a plurality of microbes; receiving first test data representing a presence of and quantities of microbes present in a microbiome associated with an individual at a first time; inputting the test data into one or more second machine learned models which use at least in part the one or more weighted values; receiving as a first output of the one or more second machine learned models a first microbiome score representing a quality of the microbiome associated with the individual at the first time; and sending the microbiome score to storage location accessible by a device associated with the individual.

In embodiments of the invention, a system includes: inputting the test data into one or more third machine learned models trained at least in part on observed changes in microbiome data for a plurality of test subjects over time; and receiving as an output of the one or more third machine learned models a recommended action, the recommended action intended to increase a relative quantity of pro-health indicator microbes or decrease a relative quantity of poor health indicator microbes in the microbiome associated with the individual.

In embodiments of the invention, a system includes receiving second test data representing quantities of microbes present in the microbiome associated with the individual at a second time, the second time subsequent to the first time; inputting the second test data into the one or more second machine learned models and receiving as a second output of the one or more second machine learned models a second microbiome score representing a quality of the microbiome associated with the individual at the second time; and sending the second microbiome score to the storage location accessible by the device associated with the individual.

In embodiments of the invention, a system includes inputting the test data into one or more fourth machine learned models trained at least in part on observed changes in microbiome data for a plurality of test subjects over time; and receiving as an output of the one or more fourth machine learned models, a plurality of predicted microbiome scores, each of the plurality of predicted microbiome scores representing a quality of the microbiome of the individual at a future time in response to the individual performing a corresponding recommended action.

In embodiments of the invention, a system includes selecting specific microbes from the test data; and inputting the quantities of the specific microbes into the one or more second machine learned models.

In embodiments of the invention, a system includes receiving user data associated with the individual, the user data representing at least one of a health of the individual, a demographic associated with the individual, a diet of the individual, or a geographic region associated with the individual.

In embodiments of the invention, a method includes receiving first test data from a remote device, the first test data representing quantities of microbes present in one or more first microbiome samples; receiving second test data from a remote device, the second test data representing quantities of microbes present in one or more second microbiome samples; accessing a list of indicator microbes and their associations with pro-health versus poor health; determining which indicator microbes increase or decrease between the first and second microbiome samples; determining whether the increasing indicator microbes have stronger pro-health associations or stronger poor health associations compared to the decreasing indicator microbes, representing respectively an increase or decrease in the quality of the microbiome between the first and second microbiomes samples; and sending the comparison to a storage location.

### BRIEF DESCRIPTION OF THE DRAWINGS

FIG. 1 is a block diagram depicting an illustrative operating environment in which personalized microbiome scores and recommended actions are generated using user data, third party data, microbiome data, and predetermined weights.

FIG. 2 is a block diagram depicting an illustrative operating environment in which microbiome data is analyzed to generate microbiome fingerprints, dietary fingerprints, microbiome ancestry, dietary scores, and nutritional recommendations for users.

FIG. 3 is another block diagram depicting an illustrative operating environment in which personalized microbiome scores and recommended actions are generated using microbiome and other user data.

FIG. 4 is a block diagram depicting an illustrative operating environment in which a data ingestion service receives, and processes test data associated with at home tests and sample collections.

FIG. 5 is a flow diagram showing an example for obtaining and utilizing user microbiome data to generate microbiome fingerprints, dietary fingerprints, microbiome score, and microbiome ancestry for users.

FIG. 6 is a flow diagram showing an example process for generating a microbiome fingerprint for a user.

FIG. 7 is a flow diagram showing an example process for generating microbiome scores for individuals.

FIG. 8 is a flow diagram showing an example process for generating a microbiome score for an individual.

FIG. 9 is a flow diagram showing an example process for generating a microbiome ancestry for a user.

FIG. 10 is a flow diagram showing an example process for generating a dietary fingerprint for a user.

FIG. 11 is a flow diagram showing an example process for generating microbiome scores and recommended actions to improve microbiome scores for individuals.

FIG. 12 is a flow diagram showing an example process for generating microbiome scores and recommended actions to improve microbiome scores for individuals.

FIG. 13 is a flow diagram showing a process illustrating aspects of a mechanism disclosed herein for obtaining test data, including microbiome data, that may be utilized for generating microbiome fingerprints, dietary fingerprints, and microbiome ancestry for users.

FIG. 14 is a computer architecture diagram showing one illustrative computer hardware architecture for implementing the techniques described herein according to some implementations.

FIGS. 15A-15C. Good Bugs extracted with health and diet markers. Normalized relative abundances of the significantly changing good bugs (Adjusted p-value≤0.2) extracted with health and diet markers for the Initial Cohort (darker grey left hand column of each pair of test data) and the Retest Cohort (lighter grey; right hand column of each pair of test data). The small light colored squares within each column represent the cohort mean relative abundance for a microbe.

FIGS. 16A-16C. Bad Bugs extracted with health and diet markers. Normalized relative abundances of the significantly changing bad bugs (Adjusted p-value≤0.2) extracted with health and diet markers for the Initial Cohort (darker grey; left hand column of each pair of test data) and the Retest Cohort (lighter grey; right hand column of each pair of test data). The small light colored squares within each column represent the cohort mean relative abundance for a microbe.

FIGS. 17A-17C. Good Bugs extracted only with health markers (no diet markers). Normalised relative abundances of the significantly changing good bugs (Adjusted p-value≤0.2) extracted with health and diet markers for the Initial Cohort (darker grey; left hand column of each pair of test data) and the Retest Cohort (lighter grey; right hand column of each pair of test data). The small light colored squares represent the cohort mean relative abundance for a microbe.

FIGS. 18A-18C. Bad Bugs extracted only with health markers (no diet markers). Normalised relative abundances of the significantly changing bad bugs (Adjusted p-value≤0.2) extracted with health and diet markers for the Initial Cohort (darker grey; left hand column of each pair of test data) and the Retest Cohort (lighter grey; right hand column of each pair of test data). The small pale colored squares represent the cohort mean relative abundance for a microbe.

### DETAILED DESCRIPTION

Aspects of the current disclosure are now described with additional details and options. Any headings included in this document do not limit the interpretation of the disclosure and are provided for organizational purposes only.

Using the technologies described herein, individual microbiome data as well as demographic, geographic, lifestyle, environmental, and other health related data are analyzed to generate personalized microbiome profiles including one or more of a microbiome fingerprint, a dietary fingerprint, a microbiome score, and microbiome ancestry data for a user. As used herein, a “microbiome fingerprint” is data that uniquely identifies the microbiome of a user at a particular point in time, and a “dietary fingerprint” is data that identifies how the microbiome of a user at a particular point in time is associated with one or more different indexes associated with a diet and/or health characteristics. The indexes may include, but are not limited to a Mediterranean diet index, a vegetarian diet index, a fast food index, an internal fat index, a fat-digesting index, a carbohydrate-digesting index, a health index, a fasting index, a ketogenic index, and the like. According to some configurations, a microbiome system as described herein may generate a score, such as from 0-100, (or some other indicator) that indicates how closely the microbiome of the user is associated with a particular index. A “microbiome score” represents the quality of an individual's microbiome, that is someone who is healthy would be expected to have a higher quality microbe in comparison to someone who is unhealthy. Such a quality may be reflected by the microbiome score with higher scores indicating relatively better health. Such scores may be absolute or relative to the scores of other individuals with the same or different demographic, geographic, lifestyle, environmental, and health measures. In some aspects, the score may be compared to a threshold score with a good score being above a threshold and a less healthy score being below the threshold.

As an example, the Mediterranean diet index score for a user indicates how closely the microbiome of the user resembles the typical microbiome of someone on a Mediterranean diet. A Mediterranean diet emphasizes plant-based foods and healthy fats with fruits, vegetables, whole grains and extra virgin olive oil eaten at every meal, seafood, nuts and legumes eaten at least three times a week, poultry low-fat dairy, and eggs eaten no more than once a day and red meats and sweats limited to one serving a week. The vegetarian diet index score indicates how closely the microbiome of the user resembles someone on a vegetarian diet. The fast food index score indicates how closely the microbiome of the user resembles someone on a fast food diet. The internal fat index score indicates how closely the microbiome of the user resembles someone with high or low visceral fat. The fat-digesting index score indicates how closely the microbiome of the user resembles someone with low postprandial triacylglycerol (TAG) rises (Sabaka et al., Lipids Health Dis 12, 179, 2013. doi.org/10.1186/1476-511X-12-179). The carbohydrate-digesting index score indicates how closely the microbiome of the user resembles someone with low postprandial glucose rises. The health index score indicates how closely the microbiome of the user resembles someone that is healthy. The fasting index score indicates how closely the microbiome of the user resembles someone that fasts regularly. The ketogenic index score indicates how closely the microbiome of the user resembles someone who is ketogenic. Descriptions of exemplary diets/indicies used for scoring may be found, for example, in Asnicar et al. (Nat Med. 27:321-323, 2021). In some aspects, scores may be analyzed using a single point of comparison. In other aspects, scores may be multifactorial.

The microbiome service may utilize microbiome data generated from a microbiome sample and/or other data to generate a personalized microbiome profile including one or more of a microbiome fingerprint, dietary fingerprint, microbiome score, and/or microbiome ancestry data for a user. For example, the microbiome service may perform an analysis of the microbiome data generated from a microbiome sample to identify the microbial composition (e.g., the species, genes, taxa, and the like) of a sample; such identification may include the unique, detailed characterization of each and every microbial strain in the sample, but it is not necessary to identify every strain present in the sample. For instance, the analysis of the microbiome data may identify as few as 2% of the strains in the sample; as few as 5%, as few as 8%, as few as 10%, as few as 15%, as few as 20%, or more than 30% of the strains in the sample. In certain embodiments, the characterization will identify more than 25% of the strains; for instance, more than 30%, more than 35%, more than 40%, more than 45%, more than 50%, more than 55%, more than 60%, more than 65%, more than 70%, more than 75%, more than 80%, more than 85%, more than 90%, or even more than 95% of the strains in the sample. In some aspects, the relative abundance of a specific microbe may be calculated.

In some examples, some/all of the analysis of the microbiome service may be performed by a service provider that is external to the microbiome service. The microbiome service may obtain this portion of the microbiome data from the external service provider(s). The microbiome service may also generate reconstructed microbial genomes, determine a diversity of the microbiome, identify functions of the microbiome, identify a uniqueness of the microbiome, identify interesting species, a relative abundance of the microbes and the like.

In some examples, the microbiome data of the user is utilized with other data that is gathered about the user, as well as other users. For instance, users may provide responses to questionnaires, data about food that is eaten, data about supplements or medicines that are eaten, sleep habits, exercise habits, mental health, and the like. In some aspects, the additional information may include other tests such as genetic testing, data obtained from wearables such as a smart watch or fitness tracker, blood tests, breath tests, blood pressure readings, glucose tests, and the like. The additional data may be included in an analysis of the user's microbiome to determine all or part of a personalized microbiome profile. For example, such information may be used all or in part to calculate a microbiome ancestry or a microbiome score for the individual or to provide dietary or other health recommendations for an individual.

In some aspects, a microbiome score or fingerprint for an individual may be compared to pools of user data to determine a score or fingerprint that is relative to a population. Such a population may be grouped according to a variety of criteria, for example, geographic location, sex, activity level, age, diet, type of activity, ethnicity, national origin, behaviors, and the like. For example, a user's score may be compared to all users in a particular country. In other aspects, the comparison may be to all women or all men. In other aspects, the comparison may be to a plurality of characteristics, for example, all female runners between 30-40 years of age who run at least three times a week, live north of 60° north latitude, and are on a ketogenic diet. In some aspects, a microbiome profile may include psychological data (e.g., hunger, sleep quality, mood, . . . ).

Among other uses, data in addition to the microbiome data may be utilized to assist in determining a “microbiome ancestry” of a user. A “microbiome ancestry” for a user indicates that the user has relationships with other users and/or locations based on a similarity of the microbiome data (e.g., the microbiome fingerprint) for a particular user with other users. A microbiome ancestry may be generated by analyzing the microbiome data of the user and determining how closely the microbiome of the user is related to one or more other users, and/or locations. For instance, the microbiome service may determine a number of other users to which the microbiome of the user is most closely related to. In some configurations, the microbiome service compares the microbiome data, such as the microbiome fingerprint, of the user to microbiome data, such as the microbiome fingerprints, of other users to determine whether the user is related to any of the other users. Thus, the microbiome ancestry data may be used to elicit additional details about a user. For example, a user with a particular microbiome ancestry may be identified as someone who is on the Mediterranean diet or someone who lives in a particular region. For instance, the microbiome service may determine that the microbiome fingerprint of the user is more similar to a microbiome of a user in France even though the user is from England. Such ancestry data may allow for more focused nutritional or other health recommendations for an individual or additional resources to determine a predictive microbiome score based on the anticipated impact of nutritional or other changes by a user.

Determination of scores may include a machine learning model or neural network algorithm trained using third-party health data and microbiome data to generate weights or factors for each individual species of microbes that could potentially be present in an individual's personal microbiome. In some cases, the weights may be positive, negative, or neutral depending on determined beneficial and/or harmful tendencies. These weights may then be used to generate microbiome scores for individuals based on the presence, absence, and/or comparable quantities of individual species of microbes in the microbiome of the individual.

Machine learned models may be generated using various machine learning techniques. For example, the models may be generated using one or more neural network(s). A neural network may be a biologically inspired algorithm or technique that passes input data (e.g., image and sensor data captured by computing devices as well as microbiome and other demographic or health data) through a series of connected layers to produce an output or learned inference. Each layer in a neural network can also comprise another neural network or can comprise any number of layers (whether convolutional or not).

As an illustrative example, one or more neural network(s) may generate any number of learned inferences or leads from the captured sensor and/or image data. In some cases, the neural network may be a trained network architecture that is end-to-end. In one example, the machine learned models may include segmenting and/or classifying extracted deep convolutional features of the sensor and/or image data into semantic data. In some cases, appropriate truth outputs of the model are in the form of semantic per-pixel classifications (e.g., vehicle identifier, container identifier, driver identifier, and the like).

Although discussed in the context of neural networks, any type of machine learning can be used with this disclosure. For example, machine learning algorithms can include, but are not limited to, regression algorithms (e.g., ordinary least squares regression (LSR), linear regression, logistic regression, stepwise regression, multivariate adaptive regression splines (MARS), locally estimated scatterplot smoothing (LOESS)), instance-based algorithms (e.g., ridge regression, least absolute shrinkage and selection operator (LASSO), elastic net, least-angle regression (LARS)), decisions tree algorithms (e.g., classification and regression tree (CART), iterative dichotomiser 3 (ID3), Chi-squared automatic interaction detection (CHAID), decision stump, conditional decision trees), Bayesian algorithms (e.g., naïve Bayes, Gaussian naïve Bayes, multinomial naïve Bayes, average one-dependence estimators (AODE), Bayesian belief network (BNN), Bayesian networks), clustering algorithms (e.g., k-means, k-medians, expectation maximization (EM), hierarchical clustering), association rule learning algorithms (e.g., perceptron, back-propagation, Hopfield network, Radial Basis Function Network (RBFN)), deep learning algorithms (e.g., Deep Boltzmann Machine (DBM), Deep Belief Networks (DBN), Convolutional Neural Network (CNN), Stacked Auto-Encoders), Dimensionality Reduction Algorithms (e.g., Principal Component Analysis (PCA), Principal Component Regression (PCR), Partial Least Squares Regression (PLSR), Sammon Mapping, Multidimensional Scaling (MDS), Projection Pursuit, Linear Discriminant Analysis (LDA), Mixture Discriminant Analysis (MDA), Quadratic Discriminant Analysis (QDA), Flexible Discriminant Analysis (FDA)), Ensemble Algorithms (e.g., Boosting, Bootstrapped Aggregation (Bagging), AdaBoost, Stacked Generalization (blending), Gradient Boosting Machines (GBM), Gradient Boosted Regression Trees (GBRT), Random Forest), SVM (support vector machine), supervised learning, unsupervised learning, semi-supervised learning, etc. Additional examples of architectures include neural networks such as ResNet50, ResNet101, VGG, DenseNet, PointNet, and the like. In some cases, the system may also apply Gaussian blurs, Bayes Functions, color analyzing or processing techniques and/or a combination thereof.

In some aspects, the percentage of each microbe present in a microbiome is calculated and the logarithm percentage is created. Each microbe may be given a particular weight as determined using machine learning. Each microbe may be given the same or different weights. For example, all microbes that are viewed as good microbes may be given a positive weight, microbes that are viewed as neutral may be given a weight of zero, and microbes that are viewed as bad microbes may be given a negative weight. In other aspects, the weights may be assigned based on the presence of microbes in people with certain conditions, for example, a microbe that appears in people who are healthy may be given a higher weight than a microbe that is only present in people with diabetes. In some aspects, the weights may be assigned based on the likelihood that the presence or relative abundance of a particular microbe will change based on the individual parameter that will be changed. For example, some microbes are more responsive to dietary change and the weight assigned to those microbes reflects the likelihood of change. Other microbes may be impervious to changes in diet and are, therefore, omitted or given a weight that reflects their intransigence. In other aspects, some microbes may change based on exercise or other lifestyle changes and may be weighted accordingly for a second calculation. In other aspects, the weighting of a microbe may reflect a plurality of factors such as beneficial properties, harmful properties, and responsiveness to change. For example, good microbes that are responsive to exercise and diet may be given one weight, bad microbes that are responsive to exercise and diet may be given a second weight, good microbes that are only responsive to diet and not to exercise may be given a third weight and the like. The microbiome system may generate one or more sets of microbiome profile data using one or more sets of weights depending on the desired end result. For example, if the user indicates that they are intending to make dietary changes, a first personalized microbiome profile may be generated using microbes assigned a first set of weights. If the user indicates that they are intending to exercise more but are not intending to change their diet, a second personalized microbiome profile using a second set of weights may be generated. If the user indicates that they are intending to exercise and change their diet, a third personalized microbiome profile using a third set of weights may be generated. This may allow the system to provide recommendations for behavior changes and predictive microbiome scores that would be achieved if those recommendations were followed.

Each microbe weight in an individual may be multiplied by the log abundance and combined to determine an absolute score. The absolute score may then be converted into a relative score depending on the reference population. That is, the relative score may be based on a comparison to one or more of the geographic region or other demographic, lifestyle, or health data.

The score may be calculated using some or all of the microbes. For example, in some aspects, the scores may be calculated using the top 10% and the bottom 10% of the microbes as determined by the weights of the microbes. In other aspects, it may be different percentages including 15%, 20%, 25%, 30%, or more. In other aspects, the score may be calculated using only the most positively weighted microbes or only the most negatively weighted microbes in an individual microbiome. In other aspects, the score may be calculated using the fraction of species in the microbiome that is most responsive to diet.

In some aspects, the system may utilize one or more machine learned models and/or networks that are trained on microbiome data, the weights discussed above, known health conditions, known diet, lifestyle, demographics, health data, geographic location and the like. In other implementations, a heuristic or algorithmic technique may be used in addition to or in lieu of the machine learned models and networks to generate the microbiome score.

In some examples, the microbiome service may provide a user interface (UI), such as a graphical user interface (GUI) to allow a user to view and interact with microbiome data and/or other data associated with the microbiome fingerprints, dietary fingerprints, microbiome score, and microbiome ancestry. For instance, the GUI may display microbiome fingerprint data that shows various characteristics of the microbiome fingerprint, dietary fingerprint data that shows various characteristics of the dietary fingerprint, microbiome ancestry data that shows various characteristics of the microbiome ancestry, microbiome score data that shows the factors included in the score, recommendation data that identifies one or more recommendations relating to changing the microbiome of the user, and the like.

The systems and methods described herein may provide individualized or generic recommendations depending on the user's preferences. For instance, a microbiome fingerprint or score from the individual may be used to personalize the food guidance by taking into account the initial or current state of the individual's microbiome as well as the individual's health data, demographic data, geographic data, genetic data, and food tolerances or intolerances.

In some aspects, a user may “opt-in” to allow use of the microbiome data and/or other data associated with a user as part of a larger pool of data. In some examples, the user “opts-in” to participate in a social network and/or some other communication mechanism to discuss issues related to the microbiome data such as a microbiome ancestry (e.g., compare diets and background with other users). The microbiome service may also compare the microbiome of the user with other family members, and/or other users when the users have “opted-in” to allow this. For instance, the microbiome service may identify how many strains they share (with respect to sharing with unrelated persons) and overall how similar they are compared to the average or a specific average such as an average of users with the same or different geographic, demographic, lifestyle, and health factors.

As an example, the microbiome service may provide generic recommendations to increase the diversity of foods eaten, as there is no one good food for a healthy microbiome. The recommendations may include instructions to eat different gut-healthy foods, eat fermented foods, minimize highly processed foods (things like emulsifiers and artificial sweeteners may affect the microbiome), consume prebiotic substances, administer a probiotic preparation, or any combination thereof. The microbiome service may base the recommendations on data obtained from the user, from other users, from scientific studies, and the like.

In some aspects, methods are provided that employ correlations between food/beverage consumed and microbiome members (“bugs”) for thousands of people to identify foods that individuals should be encouraged to consume and foods that individuals should be discouraged from consuming. By way of example, foods to be promoted in a diet may include those that tend to support a healthy condition in the subject, or to move a subject more toward a healthy state.

A healthy state may be confirmed or determined by one or more health metrics including personal data including age and height, as well as physical data such as weight, BMI, ASCVD risk, systolic and diastolic blood pressure, Visceral fat, Liver fat probability, and QUICKI insulin sensitivity. In some aspects the tests may be taken after fasting. In other aspects, the tests may be conducted post prandial. In some aspects, tests may be conducted both fasting and post-prandial. Fasted tests may be, for example, Glucose, HbA1c, C-peptides, Total cholesterol, high-density lipoprotein (HDL) cholesterol, cholesterol ratio (THR) (total cholesterol/HDL), triglycerides, GlycA, HDL size, and very-low density lipoprotein (VLDL) size. Post-prandial tests may be for example, glucose, glucose iAUC, Total cholesterol, HDL cholesterol, triglycerides, GlycA, HDL size, VLDL size, and CGM variation.

Nutritional markers (diet indices): may include for example, those described in Asnicar F, et al Nat Med. 2021 February; 27 (2): 321-332. doi: 10.1038/s41591-020-01183-8. Epub 2021 Jan. 11. PMID: 33432175; PMCID: PMC8353542 including the healthy food diversity index (HFD), Healthy Eating Index 2010 (HEI-2010), plant based diet index (PDI), and the Alternate Mediterranean Diet score (aMED).

As described in Asnicar, the HFD index may consider the number, distribution, and health value of consumed foods. To obtain this index, food frequency questionnaire foods may be aggregated into 15 food groups according to the HFD (Vadiveloo M, Dixon L B, Mijanovich T, Elbel B & Parekh N Development and evaluation of the US Healthy Food Diversity index. Br. J. Nutr 112, 1562-1574 (2014)). Health values may be derived from the German Nutrition Society (DGE) dietary guidelines (dge.de/en/) and the weight of each food group may be multiplied by its corresponding health value (hv). Scores may be divided by the maximum (hv=0.26) to bind values between 0-1 before multiplication with the Berry-Index. The Healthy Eating Index 2010 (HEI-2010) (PMC8353542/) (Guenther P M et al. Update of the Healthy Eating Index: HEI-2010. J. Acad. Nutr. Diet 113, 569-580 (2013)) assesses to which extent an individual's food intake aligns with the Dietary Guidelines for Americans 2010 (McGuire S U.S. Department of Agriculture and U.S. Department of Health and Human Services, Dietary Guidelines for Americans, 2010 7th Edition, Washington, DC: U.S. Government Printing Office, January 2011. Adv. Nutr. 2, 293-294 (2011)), developed by the US Department of Agriculture. Versions of the plant-based diet index (PMC8353542) may also be considered (Satija A et al. Healthful and Unhealthful Plant-Based Diets and the Risk of Coronary Heart Disease in U.S. Adults. J. Am. Coll. Cardiol 70, 411-422 (2017)) such as the original plant-based diet index (PDI), the healthy plant-based index (h-PDI), and the unhealthy plant-based index (u-PDI). An animal-based score may categorize animal foods into “healthy” and “less-healthy”/“unhealthy” categories according to previous epidemiological studies (WHO|Effect of trans-fatty acid intake on blood lipids and lipoproteins: a systematic review and meta-regression analysis. (2016); Zhong V W et al. Associations of Dietary Cholesterol or Egg Consumption With Incident Cardiovascular Disease and Mortality. JAMA 321, 1081-1095 (2019; de Souza R J et al. Intake of saturated and trans unsaturated fatty acids and risk of all cause mortality, cardiovascular disease, and type 2 diabetes: systematic review and meta-analysis of observational studies. BMJ 351, h3978 (2015); Michaëlsson K et al. Milk intake and risk of mortality and fractures in women and men: cohort studies. BMJ 349, g6015 (2014); Mazidi M et al. Consumption of dairy product and its association with total and cause specific mortality-A population-based cohort study and meta-analysis. Clin. Nutr 38, 2833-2845 (2019); Petsini F, Fragopoulou E & Antonopoulou S Fish consumption and cardiovascular disease related biomarkers: A review of clinical trials. Crit. Rev. Food Sci. Nutr 59, 2061-2071 (2019); Rimm E B et al. Seafood Long-Chain n-3 Polyunsaturated Fatty Acids and Cardiovascular Disease: A Science Advisory From the American Heart Association. Circulation 138, e35-e47 (2018); Kim K et al. Role of Total, Red, Processed, and White Meat Consumption in Stroke Incidence and Mortality: A Systematic Review and Meta-Analysis of Prospective Cohort Studies. J. Am. Heart Assoc 6, (2017); Website, N. H. S. Dairy and alternatives in your diet. nhs.uk. .nhs.uk/live-well/eat-well/milk-and-dairy-nutrition/)). A similar approach to the PDI scoring may be applied to the animal-based food groups, with either a positive (“healthy”) or reverse (“less-healthy”/“unhealthy”) quintile scoring. Adherence to the aMED diet may be calculated by following the method outlined by Fung et al. (Fung T T et al. Diet-quality scores and plasma concentrations of markers of inflammation and endothelial dysfunction—. Am. J. Clin. Nutr 82, 163-173 (2005)) and scoring ood categories from “least” to “most” Mediterranean. Weekly food intake frequencies may be first multiplied for assigned foods by the amount in grams per serving and then divided by 7 to determine grams per day. For all food categories as well as the fatty acid intake ratio, the median intake of each category may be calculated. A score of 0 (no aMED) or 1 (aMED) may be given for each category depending on whether the participant was above or below the median intake. For alcohol intake, a range may be used for score assignment: females: 5-25 g/d; males: 10-50 g/d were assigned a score of 1, while those above or below this range were assigned a score of 0. The aMED may then be generated by summation of each category score (Asnicar F, et al Nat Med. 2021 February; 27 (2): 321-332. doi: 10.1038/s41591-020-01183-8. Epub 2021 Jan. 11. PMID: 33432175; PMCID: PMC8353542).

A move toward a healthy state would be indicated by changes in one or more of the plurality of health metrics meeting thresholds for unhealthy levels towards healthy levels. For example, normal blood pressure is lower than 120/80. Moving toward a healthy state could be indicated by a decrease in blood pressure from an elevated state towards normal blood pressure. Normal fasting blood glucose levels are between 3.9 mmol/L and 5.6 mmol/L. A movement towards a healthy state may be determined by a decrease or increase in fasting blood glucose that moves an individual's fasting blood glucose towards normal fasting glucose levels. Healthy levels of total cholesterol are less than 200 mg/dl in adults. Healthy levels of low-density lipoproteins (LDL) are less than 100 mg/dL. Healthy levels of high-density lipoproteins (HDL) are over 40 mg/dL. Healthy levels for triglycerides are less than 150 mg/dL. A movement towards a healthy state would be a change in cholesterol levels to levels approaching healthy levels. Normal HbA1c levels are below 5.7%. A move towards a healthy state would be a change to levels approaching a normal Hb1Ac level. Such changes may or may not result in an individual achieving a healthy state. For example, movement towards a healthy state may be a change of 1%, 2%, 5%, 10%, 15%, 20%, 30%, 40%, 50%, 60%, 70%, 80%, or fraction thereof in one or more health metrics. For example, a decrease in elevated blood pressure by, for example, 10%, would be a move towards a healthy state. Similar criteria for other health metrics are generally known to those of ordinary skill in the art.

By way of example, foods to be demoted in a diet may include e.g., those that do not support a healthy condition, or that tend to support an unhealthy condition in the subject, or to move a subject more toward a less healthy state, that is, foods that move a subject further away from a normal or healthy level of one or more health metrics. When a subject/individual is advised (based on these correlations) which foods to consume and which to omit in their diet, as part of a food guidance or diet program, and the subject then adheres to this food guidance program over a period of time, the subject's/individual's gut health (and metabolic health) is improved, that is their microbiome more closely resembles the microbiome of a healthy individual as determined using methods known to one of ordinary skill in the art including via the measurement of one or more health metrics. Such a change in the microbiome may be determined by analyzing the microbiome of an individual at one or more points in time. In some aspects, the microbiome may be analyzed prior to, during, or after, making recommended changes. Recommended changes may be adhered to for a variety of periods of time. In some aspects, a change in the microbiome may be apparent after following recommended changes for one, two, three, four, five, six, seven, days, weeks, months, or years. A change in the microbiome may involve a change in one or more microbiome members including a change in the presence, absence, or relative abundance of a particular microbe. In some aspects, some or all microbes in a particular microbiome may be sensitive to diet. In other aspects, some or all microbes may be sensitive to other health changes. In some aspects, some or all microbes may be sensitive to both diet and other health changes.

Improvement in gut health may include an increase in the presence or abundance or relative abundance of at least one pro-health microbe in the subject's/individual's microbiome, or a decrease in the presence or abundance or relative abundance of at least one poor health microbe in the subject's/individual's microbiome, or some combination of both. In some aspects, improvement in gut health may be determined by an increase in absolute or relative numbers of one or more pro-health microbes. In some aspects, improvement in gut health may be determined by a decrease or absence in absolute or relative numbers of one or more poor-health microbes.

The microbiome service may also track the state of the microbiome of the user over time. For example, the microbiome service may compare data from an individual taken at multiple points in time. In this way, the user may see how changes made by the user (e.g., eating different foods, changing exercise patterns, consuming prebiotic substance(s), taking a probiotic preparation, and so forth) have affected the microbiome.

For example, presence, absence, and/or relative or absolute quantity of member(s) of a gut microbiome in a subject may be impacted by altering the diet (e.g., pattern and/or content of food and beverages consumed) of the subject. Broadly speaking, the subject's diet may be altered by providing the subject with a diet or food guidance program that is different from their current intake regimen, which program is then adhered to by the subject for a period of time. Such adherence may be one, two, three, four five, or six days, weeks, months, years, or a lifetime. In some aspects, a change in the microbiome may be observed within two days of a diet change. In other aspects, a change in the microbiome may be observed three months of diet change. In some aspects, a change in the microbiome may be observed after four months of diet change. In additional aspects, a change in the microbiome may be observed after a longer period of time. The comparison of microbiome data over a period of time may result in an improved personalized microbiome profile. For example, additional pro-health microbes may appear in a sample or fewer poor health microbes may appear in a sample. In other aspects, the overall score of an individual may improve, but the presence, absence, and/or relative absolute quantity of particular member(s) of a gut microbiome may move in a direction that would not be viewed as optimal. For example, the microbiome may have more good health microbes, or a larger percentage of good health microbes overall, but levels of a particular bad microbe may have increased even though overall levels of bad microbes has decreased.

Also provided is a reiterative method, which allows for continued improvement over time. In these reiterative embodiments, measurement of change in a microbiome fingerprint or score from one sample time point to the next is used to improve generalized or personalized food guidance to that individual. In examples of such methods, how many “good” and/or “bad” bugs have changed between the two time points (and other changes in the fingerprint like diversity or score, etc.) is measured, and this is correlated with food guidance provided to the individual over time. Such correlations can be improved, for instance, by tracking what the individual consumes—including as an aspect of tracking their adherence to the food guidance program more generally. Machine learning can be used to compare the data gathered from a single subject/individual with similar data gathered from a databank of other people (for instance, thousands of other people), also following particular food guidance and for whom measurements in changes in microbiome diversity and content are available at at least two time points. The comparison is then used to revise and improve the dietary guidance (such as personalized dietary guidance) that is given to the individual, and optionally to other individuals, so as to improve on the levels of decrease in bad bugs (and increase in good bugs). It is also contemplated that in some embodiments, the individual's updated/reiterated diet program is influenced by the current and prior record of their own results and changes, rather than relying on comparison to a database of results from other individuals.

In some aspects, the system may generate future or predicted microbiome scores based on the various recommended actions. For example, the system may generate a predicted microbiome score at a predetermined time period in the future (such as a number of day(s), a number of week(s), a number of month(s), or the like) provided that the individual performs or takes various recommended actions. For instance, the system may, for a specific action, such as an increase in exercise, determine a future microbiome score provided that the individual performed the specific action as directed for the predetermined period of time. In some cases, the future microbiome scores may be provided to the individual to encourage positive actions. In other cases, the future microbiome scores may be utilized to select the recommended actions and period of times associated therewith (e.g., the specific action(s) and period(s) of time may be selected based on a highest ranking future microbiome score).

Factors that may be considered when developing a (personalized) food guidance or food guidance program include, but are not limited to: predicted hunger of an individual, the individual's predicted responses to food consumption, glucose responses, fat responses, microbiome data, genetic data, sleep data, data about the individual's overall health, potential health risks for the individual, and/or other data associated with the individual to generate the food guidance.

According to some examples, the individual's glucose responses and/or the responses of other individuals to glucose, and other data, are used to generate the food guidance. In some configurations, the food guidance may include a hunger score that predicts a hunger level of an individual at a time (or for more than one time) after the individual has or is planning to consume food.

In the methods, systems, and uses described herein, the food guidance program that is conveyed to a single individual (also referred to herein as a subject) is prepared at least in part based on or in reference to a database of biomarkers and other information related to, for instance, general health, gut health, microbiome content (such as presence, absence, abundance, or relative abundance of specific microbes, and/or overall diversity, and other recognized measures of microbiome health), nutritional information, circadian rhythm, gathered from a plurality of individuals. Methods to prepare such an aggregated database of information are known, including methods developed by Zoe Limited. Additional guidance regarding representative methods to generate food guidance of a group or an individual (e.g., a personalized food guidance) may be found for instance in: WO 2019/155436 “Generating Predicted Values of Biomarkers For Scoring Food”; WO 2019/155437 “Generating Personalized Nutritional Recommendations Using Predicted Values Of Biomarkers”; WO 2019/224308 “Improving the Accuracy of Measuring Nutritional Responses in a Non-Clinical Setting”; WO 2020/043702 “Generating Personalized Food Recommendations from Different Food Sources”; WO 2020/043705 “Improving The Accuracy of Test Data Outside the Clinic”; WO 2020/043706 “Using at Home Measures to Predict Clinical State and Improving the Accuracy of At Home Measurements/Predictions Data Associated with Circadian Rhythm and Meal Timing”; WO 2021/038530 “Generalized Personalized Food Guidance Using Predicted Food Responses”; and US 2021-0065873 A1 “Generating Personalized Food Guidance Using Predicted Food Responses”.

It will be appreciated that the subject matter presented herein may be implemented as a computer process, a computer-controlled apparatus, a computing system, or an article of manufacture, such as a computer-readable storage medium. While the subject matter described herein is presented in the general context of program modules that execute on one or more computing devices, those skilled in the art will recognize that other implementations may be performed in combination with other types of program modules. Generally, program modules include routines, programs, components, data structures, and other types of structures that perform particular tasks or implement particular abstract data types.

Those skilled in the art will also appreciate that aspects of the subject matter described herein may be practiced on or in conjunction with other computer system configurations beyond those described herein, including multiprocessor systems, microprocessor-based or programmable consumer electronics, minicomputers, mainframe computers, handheld computers, personal digital assistants, e-readers, mobile telephone devices, tablet computing devices, special-purposed hardware devices, network appliances and the like.

In the following detailed description, references are made to the accompanying drawings that form a part hereof, and that show, by way of illustration, specific examples. The drawings herein are not drawn to scale. Like numerals represent like elements throughout the several figures (which may be referred to herein as a “FIG.” or “FIGs.”). Additional details regarding the various components and processes described above relating to generating microbiome fingerprints, dietary fingerprints, microbiome score, and microbiome ancestry are presented below.

Provided below is additional description in support of this technology, which is organized in the following sections: (I) Representative Methods for Generation, Collection, & Analysis of Microbiome Data; (II) Representative Computer Architecture; (III) Detection and Identification of Individual Microbes; (IV) Methods of Use; (V) Kits and Arrays; (VI) Systems; (VII) Exemplary Embodiments; (VIII) Example(s); and (IX) Closing Paragraphs.

### (I) REPRESENTATIVE METHODS FOR GENERATION, COLLECTION, & ANALYSIS OF MICROBIOME DATA

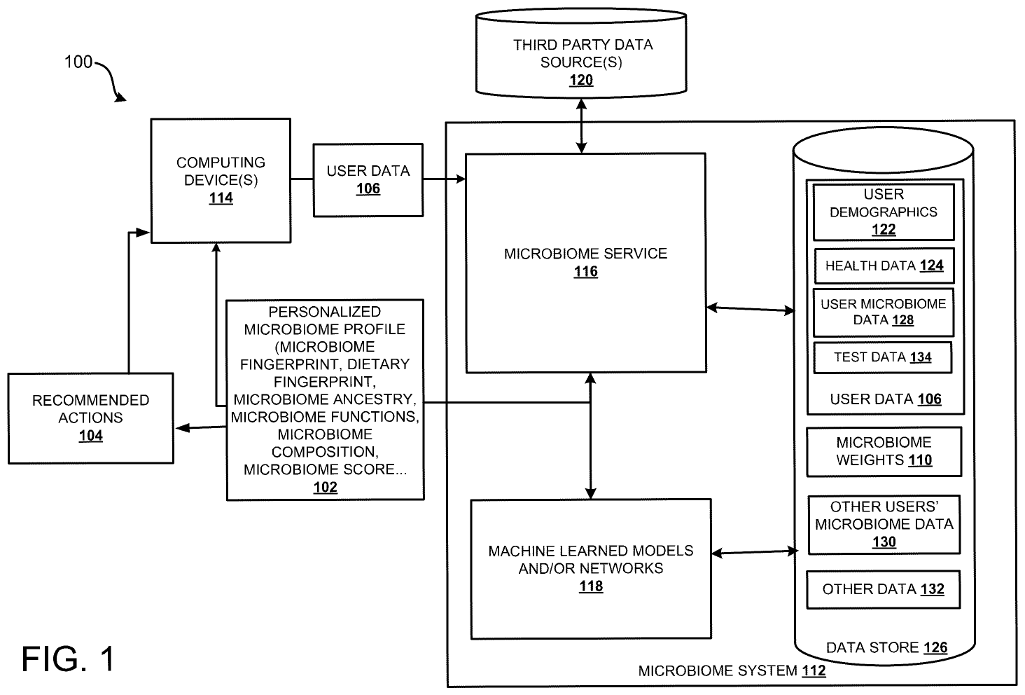

FIG. 1 is a block diagram depicting an illustrative operating environment 100 in which personalized microbiome data 102 including microbiome fingerprints, dietary fingerprints, microbiome ancestry, microbiome functions, microbiome composition, and microbiome scores and recommended actions are generated using user data 106, user demographics 122, health data 124, user microbiome data 128, other user's microbiome data 130, predetermined weights 110, and other data 132 located in the data store 126. For instance, an individual user, such as an individual requesting a personalized microbiome profile 102 or recommended actions 104, may communicate with the microbiome system 112 using a computing device 114. In some configurations, the individual may be a customer of the microbiome system 112 or a party related to the microbiome system 112.

As illustrated in FIG. 1, the microbiome system 112 includes one or more microbiome service 116 in communication with one or more machine learned models or networks 118 to generate a personalized microbiome profile 102 based on a received test data 122 representing presence and quantity of microbes in the individual's microbiome. In some examples, the microbiome system 112 may be associated with and/or implemented by resources provided by a service provider network such as provided by a cloud computing company. As one example, the microbiome system 112 includes a microbiome service 116, one or more machine learned models or networks 118, and a data store 126. The microbiome system 112 may include a collection of computing resources (e.g., computing devices such as servers). The computing resources may include a number of computing, networking, and storage devices in communication with one another. In some examples, the computing resources may correspond to physical computing devices and/or virtual computing devices implemented by one or more physical computing devices.

It should be appreciated that the microbiome system 112 may be implemented using fewer or more components than are illustrated in FIG. 1. For example, all or a portion of the components illustrated in the microbiome system 112 may be provided by a service provider network (not shown). In addition, the microbiome system 112 may include various Web services and/or peer to peer network configurations. Thus, the depiction of the microbiome system 112 in FIG. 1 should be taken as illustrative and not limiting to the present disclosure.

The microbiome service 116 may facilitate submission or receipt of test data 122, health data 124, third party data from one or more third party data sources 120 (such as medical data, microbe data—e.g., known benefits and harms, data related to how health, age, or other demographics affects individual microbes, and the like), microbiome data 110, and the like. For example, utilizing the computing device 114, an individual may submit user data 106, such as test data 134 representing presence and quantity of microbes, and/or health data 124 representing a status, medical conditions, or health of the individual as well as demographic data 122 related to the individual (e.g., age, sex, and the like) and historical user biome data 128. The microbiome service 116 may then analyze the user data 106 to determine a presence and percentage or relative abundance of individual microbes in the individual's microbiome. The microbiome service 116, either itself (e.g., via an algorithmic or heuristic technique) or in conjunction with the machine learned models and/or networks 118, may generate a personalized microbiome profile 102 for the individual. Such a personalized microbiome profile 102 may be one or more of a microbiome fingerprint, dietary fingerprint, microbiome ancestry, microbiome functions, microbiome composition, or microbiome score.

In some examples, the microbiome service 116 and/or the machine learned models and/or networks 118 may determine microbiome weights 110 representing a value for each good microbe and bad microbe. Such weights 110 may be based at least in part on user data 106, other users' microbiome data 130 and additional data 132 such as dietary data or other data received from the one or more third party data sources 120. In this manner, the microbiome weights 110 may be customized for each individual. In other cases, the microbiome weights 110 may be predetermined, such as via one or more machine learned models and networks trained on the microbiome data 110 or using other known techniques. In this example, the microbiome weights 110 may be more universal. For example, some weights may be based on all microbes found in the gut microbiome whereas other weights may be based on the microbes of a specific individual or fractions thereof.

The microbiome service 116 may also be used to generate or determine recommended actions 104 based on the personalized microbiome profile. For example, the microbiome service 116 may recommend dietary regimens (e.g., restrictions, additions, or other alterations), lifestyle regimens (e.g., exercise regimens), supplement regimens, and the like. These recommended actions 104 may be tailored for the individual and designed to encourage growth and reproduction of beneficial microbes (or pro-health microbes), reduce, or discourage or disrupt growth and reproduction of harmful microbes, introduce additional species such as additional pro-health microbes, and the like. In some cases, the microbiome service 116 may generate recommended actions 104 when the microbiome score of the personalized microbiome profile 102 fails to meet or exceed one or more thresholds. For example, if the microbiome score of the personalized microbiome profile 102 is between 0 and 100, the system 112 may utilize one or more thresholds, such as a first category of recommended actions 104 if the microbiome score of the personalized microbiome profile 102 is less than or equal to 50, a second category of recommended actions 104 if the microbiome score of the personalized microbiome profile 102 is less than or equal to 70, a third category of recommended actions 104 if the microbiome score 102 is less than or equal to 90, or the like.

In some cases, in addition to generating a current microbiome score of the personalized microbiome profile 102 based on the test data of an individual, the microbiome service 116 may generate future or predicted microbiome scores based on the individual adopting various recommended actions 104. For example, the microbiome service 116 may generate a predicted microbiome score or other aspect of a personalized microbiome profile 102 at a predetermined time period in the future (such as a number of day(s), a number of week(s), a number of month(s), or the like) provided that the individual performs or takes various recommended actions 104. For instance, the microbiome service 116 may, for a specific action, such as increase in exercise or an increase in the consumption of good fats, determine a future microbiome score in a personalized microbiome profile 102 provided that the individual performed the specific action as directed for the predetermined period of time. In some cases, the future microbiome scores or other parts of the personalized mirobiome profile 102 may be provided to the individual to encourage positive actions. In other cases, the future microbiome scores may be utilized to select the recommended actions 104 and period of times associated therewith (e.g., the specific action(s) and period(s) of time may be selected based on a highest ranking future microbiome score).

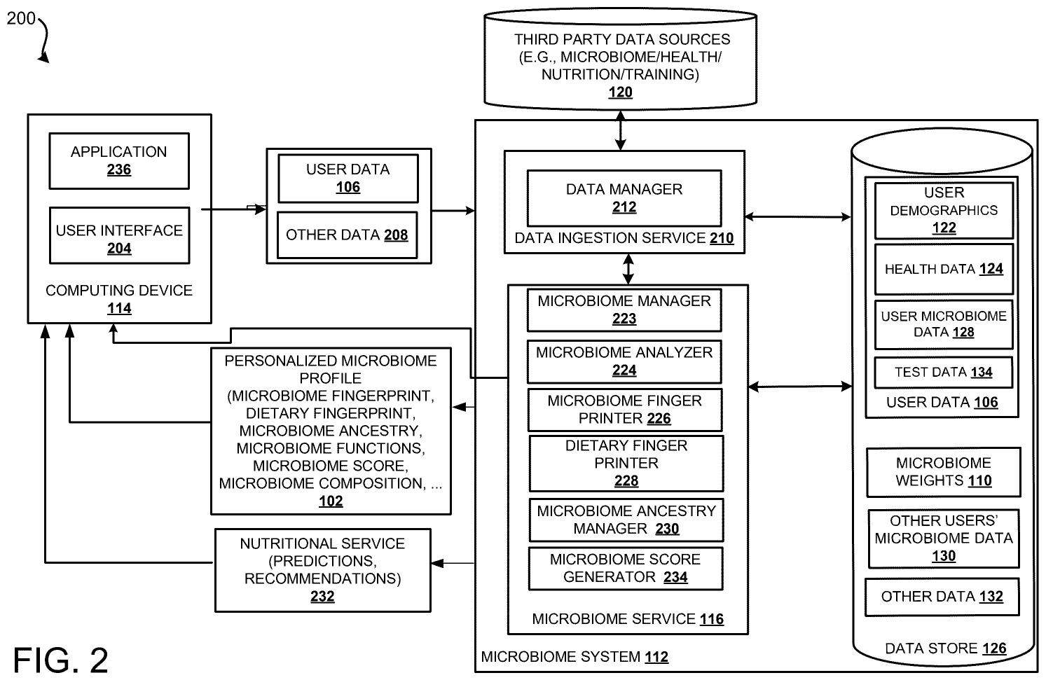

FIG. 2 is a block diagram depicting an illustrative operating environment 200 in which microbiome data is analyzed to generate a personalized microbiome profile 102 including one or more of microbiome fingerprints, dietary fingerprints, microbiome ancestry, and microbiome scores for users. An individual, such as an individual interested in obtaining microbiome fingerprints, dietary fingerprints, microbiome ancestry and microbiome score information, may communicate with the microbiome system 112 using a computing device 114 and possibly other computing devices, such as mobile electronic devices.

In some configurations, an individual may generate and provide data including user data 106 and other data 208. Such data may include microbiome data, test data, demographic data, and/or other data. According to some examples, the user may utilize a variety of at home biological collection devices, which collect a biological sample. These devices may include but are not limited to “At Home Blood Tests” which use blood extraction devices such as finger pricks which in some examples are used with dried blood spot cards, button operated blood collection devices using small needles and vacuum to collect liquid capillary blood and the like. In some examples there may be home biological collection devices such as a stool test which is then assayed to produce biomarker test data such as gut microbiome data. As exemplified herein, the subject from which the biological sample is obtained may be a human subject. Other animal subjects are also contemplated, including non-human primates, companion animals, domestic animals, livestock, endangered and threatened animals, laboratory animals, and so forth. The results of such tests and/or the samples themselves may form all or part of the user data 106.

A computing device, such as a mobile phone or a tablet computing device can also be used to provide additional data. For instance, instead of relying on an individual to accurately record the time a test was taken or a sample was obtained, the computing device 114 can record information that is associated with the event. The computing device 114 may also be utilized to capture the timing data associated with the test (e.g., the time the test was performed, . . . ), or the sample was collected, and provide that data to a data ingestion service 210. As an example, a clock (or some other timing device) of the computing device 114 may be used to record the time the measurement(s) were collected and/or samples were obtained.

As illustrated in FIG. 2, the operating environment 200 includes one or more computing devices 114, in communication with a microbiome system 112. In some examples, the microbiome system 112 may be associated with and/or implemented by resources provided by a service provider network such as provided by a cloud computing company. The microbiome system 112 includes a data ingestion service 210, a microbiome service 116, a nutritional service 232, and a data store 126. The nutritional service 232 can be utilized to generate personalized nutritional recommendations. For example, the personalized nutritional recommendations can be generated using techniques described in U.S. Patent Publication No. US 2019-0252058 A1, published Aug. 15, 2019. According to some examples, the nutritional service 232 may provide recommendations based on the microbiome fingerprint, dietary fingerprint, microbiome ancestry data, microbiome score, and/or other data.

The microbiome system 112 may include a collection of computing resources (e.g., computing devices such as servers). The computing resources may include a number of computing, networking, and storage devices in communication with one another. In some examples, the computing resources may correspond to physical computing devices and/or virtual computing devices implemented by one or more physical computing devices.

It should be appreciated that the microbiome system 112 may be implemented using fewer or more components than are illustrated in FIG. 2 or the combination of FIG. 2 and FIG. 1. For example, all or a portion of the components illustrated in the microbiome system 112 may be provided by a service provider network (not shown). In addition, the microbiome system 112 could include various Web services and/or peer to peer network configurations. Thus, the depiction of the microbiome system 112 in FIG. 2 should be taken as illustrative and not limiting to the present disclosure.

The data ingestion service 210 facilitates submission of data utilized by the microbiome manager 223 and, in some configurations, the nutritional service 232. Accordingly, utilizing a computing device 114, an electronic collection device, an at home biological collection device or via in clinic biological collection, an individual may submit data 208 to the microbiome system 112 via the data ingestion service 210. Some of the data 106 may be sample data, biomarker test data, and some of the data 208 may be non-biomarker test data such as photos, barcode scans, timing data, and the like.

A “biomarker” or biological marker generally refers to one or more measurable indicators (that may be combined using various techniques) of some biological state or condition associated with an individual. Stated another way, a biomarker may be anything that can be used as an indicator of a particular disease, condition, health, state, or some other physiological state of an organism. A biomarker typically can be measured accurately (either objectively and/or subjectively) and the measurement is reproducible. By way of example, the following are considered biomarkers: blood glucose, triglycerides (TG), insulin, c-peptides, ketone body ratios, cholesterol, IL-6 inflammation markers, the expression of any specified gene or protein, hunger, fullness, body mass index (BMI), composition of a microbiome (including not only what strains are present, but the relative abundance of two or more strains in a microbiome), and the like. In practice, a good biomarker is often a combination of two or more measurable indicators combined in a simple or complex way; in some cases, the combination of more than one measurable indicator makes the biomarker more closely linked to the disease, condition, health, state, or some other physiological state of an organism.

The measured biomarkers may include many different types of health data such as microbiome data which may be referred to herein as “microbiome data”, blood data, glucose data, lipid data, nutrition data, wearable data, genetic data, biometric data, questionnaire data, psychological data (e.g., hunger, sleep quality, mood, . . . ), objective health data (e.g., age, sex, height, weight, diet, medical history, BMI, waist circumference, glucose levels, blood pressure, cholesterol data, . . . ), personal data (e.g. activity level, location, self-reported surveys), as well as other types of data. Generally, “health data” refers to any psychological, subjective, and/or objective data that relates to and is associated with one or more individuals. The health data might be obtained through testing, self-reporting, and the like. Some biomarkers change in response to eating food, such as blood glucose, insulin, c-peptides, and triglycerides and their lipoprotein components.

To understand the differences in nutritional responses for different users, dynamic changes in biomarkers caused by eating food such as a standardized meal (“postprandial responses”) can be measured. By understanding an individual's nutritional responses, in terms of blood biomarkers such as glucose, insulin, and triglyceride levels, or non-blood biomarkers such as the microbiome, a nutritional service 232 may be able to choose or recommend food(s) and/or eating pattern that is/are more suited for that particular person. For example, the microbiomes of some individuals may improve if they have small frequent meals. The microbiomes of other individuals may improve if subjected to periods of fasting.

Data may also be obtained by the data ingestion service 210 from other data sources, such as data source(s) 120. For example, the data source(s) 120 can include, but are not limited to, microbiome data associated with one or more users, nutritional data (e.g., nutrition of particular foods, nutrition associated with the individual, and the like), health data records associated with the individual and/or other individuals, and the like.

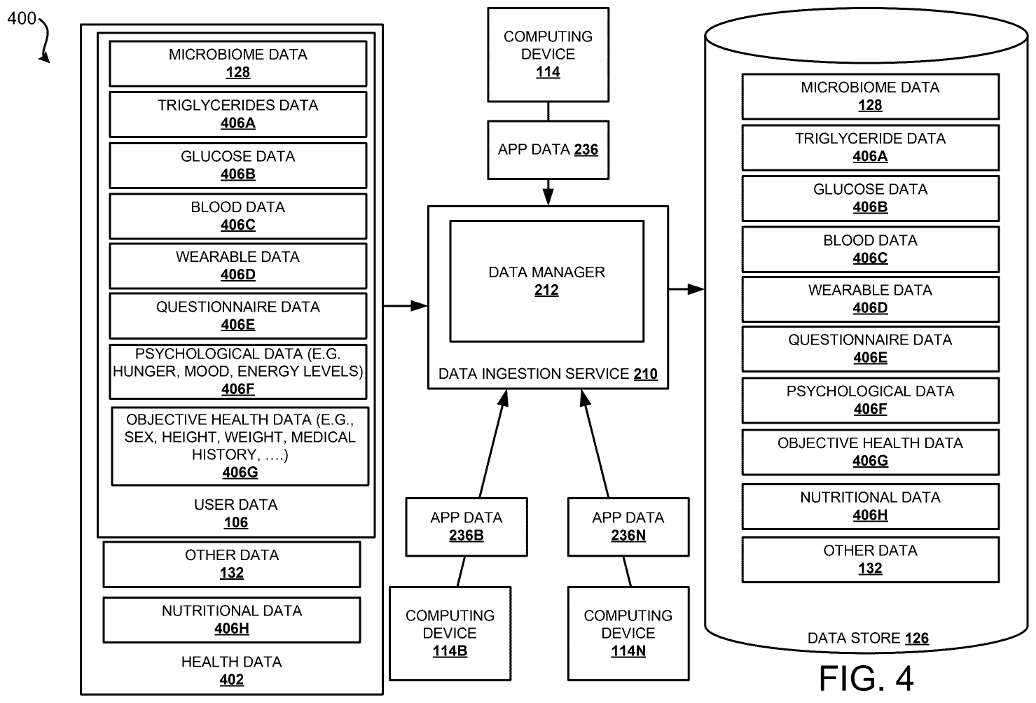

The data, such as data 106 or 208, or data obtained from one or more data sources 120, may then be processed by the data manager 212 and/or the microbiome manager 223 and included in a memory, such as the data store 126. As illustrated, the data store 126 can be configured to store user microbiome data 128, other users' microbiome data 130, and other data 132 (see FIG. 4 for more details on the data ingestion service 210).

As discussed in more detail below, the microbiome service 116 utilizing the microbiome manager 223, the microbiome analyzer 224, the microbiome finger printer 226, the microbiome dietary finger printer 228, the microbiome ancestry manager 230, and the microbiome score generator 234 analyzes the data 106 and the data 208 and generates a microbiome fingerprint, a dietary fingerprint, microbiome score, and microbiome ancestry data for the user. According to some configurations, the microbiome service 116 utilizes both data 106 and data 208 associated with the user and data from other users.

In some examples, the data manager 212 may utilize one or more machine learning mechanisms. For example, the data manager 212 can use a classifier to classify the microbiome within a classification category (e.g., associate with a particular dietary index, a geographic location, genetic makeup . . . ). In other examples, the data manager 212 may use a scorer to generate scores that may provide an indication of the dietary index associated with a user, how closely related the user is to other users based on the microbiome data, and the like.

The data ingestion service 210 and/or the microbiome service 116 can generate one or more user interfaces, such as a user interface 204, through which an individual, utilizing the computing device 114, or some other computing device, may provide/receive data from the microbiome system 112. For example, the data ingestion service 210 may provide a user interface 204 that allows an individual of the computing device 114 to submit data 106 and data 208 to the microbiome system 112.

In some cases, the individual can also provide biological samples to a lab for testing, for instance using a biological collection device. According to some configurations, this will include At Home Blood Tests. According to some configurations, individuals can provide a sample (such as a stool sample) for microbiome analysis. As an example, metagenomic testing can be performed using the sample to allow the DNA of the microbes in the microbiome of an individual to be digitalized. Generally, a microbiome analysis includes determining the composition and functional potential (here called just “function”) of a community of microbes in a particular location, such as within the gut of an individual. An individual's microbiome appears to have a strong relationship to metabolism, weight, and health, yet only ten to thirty percent of the bacterial species in a microbiome is estimated to be common across different individuals. Embodiments described herein combine different techniques to assist in improving the accuracy of the data captured outside of a clinical setting, such as calculating accurate glucose responses to individual meals, which can then be linked to measures like the microbiome.

According to some configurations, individuals can provide a sample or samples of their stool for microbiome analysis as part of the at home biological collection. In some cases, this sample may be collected without using a chemical buffer. The sample can then be used to culture live microbes, or for chemical analysis such as for metabolites or for genetic related analysis such as metagenomic or metatranscriptomic sequencing. In such cases, the sample may suffer from changes in microbial composition due to causes including microbial blooming from oxygen in the period between being collected and when it is received in the lab, where it usually will be immediately assayed or frozen. In some cases, to avoid this change in bacterial composition after collection, the sample obtained at home may be frozen at low temperatures very rapidly after collection. The sample can then be used to culture live bacteria, or for chemical analysis or for metagenomic sequencing. This collection can be done as part of an in clinic biological collection or at home where the collection kit is configured to deliver such low temperatures and maintain them until a courier has taken the sample to a lab.

A stool sample may be combined with a chemical preservation buffer, such as ethanol, as part of the at home collection process to stop further microbial activity, which allows a sample to be kept at room temperature before being received at the lab where the assay is done. In some examples, the buffer may be a proprietary chemical product sold and validated by another company for the task of freezing microbial activity while still allowing the sample to be processed for metagenomics sequencing. A buffer allows for such a sample to be posted in the mail without (or minimizing) issues of microbial blooming or other continuing changes in microbial composition. The buffer may however prevent some biochemical analyses from being done, and because preservation buffers are likely to kill a large fraction of the microbial population, it is unlikely that samples conserved in preservation buffers can be used for cultivation assays.

In some cases, a user may do multiple stool tests over time, so that changes in the microbiome over time can be measured, or changes in the microbiome in response to meals, or changes in the microbiome in response to other clinical or lifestyle variations may be identified.

In some examples, the stool sample is collected by the user using a stool collection kit that prevents the stool from contamination, such as for instance the contamination that would occur from stool falling into a toilet. Because there is a very high microbial load in the gut microbiome compared, for example, to the skin microbiome, it is also possible that in some cases the stool sample is taken from paper that is used to clean the user's behind after they have passed a stool. This is only possible if the quantity of stool is large enough that the microbes from the stool greatly exceed the microbes that will be picked up from the user's skin or environmental contaminants. In any of these cases the scoop, swab, or tissue may be placed inside a collection device, such as a vial that contains a buffer solution. If the user ensures the stool comes into contact with the buffer, for example by shaking, then further microbial activity is stopped and the solution can be kept at room temperature without a significant change in microbial composition.

In some cases, a sterile synthetic tissue is used that does not have biological origins such as paper, so that when the DNA of the sample is extracted there is no contamination from DNA originating in the tissue.

According to some examples, the tissue is impregnated with a liquid to help capture more stool from the user's skin, where the liquid does not interfere with the results of the stool test and is not potentially dangerous for the human body.

In some cases, the timing and quality of the stool sample can be recorded using the computing device 114, for example using a camera. Where there are multiple stool tests, the computing device 114 can use a barcode (or some other identifier) to confirm the timing and identity of that particular sample. Other data can also be collected. For example, data about how the sample was stored, how long the sample was stored before being supplied to the lab for analysis, and the like.

While the data ingestion service 210, the microbiome service 116, the microbiome system 112 are illustrated separately, all or a portion of these services may be located in other locations or together with other components. For example, the data ingestion service 210 may be located within the microbiome service 116. Similarly, the microbiome manager 223 may be part of a different service, and the like.

According to some examples, some individuals may be asked to visit a clinic to combine at home data with data collected at a clinic. The purpose of the clinic visit is to allow much higher accuracy of measurement for a subset of the individual's data, which can then be combined with the lower quality at home data. This may be used by the microbiome service 116 to improve the quality of the at home data.

According to some examples, the day before the visit to the clinic, the individuals are asked to avoid taking part in any strenuous exercise and to limit the intake of alcohol. In some configurations, the microbiome service 116 can analyze the data 106, such as data obtained from an activity tracker, to determine whether the individual followed the instructions of avoiding strenuous exercise. Similarly, the microbiome system 112, or some other device or component, may analyze the foods eaten by the individual by analyzing food data that indicates the foods eaten by the user. Individuals may be provided with instructions for the tests (e.g., avoid eating high fat or high fiber meals that may interfere with test results, fasting, drinking water, . . . ).

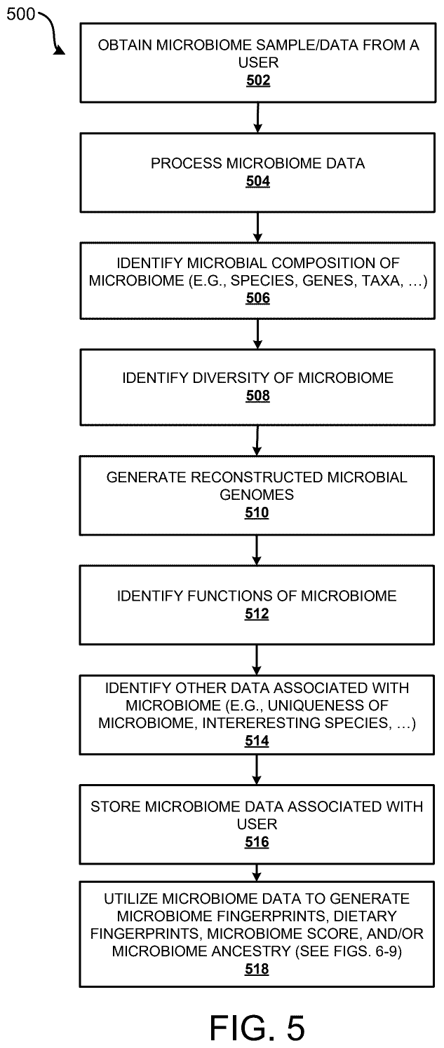

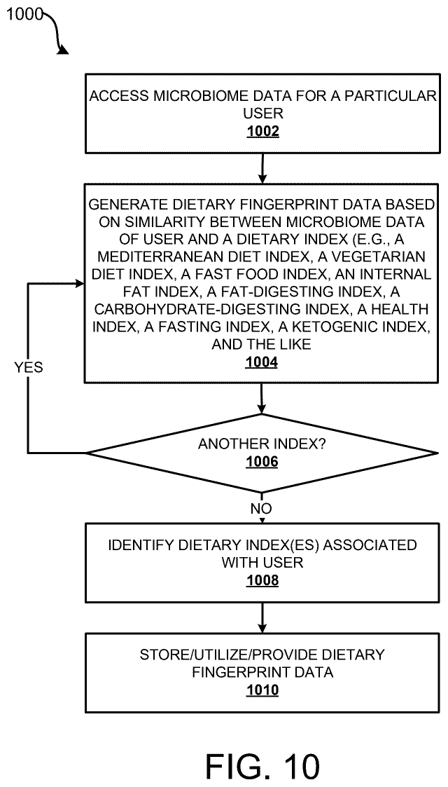

As described in more detail below with regard to FIGS. 5 and 10, the microbiome service 116 may use the microbiome manager 223 to generate a microbiome fingerprint, and a dietary fingerprint for a user. As discussed above, a “microbiome fingerprint” is data that uniquely identifies the microbiome of a user at a particular point in time. According to some configurations, the microbiome finger printer 226 generates a microbiome fingerprint from a user based on different profiles generated from the microbiome data, such as but not limited to quantitative taxonomic profiles, quantitative functional potential profiles, and strain-level genomic profiles. In some examples, the profiles are generated by the microbiome finger printer 226 and/or the microbiome analyzer 224.

According to some configurations, the microbiome fingerprint is a combination of descriptors, including, but not limited to (1) the quantitative (i.e. relative abundance) taxonomic profiles (i.e., the names or more generally identifiers (IDs) in case of unknown entities of microbial species or other taxonomic units), (2) the quantitative (i.e. relative abundance) functional potential profiles, (i.e., the names or generally identifiers (IDs) in case of unknown entities of microbial gene families, microbial pathways, and microbial functional modules), and (3) the strain-level genomic profiles (i.e., the reconstruction of the genomes or part of the genomes of as many microbes present in the microbiome as possible).

The microbiome fingerprint may be generated by the microbiome finger printer 226 using various techniques and methods. In some configurations, generation of the microbiome fingerprint includes obtaining the microbiome sample, generating DNA from the sample, preprocessing the raw sequencing data to the generate quality-screened sequencing data, and transforming the sequencing data is transformed into the numerical and genomics sets for the descriptors utilized to generate the microbiome fingerprint (e.g., quantitative taxonomic profiles, quantitative functional potential profiles, and strain-level genomic profiles).

The microbiome analyzer 224 may also be configured to perform processing associated with the microbiome data. For example, the microbiome analyzer 224 may be configured to generate and/or process sequencing data associated with the microbiome of the user. See FIG. 5 for more details on generating the profiles. After generating the profiles, the microbiome finger printer 226 may generate the microbiome fingerprint for the user. In some examples, the dietary finger printer 228 combines the data associated with the different profiles generated.

The dietary finger printer 228 is configured to generate a dietary fingerprint for the user. As discussed above, the “dietary fingerprint” of a user indicates how the microbiome of a user is associated with one or more different indexes that may be associated with a particular diet and/or a health characteristic. The indexes may include, but are not limited to a Mediterranean diet index, a vegetarian diet index, a fast food index, an internal fat index, a fat-digesting index, a carbohydrate-digesting index, a health index, a fasting index, a ketogenic index, and the like.

According to some configurations, the dietary finger printer 228 generates a score for each of the different indexes, such as from 0-100 (or some other indicator), to indicate how closely the microbiome of the user is associated with a particular index. For example, the dietary finger printer 228 may generate a score for each of the indexes based on how closely the microbiome of the user resembles a typical microbiome of someone that is known to follow a specific diet. For example, a score of 100 may indicate that the diet is strongly correlated to a particular diet, a score of 0 would indicate no correlation, and a score between 0 and 100 would indicate a different correlation. According to some configurations, the dietary finger printer 228 generates a Mediterranean diet index score, a vegetarian diet index score, a fast food index score, an internal fat index score, a fat-digesting index score, a carbohydrate-digesting index score, a health index score, fasting index score, ketogenic index score, and the like.

The Mediterranean diet index score for a user indicates how closely the microbiome of the user resembles the typical microbiome of someone on a Mediterranean diet. The vegetarian diet index score indicates how closely the microbiome of the user resembles someone on a vegetarian diet. The fast food index score indicates how closely the microbiome of the user resembles someone on a fast food diet. The internal fat index score indicates how closely the microbiome of the user resembles someone with high or low visceral fat. The fat-digesting index score indicates how closely the microbiome of the user resembles someone with low postprandial triacylglycerol (TAG) rises. The carbohydrate-digesting index score indicates how closely the microbiome of the user resembles someone with low postprandial glucose rises. The health index score indicates how closely the microbiome of the user resembles someone that is healthy. The fasting index score indicates how closely the microbiome of the user resembles someone that fasts regularly. The ketogenic index score indicates how closely the microbiome of the user resembles someone who is ketogenic.

In other configurations, the dietary finger printer 228, or some other service or component may utilize different mechanisms to determine whether the microbiome of the user resembles a particular diet and/or group. For instance, the dietary finger printer 228 may utilize a machine learning mechanism to classify the microbiome of the user within a classification and/or generate a score, or some other indicator that indicates how closely the microbiome data of the user matches the microbiome data of a representative user associated with the particular index.

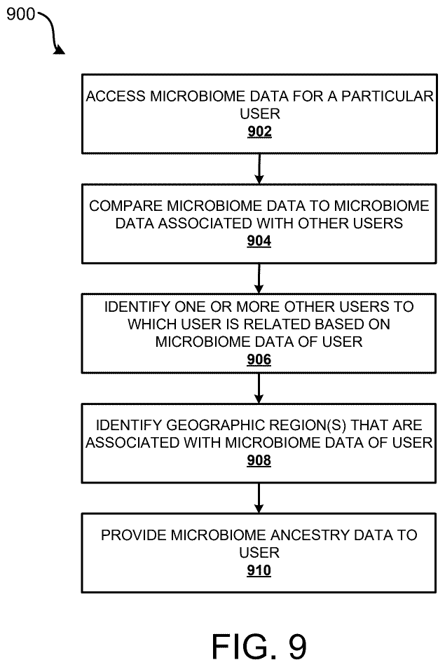

The microbiome ancestry manager 230 is configured to generate microbiome ancestry data for a user, for example, using the process described in further detail with reference to FIG. 9. A “microbiome ancestry” refers to microbiome data that indicates that the user has relationships with other users and/or locations. In some examples, the microbiome service analyzes the microbiome data of the user and determines how closely the microbiome of the user is related to other users, and/or locations. For instance, the microbiome service may determine a number of other users to which the microbiome of the user is most closely related. In some configurations, the microbiome ancestry manager 230 compares the microbiome data of the user to microbiome data of other users to identify a relationship. Similar to generating the scores for the different indexes performed by the dietary finger printer 228, the microbiome ancestry manager 230 may generate a score for each comparison between the user and the other users. The scores that indicate a close relationship (e.g., above a specified value) with the user may be identified as related.

The microbiome score generator 234 may determine scores for an individual using a machine learning model or neural network algorithm trained using third-party health data and microbiome data to generate weights or factors for each individual species of microbes that could potentially be present in an individual's personal microbiome. In some cases, the weights may be positive, negative, or neutral depending on determined beneficial and/or harmful tendencies. These weights may then be used to generate microbiome scores for individuals based on the presence, absence, and/or comparable quantities of individual species of microbes.

Each microbe may be given the same or different weights as part of generating one or more aspects of a personalized microbiome profile. For example, all microbes that are viewed/designated as good microbes may be given a positive weight, microbes that are viewed/designated as neutral may be given a weight of zero, and microbes that are viewed/designated as bad microbes may be given a negative weight. In other aspects, the weights may be assigned based on the likelihood that the presence or relative abundance of a particular microbe will change based on the individual parameter that will be changed. For example, some microbes may be sensitive to changes in diet may be given a weight that reflects the likelihood of change whereas some microbes may be impervious to changes in diet and are therefore omitted or given a weight that reflects their intransigence. In other aspects, some microbes may change based on exercise or other lifestyle changes and may be weighted accordingly for a second calculation. In other aspects, the weighting of a microbe may reflect a plurality of factors including the prevalence of the microbe in certain segments of the population. For example, microbes that are more likely to be found in populations that are viewed as healthy may be given one weight and microbes that are more likely to be found in populations that are unhealthy may be given a different weight. Some or all of the microbes may be weighted and some or all of the microbes in a particular sample may be used to generate all or part of a personal microbiome profile.

The microbiome service 116 may also identify one or more locations to which the microbiome of the user is associated. For example, the microbiome service may identify the countries the microbiome of the user is associated with. For example, the user's microbiome is 75% similar to microbiomes found in Country 1, and 25% similar to microbiomes found in Country 2. In other aspects, the user's microbiome could be characterized by similarity to location, for example, the user's microbiome is seen in 75% of individuals in North America and 25% of individuals in France. This identification may be based on microbiome data of users at different locations and/or different populations (e.g., English, American, French, Mexican, Italian, . . . ). See FIG. 9 for additional details for generating the microbiome ancestry data.

The microbiome analyzer 224, or some other device or component, may analyze the microbiome data of a user before/after generating the microbiome fingerprint, dietary fingerprint, microbiome score, and/or microbiome ancestry for a user. For example, the microbiome analyzer 224 may perform an analysis of the microbiome data to identify the microbial composition of the microbiome (e.g., the species, genes, taxa, and the like). The microbiome service 116 may also generate reconstructed microbial genomes, determine a diversity of the microbiome, identify functions of the microbiome, identify a uniqueness of the microbiome, identify interesting species, and the like.

In some examples, the microbiome data of the user is compared (e.g., by the microbiome service 116) with other data that is gathered about the user, as well as other users. For instance, users may provide responses to questionnaires, data about food that is eaten, sleep habits, mental health, and the like. Among other uses, this data may be utilized to determine a “microbiome ancestry” of a user.

In some examples, the microbiome service may provide a user interface (UI), such as a graphical user interface (GUI) 204 for a user to view and interact with data associated with the microbiome fingerprints, dietary fingerprints, and microbiome ancestry. For instance, the GUI may display microbiome fingerprint data that shows various characteristics of the microbiome fingerprint, dietary fingerprint data that shows various characteristics of the dietary fingerprint, microbiome ancestry data that shows various characteristics of the microbiome ancestry, recommendation data that identifies one or more recommendations relating to changing the microbiome of the user, and the like. In some configurations, the user may utilize an application 236 on the computing device 114 to interact with the nutritional environment. In some configurations, the application 236 may include functionality relating to processing at least a portion of the data 106 and the data 208.

As an example, the microbiome service 116 may provide recommendations generated by the microbiome system 112 to increase the diversity of foods eaten as there is no one good food for a microbiome. The recommendations may include to eat different gut-healthy foods, eat fermented foods, minimize highly processed foods (things like emulsifiers and artificial sweeteners may affect the microbiome). The microbiome service may base the recommendations on data obtained from the user, and other users.

The microbiome service 116 may also track the state of the microbiome of the user over time. For example, the microbiome service may provide data related to different microbiome analysis. In this way, the user may see how changes made by the user (e.g., eating different foods, changing exercise patterns, . . . ) have affected the microbiome.

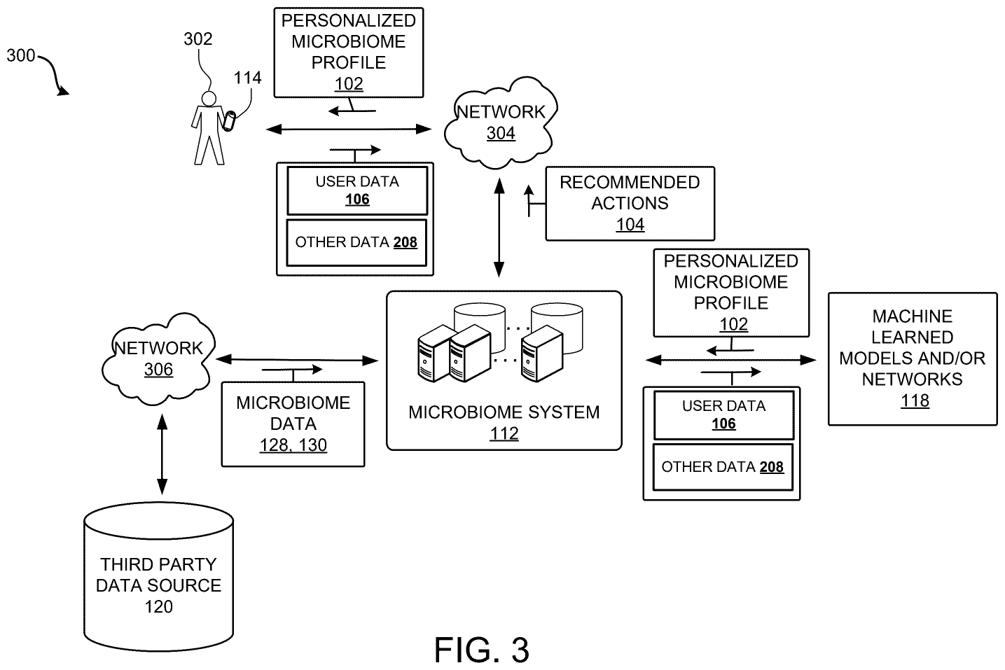

FIG. 3 is another block diagram depicting an illustrative operating environment 300 in which personalized microbiome profile 102 (e.g., microbiome fingerprint, dietary fingerprint, microbiome ancestry, and microbiome score) and recommended actions 104 (or nutritional service 232) are generated using user data 106 and other data 208. For instance, an individual user 302, such as an individual requesting one or more aspects of a personalized microbiome profile 102 or recommended actions 104, may communicate with a cloud-based microbiome system 112 using a computing device 114 via one or more networks 304. Similarly, the cloud-based microbiome system 112 may receive the microbiome data 128 or 130 via one or more networks 306. In the current example, the networks 304 and 306 are illustrated as separate networks but it should be understood that some or all of the networks 304 and 306 may be the same.

In some cases, the microbiome system 112 may determine or maintain microbiome scores for individual microbes that may be included in a human's microbiome. For example, the microbiome system 112 may process test data (e.g., microbiome data and/or health data) from a plurality of participants (hundreds to thousands of test individuals) to select and weight various microbes. In some cases, the weighting of individual microbes may randomly subsample microbe DNA sequences obtained from the plurality participants. In some cases, the microbe DNA sequences may include original tests and retests of the plurality of participants. The microbiome system 112 may then filter any participants' data that did not meet or exceed a threshold of clean reads. The microbiome system 112 may then assign weights (either positive or negative) to any microbes based at least in part on correlations with a selection of known markers of nutritional and cardiometabolic health. In some cases, the microbe DNA sequences test data and the markers of nutritional and cardiometabolic health may be used to train one or more machine learned models and/or networks that output weights for individual microbes and/or the resulting aspect of the personalized microbiome profile 102 for an individual user 302 of the system 112.

In the current example, the microbiome system 112 may receive the user data 106 and other data 208 from the computing device 114 as shown. The microbiome system 112 may then determine a presence and/or relative quantity of microbes within the users 302 microbiome.

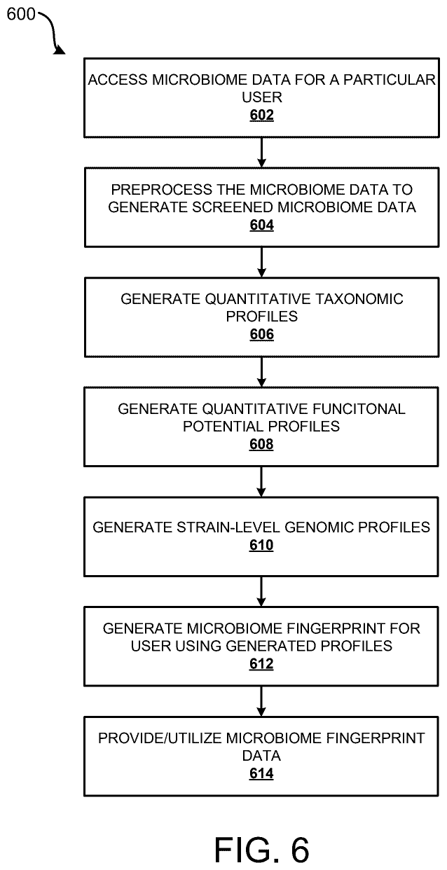

The microbiome system 112 may generate one or more aspects of a personalized microbiome profile 102 including a microbiome fingerprint, a dietary fingerprint, microbiome ancestry, microbiome function, microbiome score or microbiome composition. As described in more detail below with regard to FIGS. 6 and 10, the microbiome system 112 generates a microbiome fingerprint, and a dietary fingerprint for a user. As discussed above, a “microbiome fingerprint” is data that uniquely identifies the microbiome of a user at a particular point in time. According to some configurations, the microbiome fingerprint is based on different profiles generated from the microbiome data, such as but not limited to quantitative taxonomic profiles, quantitative functional potential profiles, and strain-level genomic profiles.

According to some configurations, the microbiome fingerprint is a combination of descriptors, including, but not limited to (1) the quantitative (i.e. relative abundance) taxonomic profiles (i.e., the names or more generally identifiers (IDs) in case of unknown entities of microbial species or other taxonomic units), (2) the quantitative (i.e. relative abundance) functional potential profiles, (i.e., the names or generally identifiers (IDs) in case of unknown entities of microbial gene families, microbial pathways, and microbial functional modules), and (3) the strain-level genomic profiles (i.e., the reconstruction of the genomes or part of the genomes of as many microbes present in the microbiome as possible).

As discussed above, the “dietary fingerprint” of a user indicates how the microbiome of a user is associated with one or more different indexes that may be associated with a particular diet and/or a health characteristic. The indexes may include, but are not limited to a Mediterranean diet index, a vegetarian diet index, a fast food index, an internal fat index, a fat-digesting index, a carbohydrate-digesting index, a health index, a fasting index, a ketogenic index, and the like.

According to some configurations, the dietary print is a Mediterranean diet index score, a vegetarian diet index score, a fast food index score, an internal fat index score, a fat-digesting index score, a carbohydrate-digesting index score, a health index score, fasting index score, ketogenic index score, and the like.

A “microbiome ancestry” refers to microbiome data that indicates that the user has relationships with other users and/or locations. In some examples, the microbiome service analyzes the microbiome data of the user and determines how closely the microbiome of the user is related to other users, and/or locations. For instance, the microbiome service may determine a number of other users to which the microbiome of the user is most closely related.

The “microbiome score” uses a machine learning model or neural network algorithm trained using third-party health data and microbiome data to generate weights or factors for each individual species of microbes that could potentially be present in an individual's personal microbiome. In some cases, the weights may be positive, negative, or neutral depending on determined beneficial and/or harmful tendencies. These weights may then be used to generate microbiome scores for individuals based on the presence, absence, and/or comparable quantities of individual species of microbes.

Each microbe may be given the same or different weights. For example, all microbes that are viewed as good microbes may be given a positive weight, microbes that are viewed as neutral may be given a weight of zero, and microbes that are viewed as bad microbes may be given a negative weight. In other aspects, the weights may be assigned based on the likelihood that the presence or relative abundance of a particular microbe will change based on the individual parameter that will be changed. For example, some microbes may change based on diet and may be given a weight that reflects the likelihood of change whereas some microbes may be impervious to changes in diet and are therefore omitted or given a weight that reflects their intransigence. In other aspects, some microbes may change based on exercise or other lifestyle changes and may be weighted accordingly for a second calculation. In other aspects, the weighting of a microbe may reflect a plurality of factors.

In some examples, the microbiome system 112 may also be used to generate or determine recommended actions 104 based on the personalized microbiome profile 102. For example, the microbiome system 112 may recommend dietary regimens (e.g., restrictions, additions, or other alterations), lifestyle regimens (e.g., exercise regimens), supplement regimens, and the like. These recommended actions 104 may be tailored for the individual user 302 and designed to encourage growth and reproduction of beneficial microbes, reduce, or discourage or disrupt growth and reproduction of harmful microbes, introduce additional species, and the like.

In some cases, the microbiome system 112 may generate recommended actions 104 when one or more aspects of the personalized microbiome profile 102 fails to meet or exceed one or more threshold(s). For example, if the microbiome score of the personalized microbiome profile 102 is between 0 and 100, the system 112 may utilize one or more thresholds, such as a first category of recommended actions 104 if the microbiome score the personalized microbiome profile 102 is less than or equal to 50, a second category of recommended actions 104 if the microbiome score of the personalized microbiome profile 102 is less than or equal to 70, a third category of recommended actions 104 if the microbiome score of the personalized microbiome profile 102 is less than or equal to 90, or the like.

FIG. 4 is a block diagram depicting an illustrative operating environment 400 in which a data ingestion service 210 receives and processes data associated with data associated with at home tests and sample collections. As illustrated in FIG. 4, the operating environment 400 includes the data ingestion service 210 that may be utilized in ingesting data utilized by the microbiome service 116.

In some configurations, the data manager 212 is configured to receive data such as, health data 402 that can include, but is not limited to microbiome data 128, triglycerides data 406A, glucose data 406B, blood data 406C, wearable data 406D, questionnaire data 406E, psychological data (e.g., hunger, sleep quality, mood, . . . ) 406F, objective health data (e.g., height, weight, medical history, . . . ) 406G, nutritional data 240B, and other data 132.

According to some examples, the microbiome data 128 includes data about the gut microbiome of an individual. The gut microbiome can host a large number of microbial species (e.g., >1000) that together have millions of genes. Microbial species include bacteria, fungi, parasites, viruses, and archaea. Imbalance of the normal gut microbiome has been linked with gastrointestinal conditions such as inflammatory bowel disease (IBD) and irritable bowel syndrome (IBS), and wider systemic manifestations of disease such as obesity and type 2 diabetes (T2D). The microbes of the gut undertake a variety of metabolic functions and are able to produce a variety of vitamins, synthesize essential and nonessential amino acids, and provide other functions. Amongst other functions, the microbiome of an individual provides biochemical pathways for the metabolism of non-digestible carbohydrates; some oligosaccharides that escape digestion; unabsorbed sugars and alcohols from the diet; and host-derived mucins.

The triglycerides data 406A may include data about triglycerides for an individual. In some examples, the triglycerides data 406A can be determined from an At Home Blood Test which in some cases is a finger prick on to a dried blood spot card.

The glucose data 406B includes data about blood glucose. The glucose data 406B may be determined from various testing mechanisms, including at home measurements, such as a continuous glucose meter.

The blood data 406C may include blood tests relating to a variety of different biomarkers. As discussed above, at least some blood tests can be performed at home. In some configurations, the blood data 406C is associated with measuring blood sugar, insulin, c-peptides, triglycerides, IL-6 inflammation, ketone bodies, nutrient levels, allergy sensitivities, iron levels, cholesterol, blood count levels, HbA1c, and the like.

The wearable data 406D can include any data received from a computing device associated with an individual. For instance, an individual may wear an electronic data collection device 403, such as an activity-monitoring device or other wearable device, that monitors motion, heart rate, determines how much an individual has slept, the number of calories burned, activities performed, blood pressure, body temperature, and the like. The individual may also wear a continuous glucose meter that monitors blood glucose levels.

The questionnaire data 406F can include data received from one or more questionnaires, and/or surveys received from one or more individuals. The psychological data 406F, that may be subjectively obtained, may include data received from the individual and/or a computing device that generates data or input based on a subjective determination (e.g., the individual states that they are still hungry after a meal, or a device estimates sleep quality based on the movement of the user at night perhaps combined with heart rate data). The objective health data 406H includes data that can be objectively measured, such as but not limited to height, weight, medical history, and the like.

The nutritional data 406H can include data about food, which is referred to herein as “food data”. For example, the nutritional data can include nutritional information about different food(s) such as their macronutrients and micronutrients or the bioavailability of its nutrients under different conditions (raw vs cooked, or whole vs ground up). In some examples, the nutritional data 406H can include data about a particular food. For instance, before an individual consumes a particular meal, information about that food can be determined. As briefly discussed, the user might scan a barcode on the food item(s) being consumed and/or take one or more pictures of the food to determine the food, as well as the amount of food, being consumed.

The nutritional data can include food data that identifies foods consumed, a quantity of the foods consumed, food nutrition (e.g., obtained from a nutritional database), food state (e.g., cooked, reheated, frozen, etc.), food timing data (e.g., what time was the food consumed, how long did it take to consume, . . . ), and the like. The food state can be relevant for foods such as carbohydrates (e.g., pasta, bread, potatoes, or rice), since carbohydrates may be altered by processes such as starch retrogradation. The food state can also be relevant for quantity estimation of the foods, since foods can change weight dramatically during cooking. In some instances, the user may also take a picture before and/or after consuming a meal to determine what food was consumed as well as how much of the food was consumed. The picture can also provide an indication as to the food state.

The other data 132 can include other data associated with the individual. For example, the other data 132 can include data that can be received directly from a computer application that logs information for an individual (e.g., food eaten, sleep, . . . ) and/or from the user via a user interface.

In some examples, different computing devices 114 associated with different users provide application data 404 to the data manager 212 for ingestion by the data ingestion service 210. As illustrated, computing device 114 provides app data 236 to the data manager 212, computing device 114B provides app data 236B to the data manager 212, and the nth computing device 114N provides app data 236N to the data manager 212. There may be any number of computing devices utilized.

As discussed briefly above, the data manager 212 receives data from different data sources, processes the data when needed (e.g., cleans up the data for storage in a uniform manner), and stores the data within one or more data stores, such as the data store 126.

The data manager 212 can be configured to perform processing on the data before storing the data in the data store 126. For example, the data manager 212 may receive data for ketone bodies and then use that data to generate ketone body ratios. Similarly, the data manager 212 may process food eaten and generate meal calories, number of carbohydrates, fat to carbohydrate rations, how much fiber consumed during a time period, and the like. The data stored in the data store 126, or some other location, can be utilized by the microbiome service 116 to determine an accuracy of at home measurements of nutritional responses performed by users. The data outputted by the microbiome service 116 to the nutritional service may therefore contain different values than are stored in the data store 126, for example if a food quantity is adjusted.

FIGS. 5-13 are flow diagrams showing processes 500, 600, 700, 800, 900, 1000, 1100, 1200, and 1300 respectively that illustrate aspects of generating microbiome fingerprints, dietary fingerprints, microbiome ancestry data, and microbiome scores in accordance with examples described herein. It should be appreciated that at least some of the logical operations described herein with respect to FIGS. 5-13, and the other FIGs., may be implemented (1) as a sequence of computer implemented acts or program modules running on a computing system and/or (2) as interconnected machine logic circuits or circuit modules within the computing system.

The implementation of the various components described herein is a matter of choice dependent on the performance and other requirements of the computing system. Accordingly, the logical operations described herein are referred to variously as operations, structural devices, acts, or modules. These operations, structural devices, acts, and modules may be implemented in software, in firmware, in special purpose digital logic and any combination thereof. It should also be appreciated that more or fewer operations may be performed than shown in the FIGs. and described herein. These operations may also be performed in parallel, or in a different order than those described herein.

FIG. 5 is a flow diagram showing a process 500 illustrating aspects of a mechanism disclosed herein for obtaining and utilizing microbiome data for a user to generate microbiome fingerprints, dietary fingerprints, and microbiome ancestry for users.

The process 500 may begin at 502, where microbiome sample/data is obtained from a user. As discussed above, a user may provide one or more microbiome samples that may be obtained at home or in a clinical setting. For example, the user may provide a sample or samples of their stool for microbiome analysis as part of the at home biological collection, and/or the sample(s) may be collected in a lab, or other clinical setting. In some configurations, the user may also provide other data that may be utilized when processing the sample. For instance, the user may provide timing data indicating when the sample was taken, conditions under which the sample was obtained, and/or other health data.

At 504, the microbiome data is processed. As discussed above, microbiome service 116 may generate DNA data from the sample. In some examples, the DNA is extracted from the cells of the microbiome sample and purified. Different techniques that are commercially available can be utilized for DNA extraction from the microbiome sample. Generally, the use of different extraction techniques may result in different biases that may affect an accurate microbial representation.

At 506, the microbial composition of the microbiome sample may be identified. According to some configurations, the microbiome service 116, or some other device or component, identifies the microbial composition of the microbiome (e.g., the species, genes, taxa, and the like). The microbiome service 116 may also generate reconstructed microbial genomes, determine a diversity of the microbiome, identify functions of the microbiome, identify a uniqueness of the microbiome, identify interesting species, and the like.

At 508, the diversity of the microbiome may be determined. As discussed above, the microbiome service 116 may determine the diversity of the microbiome associated with a user. In some examples, the diversity determined by the microbiome service 116 is the number of individual bacteria from each of the bacterial species present in the microbiome. Having a more diverse microbiome may have health benefits. According to some configurations, the microbiome service 116 may provide this data, possibly along with recommendations, to the user via a UI, or some other interface.

At 510, reconstructed microbial genomes are generated. The microbiome service 116, or some other component or device may generate the reconstructed microbial genomes. Reconstruction of DNA fragments into genomes may utilize different techniques and methods and generally incorporates sequence assembly and sorting/clustering of assembled sequences into different bins associated with characteristics of a genome.

At 512, the functions of a microbiome may be determined. As discussed above, the microbiome service 116, or some other device or component, may determine the functions of a microbiome. Different techniques and methods may be utilized to determine the functions. Generally, the microbiome service 116 may map the sequencing reads against sequences of DNA (or amino acids) representing known genes (or proteins) and gene families (or protein families) to determine the functional potential of the microbiome.

At 514, other data associated with the microbiome of the user may be determined. As discussed above, the microbiome service 116, or some other device or component, may determine data such as the uniqueness of the microbiome (e.g., compared to the microbiome of other users), species identified as interesting, and the like.

At 516, the microbiome data associated with the user is stored. As discussed above, the microbiome service 116, or some other device or component, may store the microbiome data in a data store, such as user microbiome data 128 within data store 126.

At 518, the microbiome data associated with the user is utilized to generate microbiome fingerprints, dietary fingerprints, and microbiome ancestry for the user. As discussed above, the microbiome service 116, or some other device or component, may perform these tasks. See FIGS. 5-13 and related discussion for more details.

FIG. 6 is a flow diagram showing a process 600 illustrating aspects of a mechanism disclosed herein for generating a microbiome fingerprint for a user. As discussed above, the microbiome fingerprint may be generated using various techniques and methods. The following process is an example of generating a microbiome fingerprint.

At 602, microbiome data for a particular user is accessed. As discussed above, the microbiome service 116, or some other device or component, may access the microbiome data 128 within data store 126 to obtain the microbiome data for a user. In other examples, the microbiome data may be obtained/accessed using some other technique (e.g., accessing a different memory, receiving the data from some other source, such as data source(s) 120, and the like).

At 604, the microbiome data may be preprocessed to generate screened microbiome data. As discussed above, the microbiome service 116, or some other device or component, may process the sequencing data to generate screened sequencing data. The screened sequence data may make the generation of the different profiles described below be more accurate.

At 606, the quantitative taxonomic profiles are generated. As discussed above, the microbiome service 116, or some other device or component, may generate the quantitative taxonomic profiles. The quantitative taxonomic profiles can be obtained by mapping (i.e. matching the sequences) the sequencing reads against sequences representing the known microbial organisms. The mapping is then processed to produce relative abundances of the reference microbes. Many open source algorithms and corresponding implementations are available for this step, including for example, the techniques as described by Truong et al. (Nature Methods 12 (10): 902-3, 2015) and the newer versions of the associated software, including MetaPhlAn 4 (Blanco-Míguez et al., Nat Biotechnol, 2023, doi.org/10.1038/s41587-023-01688-w).

At 608, the quantitative functional potential profiles are generated. As discussed above, the microbiome service 116, or some other device or component, may generate the quantitative functional potential profiles. The quantitative functional potential profiles can be obtained by mapping the sequencing reads against sequences of DNA (or amino acids) representing known genes (or proteins) and gene families (or protein families). Based on the number of reads matching each gene or gene family the presence and abundance of the gene families and pathways are inferred. Several open source algorithms and corresponding implementations are available for this step, including for example the technique HUMAnN2 as described by Abubucker et al. (PLOS Computational Biology 8 (6), 2012) and Franzosa et al. (Nature Methods, 15 (11), 962, 2018) and any newer versions of the associated software.

At 610, the strain-level genomic profiles are generated. As discussed above, the microbiome service 116, or some other device or component, may generate the strain-level genomic profiles. The strain-level genomic profiles, or the third descriptor, can be obtained with reference-based and assembly-based approaches. For reference-based approaches the methods use specific genetic markers against which the reads are mapped, and single-nucleotide polymorphisms are inferred. The combinations of single-nucleotide polymorphisms provide strain-specific profiles. Some open source algorithms and implementations for this step are available, including for example the techniques described by Truong et al. (Genome Research 27 (4): 626-38, 2017), and MetaPhlAn 4 (Blanco-Míguez et al., Nat Biotechnol, 2023, doi.org/10.1038/s41587-023-01688-w). In assembly-based approaches, reads may be first concatenated to form longer contiguous sequences such as described by Li et al. (Bioinformatics 31 (10): 1674-76, 2015).

Contigs may then be clustered in bins representing the sequences of whole genomes, such as described by Kang et al. (PeerJ 7: e7359, 2019). The resulting draft genomes may be quality controlled using for example the techniques described by Parks et al. (Genome Research 25 (7): 1043-55, 2015). The quality-controlled genomes represent single strains in the microbiome.

At 612, the microbiome fingerprint for the user is generated. As discussed above, the microbiome service 116, or some other device or component, may combine the data associated with the different indexes generated at 606, 608, and 610 to generate the microbiome fingerprint for the user.

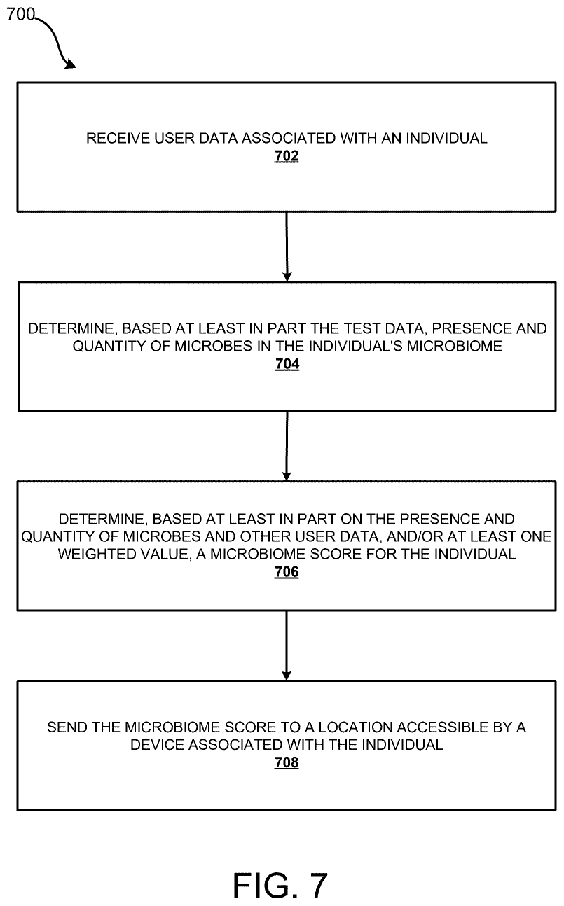

FIG. 7 is a flow diagram showing an example process 700 for generating microbiome scores for individuals. As discussed above, the microbiome score may include a single metric that represents the overall health and quality of an individual's microbiome including the beneficial and harmful microbe. In some cases, a cloud-based or other type of service or system may be configured to receive user data, such as health data, diet data, and/or test data associated with an individual's gut, and to generate the microbiome score based on the user data.

At 702, the system may receive user data associated with an individual. In some cases, the individual may be provided with a testing kit or product to provide a sample. In other cases, the user data may include test data received from a lab or other test facility. The user data may also include health data, diet data, demographic data, lifestyle data, geographical data (e.g., region that the individual lives) and the like associated with user.

At 704, the system may determine, based at least in part on the user data, a presence and quantity of microbes in the individual's microbiome. For example, the system may determine the presence based at least in part on the test data associated with the individual's gut. In some cases, the system may identify the presence of a subset or selected microbes. For example, the system may have categorized microbes as beneficial, harmful, and/or neutral. In these cases, the system may determine the presence of microbes that have a beneficial nature and/or harmful nature. In some cases, the quantity of each of the microbes may be a relative quantity such as a percentage of the microbiome that the individual species occupies.

At 706, the system may determine, based at least in part on the presence and quantity of microbe, other user data (e.g., health data, diet data, demographic data, geographical data, and the like), and/or at least one weighted value, a microbiome score for the individual. For example, as discussed herein, the system may determine weighted values for each of the harmful and/or beneficial microbes that may or may not be present in a human's microbiome. For example, each weighted value may be between negative one and positive one, where positive values represent beneficial (“good”) microbes and negative values represent harmful (“bad”) microbes.

At 708, the system may send the microbiome score to a location accessible by a device associated with the individual. For example, the system may send a notice or alert to an application hosted by the device, the microbiome score, and/or link usable to access the microbiome score via a secured storage location.

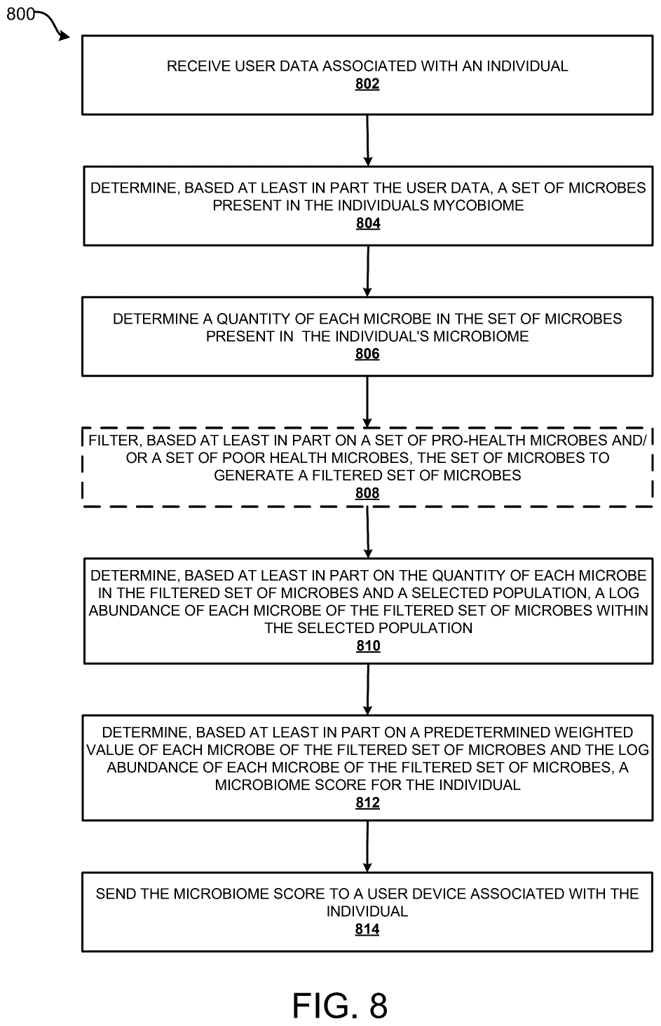

FIG. 8 is a flow diagram showing an example process 800 for generating microbiome scores for individuals. As discussed above, the microbiome score may include a single metric that represents the overall health and quality of an individual's microbiome including the beneficial and harmful microbe. In some cases, a cloud-based or other type of service or system may be configured to receive user data, such as health data, diet data, and/or test data associated with an individual's gut, and to generate one or more microbiome score based on the user data.

At 802, the system may receive user data associated with an individual. In some cases, the individual may be provided with a testing kit or product. In other cases, the user data may include test data received from a lab or other test facility. The user data may also include health data, diet data, demographic data, diet data, geographical data (e.g., region that the individual lives) and the like associated with user. In some specific examples, the user data may be testing data generated by a lab of the individual's gut or microbiome.

At 804, the system may determine, based at least in part on the user data, a set of microbes present in the individual's microbiome. For example, the system may determine the presence based at least in part on the lab test data associated with the individual's gut. In some cases, the system may include a set of classified or known microbes that are also present in the individual's gut.

At 806, the system may determine a quantity of each microbe in the set of microbes present in the individual's microbiome. For example, the quantity may be a percentage-based quantity as compared with each other microbe in the gut or a quantitative number as to amount of each microbe present. In some cases, the quantity of each of the microbes may be a relative quantity such as a percentage of the microbiome that the individual species occupies.

At 808, the system may filter, based at least in part on a set of pro-health microbes and/or a set of poor health microbes, the set of microbes to generate a filtered set of microbes. In some cases, the step 808 may be optional and the full set of microbes present in the gut of the individual may be utilized at 810-812 below. In other cases, the system may filter the microbes to identify the presence of a subset or selected microbes. For example, the system may have categorized microbes as beneficial, harmful, and/or neutral. In these cases, the system may determine the presence of microbes that have a beneficial nature and/or harmful nature and filter out the microbes that have a neutral nature. In other aspects, the system may have categorized the most responsive microbes, that is the microbes for which the absence, presence, or relative abundance is most likely to change based on a change in a particular parameter such as diet or exercise. Thus, the system may filter the microbes in a sample to obtain a fractional representation of the microbes. The fractional representation may be a portion of microbes, for example, a portion of the diet ranked list or health ranked list as shown in Table 1A.

At 810, the system may determine, based at least in part on the quantity of each microbe in the filtered set of microbes and a selected population, a log abundance of each microbe of the filtered set of microbes within the selected population. For example, the selected population may be a population of individuals with similar diet, similar demographic information (such as age, sex, or the like), similar lifestyles, or the like. In other cases, the selected population may be based on geographic areas, such as countries, states, cities, or other geographic boundaries (such as distance from an ocean or sea).

At 812, the system may determine, based at least in part on a predetermined weighted value of each microbe of the filtered set of microbes and the log abundance of each microbe of the filtered set of microbes, a microbiome score for the individual. For example, the log abundance within the population for each microbe may then have the corresponding microbe weighted value applied. The resulting weighted microbe scores may then be considered to determine the microbiome score. For example, the total or summed resulting weighted microbe scores may be ranked or otherwise organized to generate the microbiome score. For example, if a ranking is used the microbiome score may be a percentage rank for the individual with respect to the corresponding selected population (e.g., better than 85% of the population, 50% of the population, 25% of the population or the like). In other aspects, the microbiome score may be an absolute value.

In the case in which the microbes are not filtered, or all of the identified microbes are utilized, the predetermined weighted value of 812 may be 0 for neutral microbes, positive for beneficial microbes, and negative for harmful microbes. In various examples, one or more machine learned model and/or scoring technique may be utilized to generate the predetermined weighted value for each microbe (including positive weights for beneficial microbes, negative weights for harmful microbes, and zero weights for neutral microbes). In other aspects, the predetermined weighted value 812 may be based on all possible gut microbes in a population. That is, the weighting may be a continuum with each microbe having a weight. In various cases, the one or more machine learned models generating the weighted values may be trained using health data and microbiome data of various individuals having different health status, lifestyles, geographic locations, demographic data, and the like.

At 814, the system may send the microbiome score to a location accessible by a device associated with the individual. For example, the system may send a notice or alert to an application hosted by the device, the microbiome score, and/or link usable to access the microbiome score via a secured storage location.

FIG. 9 is a flow diagram showing a process 600 illustrating aspects of a mechanism disclosed herein for generating a microbiome ancestry for a user.

The process 900 may begin at 902, where microbiome data for a particular user is accessed. As discussed above, the microbiome service 116, or some other device or component, may access the microbiome data 128 within data store 126 to obtain the microbiome data for a user. In other examples, the microbiome data may be obtained/accessed using some other technique (e.g., accessing a different memory, receiving the data from some other source, such as data source(s) 120, and the like).

At 904, the microbiome data is compared to microbiome data from other users. As discussed above, the microbiome service 116, or some other device or component, may utilize the microbiome data, such as the microbiome fingerprint data of a particular user, and compare microbiome fingerprint data of other users. According to some configurations, the microbiome service 116 may generate one or more indicators that identify how close another user is to the user based on a similarity of the microbiome data.

At 906, one or more other users are identified based on a similarity of the microbiome data between the users. As discussed above, the microbiome service 116, or some other device or component, may identify the related users based on a score generated by the microbiome service 116, or some other indicators.

At 908, the geographic region(s) that are commonly associated with the microbiome data of a user are identified. As discussed above, the microbiome service 116, or some other device or component, may identify that different geographic regions are more closely linked to certain microbiomes.

At 910, the microbiome ancestry data may be utilized. As discussed above, the microbiome service 116, or some other device or component, may utilize the microbiome ancestry data when providing data to the user, when generating the microbiome ancestry data, generating recommendations for the user (e.g., nutritional), and/or performing some other task.

FIG. 10 is a flow diagram showing a process 1000 illustrating aspects of a mechanism disclosed herein for generating a dietary fingerprint for a user.

The process 1000 may begin at 1002, where microbiome data for a particular user are accessed. As discussed above, the microbiome service 116, or some other device or component, may access the microbiome data 128 within data store 126 to obtain the microbiome data for a user. In other examples, the microbiome data may be obtained/accessed using some other technique (e.g., accessing a different memory, receiving the data from some other source, such as data source(s) 150, and the like).

At 1004, dietary fingerprint data is generated. As discussed above, the microbiome service 116, or some other device or component, may generate dietary fingerprint data that identifies a similarity between the microbiome of a particular user and a “dietary fingerprint” is data that identifies how the microbiome of a user is associated with one or more different indexes. The indexes may include, but are not limited to a Mediterranean diet index, a vegetarian diet index, a fast food index, an internal fat index, a fat-digesting index, a carbohydrate-digesting index, a health index, a fasting index, a ketogenic index, and the like. According to some configurations, one or more computers of a microbiome service generate a score, such as from 0-100, (or some other indicator) that indicates how closely the microbiome of the user is associated with a particular index.

As an example, the Mediterranean diet index score for a user indicates how closely the microbiome of the user resembles the typical microbiome of someone on a Mediterranean diet. The vegetarian diet index score indicates how closely the microbiome of the user resembles someone on a vegetarian diet. The fast food index score indicates how closely the microbiome of the user resembles someone on a fast food diet. The internal fat index score indicates how closely the microbiome of the user resembles someone with high or low visceral fat. The fat-digesting index score indicates how closely the microbiome of the user resembles someone with low postprandial triacylglycerol (TAG) rises. The carbohydrate-digesting index score indicates how closely the microbiome of the user resembles someone with low postprandial glucose rises. The health index score indicates how closely the microbiome of the user resembles someone that is healthy. The fasting index score indicates how closely the microbiome of the user resembles someone that fasts regularly. The ketogenic index score indicates how closely the microbiome of the user resembles someone who is ketogenic.

At 1006, a determination is made as to whether another dietary index is to be compared. As discussed above, there may be a variety of dietary indexes, including but not limited to a Mediterranean diet index, a vegetarian diet index, a fast food index, an internal fat index, a fat-digesting index, a carbohydrate-digesting index, a health index, a fasting index, a ketogenic index, and the like. When there is another index, the process 1000 returns to 1004. When there is not another index, the process 1000 moves to 1008.

At 1008, the dietary index(es) associated with the user are identified. As discussed above, the microbiome service 116, or some other device or component, may identify one or more diets that resemble the microbiome of the user. In some examples, the microbiome service 116 identifies the closest dietary index (e.g., based on a score). In other examples, the microbiome service 116 may rank the dietary index.

At 1010, the dietary fingerprint data may be utilized. As discussed above, the microbiome service 116, or some other device or component, may utilize the dietary fingerprint data when providing data to the user, when generating the microbiome ancestry data, generating recommendations for the user (e.g., nutritional), and/or performing some other task.

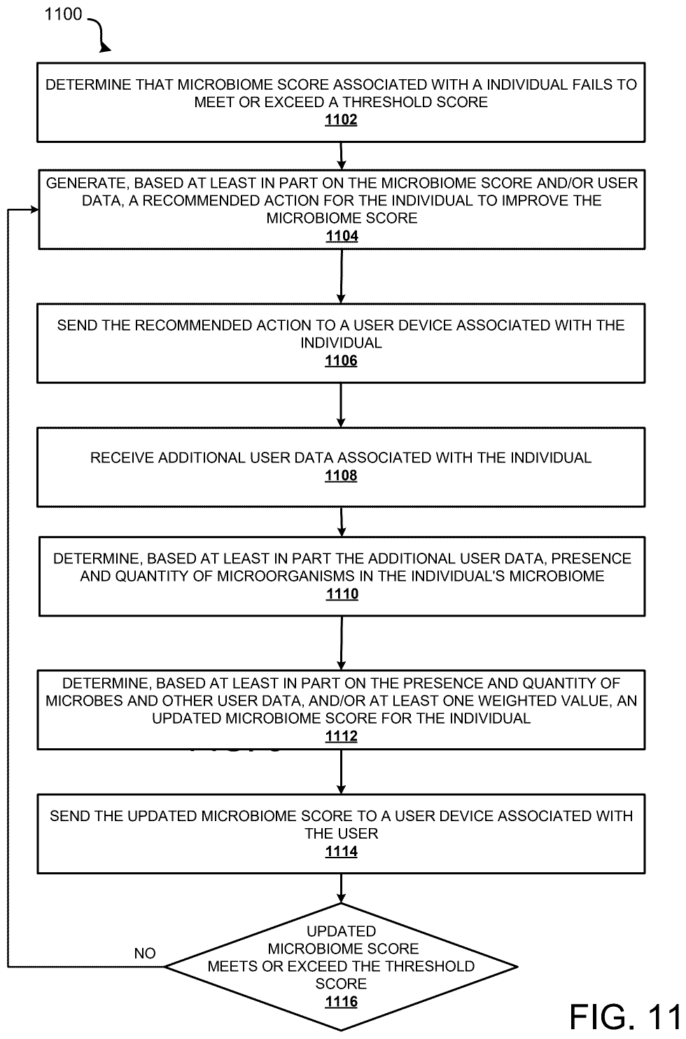

FIG. 11 is a flow diagram showing an example process 1100 for generating microbiome scores and recommended actions to improve microbiome scores for individuals. As discussed above, in some examples, in addition to the microbiome score the system may generate recommended actions, such as diet regimens, exercise regimens, supplement regimens, and the like to improve the overall health and quality of the individual's microbiome.

At 1102, the system may determine that a microbiome score associated with an individual fails to meet or exceed a threshold score. For example, the microbiome score as determined with respect to process 700 may be used to determine if the microbiome score meets or exceeds the threshold score. In some cases, the system may utilize multiple threshold scores that may, when compared to an individual's microbiome score, trigger various different recommended actions for the individual.

At 1104, the system may generate, based at least in part on the microbiome score and/or the user data (e.g., the test data, diet data, or the like), a recommended action for the individual to improve the microbiome score. For example, the system may recommend diet changes, exercise changes, supplements, or the like. For example, the system may recommend increasing fiber intake, eating higher quality fats, eating less processed food or increasing polyphenol intake.

At 1106, the system may send the recommended action to a user device associated with the individual. For example, the system may provide the recommended action via an application hosted by the user device that tracks the individual's microbiome scores over time and may assist with helping the individual to follow the recommended actions. For example, the recommended actions may include a daily recommended action, a weekly regime, a one time action, or the like.

In some cases, the individual may provide feedback via the user device and/or hosted application. In some cases, the individual may enter the data, such as movement, activity, or exercise data, diet data, supplement data, or the like via a user interface on the user device/application. In other cases, the user device may include sensors, such as motion sensors, gyroscopes, accelerometers, magnetometer, or the like that may provide data to the system in order to monitor and track the execution of the recommended actions. In some examples, the system may update the recommended actions based on the individual's progress or success in completing prior recommended actions.

At 1108, the system may receive additional user data associated with the individual. For instance, the system may receive updated test data from the user device and/or a testing lab. In some examples, the individual may perform the recommended actions for a predetermined period of time (such as a number of weeks, a number of months, or the like) and/or until target goals (such as a weight target, a distance traversed target, a speed target, a strength target, or the like) are achieved. In these cases, the individual may perform an updated test associated with the individual's microbiome.

At 1110, the system may determine, based at least in part on the additional user data, a presence and quantity of microbes in the individual's microbiome. For example, the system may determine the presence based at least in part on the test data associated with the individual's gut. In some cases, the system may identify the presence of a subset or selected microbes. For example, the system may have categorized microbes as beneficial, harmful, and/or neutral. In these cases, the system may determine the presence of microbes that have a beneficial nature and/or harmful nature. In some cases, the quantity of each of the microbes may be a relative quantity such as a percentage of the microbiome that the individual species occupies.

At 1112, the system may determine, based at least in part on the presence and quantity of microbe, other user data (e.g., health data, diet data, demographic data, geographical data, and the like), and/or at least one weighted value, an updated microbiome score for the individual. For example, as discussed herein, the system may determine weighted values for each of the harmful and/or beneficial microbes that may or may not be present in a human's microbiome. For example, each weighted value may be between negative one and positive one, where positive values represent beneficial microbes and negative values represent harmful microbes.

At 1114, the system may send the updated microbiome score to a location accessible by a device associated with the individual. For example, the system may send a notice or alert to an application hosted by the device, the microbiome score, and/or link usable to access the microbiome score via a secured storage location. In some cases, the system may send comparisons between the original microbiome score and the updated microbiome score. In other cases, the system may track the changes in the microbiome scores over time, such as when the individual obtains an updated microbiome score on a periodic basis (such as monthly, quality, yearly, or the like).

At 1116, the system may determine if the updated microbiome score meets or exceeds the threshold score or scores. For example, the system may determine if the updated microbiome score meets or exceeds one or more desired threshold or criterion. In some cases, the threshold scores may include thresholds or criterion associated with the overall score, individual species scores or percentages (e.g., one or more harmful microbes above a threshold percentage or quantity, one or more beneficial microbes below a threshold percentage or quantity, or some combination thereof). In the current example, if the updated microbiome fails to meet or exceed the threshold score or scores, the process 1100 returns to 1104 and the system generates additional or updated recommended actions. Otherwise, the system may recommend additional testing at a future date and/or time.

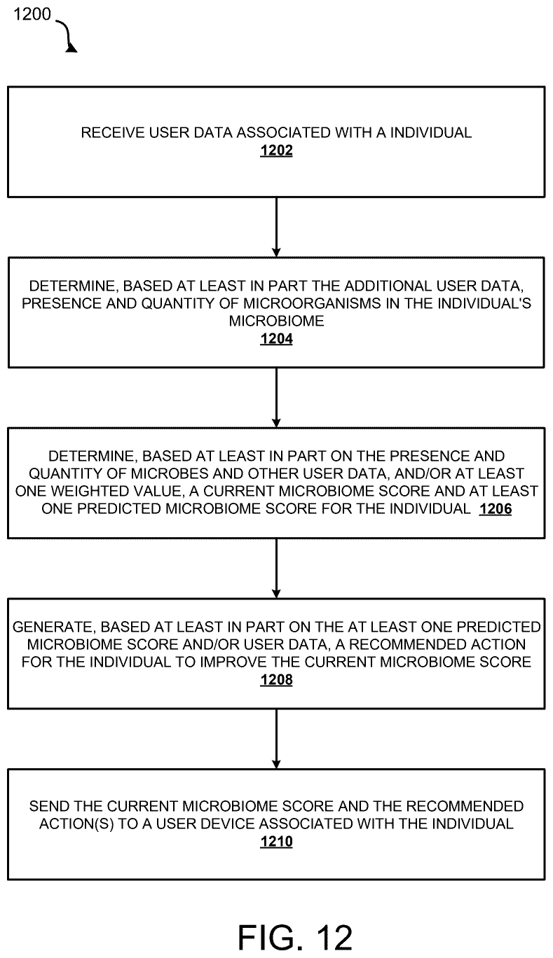

FIG. 12 is a flow diagram showing an example process 1200 for generating microbiome scores and recommended actions to improve microbiome scores for individuals. As discussed above, in some examples, in addition to the microbiome score the system may generate recommended actions, such as diet regimens, exercise regimens, supplement regimens, and the like to improve the overall health and quality of the individual's microbiome.

At 1202, the system may receive user data associated with an individual. In some cases, the individual may be provided with a testing kit or product. In other cases, the user data may include test data received from a lab or other test facility. The user data may also include health data, demographic data, diet data, geographical data (e.g., region that the individual lives) and the like associated with user.

At 1204, the system may determine, based at least in part on the user data, a presence and quantity of microbes in the individual's microbiome. For example, the system may determine the presence based at least in part on the test data associated with the individual's gut. In some cases, the system may identify the presence of a subset or selected microbes. For example, the system may have categorized microbes as beneficial, harmful, and/or neutral. In these cases, the system may determine the presence of microbes that have a beneficial nature and/or harmful nature. In some cases, the quantity of each of the microbes may be a relative quantity such as a percentage of the microbiome that the individual species occupies.

At 1206, the system may determine, based at least in part on the presence and quantity of microbe, other user data (e.g., health data, diet data, demographic data, geographical data, and the like), and/or at least one weighted value, a current microbiome score and at least one predicted microbiome score for the individual. For example, the system may predict changes in the composition of the individual's microbiome if the individual performs various recommended actions. In some cases, the predicted changes may be at various future times, such as a one week, one month, and the like. In other cases, the predicted changes may be based at least in part on achieving a target goal, such as a reduction in weight. In some cases, the predicted changes may be the output of a one or more machine learned models and/or networks that were trained based on data associated with microbiomes collected from individuals performing recommend actions over time.

At 1208, the system may generate, based at least in part on the at least one precited microbiome score, a recommended action for the individual to improve the current microbiome score. For example, the system may select the recommended action that is associated with the highest ranking improvement in microbiome score (e.g., the highest ranking precited microbiome score). In some cases, the system may combine multiple actions into the recommended action(s) based on changes in the predicted microbiome scores relative to the current microbiome score. For instance, if a first recommended action correlates to a first predicted microbiome scores that improve a first species of microbe and a second recommended action correlates to a second predicted microbiome scores that improve a second species of microbe and both species are beneficial, the system may send both the first and second recommended actions to the user device associated with the individual.

At 1210, the system may send the current microbiome score and the recommended action(s) to a location accessible by a device associated with the individual. For example, the system may send a notice or alert to an application hosted by the device, the current microbiome score, and/or a link usable to access the microbiome score via a secured storage location. In some examples, the system may also send the predicted microbiome scores and/or one or more graphical representations (e.g., icon, chart, graph, or the like) of a predicted result if the individual follows the recommended action(s).

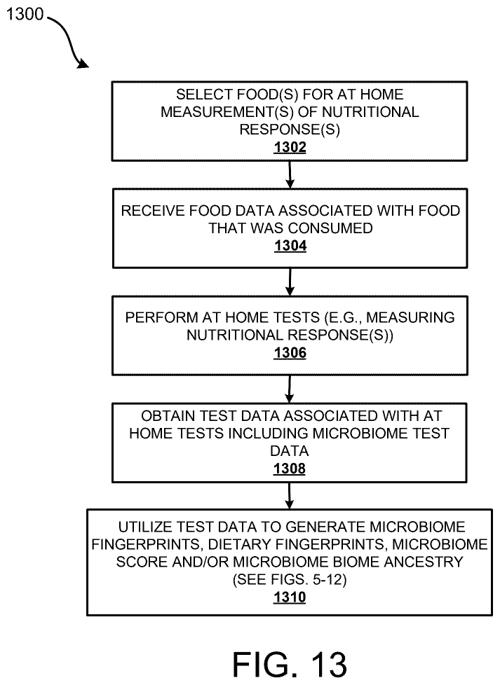

FIG. 13 is a flow diagram showing a process 1300 illustrating aspects of a mechanism disclosed herein for obtaining test data, including microbiome data, to be utilized for generating microbiome fingerprints, dietary fingerprints, and microbiome ancestry for users.

At 1302, food(s) for at home measurements of nutritional responses may be selected. As briefly discussed above, different foods may be selected for a user to eat before a test is performed in order to evoke a desired response. The foods can include foods for a series of standardized meals, a single food, or some other combination of foods.

At 1304, food data is received. As discussed above, the food data is associated with foods that are utilized to evoke a nutritional response. The food data can include foods for a series of standardized meals, a single food, or some other combination of foods. The food data can include data such as foods consumed, a quantity of the foods consumed, food nutrition (e.g., obtained from a nutritional database), food state (e.g., cooked, reheated, frozen, etc.), food timing data (e.g., what time was the food consumed, how long did it take to consume, . . . ), and the like. The food state can be relevant for foods such as carbohydrates (e.g., pasta, bread, potatoes, or rice), since carbohydrates may be altered by processes such as starch retrogradation. The food state can also be relevant for quantity estimation of the foods, since foods can change weight significantly during cooking.

At 1306, at home test(s) are performed. The tests may include at home tests as described above and/or the collection of one or more samples (e.g., stool for microbiome analysis or blood for glucose or cholesterol analysis).

At 1308, test data associated with the at home tests including microbiome data is received. As discussed above, microbiome data may be associated with one or more tests. In some configurations, the microbiome data includes a stool sample, timing data for the sample (e.g., when collected, how long stored before providing to a lab), data associated with collection of the sample (e.g., how was sample stored, was the sample contaminated), as well as other data. For example, a user may be instructed to take a picture of the sample and provide the image to the service.

At 1310, the test data is utilized to generate microbiome fingerprints, dietary fingerprints, microbiome scores, and microbiome ancestry. In some examples, the test data is used by the microbiome service 116 to generate the microbiome fingerprints, dietary fingerprints, and microbiome ancestry. The microbiome system 112 may also use the test data to generate nutritional recommendations (for instance in the form of a diet program or food guidance program) that are personalized for a particular user.

### (II) REPRESENTATIVE COMPUTER ARCHITECTURE

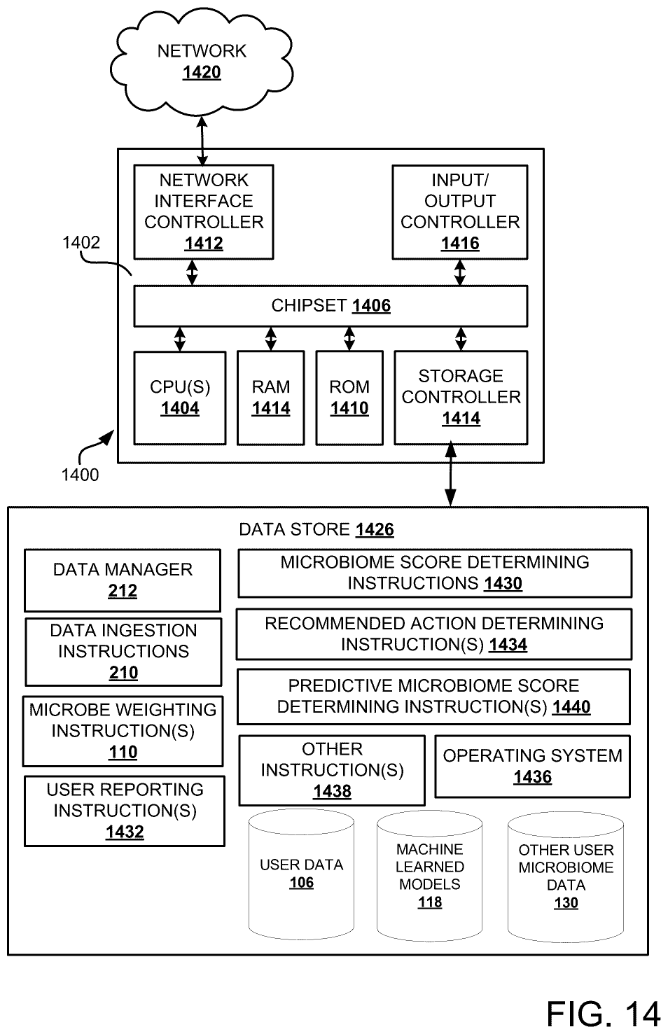

FIG. 14 shows an example computer architecture for a computer 1400 capable of executing program components for generating microbiome fingerprints, dietary fingerprints, and microbiome ancestry for users in the manner described above. The computer architecture shown in FIG. 14 illustrates a conventional server computer, workstation, desktop computer, laptop, tablet, network appliance, digital cellular phone, smart watch, or other computing device, and may be utilized to execute any of the software components presented herein. For example, the computer architecture shown in FIG. 14 may be utilized to execute software components for performing operations as described above. The computer architecture shown in FIG. 14 might also be utilized to implement a computing device 114, or any other of the computing systems described herein.

The computer 1400 includes a baseboard 1402, or “motherboard,” which is a printed circuit board to which a multitude of components or devices may be connected by way of a system bus or other electrical communication paths. In one illustrative example, one or more central processing units (CPUs) 804 operate in conjunction with a chipset 1406. The CPUs 1404 may be standard programmable processors that perform arithmetic and logical operations necessary for the operation of the computer 1400.

The CPUs 1404 perform operations by transitioning from one discrete, physical state to the next through the manipulation of switching elements that differentiate between and change these states. Switching elements may generally include electronic circuits that maintain one of two binary states, such as flip-flops and electronic circuits that provide an output state based on the logical combination of the states of one or more other switching elements, such as logic gates. These basic switching elements may be combined to create more complex logic circuits, including registers, adders-subtractors, arithmetic logic units, floating-point units, and the like.

The chipset 1406 provides an interface between the CPUs 1404 and the remainder of the components and devices on the baseboard 1402. The chipset 1406 may provide an interface to a random-access memory (RAM) 808, used as the main memory in the computer 800. The chipset 806 may further provide an interface to a computer-readable storage medium such as a read-only memory (ROM) 1410 or non-volatile RAM (NVRAM) for storing basic routines that help to startup the computer 1400 and to transfer information between the various components and devices. The ROM 810 or NVRAM may also store other software components necessary for the operation of the computer 1400 in accordance with the examples described herein. In some aspects, the computer-readable media may include flash memory, optical disks, magnetic disks, and the like. While RAM and ROM are referenced, the computer 1400 may include fixed media (e.g., GPU, NPU, RAM, ROM, a fixed hard drive, and so on) as well as removable media (e.g., Flash memory, a removable hard drive, an optical disc, and so forth).

The computer 1400 may operate in a networked environment using logical connections to remote computing devices and computer systems through a network, such as the network 1420. The chipset 1406 may include functionality for providing network connectivity through a network interface controller (NIC) 1412, such as a mobile cellular network adapter, Wi-Fi network adapter or gigabit Ethernet adapter. The NIC 1412 is capable of connecting the computer 1400 to other computing devices over the network 1420 to one or more other local or remote computing device(s) or remote services. For instance, the NIC 1412 can facilitate communication with other proximity sensor systems, a central control system, or other facility systems. NIC 1412 may enable Wi-Fi-based communication such as via frequencies defined by the IEEE 802.11 standards, short range wireless frequencies such as Bluetooth, cellular communication (e.g., 2G, 3G, 4G, 4G LTE, 6G, etc.), satellite communication, dedicated short-range communications (DSRC), or any suitable wired or wireless communications protocol that enables the respective computing device to interface with the other computing device(s). It should be appreciated that multiple NICs 1412 may be present in the computer 1400, connecting the computer to other types of networks and remote computer systems.

Several modules such as instructions, data stores, and so forth may be stored within the ROM 1410 and configured to execute on the processors 1404. For example, as illustrated, the computer-readable media 1410 stores data ingestion instructions 210, microbe weighting instructions 110, microbiome score determining instructions 1430, recommended action determining instructions 1434, predicted microbiome score determining instructions 616, user reporting instructions 618, as well as other instructions 620, such as an operating system. The computer-readable media 606 may also be configured to store data, such as user data 622 and machine learned models 624, microbiome data (e.g., benefits, harms, weight values, and the like), as well as other data.

The computer 1400 may be connected to a mass storage device 1418 that provides non-volatile storage for the computer. The mass storage device 1418 may store system programs, application programs, other program modules and data, which have been described in greater detail herein. The mass storage device 1418 may be connected to the computer 1400 through a storage controller 1414 connected to the chipset 1406. The mass storage device 1418 may include one or more physical storage units. The storage controller 1414 may interface with the physical storage units through a serial attached SCSI (SAS) interface, a serial advanced technology attachment (SATA) interface, a fiber channel (FC) interface, or other type of interface for physically connecting and transferring data between computers and physical storage units.

The computer 1400 may store data on the mass storage device 1418 by transforming the physical state of the physical storage units to reflect the information being stored. The specific transformation of physical state may depend on various factors, in different implementations of this description. Examples of such factors may include, but are not limited to, the technology used to implement the physical storage units, whether the mass storage device 1418 is characterized as primary or secondary storage and the like.

For example, the computer 1400 may store information to the data store 1426 by issuing instructions through the storage controller 1414 to alter the magnetic characteristics of a particular location within a magnetic disk drive unit, the reflective or refractive characteristics of a particular location in an optical storage unit, or the electrical characteristics of a particular capacitor, transistor, or other discrete component in a solid-state storage unit. Other transformations of physical media are possible without departing from the scope and spirit of the present description, with the foregoing examples provided only to facilitate this description. The computer 1400 may further read information from the mass storage device 1418 by detecting the physical states or characteristics of one or more particular locations within the physical storage units.

In addition to the mass storage device 1418 described above, the computer 1400 may have access to other computer-readable storage media to store and retrieve information, such as program modules, data structures, or other data. It should be appreciated by those skilled in the art that computer-readable storage media is any available media that provides for the non-transitory storage of data and that may be accessed by the computer 1400.

By way of example, and not limitation, computer-readable storage media may include volatile and non-volatile, removable, and non-removable media implemented in any method or technology. Computer-readable storage media includes, but is not limited to, RAM, ROM, erasable programmable ROM (EPROM), electrically-erasable programmable ROM (EEPROM), flash memory or other solid-state memory technology, compact disc ROM (CD-ROM), digital versatile disk (DVD), high definition DVD (HD-DVD), BLU-RAY, or other optical storage, magnetic cassettes, magnetic tape, magnetic disk storage or other magnetic storage devices, or any other medium that can be used to store the desired information in a non-transitory fashion.

The data store device 1426 may store an operating system 1436 utilized to control the operation of the computer 1400. According to one example, the operating system includes the LINUX® (Linus Torvalds, Boston, MA) operating system. According to another example, the operating system includes the WINDOWS® SERVER® (Microsoft Corporation, Redmond, WA) operating system from MICROSOFT® (Microsoft Corporation, Seattle, WA). According to another example, the operating system includes the iOS® (Cisco Technology Inc., San Jose, CA) operating system from Apple® (Apple Inc., Cupertino, CA). According to another example, the operating system includes the Android® (Google LLC, Mountain View, CA) operating system from Google® (Google LLC) or its ecosystem partners. According to further examples, the operating system may include the UNIX® (The Open Group Limited, Reading, Berkshire, England) operating system. It should be appreciated that other operating systems may also be utilized. The mass storage device 1418 may store other system or application programs and data utilized by the computer 1400, such as components that include the microbiome system 122 and/or any of the other software components and data described above. The data store 1426 might also store other programs and data not specifically identified herein.

In one example, the data stored in the data store 1426 or other computer-readable storage media is encoded with computer-executable instructions that, when loaded into the computer 1400, create a special-purpose computer capable of implementing the examples described herein. These computer-executable instructions transform the computer 1400 by specifying how the CPUs 804 transition between states, as described above. According to one example, the computer 1400 has access to computer-readable storage media storing computer-executable instructions which, when executed by the computer 1400, perform the various processes described above with regard to FIGS. 5-13. The computer 1400 might also include computer-readable storage media for performing any of the other computer-implemented operations described herein.

The data ingestion instructions 210 may be configured to extract, segment, classify or otherwise process user data and/or test data to determine presence and quantity of microbes in an individual's microbiome. For example, the data ingestion instructions 608 may utilize one or more of the machine learned models and/or networks to process the user data and test data to assist in determining the composition of the individual's microbiome. In some cases, the data ingestion instructions 608 may identify specific microbes and/or species within the user test data.

The microbe weighting instructions 110 may be configured to determine correlations between microbe and/or species to harmful or beneficial properties. The system may then determine weighting values for each of the harmful and beneficial microbe based on the specific harm or benefit to an individual's health, microbiome quality, and the like. In some cases, the weighting values determined by the microbe weighting instructions 110 may be based on an output of one or more machine learned model and/or network that is trained on health data and microbiome test data over a large number of individuals and/or over a period of testing (such as a number of weeks, a number of months, a number of years, or the like).

The microbiome score determining instructions 1430 may be configured to determine a score representing the overall quality of an individual's microbiome based at least in part on test data associated with the individual's microbiome (e.g., presence and quantity or relative quantity of specific microbe), the weighted values assigned by the microbe weighting instructions 110, and other user data (e.g., diet, exercise, age, other demographic data, and the like).

The recommended action determining instructions 1434 may be configured to determine recommended actions for an individual to improve an overall microbiome score. For example, the recommended action determining instructions 1434 may determine the recommended action based on relative quantities of specific species or organisms within the individual's microbiome. For instance, the recommended action may be known to increase the quantity of specific species or organisms that have beneficial properties with regards to the individual's health. As another example, the recommended action may be known to reduce the quantity of specific species or organisms that provide one or more harmful properties to the individual's health.

The predicted microbiome score determining instructions 1440 may be configured to predict microbiome scores for the individual based on the current microbiome, current microbiome score, user data (such as health, age, and the like of the individual), and execution of specific recommended actions (such as for a period of time or until definable targets are achieved). The predicted microbiome scores may be used to select the recommended actions for the individual and/or to motivate the individual to perform the recommended actions.

The user reporting instructions 1432 may be configured to send the microbiome scores and the recommended actions to the individual. For example, the user reporting instructions 1432 may be configured to provide the microbiome scores and the recommended actions to an application hosted on a user device associated with the individual, to a location or account accessible to the individual, or otherwise provide the microbiome scores and the recommended actions to the individual.

The computer 1400 may also include one or more input/output controllers 1416 for receiving and processing input from a number of input devices, such as a keyboard, a mouse, a touchpad, a touch screen, an electronic stylus, or other type of input device. Similarly, the input/output controller 1416 may provide output to a display, such as a computer monitor, a flat-panel display, a digital projector, a printer, a plotter, or other type of output device. It will be appreciated that the computer 1400 may not include all of the components shown in FIG. 14, may include other components that are not explicitly shown in FIG. 14, or may utilize an architecture completely different than that shown in FIG. 14.

### (III) DETECTION AND IDENTIFICATION OF INDIVIDUAL MICROBES

Described herein are specific methods for detecting and identifying individual member microbes in the microbiome of a subject, as well as methods for identifying and quantifying (in relative or absolute terms) the members of a microbiome. It will be understood, however, that myriad methods exist for detecting and identifying individual member microbes in the microbiome of a subject, as well as methods for identifying and quantifying (in relative or absolute terms) the members of a microbiome. See, for instance: Asnicar et al. (Nat Med. 27:321-323, 2021), Davidson & Epperson (Methods Mol. Biol., 1706:77-90, 2018), Nagpal et al. (Front Microbiol., 8:2897, doi: 10.3389/fmicb.2018.02897, 2018), Nagpal et al. (Sci Rep. 8 (1): 12649, 2018), The Integrative HMP (iHMP) Research Network Consortium (Nature 569:641-648, 2019; and publications cited therein), Wu et al. (Gut. 65 (1): 63-72, 2016). Additional resources are available online, for instance, through the NIH Human Microbiome Project (at hmpdacc.org), including tools and protocols related to Microbial Reference Genomes, Sampling, Sequence & Analysis of 16S RNA, and Sampling, Sequencing & Analysis of Whole Metagenomic Sequence. See also: WO 2021/165494 “Generating Microbiome Fingerprints, Dietary Fingerprints, and Microbiome Ancestry”; and WO 2021/186047 “Microbiome Fingerprints, Dietary Fingerprints, and Microbiome Ancestry, and Methods of Their Use”.

### Defining the ‘Good Bugs’ and the ‘Bad Bugs’

Essentially following the method described in FIG. 6 of the Asnicar et al. (Nat Med 27:321-332, 2021; doi.org/10.1038/s41591-020-01183-8), gut microbes were selected with the strongest overall correlations with a selection of markers of nutritional and cardiometabolic health. These microbes are listed in Table 1B (representative microbes allocated with good health—“GOOD BUGS”) and Table 1C (representative microbes allocated with good health—“BAD BUGS”) though other subset from Table 1A may also be used.

The main changes in the current protocol in comparison with the data extraction done in Asnicar et al., 2021 were:

An increased cohort from the initial 1,098 participants to ˜34,000

For the additional participants, only subsets of the original health and diet markers are available and have been used in the extraction of good and bad bugs

An updated algorithm, MetaPhlAn 4 (github.com/biobakery/MetaPhlAn, doi.org/10.1038/s41587-023-01688-w), that identifies clusters of genomes (species-level genome bins (SGBs); defined in Pasolli et al., Cell 176 (3): P659-662.E20, 2019; doi.org/10.1016/j.cell.2019.01.001) based on their full-genome genomic distance (ANI, >=95%) instead of species. Thus, the microbes (“bugs”) identified herein are primarily identified by an Species-level genome bin (SGB) number, e.g. SGB6340. Species-level genome bins are clusters of similar genomes-see segatalab.cibio.unitn.it/data/Pasolli_et_al.html. Some SGBs correspond 1-1 with a known species. Some known species sub-divide into multiple SGBs. Some SGBs are for species which are not yet characterized.

Two types of microbe lists were defined: list of microbes extracted for their correlations to both cardiometabolic health and nutritional markers and list of microbes extracted only for their correlations to cardiometabolic health markers.

Health metrics may include for example, personal data including age and height, as well as physical data such as weight, BMI, ASCVD risk, systolic and diastolic blood pressure, Visceral fat, Liver fat probability, and QUICKI insulin sensitivity. In some aspects the tests may be taken after fasting. In other aspects, the tests may be conducted post prandial. In some aspects, tests may be conducted both fasting and post-prandial. Fasted tests may be, for example, Glucose, HbA1c, C-peptides, Total cholesterol, HDL cholesterol, THR, triglycerides, GlycA, HDL size, and VLDL size. Post-prandial tests may be for example, glucose, glucose iAUC, Total cholesterol, HDL cholesterol, triglycerides, GlycA, HDL size, VLDL size, and CGM variation. Nutritional markers (diet indices): may include aMed, HEI, HFD, hPDI, PDI, and uPDI as described in further detail in Asnicar.

For both types of microbe lists, the top 60 microbes positively (negatively) correlated with good health outcomes (and healthy diet) were identified, and in the following these are referred to as the ‘good bugs’ (‘bad bugs’) though other subsets may also be used.

Having provided in this disclosure specific individual microbes and sets of microbes associated with and/or linked to poor health and others associated with and/or linked to pro-health conditions, profiles can now be detected without needing to sequence or otherwise assay the entire microbiome of the subject. For instance, the following are pro-health linked/indicator microbes (“good bugs”): SGB15249, SGB6340, SGB4964, SGB14252, SGB15229, SGB6174 group, SGB15317, SGB14179, SGB15225, SGB4894, SGB4643, SGB4963, SGB79840, SGB4893, SGB6276, SGB3952, SGB4638, SGB15236, SGB4191, SGB15053 group, SGB15368, SGB4782, SGB14042, SGB4706, SGB4644, SGB49188, SGB4781, SGB4777, SGB14921, SGB15234, SGB8601, SGB5087, SGB14311, SGB4953, SGB7258, SGB4882, SGB6367, SGB15106, SGB4778, SGB15131, SGB4198 group, SGB15031, SGB13981, SGB15123, SGB54300, SGB4665, SGB13979, SGB15410, SGB2290, SGB14954, SGB14306, SGB4805, SGB14899, SGB4803, SGB13982, SGB15265 group, SGB14114, SGB47656, SGB6749, SGB14253, SGB15346, SGB4810, SGB4770, SGB25497, SGB4957, SGB4654, SGB15373, SGB15254, SGB15323, SGB71759, SGB15180, SGB49168, SGB15051, SGB15145, SGB4966, SGB4780, SGB15291, SGB4816, and SGB4714; and the following are poor health linked/indicator microbes (“bad bugs”): SGB7253, SGB6769, SGB4721, SGB14837, SGB14546 group, SGB4763, SGB5193, SGB7985, SGB4699, SGB19850 group, SGB17137, SGB4785, SGB15078, SGB53821, SGB15452, SGB15271, SGB4724, SGB8255 group, SGB79823, SGB8028 group, SGB4573 group, SGB14874, SGB4988, SGB5184, SGB4786, SGB4826 group, SGB4041, SGB7984, SGB4761, SGB4447, SGB6744, SGB1836 group, SGB1814, SGB4630 group, SGB8163, SGB4617, SGB4588 group, SGB4742, SGB4572, SGB10115, SGB15158, SGB29328, SGB4791, SGB4688, SGB10068, SGB71883, SGB4760, SGB4037 group, SGB4529, SGB4837 group, SGB79883, SGB4762, SGB4797, SGB14809, SGB4758 group, SGB4703, SGB4606, SGB4584, SGB15132, SGB4746, SGB4862, SGB4798, SGB4861, SGB4608, SGB4035, SGB4794 group, SGB4753, and SGB4583. These strains are identified by their respective SGB designation, as shown in Table 1B (GOOD BUGS) and Table 1C (BAD BUGS). Additional specific taxonomic information can be found, for instance, using MetaPhlAn 4 (Metagenomic Phylogenetic Analysis; version 4 and marker database release vJan21_CHOCOPhlAnSGB_202103; Blanco-Miguez z et al., Nat Biotechnol, 2023, doi.org/10.1038/s41587-023-01688-w).

In the analyses described herein, microbes are primarily identified by a Species-level genome bin (SGB) number, e.g., SGB6340. Species-level genome bins are clusters of similar genomes, described by Segata Lab of Computational Metagenomics, University of Trento (available online at segatalab.cibio.unitn.it/data/Pasolli_et_al.html). Some SGBs correspond 1-1 with a known species. Some known species sub-divide into multiple SGBs. Some SGBs are for species which are not yet known.

SGBs are defined in Pasolli et al. (Cell 176 (3): P659-662.E20, 2019; doi.org/10.1016/j.cell.2019.01.001). In brief, all genomes are organized into clusters (SGBs) based on their full-genome genomic distance (ANI, >=95%). SGB clusters with a reference genome are considered as ‘known’ (kSGBs) and SGB clusters of only computationally reconstructed genomes are considered as ‘unknown’ (uSGBs). SGBs are further clustered into genus-level genome bins (GGBs) and family-level genomes bins (FGBs) based on ANI>=85% and >=70%, respectively. This is also exploited to consider taxonomic labels of known genomes down-to either the genus or family level and propagated to all SGBs clustered in the same GGB or FGB cluster.

SGBs can be profiled using MetaPhlAn 4 (Metagenomic Phylogenetic Analysis; Blanco-Míguez et al., Nat Biotechnol, 2023, doi.org/10.1038/s41587-023-01688-w) that uses in its database some small pieces of unique genomic sequences that allows for the accurate identifications of the SGBs. The MetaPhlAn version used to profile microbiome samples described herein is “4.beta.3”; the SGBs database version used is “vJan21_CHOCOPhlAnSGB_202103”. In some instances, including sometimes under the strict threshold of 95% Average Nucleotide Identity (ANI), there can be a large SGB cluster surrounded by multiple small SGB clusters. In this circumstance, definition unique DNA piece(s) to allow their identifications may be difficult as the task of identifying these unique DNA pieces is hampered by the presence of these small SGB clusters. Hence, for these instances, the large SGB cluster is labeled as “group” and the closets smaller SGB clusters will be considered together with the big SGB cluster when searching for the unique DNA pieces.

The tables below provide lists of microbes including the “Good Bug” SGBs and “Bad Bug” SGBs. Table 1A is a ranked list of 661 microbes as evaluated by health alone and by diet alone. Tables 1B and 1C provide the union of the top/bottom microbes from the microbe rankings as evaluated by health with and without diet; Table 1B shows the 79 collective “GOOD BUG” SGBs, and Table 1C shows the 68 collective “BAD BUG” SGBs. These tables show the respective rank of each listed microbe, along with its identification based on SGB designation (first column); the “group” designation is discussed above. “Known/Unknown” indicates whether the assigned SGB is defined as ‘known’ (k) or ‘unknown’ (u); this reflects whether such a defined cluster represents an already known species or it is only represented by computationally reconstructed genomes. The “Level taxonomy” column reports the level to which the taxonomy is assigned. For kSGB this will always be “Species” because (being known) there is a reference genome with a species taxonomic label assigned to it. For categorization “uSGB”, the taxonomy level instead can be one of “Genus”, “Family”, or “Other”; this represents how close a known SGB is to that uSGB. The final column “species label” reports an informal way to quickly identify the SGBs, which may or may not correspond to a previously recognized taxonomic species.

TABLE 1A
Identification and Ranking of Spectrum
of Good, Bad, and Neutral microbes

| | Health | Diet |
|---|---|---|
| SGB | Rank | Rank |
| SGB15249 | 0.013 | 0.218 |
| SGB6340 | 0.014 | 0.094 |
| SGB4964 | 0.036 | 0.095 |
| SGB14252 | 0.047 | 0.233 |
| SGB15229 | 0.052 | 0.281 |
| SGB6174_group | 0.057 | 0.247 |
| SGB15317 | 0.059 | 0.188 |
| SGB14179 | 0.071 | 0.290 |
| SGB15225 | 0.072 | 0.169 |
| SGB4894 | 0.072 | 0.197 |
| SGB4643 | 0.072 | 0.142 |
| SGB4963 | 0.073 | 0.145 |
| SGB79840 | 0.073 | 0.180 |
| SGB4893 | 0.075 | 0.126 |
| SGB6276 | 0.079 | 0.280 |
| SGB3952 | 0.089 | 0.375 |
| SGB4638 | 0.089 | 0.280 |
| SGB15236 | 0.093 | 0.246 |
| SGB4191 | 0.094 | 0.227 |
| SGB15053_group | 0.099 | 0.180 |
| SGB15368 | 0.101 | 0.235 |
| SGB4782 | 0.103 | 0.127 |
| SGB14042 | 0.103 | 0.196 |
| SGB4706 | 0.109 | 0.084 |
| SGB4644 | 0.116 | 0.262 |
| SGB49188 | 0.116 | 0.445 |
| SGB4781 | 0.119 | 0.200 |
| SGB4777 | 0.119 | 0.056 |
| SGB14921 | 0.121 | 0.285 |
| SGB15234 | 0.124 | 0.404 |
| SGB8601 | 0.128 | 0.201 |
| SGB5087 | 0.129 | 0.184 |
| SGB14311 | 0.131 | 0.493 |
| SGB4953 | 0.132 | 0.229 |
| SGB7258 | 0.132 | 0.116 |
| SGB4882 | 0.132 | 0.152 |
| SGB6367 | 0.133 | 0.163 |
| SGB15106 | 0.134 | 0.243 |
| SGB4778 | 0.140 | 0.106 |
| SGB15131 | 0.142 | 0.461 |
| SGB4198_group | 0.144 | 0.585 |
| SGB15031 | 0.150 | 0.428 |
| SGB13981 | 0.154 | 0.220 |
| SGB15123 | 0.162 | 0.282 |
| SGB54300 | 0.164 | 0.397 |
| SGB4665 | 0.166 | 0.138 |
| SGB13979 | 0.166 | 0.216 |
| SGB15410 | 0.169 | 0.215 |
| SGB2290 | 0.169 | 0.314 |
| SGB14954 | 0.170 | 0.274 |
| SGB14306 | 0.173 | 0.263 |
| SGB4805 | 0.173 | 0.604 |
| SGB14899 | 0.174 | 0.759 |
| SGB4803 | 0.174 | 0.491 |
| SGB13982 | 0.177 | 0.364 |
| SGB15265_group | 0.177 | 0.289 |
| SGB14114 | 0.180 | 0.452 |
| SGB47656 | 0.180 | 0.189 |
| SGB6749 | 0.182 | 0.192 |
| SGB14253 | 0.183 | 0.320 |
| SGB15346 | 0.184 | 0.244 |
| SGB4810 | 0.189 | 0.129 |
| SGB4770 | 0.190 | 0.320 |
| SGB14043 | 0.191 | 0.401 |
| SGB4886 | 0.192 | 0.464 |
| SGB25497 | 0.192 | 0.224 |
| SGB4815_group | 0.194 | 0.372 |
| SGB25416 | 0.194 | 0.657 |
| SGB4957 | 0.195 | 0.204 |
| SGB4654 | 0.196 | 0.144 |
| SGB15373 | 0.199 | 0.257 |
| SGB15254 | 0.205 | 0.118 |
| SGB6571 | 0.205 | 0.293 |
| SGB15323 | 0.207 | 0.355 |
| SGB71759 | 0.207 | 0.164 |
| SGB4648 | 0.207 | 0.388 |
| SGB15180 | 0.209 | 0.165 |
| SGB15413 | 0.209 | 0.343 |
| SGB49168 | 0.212 | 0.372 |
| SGB14960 | 0.215 | 0.346 |
| SGB4133 | 0.215 | 0.451 |
| SGB15051 | 0.218 | 0.229 |
| SGB4993 | 0.218 | 0.513 |
| SGB15395 | 0.220 | 0.566 |
| SGB15145 | 0.223 | 0.257 |
| SGB5111 | 0.223 | 0.147 |
| SGB6317 | 0.224 | 0.467 |
| SGB4966 | 0.224 | 0.224 |
| SGB4780 | 0.226 | 0.200 |
| SGB14198 | 0.227 | 0.238 |
| SGB63101 | 0.228 | 0.501 |
| SGB4779 | 0.230 | 0.471 |
| SGB15233 | 0.233 | 0.325 |
| SGB4769 | 0.234 | 0.250 |
| SGB2295 | 0.238 | 0.383 |
| SGB72336 | 0.240 | 0.609 |
| SGB4658 | 0.241 | 0.170 |
| SGB14770 | 0.245 | 0.446 |
| SGB6148 | 0.247 | 0.603 |
| SGB25493 | 0.247 | 0.301 |
| SGB4831_group | 0.248 | 0.424 |
| SGB14965 | 0.249 | 0.584 |
| SGB15224 | 0.250 | 0.332 |
| SGB4938 | 0.251 | 0.410 |
| SGB15402 | 0.252 | 0.633 |
| SGB15291 | 0.253 | 0.087 |
| SGB9333 | 0.254 | 0.209 |
| SGB4664 | 0.254 | 0.066 |
| SGB4906 | 0.254 | 0.216 |
| SGB4711 | 0.254 | 0.202 |
| SGB15065 | 0.256 | 0.177 |
| SGB714_group | 0.256 | 0.466 |
| SGB4772 | 0.257 | 0.285 |
| SGB3958 | 0.257 | 0.202 |
| SGB4629 | 0.258 | 0.212 |
| SGB14048 | 0.261 | 0.365 |
| SGB15052 | 0.263 | 0.369 |
| SGB14861 | 0.265 | 0.722 |
| SGB9205 | 0.265 | 0.446 |
| SGB4280 | 0.265 | 0.424 |
| SGB4829 | 0.265 | 0.244 |
| SGB4816 | 0.266 | 0.062 |
| SGB2317 | 0.266 | 0.612 |
| SGB15411 | 0.266 | 0.239 |
| SGB5117 | 0.267 | 0.315 |
| SGB14250 | 0.268 | 0.230 |
| SGB14924 | 0.269 | 0.338 |
| SGB4767 | 0.270 | 0.171 |
| SGB6376 | 0.271 | 0.226 |
| SGB4714 | 0.271 | 0.128 |
| SGB4691 | 0.274 | 0.292 |
| SGB14341 | 0.275 | 0.433 |
| SGB15244 | 0.275 | 0.156 |
| SGB5082_group | 0.276 | 0.115 |
| SGB4910 | 0.277 | 0.431 |
| SGB4914 | 0.277 | 0.232 |
| SGB8599 | 0.279 | 0.275 |
| SGB4936 | 0.282 | 0.138 |
| SGB15374 | 0.284 | 0.290 |
| SGB72916 | 0.284 | 0.298 |
| SGB4909 | 0.286 | 0.180 |
| SGB15390 | 0.287 | 0.457 |
| SGB15164 | 0.287 | 0.642 |
| SGB15093 | 0.289 | 0.359 |
| SGB13983 | 0.291 | 0.452 |
| SGB5042 | 0.292 | 0.425 |
| SGB4771 | 0.293 | 0.194 |
| SGB15356 | 0.295 | 0.114 |
| SGB72479 | 0.296 | 0.247 |
| SGB4557 | 0.296 | 0.218 |
| SGB3988 | 0.297 | 0.525 |
| SGB15041 | 0.298 | 0.265 |
| SGB14128 | 0.298 | 0.427 |
| SGB15385 | 0.299 | 0.296 |
| SGB6750 | 0.300 | 0.287 |
| SGB4184 | 0.302 | 0.331 |
| SGB3573 | 0.303 | 0.562 |
| SGB66170 | 0.304 | 0.680 |
| SGB15201 | 0.305 | 0.653 |
| SGB15203 | 0.306 | 0.239 |
| SGB79798 | 0.307 | 0.574 |
| SGB15382 | 0.309 | 0.533 |
| SGB4652 | 0.310 | 0.300 |
| SGB9346 | 0.311 | 0.343 |
| SGB14969 | 0.311 | 0.191 |
| SGB4262 | 0.313 | 0.440 |
| SGB4394 | 0.313 | 0.248 |
| SGB61601 | 0.314 | 0.529 |
| SGB15216 | 0.317 | 0.468 |
| SGB14027 | 0.318 | 0.486 |
| SGB4674 | 0.319 | 0.708 |
| SGB14937 | 0.320 | 0.504 |
| SGB15090 | 0.321 | 0.262 |
| SGB9391 | 0.323 | 0.219 |
| SGB15383 | 0.324 | 0.231 |
| SGB29347 | 0.324 | 0.693 |
| SGB14991 | 0.324 | 0.311 |
| SGB14940 | 0.324 | 0.360 |
| SGB4809 | 0.326 | 0.440 |
| SGB6141 | 0.327 | 0.384 |
| SGB4687 | 0.332 | 0.370 |
| SGB63163 | 0.333 | 0.607 |
| SGB14177 | 0.334 | 0.407 |
| SGB4832 | 0.334 | 0.244 |
| SGB15160 | 0.335 | 0.234 |
| SGB48024 | 0.335 | 0.237 |
| SGB6179 | 0.336 | 0.460 |
| SGB4768 | 0.342 | 0.308 |
| SGB5090_group | 0.343 | 0.177 |
| SGB29302 | 0.345 | 0.701 |
| SGB9712_group | 0.345 | 0.353 |
| SGB3813 | 0.346 | 0.423 |
| SGB79833 | 0.346 | 0.273 |
| SGB4659 | 0.350 | 0.256 |
| SGB4328 | 0.350 | 0.283 |
| SGB4776 | 0.350 | 0.722 |
| SGB1790 | 0.351 | 0.783 |
| SGB14313 | 0.351 | 0.315 |
| SGB5043 | 0.351 | 0.364 |
| SGB15127 | 0.352 | 0.626 |
| SGB15049 | 0.352 | 0.229 |
| SGB42321 | 0.353 | 0.453 |
| SGB15403 | 0.353 | 0.592 |
| SGB15115 | 0.363 | 0.191 |
| SGB4905 | 0.363 | 0.186 |
| SGB14838 | 0.363 | 0.878 |
| SGB15012 | 0.365 | 0.189 |
| SGB9202 | 0.368 | 0.350 |
| SGB80143 | 0.369 | 0.144 |
| SGB3992 | 0.370 | 0.757 |
| SGB7259 | 0.372 | 0.625 |
| SGB4546 | 0.373 | 0.341 |
| SGB14974 | 0.373 | 0.188 |
| SGB13976 | 0.374 | 0.613 |
| SGB15342 | 0.376 | 0.529 |
| SGB2296 | 0.377 | 0.484 |
| SGB14941 | 0.377 | 0.493 |
| SGB3996 | 0.379 | 0.508 |
| SGB53497 | 0.380 | 0.739 |
| SGB15470 | 0.381 | 0.727 |
| SGB14020 | 0.381 | 0.427 |
| SGB1858 | 0.382 | 0.342 |
| SGB14851 | 0.386 | 0.919 |
| SGB6305 | 0.388 | 0.439 |
| SGB14932 | 0.388 | 0.636 |
| SGB15089 | 0.389 | 0.577 |
| SGB1862 | 0.390 | 0.417 |
| SGB15401 | 0.392 | 0.680 |
| SGB4027 | 0.392 | 0.246 |
| SGB15140 | 0.392 | 0.455 |
| SGB2325 | 0.393 | 0.433 |
| SGB14317 | 0.393 | 0.347 |
| SGB4628 | 0.394 | 0.626 |
| SGB4669 | 0.395 | 0.216 |
| SGB15299 | 0.395 | 0.442 |
| SGB6478 | 0.395 | 0.393 |
| SGB14262 | 0.397 | 0.521 |
| SGB63342 | 0.399 | 0.536 |
| SGB4960 | 0.400 | 0.178 |
| SGB63333 | 0.400 | 0.365 |
| SGB15316_group | 0.401 | 0.213 |
| SGB4651 | 0.404 | 0.284 |
| SGB1965 | 0.404 | 0.452 |
| SGB15081 | 0.406 | 0.238 |
| SGB59819 | 0.408 | 0.608 |
| SGB2326 | 0.410 | 0.391 |
| SGB14912 | 0.413 | 0.444 |
| SGB14322_group | 0.416 | 0.244 |
| SGB3940 | 0.416 | 0.319 |
| SGB4029 | 0.417 | 0.686 |
| SGB2301 | 0.419 | 0.684 |
| SGB63167 | 0.421 | 0.556 |
| SGB14797_group | 0.422 | 0.353 |
| SGB5200 | 0.428 | 0.577 |
| SGB17347 | 0.431 | 0.377 |
| SGB4868 | 0.432 | 0.667 |
| SGB15067 | 0.433 | 0.452 |
| SGB53515 | 0.434 | 0.469 |
| SGB15075 | 0.435 | 0.359 |
| SGB4421 | 0.436 | 0.380 |
| SGB5121 | 0.437 | 0.131 |
| SGB9226 | 0.438 | 0.372 |
| SGB2318 | 0.439 | 0.773 |
| SGB14894 | 0.441 | 0.815 |
| SGB4817 | 0.443 | 0.257 |
| SGB14966 | 0.443 | 0.278 |
| SGB3989 | 0.444 | 0.310 |
| SGB15370 | 0.444 | 0.201 |
| SGB14975 | 0.444 | 0.559 |
| SGB4436 | 0.446 | 0.416 |
| SGB14839 | 0.446 | 0.561 |
| SGB14993_group | 0.451 | 0.127 |
| SGB15322 | 0.452 | 0.403 |
| SGB9387 | 0.454 | 0.454 |
| SGB3959 | 0.454 | 0.426 |
| SGB6362 | 0.455 | 0.459 |
| SGB4063 | 0.455 | 0.626 |
| SGB14773_group | 0.455 | 0.273 |
| SGB29334 | 0.456 | 0.398 |
| SGB14151 | 0.457 | 0.586 |
| SGB15087 | 0.457 | 0.419 |
| SGB14022 | 0.458 | 0.526 |
| SGB14972 | 0.459 | 0.166 |
| SGB15045 | 0.459 | 0.400 |
| SGB4712 | 0.460 | 0.209 |
| SGB15389 | 0.463 | 0.518 |
| SGB82503 | 0.468 | 0.741 |
| SGB15318_group | 0.469 | 0.479 |
| SGB1857 | 0.470 | 0.412 |
| SGB4828_group | 0.470 | 0.333 |
| SGB4788_group | 0.470 | 0.170 |
| SGB79822 | 0.471 | 0.344 |
| SGB4269 | 0.473 | 0.210 |
| SGB14923 | 0.473 | 0.622 |
| SGB2328 | 0.474 | 0.625 |
| SGB1784 | 0.474 | 0.350 |
| SGB14137 | 0.475 | 0.686 |
| SGB15204 | 0.478 | 0.489 |
| SGB7256 | 0.485 | 0.698 |
| SGB15295_group | 0.485 | 0.191 |
| SGB14182 | 0.486 | 0.341 |
| SGB29342 | 0.488 | 0.763 |
| SGB4438 | 0.488 | 0.614 |
| SGB14824_group | 0.489 | 0.260 |
| SGB2303 | 0.489 | 0.620 |
| SGB9262 | 0.489 | 0.309 |
| SGB14952 | 0.490 | 0.700 |
| SGB14951 | 0.491 | 0.542 |
| SGB15459 | 0.493 | 0.591 |
| SGB6358 | 0.494 | 0.271 |
| SGB14929 | 0.494 | 0.267 |
| SGB15286 | 0.495 | 0.604 |
| SGB5060 | 0.495 | 0.345 |
| SGB15332_group | 0.497 | 0.356 |
| SGB15126 | 0.497 | 0.554 |
| SGB72433_group | 0.498 | 0.297 |
| SGB4045 | 0.498 | 0.498 |
| SGB1626 | 0.499 | 0.579 |
| SGB5180 | 0.499 | 0.422 |
| SGB4867 | 0.499 | 0.426 |
| SGB4825 | 0.506 | 0.108 |
| SGB4925 | 0.506 | 0.397 |
| SGB53517 | 0.507 | 0.679 |
| SGB1844 | 0.508 | 0.335 |
| SGB14844 | 0.515 | 0.747 |
| SGB4871_group | 0.516 | 0.289 |
| SGB14050 | 0.518 | 0.460 |
| SGB25547 | 0.519 | 0.683 |
| SGB1815 | 0.519 | 0.292 |
| SGB3957 | 0.519 | 0.631 |
| SGB54347 | 0.519 | 0.729 |
| SGB6140 | 0.520 | 0.638 |
| SGB14127 | 0.521 | 0.893 |
| SGB29339 | 0.524 | 0.752 |
| SGB1832 | 0.524 | 0.315 |
| SGB9342_group | 0.524 | 0.619 |
| SGB15278 | 0.528 | 0.413 |
| SGB9203 | 0.528 | 0.395 |
| SGB1962 | 0.529 | 0.391 |
| SGB5076 | 0.531 | 0.315 |
| SGB4808 | 0.532 | 0.227 |
| SGB15119 | 0.532 | 0.157 |
| SGB3962 | 0.535 | 0.795 |
| SGB14892 | 0.536 | 0.778 |
| SGB4775 | 0.536 | 0.837 |
| SGB4166 | 0.537 | 0.534 |
| SGB7144 | 0.537 | 0.532 |
| SGB4959 | 0.538 | 0.562 |
| SGB63353 | 0.539 | 0.640 |
| SGB14953 | 0.542 | 0.668 |
| SGB6139 | 0.545 | 0.447 |
| SGB16971 | 0.545 | 0.295 |
| SGB15300 | 0.545 | 0.463 |
| SGB3574 | 0.545 | 0.847 |
| SGB47850 | 0.545 | 0.143 |
| SGB63327 | 0.545 | 0.896 |
| SGB4181 | 0.547 | 0.652 |
| SGB15506 | 0.547 | 0.537 |
| SGB5190 | 0.548 | 0.272 |
| SGB14181 | 0.548 | 0.605 |
| SGB1846 | 0.548 | 0.435 |
| SGB15068 | 0.549 | 0.432 |
| SGB4537 | 0.550 | 0.367 |
| SGB14906 | 0.551 | 0.466 |
| SGB9347 | 0.551 | 0.315 |
| SGB1786 | 0.551 | 0.433 |
| SGB7265 | 0.553 | 0.776 |
| SGB4571 | 0.556 | 0.375 |
| SGB29321 | 0.557 | 0.599 |
| SGB4531 | 0.557 | 0.824 |
| SGB15073 | 0.560 | 0.372 |
| SGB14933 | 0.562 | 0.424 |
| SGB15154 | 0.564 | 0.549 |
| SGB6747 | 0.565 | 0.794 |
| SGB15273 | 0.568 | 0.723 |
| SGB4367 | 0.569 | 0.530 |
| SGB15091 | 0.572 | 0.431 |
| SGB3965 | 0.573 | 0.251 |
| SGB63369 | 0.574 | 0.666 |
| SGB9224 | 0.575 | 0.648 |
| SGB3964 | 0.575 | 0.618 |
| SGB6952 | 0.580 | 0.503 |
| SGB4765 | 0.581 | 0.270 |
| SGB29305 | 0.581 | 0.640 |
| SGB4285_group | 0.581 | 0.627 |
| SGB5089 | 0.584 | 0.400 |
| SGB4581 | 0.584 | 0.649 |
| SGB59869 | 0.586 | 0.628 |
| SGB4727 | 0.587 | 0.700 |
| SGB25431 | 0.591 | 0.664 |
| SGB2299 | 0.592 | 0.320 |
| SGB1829 | 0.593 | 0.495 |
| SGB4990 | 0.597 | 0.600 |
| SGB1891_group | 0.597 | 0.475 |
| SGB2286 | 0.600 | 0.333 |
| SGB17278 | 0.601 | 0.141 |
| SGB1949 | 0.602 | 0.867 |
| SGB63343 | 0.602 | 0.697 |
| SGB5045 | 0.603 | 0.536 |
| SGB5075_group | 0.603 | 0.571 |
| SGB8007_group | 0.604 | 0.641 |
| SGB29375 | 0.605 | 0.315 |
| SGB6847 | 0.606 | 0.667 |
| SGB1798 | 0.607 | 0.401 |
| SGB4670 | 0.609 | 0.218 |
| SGB4582_group | 0.610 | 0.299 |
| SGB4290 | 0.612 | 0.527 |
| SGB14891 | 0.612 | 0.596 |
| SGB1785 | 0.612 | 0.329 |
| SGB15209 | 0.614 | 0.693 |
| SGB14150 | 0.620 | 0.718 |
| SGB33551 | 0.620 | 0.227 |
| SGB14958 | 0.621 | 0.653 |
| SGB14807 | 0.622 | 0.568 |
| SGB4820 | 0.624 | 0.556 |
| SGB1812 | 0.624 | 0.460 |
| SGB4716 | 0.627 | 0.281 |
| SGB4425_group | 0.627 | 0.461 |
| SGB9340 | 0.628 | 0.377 |
| SGB14779 | 0.629 | 0.716 |
| SGB14125 | 0.630 | 0.634 |
| SGB14259 | 0.631 | 0.567 |
| SGB4784 | 0.631 | 0.862 |
| SGB15260 | 0.633 | 0.369 |
| SGB14741 | 0.634 | 0.821 |
| SGB4577_group | 0.635 | 0.640 |
| SGB71281 | 0.637 | 0.484 |
| SGB14307 | 0.638 | 0.640 |
| SGB5803 | 0.638 | 0.460 |
| SGB58519 | 0.641 | 0.607 |
| SGB5077 | 0.643 | 0.523 |
| SGB15125 | 0.645 | 0.851 |
| SGB15143 | 0.647 | 0.683 |
| SGB4116 | 0.649 | 0.912 |
| SGB4677 | 0.652 | 0.804 |
| SGB15467_group | 0.654 | 0.523 |
| SGB7202 | 0.655 | 0.351 |
| SGB6178 | 0.656 | 0.686 |
| SGB6796_group | 0.656 | 0.586 |
| SGB6783_group | 0.658 | 0.600 |
| SGB1860 | 0.658 | 0.617 |
| SGB4059 | 0.659 | 0.529 |
| SGB63326 | 0.659 | 0.875 |
| SGB9272_group | 0.659 | 0.638 |
| SGB15350 | 0.659 | 0.517 |
| SGB14334 | 0.660 | 0.599 |
| SGB1941 | 0.665 | 0.461 |
| SGB16986 | 0.667 | 0.667 |
| SGB14909 | 0.668 | 0.768 |
| SGB1963 | 0.669 | 0.507 |
| SGB4774 | 0.671 | 0.617 |
| SGB14143 | 0.671 | 0.810 |
| SGB15272 | 0.671 | 0.729 |
| SGB29380 | 0.672 | 0.898 |
| SGB4811_group | 0.675 | 0.695 |
| SGB4595 | 0.676 | 0.524 |
| SGB4834 | 0.676 | 0.184 |
| SGB4030 | 0.679 | 0.677 |
| SGB8071 | 0.679 | 0.772 |
| SGB2311 | 0.680 | 0.470 |
| SGB3991 | 0.680 | 0.663 |
| SGB4722 | 0.681 | 0.685 |
| SGB17244 | 0.682 | 0.536 |
| SGB3993 | 0.682 | 0.648 |
| SGB4553 | 0.683 | 0.137 |
| SGB6956 | 0.683 | 0.544 |
| SGB1699 | 0.689 | 0.497 |
| SGB6962_group | 0.691 | 0.796 |
| SGB4705 | 0.691 | 0.569 |
| SGB1957 | 0.693 | 0.295 |
| SGB4532 | 0.694 | 0.410 |
| SGB4327_group | 0.695 | 0.450 |
| SGB59559 | 0.698 | 0.803 |
| SGB4594 | 0.699 | 0.683 |
| SGB5765_group | 0.700 | 0.518 |
| SGB17169 | 0.702 | 0.672 |
| SGB15120 | 0.705 | 0.710 |
| SGB1948 | 0.705 | 0.421 |
| SGB14853 | 0.706 | 0.928 |
| SGB14898 | 0.706 | 0.756 |
| SGB48424 | 0.707 | 0.720 |
| SGB5792 | 0.707 | 0.619 |
| SGB6754 | 0.707 | 0.241 |
| SGB1934 | 0.708 | 0.730 |
| SGB14854 | 0.709 | 0.743 |
| SGB6768 | 0.709 | 0.858 |
| SGB15156_group | 0.710 | 0.710 |
| SGB4750 | 0.711 | 0.599 |
| SGB14142 | 0.711 | 0.877 |
| SGB17153_group | 0.712 | 0.787 |
| SGB4552_group | 0.712 | 0.329 |
| SGB4951 | 0.716 | 0.541 |
| SGB4303 | 0.716 | 0.355 |
| SGB9286 | 0.720 | 0.504 |
| SGB5051 | 0.725 | 0.491 |
| SGB17237 | 0.726 | 0.571 |
| SGB4940 | 0.726 | 0.624 |
| SGB4597 | 0.727 | 0.668 |
| SGB4563_group | 0.731 | 0.793 |
| SGB1867 | 0.732 | 0.602 |
| SGB47515 | 0.733 | 0.863 |
| SGB59562 | 0.733 | 0.700 |
| SGB8059_group | 0.739 | 0.631 |
| SGB4991 | 0.740 | 0.780 |
| SGB4540_group | 0.741 | 0.428 |
| SGB14862 | 0.743 | 0.683 |
| SGB4422 | 0.744 | 0.560 |
| SGB15124 | 0.745 | 0.831 |
| SGB14987 | 0.747 | 0.519 |
| SGB7263 | 0.749 | 0.839 |
| SGB9283 | 0.751 | 0.600 |
| SGB4348 | 0.757 | 0.520 |
| SGB14895 | 0.758 | 0.910 |
| SGB17154 | 0.759 | 0.768 |
| SGB17167 | 0.759 | 0.664 |
| SGB4626 | 0.759 | 0.641 |
| SGB5843 | 0.762 | 0.529 |
| SGB4575 | 0.763 | 0.499 |
| SGB4613 | 0.765 | 0.793 |
| SGB15076 | 0.771 | 0.734 |
| SGB17130 | 0.772 | 0.620 |
| SGB15904 | 0.774 | 0.585 |
| SGB4080 | 0.775 | 0.814 |
| SGB6846 | 0.777 | 0.499 |
| SGB5825_group | 0.778 | 0.675 |
| SGB14808 | 0.779 | 0.618 |
| SGB4874 | 0.779 | 0.502 |
| SGB9228 | 0.782 | 0.625 |
| SGB6320 | 0.783 | 0.711 |
| SGB9260 | 0.784 | 0.656 |
| SGB8002 | 0.785 | 0.546 |
| SGB6936 | 0.787 | 0.522 |
| SGB14962 | 0.787 | 0.905 |
| SGB8053 | 0.788 | 0.897 |
| SGB4701 | 0.788 | 0.785 |
| SGB5197 | 0.789 | 0.484 |
| SGB4036 | 0.790 | 0.860 |
| SGB4744 | 0.793 | 0.805 |
| SGB1877 | 0.794 | 0.751 |
| SGB29313 | 0.799 | 0.698 |
| SGB14845 | 0.799 | 0.701 |
| SGB14963 | 0.799 | 0.856 |
| SGB8056 | 0.801 | 0.745 |
| SGB6939 | 0.803 | 0.644 |
| SGB17256 | 0.804 | 0.710 |
| SGB4749 | 0.804 | 0.428 |
| SGB3961 | 0.805 | 0.683 |
| SGB7142 | 0.805 | 0.541 |
| SGB14999 | 0.805 | 0.549 |
| SGB4747 | 0.806 | 0.813 |
| SGB15149 | 0.807 | 0.641 |
| SGB4987 | 0.807 | 0.724 |
| SGB6767 | 0.810 | 0.776 |
| SGB17248 | 0.810 | 0.657 |
| SGB4725 | 0.810 | 0.686 |
| SGB59576 | 0.810 | 0.643 |
| SGB1903_group | 0.811 | 0.301 |
| SGB5183 | 0.811 | 0.635 |
| SGB49059 | 0.815 | 0.742 |
| SGB4121 | 0.819 | 0.836 |
| SGB25538 | 0.819 | 0.707 |
| SGB3970 | 0.820 | 0.897 |
| SGB3969 | 0.822 | 0.692 |
| SGB14890 | 0.822 | 0.767 |
| SGB1830_group | 0.824 | 0.578 |
| SGB7967 | 0.824 | 0.341 |
| SGB17168 | 0.825 | 0.650 |
| SGB8047 | 0.828 | 0.839 |
| SGB4046 | 0.829 | 0.720 |
| SGB1855_group | 0.830 | 0.711 |
| SGB3922 | 0.830 | 0.937 |
| SGB14995 | 0.833 | 0.849 |
| SGB7253 | 0.838 | 0.841 |
| SGB48013 | 0.838 | 0.795 |
| SGB4741 | 0.841 | 0.696 |
| SGB4671 | 0.842 | 0.730 |
| SGB15878 | 0.844 | 0.901 |
| SGB6769 | 0.844 | 0.937 |
| SGB14180 | 0.844 | 0.770 |
| SGB29433 | 0.846 | 0.728 |
| SGB1871 | 0.846 | 0.624 |
| SGB5182 | 0.852 | 0.594 |
| SGB5736 | 0.853 | 0.875 |
| SGB6771 | 0.854 | 0.681 |
| SGB4721 | 0.857 | 0.841 |
| SGB14837 | 0.858 | 0.865 |
| SGB4044 | 0.861 | 0.685 |
| SGB8095 | 0.862 | 0.769 |
| SGB17152 | 0.863 | 0.592 |
| SGB1861 | 0.864 | 0.486 |
| SGB66069 | 0.865 | 0.898 |
| SGB4031 | 0.869 | 0.642 |
| SGB6153 | 0.871 | 0.710 |
| SGB7264 | 0.880 | 0.464 |
| SGB15121 | 0.883 | 0.747 |
| SGB25437 | 0.884 | 0.871 |
| SGB14546_group | 0.885 | 0.843 |
| SGB4933_group | 0.888 | 0.635 |
| SGB4763 | 0.888 | 0.906 |
| SGB5193 | 0.889 | 0.961 |
| SGB7985 | 0.891 | 0.674 |
| SGB4699 | 0.892 | 0.843 |
| SGB19850_group | 0.893 | 0.569 |
| SGB17137 | 0.895 | 0.811 |
| SGB4785 | 0.897 | 0.835 |
| SGB15078 | 0.898 | 0.904 |
| SGB53821 | 0.898 | 0.828 |
| SGB15452 | 0.899 | 0.839 |
| SGB15271 | 0.900 | 0.893 |
| SGB4724 | 0.902 | 0.818 |
| SGB8255_group | 0.906 | 0.857 |
| SGB79823 | 0.906 | 0.647 |
| SGB8028_group | 0.908 | 0.804 |
| SGB4573_group | 0.912 | 0.768 |
| SGB14874 | 0.913 | 0.964 |
| SGB4988 | 0.914 | 0.679 |
| SGB5184 | 0.914 | 0.894 |
| SGB4786 | 0.915 | 0.713 |
| SGB4826_group | 0.915 | 0.840 |
| SGB4041 | 0.916 | 0.918 |
| SGB7984 | 0.917 | 0.584 |
| SGB4761 | 0.919 | 0.777 |
| SGB4447 | 0.922 | 0.524 |
| SGB6744 | 0.923 | 0.757 |
| SGB1836_group | 0.926 | 0.546 |
| SGB1814 | 0.926 | 0.778 |
| SGB4630_group | 0.927 | 0.885 |
| SGB8163 | 0.930 | 0.778 |
| SGB4617 | 0.933 | 0.866 |
| SGB4588_group | 0.933 | 0.784 |
| SGB4742 | 0.937 | 0.660 |
| SGB4572 | 0.938 | 0.900 |
| SGB10115 | 0.938 | 0.731 |
| SGB15158 | 0.942 | 0.878 |
| SGB29328 | 0.943 | 0.840 |
| SGB4791 | 0.945 | 0.942 |
| SGB4688 | 0.946 | 0.901 |
| SGB10068 | 0.949 | 0.825 |
| SGB71883 | 0.957 | 0.788 |
| SGB4760 | 0.959 | 0.818 |
| SGB4037_group | 0.961 | 0.909 |
| SGB4529 | 0.964 | 0.922 |
| SGB4837_group | 0.968 | 0.781 |
| SGB79883 | 0.968 | 0.969 |
| SGB4762 | 0.968 | 0.796 |
| SGB4797 | 0.974 | 0.913 |
| SGB14809 | 0.977 | 0.872 |
| SGB4758_group | 0.978 | 0.891 |
| SGB4703 | 0.978 | 0.959 |
| SGB4606 | 0.981 | 0.874 |
| SGB4584 | 0.981 | 0.932 |
| SGB15132 | 0.981 | 0.901 |
| SGB4746 | 0.985 | 0.809 |
| SGB4862 | 0.986 | 0.893 |
| SGB4798 | 0.986 | 0.755 |
| SGB4861 | 0.989 | 0.943 |
| SGB4608 | 0.991 | 0.904 |
| SGB4035 | 0.994 | 0.968 |
| SGB4794_group | 0.996 | 0.956 |
| SGB4753 | 0.997 | 0.928 |
| SGB4583 | 1.000 | 0.965 |

TABLE 1B
Identification & Ranking of Select Pro-Health Indicator Microbes

| | | Health | | | |
|---|---|---|---|---|---|
| | | and | | | |
| | Health | Diet | Known/ | Level | |
| SGB | Rank | Rank | Unknown | taxonomy | Species Label |
| SGB15249 | 0.01295 | 0.03788 | kSGB | Species | s_{——}Ruminococcaceae_bacterium |
| SGB6340 | 0.01448 | 0.01144 | uSGB | Family | s_{——}GGB4585_SGB6340 |
| SGB4964 | 0.03551 | 0.01271 | kSGB | Species | s_{——}Lachnospiraceae_bacterium |
| SGB14252 | 0.04653 | 0.03567 | kSGB | Species | s_{——}Clostridia_bacterium |
| SGB15229 | 0.05230 | 0.04529 | uSGB | Family | s_{——}GGB9707_SGB15229 |
| SGB6174 | 0.05683 | 0.05821 | kSGB | Species | s_{——}Clostridium_sp_NSJ_42 |
| group | | | | | |
| SGB15317 | 0.05939 | 0.02016 | kSGB | Species | s_{——}Faecalibacterium_prausnitzii |
| SGB14179 | 0.07117 | 0.11379 | uSGB | Other | s_{——}GGB9237_SGB14179 |
| SGB15225 | 0.07187 | 0.07097 | uSGB | Family | s_{——}GGB9705_SGB15225 |
| SGB4894 | 0.07226 | 0.06731 | uSGB | Genus | s_{——}Lachnospiraceae_unclassified |
| | | | | | SGB4894 |
| SGB4643 | 0.07249 | 0.04395 | uSGB | Family | s_{——}GGB3478_SGB4643 |
| SGB4963 | 0.07346 | 0.05215 | kSGB | Species | s_{——}Lachnospiraceae_bacterium |
| SGB79840 | 0.07347 | 0.06468 | kSGB | Species | s_{——}Intestinimonas_gabonensis |
| SGB4893 | 0.07503 | 0.04669 | kSGB | Species | s_{——}Lachnospiraceae_bacterium |
| | | | | | OM04_12BH |
| SGB6276 | 0.07948 | 0.09152 | uSGB | Genus | s_{——}Clostridia_unclassified_SGB6276 |
| SGB3952 | 0.08883 | 0.16062 | kSGB | Species | s_{——}Clostridia_bacterium |
| SGB4638 | 0.08937 | 0.12535 | kSGB | Species | s_{——}Lachnospiraceae_bacterium |
| SGB15236 | 0.09254 | 0.11862 | uSGB | Genus | s_{——}Ruminococcaceae_unclassified |
| | | | | | SGB15236 |
| SGB4191 | 0.09427 | 0.12455 | uSGB | Genus | s_{——}Ruminococcaceae_unclassified |
| | | | | | SGB4191 |
| SGB15053 | 0.09860 | 0.07499 | uSGB | Family | s_{——}GGB9615_SGB15053 |
| group | | | | | |
| SGB15368 | 0.10061 | 0.06770 | uSGB | Family | s_{——}GGB9758_SGB15368 |
| SGB4782 | 0.10281 | 0.04146 | kSGB | Species | s_{——}Lachnospiraceae_bacterium |
| SGB14042 | 0.10312 | 0.11185 | kSGB | Species | s_{——}Clostridia_bacterium |
| SGB4706 | 0.10943 | 0.05344 | kSGB | Species | s_{——}Lachnospiraceae_bacterium |
| SGB4644 | 0.11568 | 0.10116 | kSGB | Species | s_{——}Clostridium_sp_AF36_4 |
| SGB49188 | 0.11574 | 0.23073 | kSGB | Species | s_{——}Firmicutes_bacterium |
| SGB4781 | 0.11872 | 0.11708 | kSGB | Species | s_{——}Lachnospiraceae_bacterium |
| SGB4777 | 0.11931 | 0.05191 | uSGB | Family | s_{——}GGB3570_SGB4777 |
| SGB14921 | 0.12060 | 0.16745 | uSGB | Family | s_{——}GGB9522_SGB14921 |
| SGB15234 | 0.12447 | 0.17389 | uSGB | Genus | s_{——}Ruminococcaceae_unclassified |
| | | | | | SGB15234 |
| SGB8601 | 0.12830 | 0.13841 | kSGB | Species | s_{——}Candidatus_Gastranaerophilales |
| | | | | | bacterium |
| SGB5087 | 0.12934 | 0.07516 | kSGB | Species | s_{——}Lachnospira_sp_NSJ_43 |
| SGB14311 | 0.13091 | 0.21316 | kSGB | Species | s_{——}Clostridia_bacterium |
| SGB4953 | 0.13203 | 0.06709 | kSGB | Species | s_{——}Lachnospiraceae_bacterium |
| SGB7258 | 0.13209 | 0.11251 | kSGB | Species | s_{——}Oscillibacter_sp_PC13 |
| SGB4882 | 0.13224 | 0.09517 | uSGB | Genus | s_{——}Lachnospiraceae_unclassified |
| | | | | | SGB4882 |
| SGB6367 | 0.13257 | 0.08855 | uSGB | Family | s_{——}SGGB4603_SGB6367 |
| SGB15106 | 0.13443 | 0.14450 | uSGB | Family | s_{——}GGB9635_SGB15106 |
| SGB4778 | 0.14045 | 0.08654 | uSGB | Family | s_{——}GGB3571_SGB477 |
| SGB15131 | 0.14152 | 0.25835 | kSGB | Species | s_{——}Lawsonibacter_sp_NSJ_51 |
| SGB4198 | 0.14354 | 0.25145 | kSGB | Species | s_{——}Eubacterium_siraeum |
| group | | | | | |
| SGB15031 | 0.15023 | 0.19720 | uSGB | Family | s_{——}GGB9602_SGB15031 |
| SGB13981 | 0.15359 | 0.15525 | kSGB | Species | s_{——}Clostridia_bacterium |
| SGB15123 | 0.16242 | 0.19465 | uSGB | Family | s_{——}GGB9646_SGB15123 |
| SGB54300 | 0.16384 | 0.17829 | kSGB | Species | s_{——}Clostridia_bacterium |
| SGB4665 | 0.16585 | 0.08980 | uSGB | Other | s_{——}SGGB3491_SGB4665 |
| SGB13979 | 0.16638 | 0.11780 | kSGB | Species | s_{——}Clostridia_bacterium |
| SGB15410 | 0.16878 | 0.14887 | uSGB | Family | s_{——}GGB9787_SGB15410 |
| SGB2290 | 0.16936 | 0.20167 | kSGB | Species | s_{——}Alistipes_communis |
| SGB14954 | 0.16999 | 0.23594 | kSGB | Species | s_{——}Clostridia_bacterium |
| SGB14306 | 0.17277 | 0.19560 | uSGB | Family | s_{——}SGGB9342_SGB14306 |
| SGB4805 | 0.17317 | 0.28601 | uSGB | Genus | s_{——}Blautia_SGB4805 |
| SGB14899 | 0.17381 | 0.37839 | kSGB | Species | s_{——}Ruminococcaceae_bacterium |
| SGB4803 | 0.17407 | 0.20513 | kSGB | Species | s_{——}Lachnospiraceae_bacterium |
| SGB13982 | 0.17658 | 0.21446 | kSGB | Species | s_{——}Clostridia_bacterium |
| SGB15265 | 0.17739 | 0.13848 | uSGB | Genus | s_{——}Ruminococcaceae_unclassified |
| group | | | | | SGB15265 |
| SGB14114 | 0.17952 | 0.29401 | uSGB | Family | s_{——}GGB9176_SGB14114 |
| SGB47656 | 0.17999 | 0.10019 | kSGB | Species | s_{——}Lachnospiraceae_bacterium_NSJ46 |
| SGB6749 | 0.18194 | 0.11580 | kSGB | Species | s_{——}Clostridium_saccharogumia |
| SGB14253 | 0.18296 | 0.14263 | kSGB | Species | s_{——}Clostridia_bacterium |
| SGB15346 | 0.18435 | 0.11324 | uSGB | Genus | s_{——}Faecalibacterium_SGB15346 |
| SGB4810 | 0.18853 | 0.08351 | kSGB | Species | s_{——}Blautia_sp_AF19_10LB |
| SGB4770 | 0.18992 | 0.13870 | kSGB | Species | s_{——}Clostridiaceae_bacterium |
| SGB25497 | 0.19225 | 0.11007 | kSGB | Species | s_{——}Ruminococcus_sp_AF41_9 |
| SGB4957 | 0.19498 | 0.10400 | kSGB | Species | s_{——}Lachnospiraceae_bacterium |
| SGB4654 | 0.19578 | 0.11904 | kSGB | Species | s_{——}Roseburia_sp_AM59_24XD |
| SGB15373 | 0.19896 | 0.14592 | kSGB | Species | s_{——}Clostridia_bacterium |
| SGB15254 | 0.20473 | 0.14108 | kSGB | Species | s_{——}Oscillibacter_sp_ER4 |
| SGB15323 | 0.20657 | 0.14707 | kSGB | Species | s_{——}Faecalibacterium_prausnitzii |
| SGB71759 | 0.20708 | 0.10113 | uSGB | Other | s_{——}GGB51441_SGB71759 |
| SGB15180 | 0.20880 | 0.12347 | uSGB | Family | s_{——}GGB9677_SGB15180 |
| SGB49168 | 0.21242 | 0.13318 | kSGB | Species | s_{——}Peptococcaceae_bacterium |
| SGB15051 | 0.21791 | 0.11890 | uSGB | Family | s_{——}GGB9615_SGB15051 |
| SGB15145 | 0.22275 | 0.14522 | uSGB | Genus | s_{——}Clostridiales_unclassified_SGB15145 |
| SGB4966 | 0.22442 | 0.12453 | kSGB | Species | s_{——}Lachnospiraceae_bacterium_OF096 |
| SGB4780 | 0.22571 | 0.11311 | kSGB | Species | s_{——}Lachnospiraceae_bacterium |
| SGB15291 | 0.25287 | 0.14375 | uSGB | Family | s_{——}GGB9730_SGB15291 |
| SGB4816 | 0.26564 | 0.13167 | kSGB | Species | s_{——}Blautia_glucerasea |
| SGB4714 | 0.27136 | 0.11458 | kSGB | Species | s_{——}Clostridium_sp_AF20_17LB |

TABLE 1C
Identification & Ranking of Select Poor-Health Indicator Microbes

| | | Health | | | |
|---|---|---|---|---|---|
| | | and | | | |
| | Health | Diet | Known/ | Level | |
| SGB | Rank | Rank | Unknown | taxonomy | Species Label |
| SGB7253 | 0.83757 | 0.90439 | kSGB | Species | s_{——}Massilimaliae_timonensis |
| SGB6769 | 0.84367 | 0.90635 | kSGB | Species | s_{——}Longibaculum_muris |
| SGB4721 | 0.85684 | 0.92161 | kSGB | Species | s_{——}Clostridiaceae_bacterium |
| SGB14837 | 0.85756 | 0.91502 | kSGB | Species | s_{——}Phocea_massiliensis |
| SGB14546 | 0.88488 | 0.90584 | kSGB | Species | s_{——}Collinsella_aerofaciens |
| group | | | | | |
| SGB4763 | 0.88841 | 0.90816 | kSGB | Species | s_{——}Clostridiales_bacterium_1_7_47FAA |
| SGB5193 | 0.88927 | 0.92589 | kSGB | Species | s_{——}Anaerotignum_lactatifermentans |
| SGB7985 | 0.89132 | 0.90473 | kSGB | Species | s_{——}Lactococcus_lactis |
| SGB4699 | 0.89204 | 0.91691 | kSGB | Species | s_{——}Clostridium_symbiosum |
| SGB19850 | 0.89309 | 0.86880 | uSGB | Genus | s_{——}Candidatus_Saccharibacteria |
| group | | | | | unclassified_SGB19850 |
| SGB17137 | 0.89514 | 0.89471 | kSGB | Species | s_{——}Trueperella_pyogenes |
| SGB4785 | 0.89737 | 0.90411 | kSGB | Species | s_{——}Blautia_producta |
| SGB15078 | 0.89839 | 0.94997 | kSGB | Species | s_{——}Dysosmobacter_welbionis |
| SGB53821 | 0.89842 | 0.90151 | kSGB | Species | s_{——}Ruminococcaceae_bacterium |
| SGB15452 | 0.89858 | 0.93212 | kSGB | Species | s_{——}Bilophila_wadsworthia |
| SGB15271 | 0.90009 | 0.94777 | kSGB | Species | s_{——}Ruthenibacterium_lactatiformans |
| SGB4724 | 0.90225 | 0.90878 | kSGB | Species | s_{——}Enterocloster_asparagiformis |
| SGB8255 | 0.90607 | 0.90838 | kSGB | Species | s_{——}Streptococcus_sp_263_SSPC |
| group | | | | | |
| SGB79823 | 0.90629 | 0.85359 | uSGB | Other | s_{——}SGGB9581_SGB79823 |
| SGB8028 | 0.90753 | 0.90718 | kSGB | Species | s_{——}Streptococcus_anginosus |
| group | | | | | |
| SGB4573 | 0.91196 | 0.94165 | uSGB | Family | s_{——}GGB3433_SGB4573 |
| group | | | | | |
| SGB14874 | 0.91337 | 0.94413 | kSGB | Species | s_{——}Clostridia_bacterium |
| SGB4988 | 0.91410 | 0.92728 | kSGB | Species | s_{——}Eisenbergiella_tayi |
| SGB5184 | 0.91435 | 0.94303 | kSGB | Species | s_{——}Clostridia_bacterium |
| SGB4786 | 0.91528 | 0.91554 | kSGB | Species | s_{——}Blautia_producta |
| SGB4826 | 0.91549 | 0.91263 | kSGB | Species | s_{——}Blautia_massiliensis |
| group | | | | | |
| SGB4041 | 0.91577 | 0.94821 | kSGB | Species | s_{——}Longicatena_caecimuris |
| SGB7984 | 0.91695 | 0.88474 | kSGB | Species | s_{——}Lactococcus_lactis |
| SGB4761 | 0.91928 | 0.91746 | kSGB | Species | s_{——}Enterocloster_citroniae |
| SGB4447 | 0.92222 | 0.80227 | uSGB | Genus | s_{——}Clostridia_unclassified_SGB4447 |
| SGB6744 | 0.92315 | 0.92052 | kSGB | Species | s_{——}Erysipelatoclostridium_ramosum |
| SGB1836 | 0.92607 | 0.88379 | kSGB | Species | s_{——}Bacteroides_uniformis |
| group | | | | | |
| SGB1814 | 0.92627 | 0.91327 | kSGB | Species | s_{——}Phocaeicola_vulgatus |
| SGB4630 | 0.92731 | 0.94260 | kSGB | Species | s_{——}Clostridium_scindens |
| group | | | | | |
| SGB8163 | 0.93004 | 0.94153 | kSGB | Species | s_{——}Streptococcus_mitis |
| SGB4617 | 0.93264 | 0.94966 | kSGB | Species | s_{——}Sellimonas_intestinalis |
| SGB4588 | 0.93315 | 0.94320 | kSGB | Species | s_{——}Tyzzerella_nexilis |
| group | | | | | |
| SGB4742 | 0.93729 | 0.92385 | kSGB | Species | s_{——}Hungatella_hathewayi |
| SGB4572 | 0.93762 | 0.94247 | kSGB | Species | s_{——}Dorea_phocaeensis |
| SGB10115 | 0.93780 | 0.87910 | kSGB | Species | s_{——}Klebsiella_pneumoniae |
| SGB15158 | 0.94161 | 0.97077 | kSGB | Species | s_{——}Clostridiales_bacterium |
| SGB29328 | 0.94283 | 0.93256 | kSGB | Species | s_{——}Clostridium_sp_SN20 |
| SGB4791 | 0.94507 | 0.95441 | kSGB | Species | s_{——}Blautia_producta |
| SGB4688 | 0.94623 | 0.95159 | kSGB | Species | s_{——}Lachnospiraceae_bacterium |
| SGB10068 | 0.94856 | 0.94769 | kSGB | Species | s_{——}Escherichia_coli |
| SGB71883 | 0.95708 | 0.94116 | uSGB | Other | s_{——}GGB51510_SGB71883 |
| SGB4760 | 0.95906 | 0.95790 | kSGB | Species | s_{——}Enterocloster_clostridioformis |
| SGB4037 | 0.96056 | 0.96955 | kSGB | Species | s_{——}Clostridium_innocuum |
| group | | | | | |
| SGB4529 | 0.96396 | 0.96707 | kSGB | Species | s_{——}Anaerostipes_caccae |
| SGB4837 | 0.96774 | 0.94102 | kSGB | Species | s_{——}Blautia_wexlerae |
| group | | | | | |
| SGB79883 | 0.96809 | 0.97395 | uSGB | Family | s_{——}GGB58233_SGB79883 |
| SGB4762 | 0.96812 | 0.96850 | kSGB | Species | s_{——}Enterocloster_aldensis |
| SGB4797 | 0.97424 | 0.97337 | kSGB | Species | s_{——}Blautia_argi |
| SGB14809 | 0.97745 | 0.97986 | kSGB | Species | s_{——}Eggerthella_lenta |
| SGB4758 | 0.97753 | 0.98572 | kSGB | Species | s_{——}Enterocloster_bolteae |
| group | | | | | |
| SGB4703 | 0.97771 | 0.97923 | uSGB | Family | s_{——}GGB3523_SGB4703 |
| SGB4606 | 0.98061 | 0.98800 | kSGB | Species | s_{——}Mediterraneibacter_glycyrrhizinilyticus |
| SGB4584 | 0.98107 | 0.98931 | kSGB | Species | s_{——}Ruminococcus_gnavus |
| SGB15132 | 0.98122 | 0.98972 | kSGB | Species | s_{——}Flavonifractor_plautii |
| SGB4746 | 0.98485 | 0.98698 | kSGB | Species | s_{——}Lachnospiraceae_bacterium |
| SGB4862 | 0.98588 | 0.99013 | kSGB | Species | s_{——}Blautia_caecimuris |
| SGB4798 | 0.98632 | 0.96497 | kSGB | Species | s_{——}Blautia_hansenii |
| SGB4861 | 0.98943 | 0.98632 | kSGB | Species | s_{——}Lachnospiraceae_bacterium |
| SGB4608 | 0.99077 | 0.99371 | kSGB | Species | s_{——}Ruminococcus_torques |
| SGB4035 | 0.99414 | 0.99342 | kSGB | Species | s_{——}Amedibacillus_dolichus |
| SGB4794 | 0.99616 | 0.99312 | kSGB | Species | s_{——}Blautia_hansenii |
| group | | | | | |
| SGB4753 | 0.99668 | 0.99709 | kSGB | Species | s_{——}Clostridium_sp_AT4 |
| SGB4583 | 1.00000 | 0.99928 | kSGB | Species | s_{——}Faecalimonas_umbilicata |

A collection of two or more microbes described or illustrated herein as associated with a biological status or condition can be referred to as a microbial signature, or a microbiome fingerprint. For instance, any two, any three, any four, any five, any six, any seven, any eight, any nine, any 10, any 11, any 12, any 13, any 14, any 15, or more microbes may be included in a microbial signature or microbiome score for a biological status or condition. In some aspecgs, such microbes may be selected from the Pro-Health (GOOD BUGS; Table 1B) or the Poor Health (BAD BUGS; Table 1C) indicators, or some from both. In some aspects, the rankings may be used as part of a weighting to provide a microbiome score. For example, Table 1A provides an expanded list of microbes. A microbial signature, microbiome fingerprint, or microbiome score may be calculated based on the weighted rankings of microbes on this list. For example, diet ranks may be used to determine the microbiome score of the specific microbial profile of a subject. In other aspects health ranks may be used. In some aspects, both diet and health ranks may be used. In some aspects, a threshold may be identified among the ranked microbes with pro-health correlated microbes above a threshold and poor-health correlated microbes below a threshold.

Full GOOD BUGS (pro-health indicators) list (from Table 1B; 79): SGB15249, SGB6340, SGB4964, SGB14252, SGB15229, SGB6174 group, SGB15317, SGB14179, SGB15225, SGB4894, SGB4643, SGB4963, SGB79840, SGB4893, SGB6276, SGB3952, SGB4638, SGB15236, SGB4191, SGB15053 group, SGB15368, SGB4782, SGB14042, SGB4706, SGB4644, SGB49188, SGB4781, SGB4777, SGB14921, SGB15234, SGB8601, SGB5087, SGB14311, SGB4953, SGB7258, SGB4882, SGB6367, SGB15106, SGB4778, SGB15131, SGB4198 group, SGB15031, SGB13981, SGB15123, SGB54300, SGB4665, SGB13979, SGB15410, SGB2290, SGB14954, SGB14306, SGB4805, SGB14899, SGB4803, SGB13982, SGB15265 group, SGB14114, SGB47656, SGB6749, SGB14253, SGB15346, SGB4810, SGB4770, SGB25497, SGB4957, SGB4654, SGB15373, SGB15254, SGB15323, SGB71759, SGB15180, SGB49168, SGB15051, SGB15145, SGB4966, SGB4780, SGB15291, SGB4816, and SGB4714.

Full BAD BUGS (poor health indicators) list (from Table 1C; 68): SGB7253, SGB6769, SGB4721, SGB14837, SGB14546 group, SGB4763, SGB5193, SGB7985, SGB4699, SGB19850 group, SGB17137, SGB4785, SGB15078, SGB53821, SGB15452, SGB15271, SGB4724, SGB8255 group, SGB79823, SGB8028 group, SGB4573 group, SGB14874, SGB4988, SGB5184, SGB4786, SGB4826 group, SGB4041, SGB7984, SGB4761, SGB4447, SGB6744, SGB1836 group, SGB1814, SGB4630 group, SGB8163, SGB4617, SGB4588 group, SGB4742, SGB4572, SGB10115, SGB15158, SGB29328, SGB4791, SGB4688, SGB10068, SGB71883, SGB4760, SGB4037 group, SGB4529, SGB4837 group, SGB79883, SGB4762, SGB4797, SGB14809, SGB4758 group, SGB4703, SGB4606, SGB4584, SGB15132, SGB4746, SGB4862, SGB4798, SGB4861, SGB4608, SGB4035, SGB4794 group, SGB4753, and SGB4583.

Also contemplated are specific subsets of the provided full lists of pro-health and/or poor health indicator microbes (identified herein by SGB designation). These indicator subsets may include, in various embodiments, those SGBs that are demonstrated herein to show statistically significant alteration in abundance between two different samplings or testing series, such as those SGBs showing a significant change in a favorable direction for health (e.g., pro-health SGBs that increase and poor-health SGBs that decrease after intervention), and/or aggregations across microbes (SGBs) that change based on health markers alone, or based on health markers plus diet markers, and so forth. Specific additional subsets of microbes (identified by SGB designation) are provided below; also contemplated are any two, any three, any four, any five, any six, any seven, any eight, any nine, any 10, any 11, any 12, any 13, any 14, any 15, or more SGBs (microbes) from any of these subsets (up to the full number in each subset).

GOOD & BAD microbes showing a statistically significant change in frequency, selected using both health metrics and diet markers (from Tables 2 & 3): SGB4964, SGB4782, SGB15053_group, SGB15265_group, SGB15180, SGB4706, SGB4714, SGB4953, SGB4816, SGB15368, SGB25497, SGB4810, SGB15346, SGB47656, SGB5087, SGB4770, SGB15410, SGB4654, SGB4781, SGB15373, SGB4778, SGB4643, SGB4780, SGB15051, SGB6749, SGB8601, SGB4882, SGB15249, SGB71759, SGB15323, SGB15291, SGB4963, SGB6276, SGB6367, SGB15317, SGB14253, SGB15106, SGB4966, SGB4665, SGB4893, SGB4644, SGB15254, SGB15229, SGB4957, SGB15236, SGB4608, SGB15132, SGB15078, SGB4606, SGB4584, SGB4826_group, SGB14546_group, SGB15452, SGB7985, SGB14837, SGB15271, SGB5184, SGB4573_group, SGB4791, SGB4837_group, SGB4703, SGB4037_group, SGB4753, SGB10068, SGB4721, SGB14874, SGB7253, SGB4035, SGB4746, SGB4572, SGB8163, SGB4630_group, SGB4588_group, SGB4862, SGB6744, SGB4742, SGB4758_group, and SGB4761.

GOOD microbes showing a statistically significant change in frequency, selected using both health metrics and diet markers (from Table 2): SGB4964, SGB4782, SGB15053_group, SGB15265_group, SGB15180, SGB4706, SGB4714, SGB4953, SGB4816, SGB15368, SGB25497, SGB4810, SGB15346, SGB47656, SGB5087, SGB4770, SGB15410, SGB4654, SGB4781, SGB15373, SGB4778, SGB4643, SGB4780, SGB15051, SGB6749, SGB8601, SGB4882, SGB15249, SGB71759, SGB15323, SGB15291, SGB4963, SGB6276, SGB6367, SGB15317, SGB14253, SGB15106, SGB4966, SGB4665, SGB4893, SGB4644, SGB15254, SGB15229, SGB4957, and SGB15236.

GOOD microbes showing a statistically significant increase, selected using both health metrics and diet markers (39 total, from Table 2): SGB4964, SGB4782, SGB15053_group, SGB15265_group, SGB15180, SGB4706, SGB4714, SGB4953, SGB4816, SGB15368, SGB25497, SGB4810, SGB47656, SGB5087, SGB15410, SGB4654, SGB4781, SGB15373, SGB4778, SGB4643, SGB4780, SGB15051, SGB6749, SGB8601, SGB4882, SGB15249, SGB71759, SGB15291, SGB6276, SGB6367, SGB14253, SGB15106, SGB4966, SGB4665, SGB4893, SGB15254, SGB15229, SGB4957, and SGB15236.

GOOD microbes showing a statistically significant decrease, selected using both health metrics and diet markers (6 total; Table 2): SGB15346, SGB4770, SGB15323, SGB4963, SGB15317, and SGB4644.

BAD microbes showing a statistically significant change in frequency, selected using both health metrics and diet markers (from Table 3): SGB4608, SGB15132, SGB15078, SGB4606, SGB4584, SGB4826_group, SGB14546_group, SGB15452, SGB7985, SGB14837, SGB15271, SGB5184, SGB4573_group, SGB4791, SGB4837_group, SGB4703, SGB4037_group, SGB4753, SGB10068, SGB4721, SGB14874, SGB7253, SGB4035, SGB4746, SGB4572, SGB8163, SGB4630_group, SGB4588_group, SGB4862, SGB6744, SGB4742, SGB4758_group, and SGB4761.

BAD microbe showing statistically significant increase, selected using both health metrics and diet markers (1 total; Table 3): SGB4761.

BAD microbes showing a statistically significant decrease, showing statistically significant changes, selected using both health metrics and diet markers (32 total; Table 3): SGB4608, SGB15132, SGB15078, SGB4606, SGB4584, SGB4826_group, SGB14546_group, SGB15452, SGB7985, SGB14837, SGB15271, SGB5184, SGB4573_group, SGB4791, SGB4837_group, SGB4703, SGB4037_group, SGB4753, SGB10068, SGB4721, SGB14874, SGB7253, SGB4035, SGB4746, SGB4572, SGB8163, SGB4630_group, SGB4588_group, SGB4862, SGB6744, SGB4742, and SGB4758_group.

GOOD & BAD microbes showing a statistically significant change in frequency, selected using only health metrics (from Table 4 & 5): SGB4964, SGB4782, SGB14899, SGB15053_group, SGB15265_group, SGB4706, SGB4953, SGB15368, SGB2290, SGB47656, SGB54300, SGB5087, SGB15410, SGB4781, SGB4778, SGB4643, SGB6749, SGB13981, SGB4882, SGB8601, SGB15249, SGB4198_group, SGB4963, SGB6276, SGB15317, SGB6367, SGB14954, SGB14253, SGB15106, SGB4665, SGB4893, SGB4644, SGB49188, SGB15229, SGB4608, SGB15078, SGB15132, SGB4606, SGB4584, SGB4826_group, SGB15452, SGB15271, SGB19850_group, SGB5184, SGB4573_group, SGB4791, SGB4837_group, SGB4703, SGB4037_group, SGB4753, SGB10068, SGB14874, SGB53821, SGB4035, SGB4746, SGB4572, SGB8163, SGB4630_group, SGB4588_group, SGB17137, SGB4862, SGB6744, SGB79823, SGB4742, SGB4447, and SGB4758_group.

GOOD microbes showing a statistically significant change in frequency, selected using only health metrics (from Table 4): SGB4964, SGB4782, SGB14899, SGB15053_group, SGB15265_group, SGB4706, SGB4953, SGB15368, SGB2290, SGB47656, SGB54300, SGB5087, SGB15410, SGB4781, SGB4778, SGB4643, SGB6749, SGB13981, SGB4882, SGB8601, SGB15249, SGB4198_group, SGB4963, SGB6276, SGB15317, SGB6367, SGB14954, SGB14253, SGB15106, SGB4665, SGB4893, SGB4644, SGB49188, and SGB15229.

GOOD microbes showing a statistically significant increase, selected using only health metrics (27 total; Table 4): SGB4964, SGB4782, SGB15053_group, SGB15265_group, SGB4706, SGB4953, SGB15368, SGB2290, SGB47656, SGB54300, SGB5087, SGB15410, SGB4781, SGB4778, SGB4643, SGB6749, SGB13981, SGB4882, SGB8601, SGB15249, SGB6276, SGB6367, SGB14253, SGB15106, SGB4665, SGB4893, and SGB15229.

GOOD microbes showing statistically significant decrease, selected using only health metrics (7 total; Table 4): SGB14899, SGB4198_group, SGB4963, SGB15317, SGB14954, SGB4644, and SGB49188.

BAD microbes showing a statistically significant changes, selected using only health metrics (from Table 5): SGB4608, SGB15078, SGB15132, SGB4606, SGB4584, SGB4826_group, SGB15452, SGB15271, SGB19850_group, SGB5184, SGB4573_group, SGB4791, SGB4837_group, SGB4703, SGB4037_group, SGB4753, SGB10068, SGB14874, SGB53821, SGB4035, SGB4746, SGB4572, SGB8163, SGB4630_group, SGB4588_group, SGB17137, SGB4862, SGB6744, SGB79823, SGB4742, SGB4447, and SGB4758_group.

BAD microbes showing statistically significant increase, selected using only health metrics (1 total; Table 5): SGB4447.

BAD microbes showing a statistically significant decrease, selected using only health metrics (31 total; Table 5): SGB4608, SGB15078, SGB15132, SGB4606, SGB4584, SGB4826_group, SGB15452, SGB15271, SGB19850_group, SGB5184, SGB4573_group, SGB4791, SGB4837_group, SGB4703, SGB4037_group, SGB4753, SGB10068, SGB14874, SGB53821, SGB4035, SGB4746, SGB4572, SGB8163, SGB4630_group, SGB4588_group, SGB17137, SGB4862, SGB6744, SGB79823, SGB4742, and SGB4758_group.

### (IV) METHODS OF USE

Based on the research reported herein, there are now enabled a number of methods of using the results of the microbiome metagenomic analyses.

For instance, one embodiment is a method of using a group of microbes to determine a health condition in a human subject. By way of example, the group of microbes includes: at least two pro-health indicator microbes; or at least two poor health indicator microbes; or at least two pro-health indicator microbes and at least two poor health indicator microbes. Lists of gut biome microbes including pro-health and poor health indicator microbes are described herein, for instance in Example 1 and Table 1A, 1B, and 1C. By way of example, in some embodiments the pro-health indicator microbes are selected from the group including GOOD BUGS (as listed in Table 1B, or another subset of pro-health/good microbes described herein). By way of further example, in some embodiments the poor health indicator microbes are selected from the group including BAD BUGS (as listed in Table 1C, or another subset of poor-health/bad microbes described herein). In another example embodiment, at least one of the pro-health indicator microbes is selected from the group including GOOD BUGS (as listed in Table 1B, or another subset of pro-health/good microbes described herein); and at least one of the poor health indicator microbes is selected from the group including BAD BUGS (as listed in Table 1C, or another subset of poor-health/bad microbes described herein). While Tables 1B and 1C provide exemplary GOOD BUGS and BAD BUGS respectively, other thresholds may be used depending on the health condition being modified or the microbiome of the user. For example, it may be useful to include additional pro-health microbes in a population with poor microbiome health where the microbes listed in Table 1B are rare. Furthermore, in other embodiments, the complete list of microbes in Table 1A may be used to capture a broad assessment of the subject's microbiome, not only a view of the subject's most pro-health and most poor-health microbes.

In further examples of such methods, the method of using a group of microbes to determine a health condition in a human subject includes obtaining a biological sample from the human subject (for instance, a microbiome sample, such as a stool sample); and analyzing the biological sample to determine presence, absence, or abundance of the at least two pro-health indicator microbes and/or the at least two poor health indicator microbes.

In additional examples of such methods, the method of using a group of microbes to determine a health condition in a human subject includes obtaining a biological sample from the human subject; identifying in the biological sample at least 10, at least 20, at least 30, at least 40, at least 50, at least 60, at least 70, at least 80, at least 90, at least 100, at least 125, at least 150, at least 175, at least 200, or more than 200 different microbes in the biological sample; and determining the health condition of the human subject based on presence, absence, and/or absolute or relative abundance of the identified microbes in the biological sample.

In any of these methods using a group of microbes to determine a health condition in a human subject, the group of microbes may include at least three pro-health indicator microbes; at least five pro-health indicator microbes; at least ten pro-health indicator microbes; or more than 10 pro-health indicator microbes. Optionally, the group of microbes includes all of the following pro-health indicator microbes: GOOD BUGS (as listed in Table 1B, or another subset of pro-health/good microbes described herein). In another example, the group of microbes includes all of the following pro-health indicator microbes: any of the listed specific subsets of GOOD BUGS described herein.

In any of these methods using a group of microbes to determine a health condition in a human subject, the group of microbes may include: at least three poor health indicator microbes; at least five poor health indicator microbes; at least ten poor health indicator microbes; or more than 10 poor health indicator microbes. Optionally, the group of microbes includes all of the following poor health indicator microbes: BAD BUGS listed in Table 1C, or a subset of poor-health/bad microbes described herein. In another example, the group of microbes includes all of the following poor health indicator microbes: any of the listed specific subsets of BAD BUGS described herein. In some aspects, the group may include some or all of the indicator microbes as listed in Table 1A.

In any of these methods of using a group of microbes to determine a health condition in a human subject, the health condition may include at least one of: overall good health, overall poor health, obesity, BMI, diabetes risk, cardiometabolic risk, cardiovascular disease risk, or postprandial response to food intake. In some aspects, the overall health of the individual may be reflected as a quality of the microbiome. For example, someone with overall good health would be expected to have a higher quality microbiome than someone with diabetes. In some aspects this may be identified as having a higher score.

Optionally, any of the provided methods of using a group of microbes to determine a health condition in a human subject may include detecting the presence, absence, or relative abundance of at least one of the microbes in a microbiome sample from the human subject. For instance, in this context the detecting may include one or more of: sequencing one or more nucleic acids of a pro-health or poor health microbe, hybridizing a nucleic acid probe to a nucleic acid of a pro-health or poor health microbe, detecting one or more proteins from a pro-health or poor health microbe, or measuring activity of one or more proteins a pro-health or poor health microbe. For instance, the detecting may include shotgun metagenomics.

Also provided herein are methods of predicting a health condition in a subject. Such methods involve determining presence, absence, or relative abundance of at least three pro-health indicator microbes in a microbiome of the subject; determining presence, absence, or relative abundance of at least three poor health indicator microbes in a microbiome of the subject; and predicting the health condition of the subject, based on the presence, absence, or relative abundance of the pro-health and/or poor health indicator microbes in the microbiome of the subject. By way of example, in some such methods the pro-health indicator microbes are selected from the group including GOOD BUGS (as listed in Table 1B, or another subset of pro-health/good microbes described herein). By way of further example, in some such methods the poor health indicator microbes are selected from the group including BAD BUGS (as listed in Table 1C, or another subset of poor-health/bad microbes described herein). In other aspects, they may be selected from a ranked list such as the list of Table 1A with good health being determined with scores or microbes greater than a threshold level and bad health being determined by scores or microbes below a threshold level.

It is contemplated that in some methods of predicting a health condition in a subject, the health condition includes at least one of obesity, increased cardiometabolic risk, diabetes risk, or overall poor health; and the health condition is predicted by the presence and/or abundance of more poor health indicator microbes than pro-health indicator microbes; and/or the health condition includes at least one of overall good health or absence of obesity, reduced cardiometabolic risk, or reduced diabetes risk; and the health condition is predicted by the presence and/or abundance of more pro-health indicator microbes than poor health indicator microbes.

Another embodiment is a method to predict overall good or poor general health in a non-diseased human subject. In examples of such methods, the methods involve obtaining a microbiome sample (for instance, a stool sample) from the human subject; isolating a nucleic acid fraction from the microbiome sample; detecting, within the nucleic acid fraction, presence, absence, or relative abundance of at least one unique marker sequence indicative of: a pro-health indicator microbe selected from the group including GOOD BUGS (as listed in Table 1B, or another subset of pro-health/good microbes described herein); or a poor health indicator microbes selected from the group including BAD BUGS (as listed in Table 1C, or another subset of poor-health/bad microbes described herein); and at least one of predicting the human subject has overall good general health if the pro-health indicator microbes outnumber or are relatively more abundant than the poor-health indicator microbes; or predicting the human subject has overall poor general health if the poor health indicator microbes outnumber or are relatively more abundant than the pro-health indicator microbes.

Examples of the methods to predict overall good or poor general health in a non-diseased human subject further include providing to the human subject a dietary recommendation based on the presence, absence, or relative abundance of one or more poor health indicator microbes and/or one or more pro-health indicator microbes. Such dietary recommendation may be provided as a prescription. Optionally, the method may further include administering to the subject one or more compounds or substances intended to alter the presence or quantity or relative proportion of at least one pro-health indicator microbe or at least one poor health indicator microbe in the subject.

Also enabled by this disclosure are methods for targeting a microbiome of a human subject to promote health, which methods include (A) detecting in a microbiome sample from the human subject one or more pro-health indicator microbes selected from the group including GOOD BUGS (as listed in Table 1B, or another subset of pro-health/good microbes described herein); and administering to the human a composition that increases growth or survival of the pro-health indicator microbe(s); and/or (B) detecting in a microbiome sample from the human subject one or more poor health indicator microbe selected from the group including BAD BUGS (as listed in Table 1C, or another subset of poor-health/bad microbes described herein); and administering to the human a composition that decreases growth or survival of the poor health indicator microbe(s). In some aspects, other subsets of Table 1A may be used.

Examples of such methods for targeting a microbiome of a human subject to promote health involve detecting: at least three pro-health indicator microbes; at least five pro-health indicator microbes; at least ten pro-health indicator microbes; or more than ten pro-health indicator microbes. All of the following pro-health indicator microbes are detected in some embodiments: GOOD BUGS listed in Table 1B, or another subset of pro-health/good microbes described herein. Alternatively, the indicator microbes include at least one GOOD BUG listed in a subset of pro-health/good microbes described herein. Alternatively, the indicator microbes include all of the microbes listed in a specific subset of pro-health/good microbes described herein.

Further examples of such methods for targeting a microbiome of a human subject to promote health involve detecting: at least three poor health indicator microbes; at least five poor health indicator microbes; at least ten poor health indicator microbes; or more than ten poor health indicator microbes. All of the following poor health indicator microbes are detected in some embodiments: BAD BUGS listed in Table 1C, or another subset of poor-health/bad microbes described herein. Alternatively, the indicator microbes include all of the microbes listed in a specific subset of poor-health/bad microbes described herein.

Also provided are methods of altering abundance of one or more microbes in gut microflora of a subject, including administering to the subject a probiotic composition, or administering to the subject a prebiotic composition, or administering to the subject an antibiotic composition.

### (V) KITS AND ARRAYS

Also provided herein are various different types of kits. Examples of such kits include kits useful to gather data or information from a subject, for instance. Examples of the information/data-gathering kits include one or more device(s) to in/with which to collect a microbiome sample (for instance, a stool sample collection device, surface swab, etc.), and optionally one or more devices in/with which to collect biological samples (such as blood samples; for instance, a device for the collection of blood spots). Optionally, the kits will also include instructions for how the subject, or a health care provider, is to collect the samples; how those samples are to be treated and/or stored before they are forwarded for analysis; and additional instructions regarding recording information other than biological samples that can inform or influence the interpretation of results from analyses of the biological sample(s). For instance, kits may include instructions on how to install or access computer software useful to collect information from the subject, such as food intake, exercise, and other objective or subject information.

In some kit embodiments, the kit will further include a device or system for monitoring blood glucose of the subject. By way of example, such device may be a continues blood glucose monitor. Alternatively, the kit may provide a system for intermittently monitoring blood glucose, for instance through periodic blood sampling and analysis such as is routine for monitoring the blood glucose of Type 1 diabetics.

It is also contemplated that some kit embodiments will include instructions to enable the subject being tested to undergo one or more additional sampling or testing procedures, for instance at a laboratory or other device outside of their home. For instance, some kits may include instructions for how to provide a fasting blood sample, or more generally a blood sample useful to detect or measure metabolic action.

Additional kit embodiments are provided for the analysis of samples collect from a subject. By way of example, such testing kits include one or more marker molecules capable of detecting the presence (and/or quantity) of at least one indicator microbe in a sample (e.g., a stool or other microbiome sample) from a subject. For instance, marker molecules are nucleic acids (e.g., oligonucleotides) or amino acids (e.g., peptides) specific for a single indicator microbe. Such marker molecules may optionally be attached to a solid surface, such as an array. Marker molecules may optionally be labeled for ease of detection.

A kit can include a device as described herein, and optionally additional components such as buffers, reagents, and instructions for carrying out the methods described herein. The choice of buffers and reagents will depend on the particular application, e.g., setting of the assay (point-of-care, research, clinical), analyte(s) to be assayed, the detection moiety used, the detection system used, etc.

The kit can also include informational material, which can be descriptive, instructional, marketing, or other material that relates to the methods described herein and/or the use of the devices for the methods described herein. In embodiments, the informational material can include information about production of the device, physical properties of the device, date of expiration, batch, or production site information, and so forth.

Also contemplated are arrays of biological macromolecules (markers), such as nucleic acids (e.g., oligonucleotides) or amino acids (e.g., peptides or proteins), that enable the detection and/or quantification of microbes from a microbiome of a subject, such as a human subject. With the provision herein of lists of specific pro-health and specific poor health indicator microbes, arrays can be prepared that specifically can detect and/or quantify such indicator microbes. By way of example, an array may include markers specific for individual pro-health or poor health microbes. Such examples may be genomic sequence determined to be or recognized as being specific for an individual microbe listed, for instance, in Table 5.

Specific arrays are pro-health indicator detection arrays, which contain two or more markers each of which is specific for a pro-health indicator microbe as describe herein, including for instance microbes indicated to be associated with generally good health of the subject from which the microbe is isolated. By way of example, such pro-health indicator microbes may include: GOOD BUGS (as listed in Table 1B, or another subset of pro-health/good microbes described herein). Thus, contemplated herein are pro-health indicator arrays that include at least one marker for each of at least two of these listed pro-health indictor microbes; each of at least three; each of at least four; each of at least five; each of at least six; each of at least seven; each of at least eight; each of at least nine; each of at least ten; or more than ten of these listed pro-health indictor microbes. Some arrays will include all seventeen of the listed pro-health indictor microbes. Optionally, any of these pro-health indicator arrays may also include markers for additional microbes; these may be other pro-health indicator microarrays or poor health indictor microbes, for instance.

Additional specific arrays are poor health indicator detection arrays, which contain two or more markers each of which is specific for a poor health indicator microbe as describe herein, including for instance microbes indicated to be associated with generally poor health of the subject from which the microbe is isolated. By way of example, such poor health indicator microbes include: BAD BUGS (as listed in Table 1C, or another subset of poor-health/bad microbes described herein). Thus, contemplated herein are poor health indicator arrays that include at least one marker for each of at least two of these listed poor health indictor microbes; each of at least three; each of at least four; each of at least five; each of at least six; each of at least seven; each of at least eight; each of at least nine; each of at least ten; or more than ten of these listed poor health indictor microbes. Some arrays will include all fifteen of the listed poor health indictor microbes. Optionally, any of these poor health indicator arrays may also include markers for additional microbes; these may be other poor health indicator microarrays or pro-health indictor microbes, for instance.

The arrays may be utilized in myriad applications. For example, the arrays in some embodiments are used in methods for detecting association between a behavior (such as a food choice, or more generally, a diet) and a health condition. For instance, such a health condition may include balance (or imbalance) of the normal gut microbiome; gastrointestinal conditions such as inflammatory bowel disease (IBD) and irritable bowel syndrome (IBS); wider systemic manifestations of disease or disorder, such as obesity, type 2 diabetes (T2D), diabetes risk, metabolic syndrome, prediabetes, and obesity; as well as overall good health, overall poor health, BMI, cardiometabolic risk, cardiovascular disease risk, and postprandial response to food intake. This method typically includes incubating a sample from a subject (e.g., from the microbiome of the subject) with the array under conditions such that biomolecules in the sample may associate with marker biomolecules attached to the array. The association is then detected, using means commonly known in the art. In this context, the term association may include hybridization, covalent binding, or ionic binding, for instance. A skilled artisan will appreciate that conditions under which association occurs will vary depending on the biomolecules, the markers, the substrate, and the detection method utilized. As such, suitable conditions can be optimized for each individual array created or assay carried out with an array.

In yet another embodiment, the array is used as a tool in a method to determine whether a compound or composition is effective to modify a biological condition, such as the balance or imbalance of the microbiome in a subject, or for a treatment of a disease or disorder in a subject.

In another embodiment, the array is used as a tool in a method to determine whether a compound increases or decreases the relative abundance in a subject of any of the pro-health or poor health indicator microbes describe herein. Typically, such methods include comparing the presence, absence, and/or quantity of one or more indicator microbes in a subject's microbiome before and after administration of a compound or composition. If the abundance of biomolecule(s) associated with at least one pro-health microbe increases after treatment, or the abundance of biomolecule(s) associated with at least one poor health microbe decreases, or if the relative abundance of biomolecule(s) shifts to be more similar to a “healthy” profile or fingerprint discussed herein, the compound or composition may be effective in improving the health of the subject.

### (VI) SYSTEMS

Also provided are systems to assay a biological condition in a subject, such as a human or other mammalian subject. By way of example, such a system includes: a nucleic acid sample isolation device, which is adapted to isolate a nucleic acid sample from the subject; a sequencing device, which is connected to the nucleic acid sample isolation device and adapted to sequence the nucleic acid sample, thereby obtaining a sequencing result; and an alignment device, which is connected to the sequencing device and adapted to align the sequencing result against sequence from one or more of microbes in order to determine presence or absence of the microbe(s) based on the alignment result. In examples of such systems, the microbes include one or more of: pro-health indicator microbes selected from the group including GOOD BUGS (as listed in Table 1B, or another subset of pro-health/good microbes described herein); and/or poor health indicator microbes selected from the group including BAD BUGS (as listed in Table 1C, or another subset of poor-health/bad microbes described herein).

Optionally, the systems may further include an information delivery device capable of delivering to the subject information about the results of the alignment. Such information may include one or more of: the identity and/or relative or absolute quantity of one or more microbes, such as microbes found or not found in the microbiome of the subject; information on the subject's gut microbiome health; information on the health of the subject, for instance based the presence, absence, or relative abundance of one or microbes in the subject's microbiome; one or more recommendations for how to modify the subject's diet; a specific recommendation for a food to eat, or a food to avoid; information on general diet plan(s); options for lifestyle choices; and so forth.

The Exemplary Embodiments and Example(s) below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (VII) EXEMPLARY EMBODIMENTS

1. A method including: receiving first test data from a remote device, the test data representing quantities of microbes present in a microbiome associated with an individual at a first time; accessing a ranked list of indicator microbes; determining, based at least in part on the first test data and the list of indicator microbes, a first microbiome score representing a quality of the microbiome associated with the individual at the first time; and sending the microbiome score to a storage location accessible by a device associated with the individual.

2. The method of embodiment 1, further including: using one or more weighted values associated with types of microbes in determining the microbiome score.

3. The method of any of embodiments 1 to 2, further including: receiving first diet data from a remote device, the diet data representing the diet of the individual at the first time; and using the first diet data at least in part in determining the microbiome score.

4. The method of any of embodiments 1 to 3, further including: generating, based at least in part on the first microbiome score and the first test data, a first recommended action, the first recommended action to increase a relative quantity of pro-health associated indicator microbes or decrease a relative quantity of poor health associated indicator microbes in the microbiome associated with the individual.

5. The method of embodiment 4, wherein the first recommended action is a change in diet of the individual.

6. The method of embodiment 4, wherein the first recommended action is a consumption of prebiotics or probiotics by the individual.

7. The method of any of embodiments 1 to 6, further including: receiving second test data from the remote device, the second test data representing quantities of microbes present in the microbiome associated with the individual at a second time, the second time subsequent to the first time; determining, based at least in part on the second test data and the list of indicator microbes, a second microbiome score representing a quality of the microbiome associated with the individual at the second time; and sending the second microbiome score to the storage location accessible by the device associated with the individual.

8. The method of embodiment 7, further including: receiving tracking data associated with the first recommended action from the device associated with the individual; and wherein the second microbiome score's improvement compared against the first microbiome score is correlated to determining, based at least in part on the tracking data, that the individual has followed the recommended action for a predetermined period for time or until a target associated with the first recommended action is achieved.

9. The method of any one of embodiments 2 to 8, further including: determining the one or more weighted values based at least in part on health and/or diet data and microbiome data associated with a plurality of individuals.

10. The method of any one of embodiments 2 to 9, further including: determining the one or more weighted values based at least in part on microbiome data associated with a plurality of individuals at two or more times per individual.

11. The method of any one of embodiments 2 to 10, wherein: determining the one or more weighted values further includes: training one or more machine learned models using microbiome data associated with a plurality of individuals; and receiving, from the one or more machine learned models, at least one weighted value associated with a type of microbe.

12. The method of embodiments 1 to 11, further including: determining the first microbiome score further includes: inputting the first test data into one or more machine learned models trained using microbiome data associated with a plurality of individuals; and receiving, as an output from the one or more machine learned models, the first microbiome score.

13. The method of embodiments 7 to 12, further including: generating a graphical representation of changes in the microbiome associated with the individual over a period of time including the first time and the second time; and causing the graphical representation to be presented on a display of the device associated with the individual.

14. The method of any of embodiments 1 to 13, wherein the quantity of microbes is expressed as a percentage of the microbiome.

15. The method of any of embodiments 1 to 14, wherein at least ten of the indicator microbes (identified by its species-level genome bin (SGB) designation) are selected from: SGB15249, SGB6340, SGB4964, SGB14252, SGB15229, SGB6174_group, SGB15317, SGB14179, SGB15225, SGB4894, SGB4643, SGB4963, SGB79840, SGB4893, SGB6276, SGB3952, SGB4638, SGB15236, SGB4191, SGB15053_group, SGB15368, SGB4782, SGB14042, SGB4706, SGB4644, SGB49188, SGB4781, SGB4777, SGB14921, SGB15234, SGB8601, SGB5087, SGB14311, SGB4953, SGB7258, SGB4882, SGB6367, SGB15106, SGB4778, SGB15131, SGB4198_group, SGB15031, SGB13981, SGB15123, SGB54300, SGB4665, SGB13979, SGB15410, SGB2290, SGB14954, SGB14306, SGB4805, SGB14899, SGB4803, SGB13982, SGB15265_group, SGB14114, SGB47656, SGB6749, SGB14253, SGB15346, SGB4810, SGB4770, SGB14043, SGB4886, SGB25497, SGB4815_group, SGB25416, SGB4957, SGB4654, SGB15373, SGB15254, SGB6571, SGB15323, SGB71759, SGB4648, SGB15180, SGB15413, SGB49168, SGB14960, SGB4133, SGB15051, SGB4993, SGB15395, SGB15145, SGB5111, SGB6317, SGB4966, SGB4780, SGB14198, SGB63101, SGB4779, SGB15233, SGB4769, SGB2295, SGB72336, SGB4658, SGB14770, SGB6148, SGB25493, SGB4831_group, SGB14965, SGB15224, SGB4938, SGB15402, SGB15291, SGB9333, SGB4664, SGB4906, SGB4711, SGB15065, SGB714_group, SGB4772, SGB3958, SGB4629, SGB14048, SGB15052, SGB14861, SGB9205, SGB4280, SGB4829, SGB4816, SGB2317, SGB15411, SGB5117, SGB14250, SGB14924, SGB4767, SGB6376, SGB4714, SGB4691, SGB14341, SGB15244, SGB5082_group, SGB4910, SGB4914, SGB8599, SGB4936, SGB15374, SGB72916, SGB4909, SGB15390, SGB15164, SGB15093, SGB13983, SGB5042, SGB4771, SGB15356, SGB72479, SGB4557, SGB3988, SGB15041, SGB14128, SGB15385, SGB6750, SGB4184, SGB3573, SGB66170, SGB15201, SGB15203, SGB79798, SGB15382, SGB4652, SGB9346, SGB14969, SGB4262, SGB4394, SGB61601, SGB15216, SGB14027, SGB4674, SGB14937, SGB15090, SGB9391, SGB15383, SGB29347, SGB14991, SGB14940, SGB4809, SGB6141, SGB4687, SGB63163, SGB14177, SGB4832, SGB15160, SGB48024, SGB6179, SGB4768, SGB5090_group, SGB29302, SGB9712_group, SGB3813, SGB79833, SGB4659, SGB4328, SGB4776, SGB1790, SGB14313, SGB5043, SGB15127, SGB15049, SGB42321, SGB15403, SGB15115, SGB4905, SGB14838, SGB15012, SGB9202, SGB80143, SGB3992, SGB7259, SGB4546, SGB14974, SGB13976, SGB15342, SGB2296, SGB14941, SGB3996, SGB53497, SGB15470, SGB14020, SGB1858, SGB14851, SGB6305, SGB14932, SGB15089, SGB1862, SGB15401, SGB4027, SGB15140, SGB2325, SGB14317, SGB4628, SGB4669, SGB15299, SGB6478, SGB14262, SGB63342, SGB4960, SGB63333, SGB15316_group, SGB4651, SGB1965, SGB15081, SGB59819, SGB2326, SGB14912, SGB14322_group, SGB3940, SGB4029, SGB2301, SGB63167, SGB14797_group, SGB5200, SGB17347, SGB4868, SGB15067, SGB53515, SGB15075, SGB4421, SGB5121, SGB9226, SGB2318, SGB14894, SGB4817, SGB14966, SGB3989, SGB15370, SGB14975, SGB4436, SGB14839, SGB14993_group, SGB15322, SGB9387, SGB3959, SGB6362, SGB4063, SGB14773_group, SGB29334, SGB14151, SGB15087, SGB14022, SGB14972, SGB15045, SGB4712, SGB15389, SGB82503, SGB15318_group, SGB1857, SGB4828_group, SGB4788_group, SGB79822, SGB4269, SGB14923, SGB2328, SGB1784, SGB14137, SGB15204, SGB7256, SGB15295_group, SGB14182, SGB29342, SGB4438, SGB14824_group, SGB2303, SGB9262, SGB14952, SGB14951, SGB15459, SGB6358, SGB14929, SGB15286, SGB5060, SGB15332_group, SGB15126, SGB72433_group, SGB4045, SGB1626, SGB5180, SGB4867, SGB4825, SGB4925, SGB53517, SGB1844, SGB14844, SGB4871_group, SGB14050, SGB25547, SGB1815, SGB3957, SGB54347, SGB6140, SGB14127, SGB29339, SGB1832, SGB9342_group, SGB15278, SGB9203, SGB1962, SGB5076, SGB4808, SGB15119, SGB3962, SGB14892, SGB4775, SGB4166, SGB7144, SGB4959, SGB63353, SGB14953, SGB6139, SGB16971, SGB15300, SGB3574, SGB47850, SGB63327, SGB4181, SGB15506, SGB5190, SGB14181, SGB1846, SGB15068, SGB4537, SGB14906, SGB9347, SGB1786, SGB7265, SGB4571, SGB29321, SGB4531, SGB15073, SGB14933, SGB15154, SGB6747, SGB15273, SGB4367, SGB15091, SGB3965, SGB63369, SGB9224, SGB3964, SGB6952, SGB4765, SGB29305, SGB4285_group, SGB5089, SGB4581, SGB59869, SGB4727, SGB25431, SGB2299, SGB1829, SGB4990, SGB1891_group, SGB2286, SGB17278, SGB1949, SGB63343, SGB5045, SGB5075_group, SGB8007_group, SGB29375, SGB6847, SGB1798, SGB4670, SGB4582_group, SGB4290, SGB14891, SGB1785, SGB15209, SGB14150, SGB33551, SGB14958, SGB14807, SGB4820, SGB1812, SGB4716, SGB4425_group, SGB9340, SGB14779, SGB14125, SGB14259, SGB4784, SGB15260, SGB14741, SGB4577_group, SGB71281, SGB14307, SGB5803, SGB58519, SGB5077, SGB15125, SGB15143, SGB4116, SGB4677, SGB15467_group, SGB7202, SGB6178, SGB6796_group, SGB6783_group, SGB1860, SGB4059, SGB63326, SGB9272_group, SGB15350, SGB14334, SGB1941, SGB16986, SGB14909, SGB1963, SGB4774, SGB14143, SGB15272, SGB29380, SGB4811_group, SGB4595, SGB4834, SGB4030, SGB8071, SGB2311, SGB3991, SGB4722, SGB17244, SGB3993, SGB4553, SGB6956, SGB1699, SGB6962_group, SGB4705, SGB1957, SGB4532, SGB4327_group, SGB59559, SGB4594, SGB5765_group, SGB17169, SGB15120, SGB1948, SGB14853, SGB14898, SGB48424, SGB5792, SGB6754, SGB1934, SGB14854, SGB6768, SGB15156_group, SGB4750, SGB14142, SGB17153_group, SGB4552_group, SGB4951, SGB4303, SGB9286, SGB5051, SGB17237, SGB4940, SGB4597, SGB4563_group, SGB1867, SGB47515, SGB59562, SGB8059_group, SGB4991, SGB4540_group, SGB14862, SGB4422, SGB15124, SGB14987, SGB7263, SGB9283, SGB4348, SGB14895, SGB17154, SGB17167, SGB4626, SGB5843, SGB4575, SGB4613, SGB15076, SGB17130, SGB15904, SGB4080, SGB6846, SGB5825_group, SGB14808, SGB4874, SGB9228, SGB6320, SGB9260, SGB8002, SGB6936, SGB14962, SGB8053, SGB4701, SGB5197, SGB4036, SGB4744, SGB1877, SGB29313, SGB14845, SGB14963, SGB8056, SGB6939, SGB17256, SGB4749, SGB3961, SGB7142, SGB14999, SGB4747, SGB15149, SGB4987, SGB6767, SGB17248, SGB4725, SGB59576, SGB1903_group, SGB5183, SGB49059, SGB4121, SGB25538, SGB3970, SGB3969, SGB14890, SGB1830_group, SGB7967, SGB17168, SGB8047, SGB4046, SGB1855_group, SGB3922, SGB14995, SGB7253, SGB48013, SGB4741, SGB4671, SGB15878, SGB6769, SGB14180, SGB29433, SGB1871, SGB5182, SGB5736, SGB6771, SGB4721, SGB14837, SGB4044, SGB8095, SGB17152, SGB1861, SGB66069, SGB4031, SGB6153, SGB7264, SGB15121, SGB25437, SGB14546_group, SGB4933_group, SGB4763, SGB5193, SGB7985, SGB4699, SGB19850_group, SGB17137, SGB4785, SGB15078, SGB53821, SGB15452, SGB15271, SGB4724, SGB8255_group, SGB79823, SGB8028_group, SGB4573_group, SGB14874, SGB4988, SGB5184, SGB4786, SGB4826_group, SGB4041, SGB7984, SGB4761, SGB4447, SGB6744, SGB1836_group, SGB1814, SGB4630_group, SGB8163, SGB4617, SGB4588_group, SGB4742, SGB4572, SGB10115, SGB15158, SGB29328, SGB4791, SGB4688, SGB10068, SGB71883, SGB4760, SGB4037_group, SGB4529, SGB4837_group, SGB79883, SGB4762, SGB4797, SGB14809, SGB4758_group, SGB4703, SGB4606, SGB4584, SGB15132, SGB4746, SGB4862, SGB4798, SGB4861, SGB4608, SGB4035, SGB4794_group, SGB4753, SGB4583.

16. The method of embodiment 15, wherein the microbes are ranked based at least in part on a diet rank or health rank.

17. The method of embodiment 15, wherein at least ten of the selected microbes are ranked in the same order relative to each other as per their diet ranks as reflected in Table 1A.

18. The method of embodiment 15, wherein at least ten of the selected microbes are ranked in the same order relative to each other as per their health ranks as reflected in Table 1A.

19. The method of any of embodiments 1 to 18, wherein the microbiome is a gut microbiome.

20. A computer program product including coded instructions that, when run on a computer, implement a method as embodied in any of embodiments 1 to 19.

21. A system including: one or more processors; and one or more non-transitory computer readable media storing instructions executable by the one or more processors, wherein the instruction, when executed, causes the one or more processors to perform operations including: receiving one or more weighted values from one or more first machine learned models trained on microbiome data associated with a plurality of individuals, each of the one or more weighted values representing at least in part a pro-health or poor health impact of a microbe or a plurality of microbes; receiving first test data representing a presence of and quantities of microbes present in a microbiome associated with an individual at a first time; inputting the test data into one or more second machine learned models which use at least in part the one or more weighted values; receiving as a first output of the one or more second machine learned models a first microbiome score representing a quality of the microbiome associated with the individual at the first time; and sending the microbiome score to storage location accessible by a device associated with the individual.

22. The system of embodiment 21, wherein the operations further include: inputting the test data into one or more third machine learned models trained at least in part on observed changes in microbiome data for a plurality of test subjects over time; and receiving as an output of the one or more third machine learned models a recommended action, the recommended action intended to increase a relative quantity of pro-health indicator microbes or decrease a relative quantity of poor health indicator microbes in the microbiome associated with the individual.

23. The system of any of embodiments 21 to 22, wherein the operations further include: receiving second test data representing quantities of microbes present in the microbiome associated with the individual at a second time, the second time subsequent to the first time; inputting the second test data into the one or more second machine learned models and receiving as a second output of the one or more second machine learned models a second microbiome score representing a quality of the microbiome associated with the individual at the second time; and sending the second microbiome score to the storage location accessible by the device associated with the individual.

24. The system of any of embodiments 21 to 23, wherein the operations further include: inputting the test data into one or more fourth machine learned models trained at least in part on observed changes in microbiome data for a plurality of test subjects over time; and receiving as an output of the one or more fourth machine learned models, a plurality of predicted microbiome scores, each of the plurality of predicted microbiome scores representing a quality of the microbiome of the individual at a future time in response to the individual performing a corresponding recommended action.

25. The system of any of embodiments 21 to 24, wherein a negative weighted value represents a poor health associated indicator microbe and a positive weighted value represents a pro-health associated indicator microbe.

26. The system of any of embodiments 21 to 25, wherein inputting the test data into one or more second machine learned models further includes: selecting specific microbes from the test data; and inputting the quantities of the specific microbes into the one or more second machine learned models.

27. The system of any of embodiments 21 to 26, wherein the operations further include: receiving user data associated with the individual, the user data representing at least one of a health of the individual, a demographic associated with the individual, a diet of the individual, or a geographic region associated with the individual.

28. The system of any of embodiments 21 to 27, wherein at least ten of the microbes (identified by its species-level genome bin (SGB) designation) are selected from: SGB15249, SGB6340, SGB4964, SGB14252, SGB15229, SGB6174_group, SGB15317, SGB14179, SGB15225, SGB4894, SGB4643, SGB4963, SGB79840, SGB4893, SGB6276, SGB3952, SGB4638, SGB15236, SGB4191, SGB15053_group, SGB15368, SGB4782, SGB14042, SGB4706, SGB4644, SGB49188, SGB4781, SGB4777, SGB14921, SGB15234, SGB8601, SGB5087, SGB14311, SGB4953, SGB7258, SGB4882, SGB6367, SGB15106, SGB4778, SGB15131, SGB4198_group, SGB15031, SGB13981, SGB15123, SGB54300, SGB4665, SGB13979, SGB15410, SGB2290, SGB14954, SGB14306, SGB4805, SGB14899, SGB4803, SGB13982, SGB15265_group, SGB14114, SGB47656, SGB6749, SGB14253, SGB15346, SGB4810, SGB4770, SGB14043, SGB4886, SGB25497, SGB4815_group, SGB25416, SGB4957, SGB4654, SGB15373, SGB15254, SGB6571, SGB15323, SGB71759, SGB4648, SGB15180, SGB15413, SGB49168, SGB14960, SGB4133, SGB15051, SGB4993, SGB15395, SGB15145, SGB5111, SGB6317, SGB4966, SGB4780, SGB14198, SGB63101, SGB4779, SGB15233, SGB4769, SGB2295, SGB72336, SGB4658, SGB14770, SGB6148, SGB25493, SGB4831_group, SGB14965, SGB15224, SGB4938, SGB15402, SGB15291, SGB9333, SGB4664, SGB4906, SGB4711, SGB15065, SGB714_group, SGB4772, SGB3958, SGB4629, SGB14048, SGB15052, SGB14861, SGB9205, SGB4280, SGB4829, SGB4816, SGB2317, SGB15411, SGB5117, SGB14250, SGB14924, SGB4767, SGB6376, SGB4714, SGB4691, SGB14341, SGB15244, SGB5082_group, SGB4910, SGB4914, SGB8599, SGB4936, SGB15374, SGB72916, SGB4909, SGB15390, SGB15164, SGB15093, SGB13983, SGB5042, SGB4771, SGB15356, SGB72479, SGB4557, SGB3988, SGB15041, SGB14128, SGB15385, SGB6750, SGB4184, SGB3573, SGB66170, SGB15201, SGB15203, SGB79798, SGB15382, SGB4652, SGB9346, SGB14969, SGB4262, SGB4394, SGB61601, SGB15216, SGB14027, SGB4674, SGB14937, SGB15090, SGB9391, SGB15383, SGB29347, SGB14991, SGB14940, SGB4809, SGB6141, SGB4687, SGB63163, SGB14177, SGB4832, SGB15160, SGB48024, SGB6179, SGB4768, SGB5090_group, SGB29302, SGB9712_group, SGB3813, SGB79833, SGB4659, SGB4328, SGB4776, SGB1790, SGB14313, SGB5043, SGB15127, SGB15049, SGB42321, SGB15403, SGB15115, SGB4905, SGB14838, SGB15012, SGB9202, SGB80143, SGB3992, SGB7259, SGB4546, SGB14974, SGB13976, SGB15342, SGB2296, SGB14941, SGB3996, SGB53497, SGB15470, SGB14020, SGB1858, SGB14851, SGB6305, SGB14932, SGB15089, SGB1862, SGB15401, SGB4027, SGB15140, SGB2325, SGB14317, SGB4628, SGB4669, SGB15299, SGB6478, SGB14262, SGB63342, SGB4960, SGB63333, SGB15316_group, SGB4651, SGB1965, SGB15081, SGB59819, SGB2326, SGB14912, SGB14322_group, SGB3940, SGB4029, SGB2301, SGB63167, SGB14797_group, SGB5200, SGB17347, SGB4868, SGB15067, SGB53515, SGB15075, SGB4421, SGB5121, SGB9226, SGB2318, SGB14894, SGB4817, SGB14966, SGB3989, SGB15370, SGB14975, SGB4436, SGB14839, SGB14993_group, SGB15322, SGB9387, SGB3959, SGB6362, SGB4063, SGB14773_group, SGB29334, SGB14151, SGB15087, SGB14022, SGB14972, SGB15045, SGB4712, SGB15389, SGB82503, SGB15318_group, SGB1857, SGB4828_group, SGB4788_group, SGB79822, SGB4269, SGB14923, SGB2328, SGB1784, SGB14137, SGB15204, SGB7256, SGB15295_group, SGB14182, SGB29342, SGB4438, SGB14824_group, SGB2303, SGB9262, SGB14952, SGB14951, SGB15459, SGB6358, SGB14929, SGB15286, SGB5060, SGB15332_group, SGB15126, SGB72433_group, SGB4045, SGB1626, SGB5180, SGB4867, SGB4825, SGB4925, SGB53517, SGB1844, SGB14844, SGB4871_group, SGB14050, SGB25547, SGB1815, SGB3957, SGB54347, SGB6140, SGB14127, SGB29339, SGB1832, SGB9342_group, SGB15278, SGB9203, SGB1962, SGB5076, SGB4808, SGB15119, SGB3962, SGB14892, SGB4775, SGB4166, SGB7144, SGB4959, SGB63353, SGB14953, SGB6139, SGB16971, SGB15300, SGB3574, SGB47850, SGB63327, SGB4181, SGB15506, SGB5190, SGB14181, SGB1846, SGB15068, SGB4537, SGB14906, SGB9347, SGB1786, SGB7265, SGB4571, SGB29321, SGB4531, SGB15073, SGB14933, SGB15154, SGB6747, SGB15273, SGB4367, SGB15091, SGB3965, SGB63369, SGB9224, SGB3964, SGB6952, SGB4765, SGB29305, SGB4285_group, SGB5089, SGB4581, SGB59869, SGB4727, SGB25431, SGB2299, SGB1829, SGB4990, SGB1891_group, SGB2286, SGB17278, SGB1949, SGB63343, SGB5045, SGB5075_group, SGB8007_group, SGB29375, SGB6847, SGB1798, SGB4670, SGB4582_group, SGB4290, SGB14891, SGB1785, SGB15209, SGB14150, SGB33551, SGB14958, SGB14807, SGB4820, SGB1812, SGB4716, SGB4425_group, SGB9340, SGB14779, SGB14125, SGB14259, SGB4784, SGB15260, SGB14741, SGB4577_group, SGB71281, SGB14307, SGB5803, SGB58519, SGB5077, SGB15125, SGB15143, SGB4116, SGB4677, SGB15467_group, SGB7202, SGB6178, SGB6796_group, SGB6783_group, SGB1860, SGB4059, SGB63326, SGB9272_group, SGB15350, SGB14334, SGB1941, SGB16986, SGB14909, SGB1963, SGB4774, SGB14143, SGB15272, SGB29380, SGB4811_group, SGB4595, SGB4834, SGB4030, SGB8071, SGB2311, SGB3991, SGB4722, SGB17244, SGB3993, SGB4553, SGB6956, SGB1699, SGB6962_group, SGB4705, SGB1957, SGB4532, SGB4327_group, SGB59559, SGB4594, SGB5765_group, SGB17169, SGB15120, SGB1948, SGB14853, SGB14898, SGB48424, SGB5792, SGB6754, SGB1934, SGB14854, SGB6768, SGB15156_group, SGB4750, SGB14142, SGB17153_group, SGB4552_group, SGB4951, SGB4303, SGB9286, SGB5051, SGB17237, SGB4940, SGB4597, SGB4563_group, SGB1867, SGB47515, SGB59562, SGB8059_group, SGB4991, SGB4540_group, SGB14862, SGB4422, SGB15124, SGB14987, SGB7263, SGB9283, SGB4348, SGB14895, SGB17154, SGB17167, SGB4626, SGB5843, SGB4575, SGB4613, SGB15076, SGB17130, SGB15904, SGB4080, SGB6846, SGB5825_group, SGB14808, SGB4874, SGB9228, SGB6320, SGB9260, SGB8002, SGB6936, SGB14962, SGB8053, SGB4701, SGB5197, SGB4036, SGB4744, SGB1877, SGB29313, SGB14845, SGB14963, SGB8056, SGB6939, SGB17256, SGB4749, SGB3961, SGB7142, SGB14999, SGB4747, SGB15149, SGB4987, SGB6767, SGB17248, SGB4725, SGB59576, SGB1903_group, SGB5183, SGB49059, SGB4121, SGB25538, SGB3970, SGB3969, SGB14890, SGB1830_group, SGB7967, SGB17168, SGB8047, SGB4046, SGB1855_group, SGB3922, SGB14995, SGB7253, SGB48013, SGB4741, SGB4671, SGB15878, SGB6769, SGB14180, SGB29433, SGB1871, SGB5182, SGB5736, SGB6771, SGB4721, SGB14837, SGB4044, SGB8095, SGB17152, SGB1861, SGB66069, SGB4031, SGB6153, SGB7264, SGB15121, SGB25437, SGB14546_group, SGB4933_group, SGB4763, SGB5193, SGB7985, SGB4699, SGB19850_group, SGB17137, SGB4785, SGB15078, SGB53821, SGB15452, SGB15271, SGB4724, SGB8255_group, SGB79823, SGB8028_group, SGB4573_group, SGB14874, SGB4988, SGB5184, SGB4786, SGB4826_group, SGB4041, SGB7984, SGB4761, SGB4447, SGB6744, SGB1836_group, SGB1814, SGB4630_group, SGB8163, SGB4617, SGB4588_group, SGB4742, SGB4572, SGB10115, SGB15158, SGB29328, SGB4791, SGB4688, SGB10068, SGB71883, SGB4760, SGB4037_group, SGB4529, SGB4837_group, SGB79883, SGB4762, SGB4797, SGB14809, SGB4758_group, SGB4703, SGB4606, SGB4584, SGB15132, SGB4746, SGB4862, SGB4798, SGB4861, SGB4608, SGB4035, SGB4794_group, SGB4753, SGB4583.

29. The system of embodiment 28, wherein the microbes are ranked based at least in part on a diet rank or health rank.

30. The system of embodiment 28, wherein at least ten of the selected microbes are ranked in the same order relative to each other as per their diet ranks as reflected in Table 1A.

31. The system of embodiment 28, wherein at least ten of the selected microbes are ranked in the same order relative to each other as per their health ranks as reflected in Table 1A.

32. The system of any of embodiments 22 to 31, wherein the microbiome data is gut microbiome data.

33. The system of any of embodiments 22-32, wherein the quantity of microbes is expressed as a percentage of the microbiome.

34. A method including: receiving first test data from a remote device, the first test data representing quantities of microbes present in one or more first microbiome samples; receiving second test data from a remote device, the second test data representing quantities of microbes present in one or more second microbiome samples; accessing a list of indicator microbes and their associations with pro-health versus poor health; determining which indicator microbes increase or decrease between the first and second microbiome samples; determining whether the increasing indicator microbes have stronger pro-health associations or stronger poor health associations compared to the decreasing indicator microbes, representing respectively an increase or decrease in the quality of the microbiome between the first and second microbiomes samples; and sending the comparison to a storage location.

35. The method of embodiment 34, wherein the microbiome is a gut microbiome.

36. The method of embodiment 34 or 35, wherein the quantity of microbes is expressed as a percentage of the microbiome.

37. The method of any of embodiments 34 to 36, wherein at least ten of the microbes (identified by its species-level genome bin (SGB) designation) are selected from: SGB15249, SGB6340, SGB4964, SGB14252, SGB15229, SGB6174_group, SGB15317, SGB14179, SGB15225, SGB4894, SGB4643, SGB4963, SGB79840, SGB4893, SGB6276, SGB3952, SGB4638, SGB15236, SGB4191, SGB15053_group, SGB15368, SGB4782, SGB14042, SGB4706, SGB4644, SGB49188, SGB4781, SGB4777, SGB14921, SGB15234, SGB8601, SGB5087, SGB14311, SGB4953, SGB7258, SGB4882, SGB6367, SGB15106, SGB4778, SGB15131, SGB4198_group, SGB15031, SGB13981, SGB15123, SGB54300, SGB4665, SGB13979, SGB15410, SGB2290, SGB14954, SGB14306, SGB4805, SGB14899, SGB4803, SGB13982, SGB15265_group, SGB14114, SGB47656, SGB6749, SGB14253, SGB15346, SGB4810, SGB4770, SGB14043, SGB4886, SGB25497, SGB4815_group, SGB25416, SGB4957, SGB4654, SGB15373, SGB15254, SGB6571, SGB15323, SGB71759, SGB4648, SGB15180, SGB15413, SGB49168, SGB14960, SGB4133, SGB15051, SGB4993, SGB15395, SGB15145, SGB5111, SGB6317, SGB4966, SGB4780, SGB14198, SGB63101, SGB4779, SGB15233, SGB4769, SGB2295, SGB72336, SGB4658, SGB14770, SGB6148, SGB25493, SGB4831_group, SGB14965, SGB15224, SGB4938, SGB15402, SGB15291, SGB9333, SGB4664, SGB4906, SGB4711, SGB15065, SGB714_group, SGB4772, SGB3958, SGB4629, SGB14048, SGB15052, SGB14861, SGB9205, SGB4280, SGB4829, SGB4816, SGB2317, SGB15411, SGB5117, SGB14250, SGB14924, SGB4767, SGB6376, SGB4714, SGB4691, SGB14341, SGB15244, SGB5082_group, SGB4910, SGB4914, SGB8599, SGB4936, SGB15374, SGB72916, SGB4909, SGB15390, SGB15164, SGB15093, SGB13983, SGB5042, SGB4771, SGB15356, SGB72479, SGB4557, SGB3988, SGB15041, SGB14128, SGB15385, SGB6750, SGB4184, SGB3573, SGB66170, SGB15201, SGB15203, SGB79798, SGB15382, SGB4652, SGB9346, SGB14969, SGB4262, SGB4394, SGB61601, SGB15216, SGB14027, SGB4674, SGB14937, SGB15090, SGB9391, SGB15383, SGB29347, SGB14991, SGB14940, SGB4809, SGB6141, SGB4687, SGB63163, SGB14177, SGB4832, SGB15160, SGB48024, SGB6179, SGB4768, SGB5090_group, SGB29302, SGB9712_group, SGB3813, SGB79833, SGB4659, SGB4328, SGB4776, SGB1790, SGB14313, SGB5043, SGB15127, SGB15049, SGB42321, SGB15403, SGB15115, SGB4905, SGB14838, SGB15012, SGB9202, SGB80143, SGB3992, SGB7259, SGB4546, SGB14974, SGB13976, SGB15342, SGB2296, SGB14941, SGB3996, SGB53497, SGB15470, SGB14020, SGB1858, SGB14851, SGB6305, SGB14932, SGB15089, SGB1862, SGB15401, SGB4027, SGB15140, SGB2325, SGB14317, SGB4628, SGB4669, SGB15299, SGB6478, SGB14262, SGB63342, SGB4960, SGB63333, SGB15316_group, SGB4651, SGB1965, SGB15081, SGB59819, SGB2326, SGB14912, SGB14322_group, SGB3940, SGB4029, SGB2301, SGB63167, SGB14797_group, SGB5200, SGB17347, SGB4868, SGB15067, SGB53515, SGB15075, SGB4421, SGB5121, SGB9226, SGB2318, SGB14894, SGB4817, SGB14966, SGB3989, SGB15370, SGB14975, SGB4436, SGB14839, SGB14993_group, SGB15322, SGB9387, SGB3959, SGB6362, SGB4063, SGB14773_group, SGB29334, SGB14151, SGB15087, SGB14022, SGB14972, SGB15045, SGB4712, SGB15389, SGB82503, SGB15318_group, SGB1857, SGB4828_group, SGB4788_group, SGB79822, SGB4269, SGB14923, SGB2328, SGB1784, SGB14137, SGB15204, SGB7256, SGB15295_group, SGB29342, SGB14182, SGB4438, SGB14824_group, SGB2303, SGB9262, SGB14952, SGB14951, SGB15459, SGB6358, SGB14929, SGB15286, SGB5060, SGB15332_group, SGB15126, SGB72433_group, SGB4045, SGB1626, SGB5180, SGB4867, SGB4825, SGB4925, SGB53517, SGB1844, SGB14844, SGB4871_group, SGB14050, SGB25547, SGB1815, SGB3957, SGB54347, SGB6140, SGB14127, SGB29339, SGB1832, SGB9342_group, SGB15278, SGB9203, SGB1962, SGB5076, SGB4808, SGB15119, SGB3962, SGB14892, SGB4775, SGB4166, SGB7144, SGB4959, SGB63353, SGB14953, SGB6139, SGB16971, SGB15300, SGB3574, SGB47850, SGB63327, SGB4181, SGB15506, SGB5190, SGB14181, SGB1846, SGB15068, SGB4537, SGB14906, SGB9347, SGB1786, SGB7265, SGB4571, SGB29321, SGB4531, SGB15073, SGB14933, SGB15154, SGB6747, SGB15273, SGB4367, SGB15091, SGB3965, SGB63369, SGB9224, SGB3964, SGB6952, SGB4765, SGB29305, SGB4285_group, SGB5089, SGB4581, SGB59869, SGB4727, SGB25431, SGB2299, SGB1829, SGB4990, SGB1891_group, SGB2286, SGB17278, SGB1949, SGB63343, SGB5045, SGB5075_group, SGB8007_group, SGB29375, SGB6847, SGB1798, SGB4670, SGB4582_group, SGB4290, SGB14891, SGB1785, SGB15209, SGB14150, SGB33551, SGB14958, SGB14807, SGB4820, SGB1812, SGB4716, SGB4425_group, SGB9340, SGB14779, SGB14125, SGB14259, SGB4784, SGB15260, SGB14741, SGB4577_group, SGB71281, SGB14307, SGB5803, SGB58519, SGB5077, SGB15125, SGB15143, SGB4116, SGB4677, SGB15467_group, SGB7202, SGB6178, SGB6796_group, SGB6783_group, SGB1860, SGB4059, SGB63326, SGB9272_group, SGB15350, SGB14334, SGB1941, SGB16986, SGB14909, SGB1963, SGB4774, SGB14143, SGB15272, SGB29380, SGB4811_group, SGB4595, SGB4834, SGB4030, SGB8071, SGB2311, SGB3991, SGB4722, SGB17244, SGB3993, SGB4553, SGB6956, SGB1699, SGB6962_group, SGB4705, SGB1957, SGB4532, SGB4327_group, SGB59559, SGB4594, SGB5765_group, SGB17169, SGB15120, SGB1948, SGB14853, SGB14898, SGB48424, SGB5792, SGB6754, SGB1934, SGB14854, SGB6768, SGB15156_group, SGB4750, SGB14142, SGB17153_group, SGB4552_group, SGB4951, SGB4303, SGB9286, SGB5051, SGB17237, SGB4940, SGB4597, SGB4563_group, SGB1867, SGB47515, SGB59562, SGB8059_group, SGB4991, SGB4540_group, SGB14862, SGB4422, SGB15124, SGB14987, SGB7263, SGB9283, SGB4348, SGB14895, SGB17154, SGB17167, SGB4626, SGB5843, SGB4575, SGB4613, SGB15076, SGB17130, SGB15904, SGB4080, SGB6846, SGB5825_group, SGB14808, SGB4874, SGB9228, SGB6320, SGB9260, SGB8002, SGB6936, SGB14962, SGB8053, SGB4701, SGB5197, SGB4036, SGB4744, SGB1877, SGB29313, SGB14845, SGB14963, SGB8056, SGB6939, SGB17256, SGB4749, SGB3961, SGB7142, SGB14999, SGB4747, SGB15149, SGB4987, SGB6767, SGB17248, SGB4725, SGB59576, SGB1903_group, SGB5183, SGB49059, SGB4121, SGB25538, SGB3970, SGB3969, SGB14890, SGB1830_group, SGB7967, SGB17168, SGB8047, SGB4046, SGB1855_group, SGB3922, SGB14995, SGB7253, SGB48013, SGB4741, SGB4671, SGB15878, SGB6769, SGB14180, SGB29433, SGB1871, SGB5182, SGB5736, SGB6771, SGB4721, SGB14837, SGB4044, SGB8095, SGB17152, SGB1861, SGB66069, SGB4031, SGB6153, SGB7264, SGB15121, SGB25437, SGB14546_group, SGB4933_group, SGB4763, SGB5193, SGB7985, SGB4699, SGB19850_group, SGB17137, SGB4785, SGB15078, SGB53821, SGB15452, SGB15271, SGB4724, SGB8255_group, SGB79823, SGB8028_group, SGB4573_group, SGB14874, SGB4988, SGB5184, SGB4786, SGB4826_group, SGB4041, SGB7984, SGB4761, SGB4447, SGB6744, SGB1836_group, SGB1814, SGB4630_group, SGB8163, SGB4617, SGB4588_group, SGB4742, SGB4572, SGB10115, SGB15158, SGB29328, SGB4791, SGB4688, SGB10068, SGB71883, SGB4760, SGB4037_group, SGB4529, SGB4837_group, SGB79883, SGB4762, SGB4797, SGB14809, SGB4758_group, SGB4703, SGB4606, SGB4584, SGB15132, SGB4746, SGB4862, SGB4798, SGB4861, SGB4608, SGB4035, SGB4794_group, SGB4753, SGB4583.

38. The method of embodiment 37, wherein the microbes are ranked based at least in part on a diet rank or health rank.

39. The method of embodiment 37, wherein at least ten of the selected microbes are ranked in the same order relative to each other as per their diet ranks as reflected in Table 1A.

40. The method of embodiment 37, wherein at least ten of the selected microbes are ranked in the same order relative to each other as per their health ranks as reflected in Table 1A.

### (VIII) EXAMPLE 1: MICROBE ABUNDANCE CHANGES AFTER PERSONALIZED DIET RECOMMENDATIONS DATA SELECTION

At the time of this application, two separate groups of participants have all of their data (metagenomic and questionnaires, prepared essentially as described in Asnicar et al. (Nat Med. 27:321-323, 2021), WO 2021/186047, and/or PCT/EP2022/076241) collected and available for analysis.

As the goal of this analysis was to understand the changes in the gut microbiome following the ZOE personalized diet recommendations, in the first group of participants, users that did not follow the recommendations at least 3 days per week were filtered out.

To allow for a consistent comparison between the initial and the retest metagenomic profiles all participants' microbe DNA sequences were randomly sub-sampled to 20 Million reads. Users for which any of the two metagenomic datasets (initial or retest) did not reach 20 Million clean reads were filtered out, to avoid microbe abundances being influenced by a different read depth between the two time points.

In total, data from a first group of 440 participants and a second group of 1124 participants were selected and analysed as described below.

Statistical analysis. The analysis considers separate cohorts of datasets. The initial cohort includes the metagenomic profiles from the first metagenomics test of the first group of 440 selected participants; the retest cohort includes the metagenomic profiles for the same 440 users at a retesting point 2 to 12 months after the first test. Table 6 reflects data from the second group of 1124 participants pre-intervention and post-intervention.

To obtain the normalized relative abundance, the relative abundances were normalised using the function: arcsin (sqrt (abundance/100)). The relative abundance distributions of the good and the bad bugs of the two cohorts were compared to each other using a Wilcoxon signed-rank test to establish whether there are statistically significant differences between the abundances in the two cohorts.

In Tables 2-5, in order to reduce type-1 errors due to the False Discovery Rate in presence of, the p-values obtained by the Wilcoxon signed-rank test were finally corrected using the Benjamini-Hochberg procedure (Adjusted p-value). In the following, any abundance change with an Adjusted p-value>=0.2 was consider as significant.

### Results

### List of Microbes Selected Using Health Metrics and Diet Markers

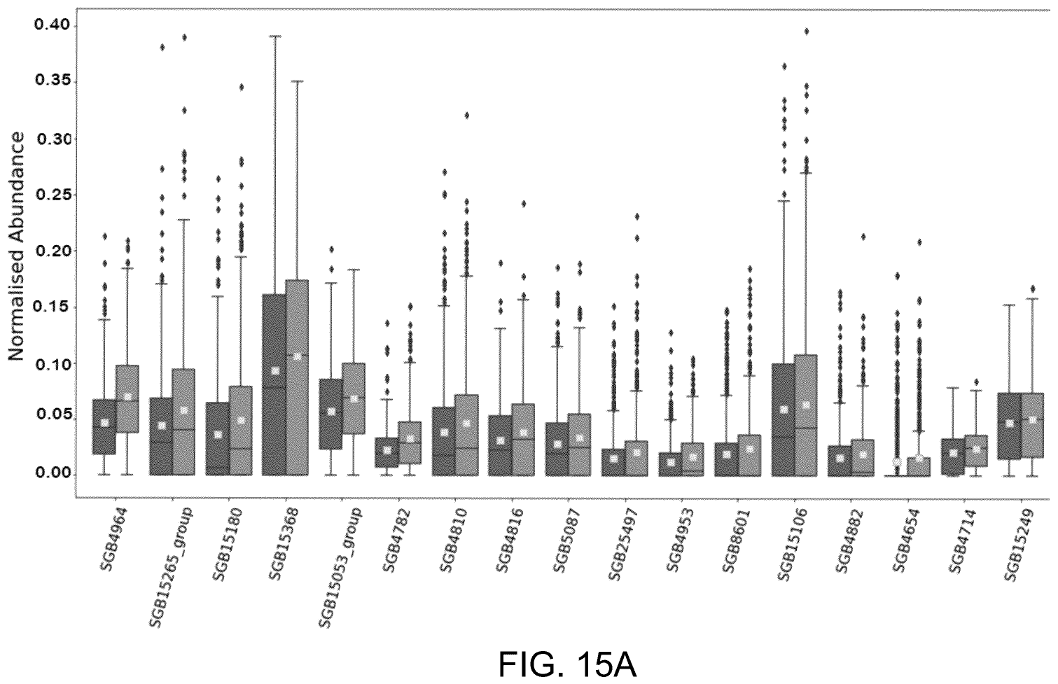

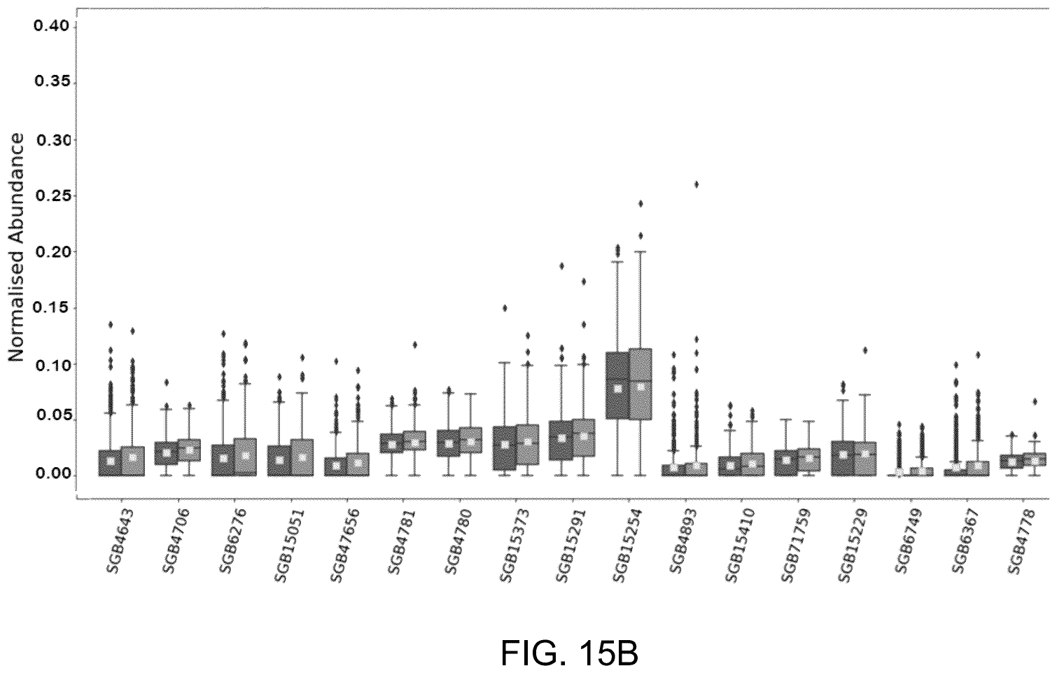

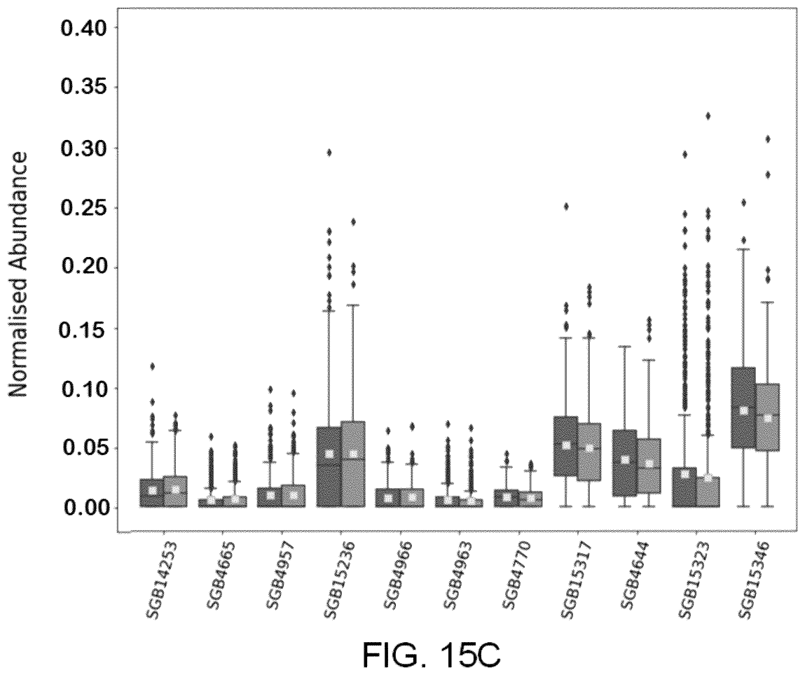

Changes in the Good Bugs in the first group of 440 participants: Of the initial 60 GOOD microbes selected with health and diet markers, 39 increased significantly at retest time compared to the baseline and 6 decreased significantly-shown in Table 2. The normalised relative abundances for the initial and retest cohorts for the 45 significantly changing good microbes extracted with diet markers are shown in FIGS. 15A-15C with the first timepoint shown to the left of each pair of measurements for a particular microbe (darker grey) and the second timepoint shown on the right of each pair of measurements for a particular microbe (lighter grey).

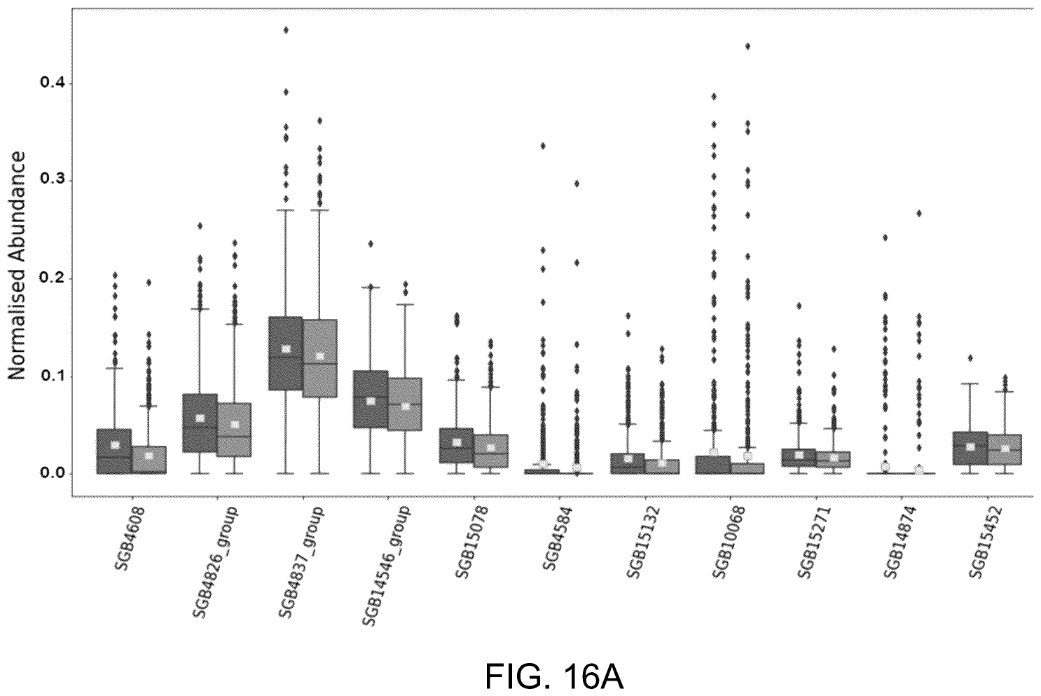

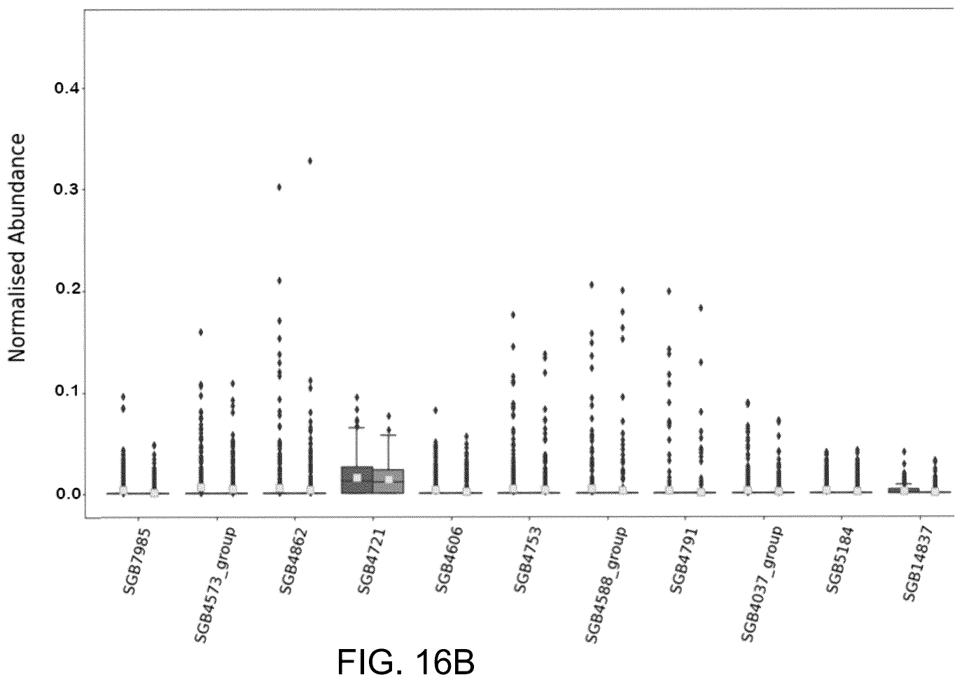

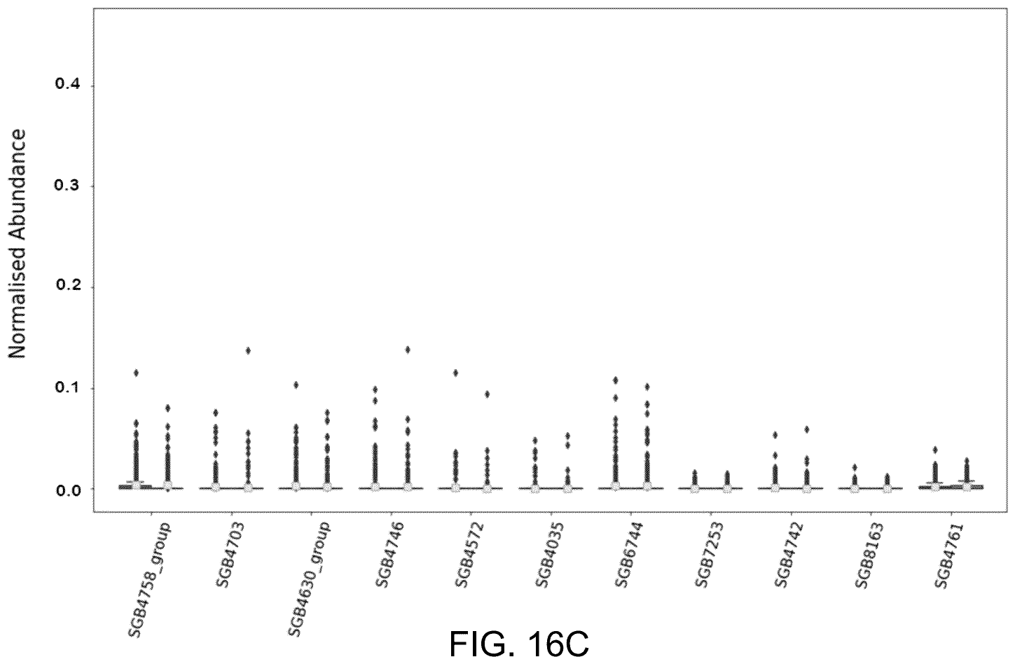

Changes in the Bad Bugs in the first group of 440 participants: Of the initial 60 BAD microbes selected with health and diet markers, 32 decreased significantly at retest time compared to the baseline and 1 increased significantly-shown in Table 3. The normalised relative abundances for the initial and retest cohorts for the 33 significantly changing bad microbes extracted with diet markers are shown in FIGS. 16A-16C with the first timepoint shown to the left of each pair of measurements for a particular microbe (darker grey) and the second timepoint shown on the right of each pair of measurements for a particular microbe (lighter gray).

### List of Microbes Selected Using Only Health Metrics

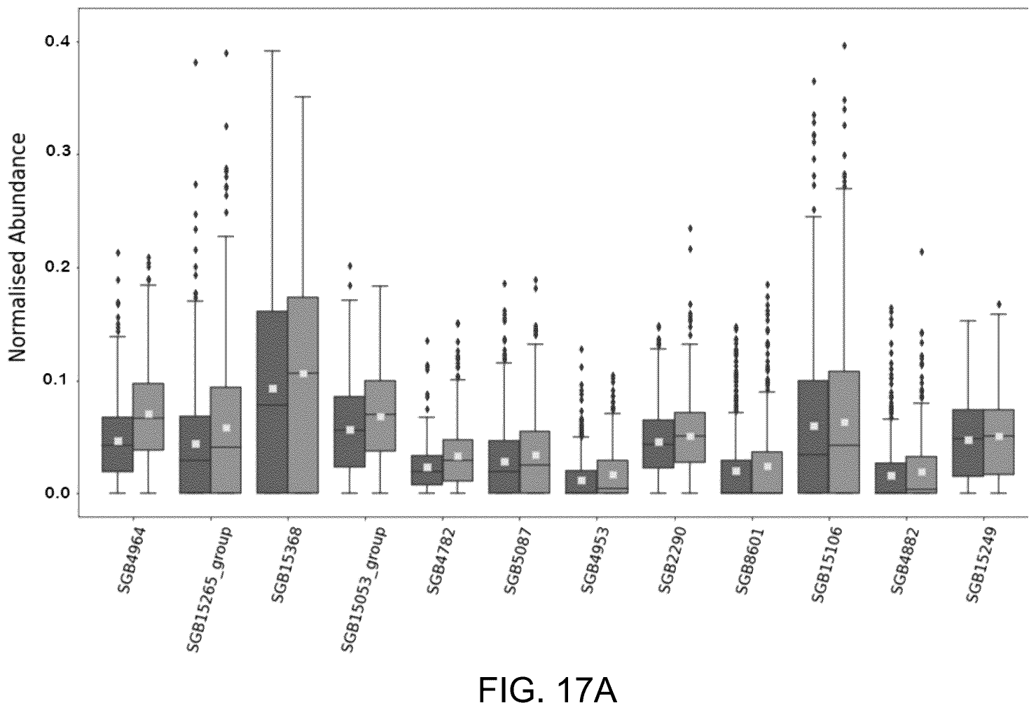

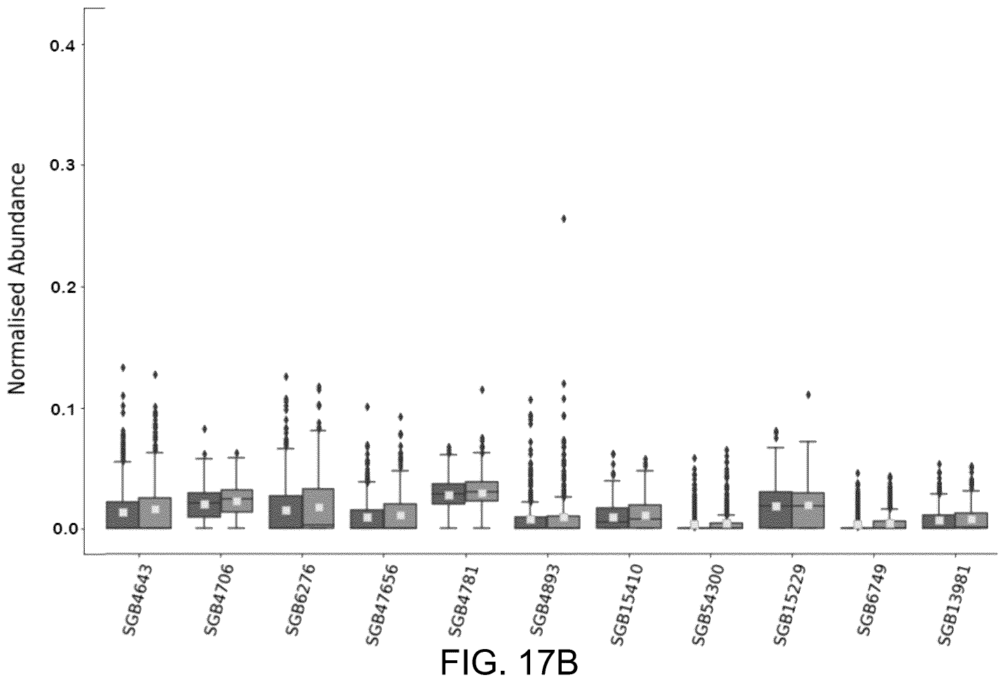

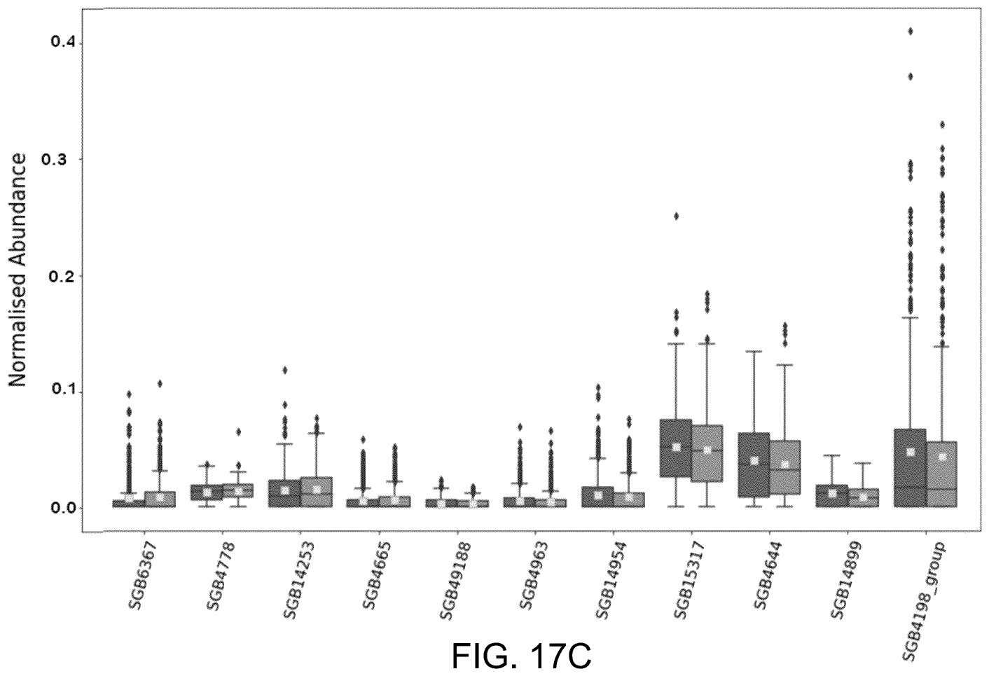

Changes in the Good Bugs in the first group of 440 participants: Of the initial 60 GOOD microbes selected only with health markers, 27 increased significantly at retest time compared to the baseline and 7 decreased significantly-shown in Table 4. The normalised relative abundances for the initial and retest cohorts for the 34 significantly changing good microbes extracted without diet markers are shown in FIGS. 17A-17C with the first timepoint shown to the left of each pair of measurements for a particular microbe and the second timepoint shown on the right of each pair of measurements for a particular microbe.

### Changes in the Bad Bugs in the First Group of 440 Participants:

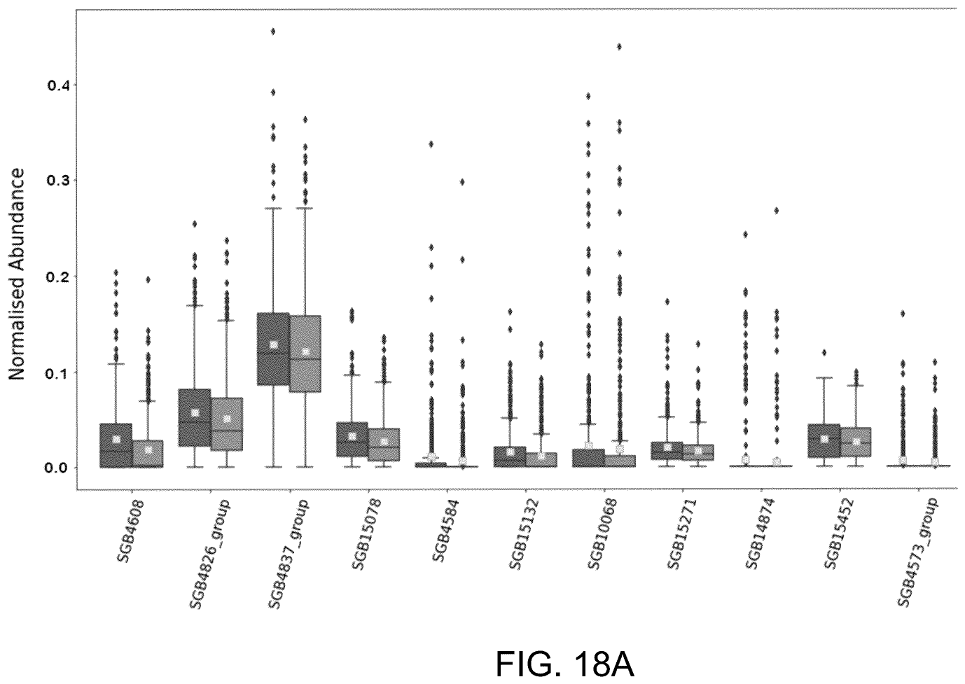

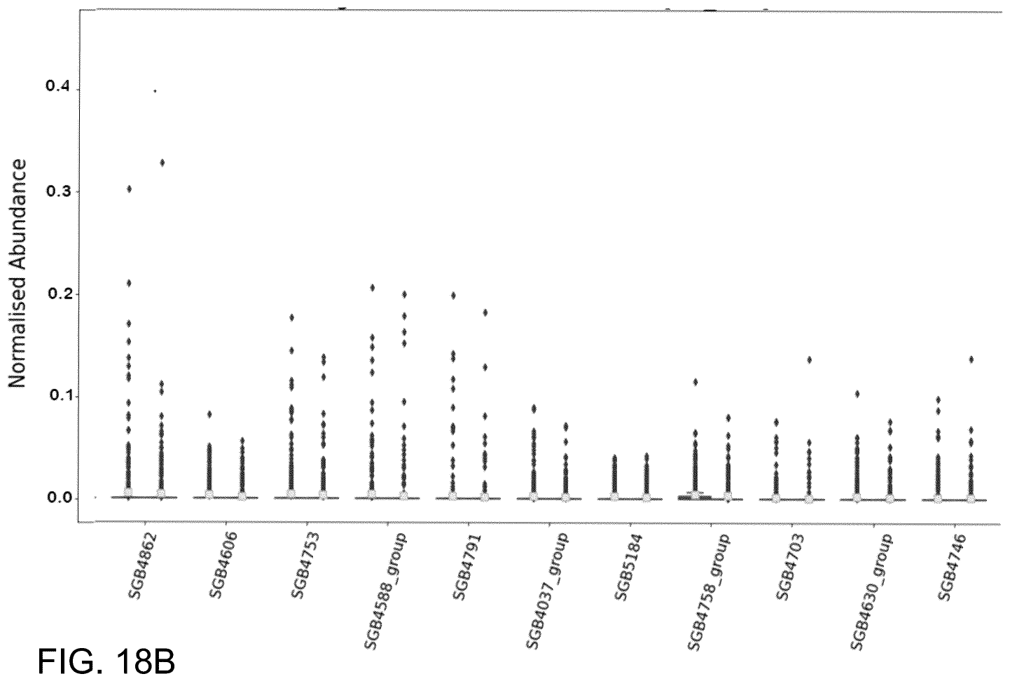

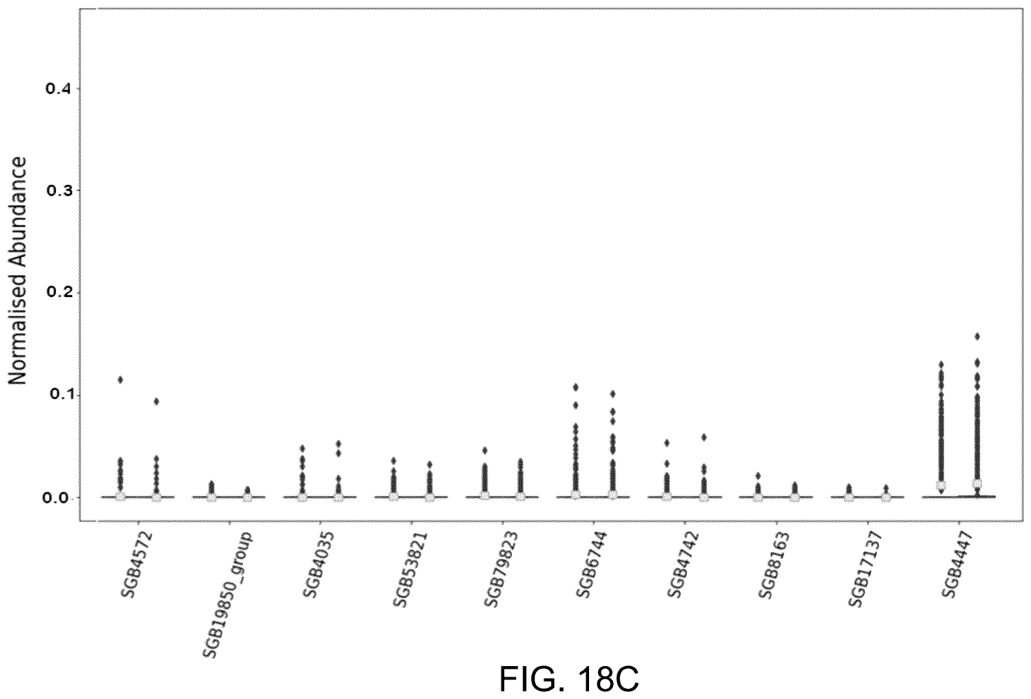

Of the initial 60 BAD microbes selected only with health markers, 31 decreased significantly at retest time compared to the baseline and 1 increased significantly-shown in Table 5. The normalised relative abundances for the initial and retest cohorts for the 32 significantly changing bad microbes extracted with diet markers are shown in FIGS. 18A-18C with the first timepoint shown to the left of each pair of measurements for a particular microbe (darker grey) and the second timepoint shown on the right of each pair of measurements for a particular microbe (lighter grey).

### Changes in the Second Group of 1124 Participants

As shown in Table 6 which includes all significant SGBs (Wilcoxon rank-sum test, Bonferroni correcter p-values<0.01) and their change in relative abundance and prevalence pre and post intervention), 268 bacteria were statistically sensitive to following the ZOE personalized diet recommendations for 2 to 12 months in the second group of 1124 individuals.

### DISCUSSION

On average, following ZOE personalized diet recommendations positively influenced the gut microbiome of the participants by modulating the relative abundance of the microbes most correlated with a selection of good and poor health markers, and good or poor initial diet.

The results are similar for the lists of microbes extracted for their correlations with health and diet markers or for those extracted for their correlations only with health markers: most of the microbes associated with good health and good diet markers (GOOD BUGS) increased their relative abundance after the program, while most of the microbes associated with poor health and poor diet markers (BAD BUGS) had their relative abundances reduced at retest time.

In particular, 27 (39) of the 60 GOOD BUGS extracted for their correlations with health (and diet) markers statistically significantly increased for the first group of 440 participants after following the ZOE program, while 7 (6) statistically significantly decreased.

Of the 60 BAD BUGS, 31 (32) statistically significantly decreased for the first group of 440 participants at retest time, and 1 (1) statistically significantly increased.

TABLE 2
Good Bugs extracted with health and diet markers from the first group
of 440 participants - changes in the first group of 440 participants
Normalised relative abundances of the 60 good bugs extracted with health and diet markers
at the first test (Initial Abundance) and at retest time (Retest Abundance). Also shown
are the Fold Change, the p-values relative to the Wilcoxon signed rank test (p-value)
and the p-values adjusted for False Discovery Rate (Adjusted p-value). The field Type
of Change reports whether after the diet intervention the relative abundance increased
or decreased. The table is sorted according to the Adjusted p-value. Italics indicates the
microbes for which the normalized relative abundance change was not considered significant.

| | Initial | Retest | Fold | Delta | | Adjusted | Type of |
|---|---|---|---|---|---|---|---|
| SGB | Abundance | Abundance | Change | Change | p-value | p-value | Change |
| SGB4964 | 0.04689 | 0.07045 | 1.50261 | 0.02357 | 1.23E−24 | 1.48E−22 | [increased] |
| SGB4782 | 0.02312 | 0.03313 | 1.43302 | 0.01001 | 6.29E−22 | 3.77E−20 | [increased] |
| SGB15053_group | 0.05702 | 0.06836 | 1.19892 | 0.01134 | 1.51E−10 | 3.03E−09 | [increased] |
| SGB15265_group | 0.04415 | 0.05845 | 1.32393 | 0.01430 | 6.60E−10 | 1.13E−08 | [increased] |
| SGB15180 | 0.03655 | 0.04961 | 1.35742 | 0.01306 | 6.56E−09 | 9.84E−08 | [increased] |
| SGB4706 | 0.02008 | 0.02263 | 1.12673 | 0.00255 | 9.50E−08 | 9.50E−07 | [increased] |
| SGB4714 | 0.02106 | 0.02433 | 1.15550 | 0.00327 | 3.37E−07 | 2.89E−06 | [increased] |
| SGB4953 | 0.01208 | 0.01690 | 1.39925 | 0.00482 | 3.23E−07 | 2.89E−06 | [increased] |
| SGB4816 | 0.03190 | 0.03882 | 1.21683 | 0.00692 | 1.81E−06 | 1.35E−05 | [increased] |
| SGB15368 | 0.09352 | 0.10651 | 1.13893 | 0.01299 | 7.62E−06 | 5.38E−05 | [increased] |
| SGB25497 | 0.01592 | 0.02089 | 1.31198 | 0.00497 | 1.61E−05 | 1.02E−04 | [increased] |
| SGB4810 | 0.03895 | 0.04672 | 1.19928 | 0.00776 | 5.23E−05 | 2.74E−04 | [increased] |
| SGB15346 | 0.08054 | 0.07384 | 0.91679 | −0.00670 | 5.25E−05 | 2.74E−04 | [decreased] |
| SGB47656 | 0.00924 | 0.01132 | 1.22517 | 0.00208 | 5.65E−05 | 2.83E−04 | [increased] |
| SGB5087 | 0.02865 | 0.03414 | 1.19141 | 0.00548 | 8.16E−05 | 3.92E−04 | [increased] |
| SGB4770 | 0.00870 | 0.00737 | 0.84700 | −0.00133 | 1.80E−04 | 7.69E−04 | [decreased] |
| SGB15410 | 0.00948 | 0.01092 | 1.15162 | 0.00144 | 2.31E−04 | 9.58E−04 | [increased] |
| SGB4654 | 0.01313 | 0.01662 | 1.26634 | 0.00350 | 5.40E−04 | 1.96E−03 | [increased] |
| SGB4781 | 0.02757 | 0.02959 | 1.07321 | 0.00202 | 7.03E−04 | 2.41E−03 | [increased] |
| SGB15373 | 0.02774 | 0.02967 | 1.06993 | 0.00194 | 8.85E−04 | 2.95E−03 | [increased] |
| SGB4778 | 0.01243 | 0.01346 | 1.08292 | 0.00103 | 1.01E−03 | 3.28E−03 | [increased] |
| SGB4643 | 0.01352 | 0.01631 | 1.20604 | 0.00279 | 1.50E−03 | 4.63E−03 | [increased] |
| SGB4780 | 0.02819 | 0.03018 | 1.07078 | 0.00200 | 2.31E−03 | 6.76E−03 | [increased] |
| SGB15051 | 0.01415 | 0.01638 | 1.15785 | 0.00223 | 2.46E−03 | 7.02E−03 | [increased] |
| SGB6749 | 0.00355 | 0.00461 | 1.29893 | 0.00106 | 2.72E−03 | 7.59E−03 | [increased] |
| SGB8601 | 0.01986 | 0.02429 | 1.22319 | 0.00443 | 6.98E−03 | 1.74E−02 | [increased] |
| SGB4882 | 0.01616 | 0.01967 | 1.21739 | 0.00351 | 6.92E−03 | 1.74E−02 | [increased] |
| SGB15249 | 0.04772 | 0.05064 | 1.06123 | 0.00292 | 7.50E−03 | 1.84E−02 | [increased] |
| SGB71759 | 0.01430 | 0.01543 | 1.07885 | 0.00113 | 8.33E−03 | 2.00E−02 | [increased] |
| SGB15323 | 0.02754 | 0.02442 | 0.88673 | −0.00312 | 1.36E−02 | 3.03E−02 | [decreased] |
| SGB15291 | 0.03296 | 0.03475 | 1.05438 | 0.00179 | 1.77E−02 | 3.72E−02 | [increased] |
| SGB4963 | 0.00562 | 0.00484 | 0.86230 | −0.00077 | 1.88E−02 | 3.89E−02 | [decreased] |
| SGB6276 | 0.01563 | 0.01806 | 1.15576 | 0.00243 | 2.23E−02 | 4.54E−02 | [increased] |
| SGB6367 | 0.00772 | 0.00875 | 1.13422 | 0.00104 | 2.47E−02 | 4.90E−02 | [increased] |
| SGB15317 | 0.05164 | 0.04905 | 0.94986 | −0.00259 | 2.49E−02 | 4.90E−02 | [decreased] |
| SGB14253 | 0.01404 | 0.01503 | 1.07082 | 0.00099 | 3.38E−02 | 6.33E−02 | [increased] |
| SGB15106 | 0.05964 | 0.06358 | 1.06600 | 0.00394 | 3.86E−02 | 7.12E−02 | [increased] |
| SGB4966 | 0.00781 | 0.00841 | 1.07756 | 0.00061 | 4.21E−02 | 7.53E−02 | [increased] |
| SGB4665 | 0.00563 | 0.00639 | 1.13538 | 0.00076 | 4.20E−02 | 7.53E−02 | [increased] |
| SGB4893 | 0.00753 | 0.00905 | 1.20194 | 0.00152 | 4.82E−02 | 8.39E−02 | [increased] |
| SGB4644 | 0.03980 | 0.03676 | 0.92359 | −0.00304 | 4.92E−02 | 8.44E−02 | [decreased] |
| SGB15254 | 0.07677 | 0.07854 | 1.02306 | 0.00177 | 7.49E−02 | 1.27E−01 | [increased] |
| SGB15229 | 0.01847 | 0.01958 | 1.06002 | 0.00111 | 9.62E−02 | 1.56E−01 | [increased] |
| SGB4957 | 0.00962 | 0.01031 | 1.07121 | 0.00069 | 1.23E−01 | 1.94E−01 | [increased] |
| SGB15236 | 0.04411 | 0.04473 | 1.01403 | 0.00062 | 1.27E−01 | 1.96E−01 | [increased] |
| SGB4638 | 0.00600 | 0.00516 | 0.86065 | −0.00084 | 1.86E−01 | 2.76E−01 | [decreased] |
| SGB14042 | 0.00884 | 0.00956 | 1.08131 | 0.00072 | 2.10E−01 | 3.08E−01 | [increased] |
| SGB6340 | 0.01590 | 0.01514 | 0.95202 | −0.00076 | 2.16E−01 | 3.13E−01 | [decreased] |
| SGB4777 | 0.01750 | 0.01789 | 1.02234 | 0.00039 | 2.70E−01 | 3.68E−01 | [increased] |
| SGB13979 | 0.00783 | 0.00839 | 1.07171 | 0.00056 | 2.87E−01 | 3.83E−01 | [increased] |
| SGB15145 | 0.01496 | 0.01539 | 1.02900 | 0.00043 | 3.51E−01 | 4.48E−01 | [increased] |
| SGB6174_{—}group | 0.01623 | 0.01547 | 0.95331 | −0.00076 | 3.54E−01 | 4.48E−01 | [decreased] |
| SGB79840 | 0.00334 | 0.00347 | 1.03700 | 0.00012 | 4.82E−01 | 5.73E−01 | [increased] |
| SGB7258 | 0.00100 | 0.00091 | 0.91305 | −0.00009 | 4.98E−01 | 5.86E−01 | [decreased] |
| SGB4191 | 0.02997 | 0.02915 | 0.97261 | −0.00082 | 5.27E−01 | 6.02E−01 | [decreased] |
| SGB4894 | 0.01494 | 0.01633 | 1.09299 | 0.00139 | 5.24E−01 | 6.02E−01 | [increased] |
| SGB14252 | 0.01124 | 0.01131 | 1.00598 | 0.00007 | 5.80E−01 | 6.50E−01 | [increased] |
| SGB14179 | 0.00546 | 0.00552 | 1.01003 | 0.00005 | 8.61E−01 | 9.14E−01 | [increased] |
| SGB15225 | 0.01667 | 0.01683 | 1.00984 | 0.00016 | 8.96E−01 | 9.38E−01 | [increased] |
| SGB49168 | 0.00453 | 0.00451 | 0.99513 | −0.00002 | 9.34E−01 | 9.66E−01 | [decreased] |

TABLE 3
Bad Bugs extracted with health and diet markers from the first group of 440 participants
Normalised relative abundances of the 60 bad bugs extracted with health and diet markers
at the first test (Initial Abundance) and at retest time (Retest Abundance). Also shown are
the Fold Change, the p-values relative to the Wilcoxon signed rank test (Wilcoxon p-value)
and the p-values adjusted for False Discovery Rate (Adjusted p-value). The field Type
of Change reports whether after the diet intervention the relative abundance increased
or decreased. The table is sorted according to the Adjusted p-value. Italics indicates the
microbes for which the normalized relative abundance change was not considered significant.

| | Initial | Retest | Fold | Delta | | Adjusted | Type of |
|---|---|---|---|---|---|---|---|
| SGB | Abundance | Abundance | Change | Change | p-value | p-value | Change |
| SGB4608 | 0.02952 | 0.01888 | 0.63975 | −0.01063 | 1.50E−16 | 6.02E−15 | [decreased] |
| SGB15132 | 0.01559 | 0.01153 | 0.73948 | −0.00406 | 1.09E−10 | 2.68E−09 | [decreased] |
| SGB15078 | 0.03215 | 0.02687 | 0.83589 | −0.00528 | 1.12E−10 | 2.68E−09 | [decreased] |
| SGB4606 | 0.00367 | 0.00215 | 0.58389 | −0.00153 | 1.23E−08 | 1.64E−07 | [decreased] |
| SGB4584 | 0.01084 | 0.00657 | 0.60576 | −0.00428 | 3.28E−08 | 3.93E−07 | [decreased] |
| SGB4826_{—}group | 0.05771 | 0.05059 | 0.87655 | −0.00712 | 7.24E−08 | 7.90E−07 | [decreased] |
| SGB14546_{—}group | 0.07510 | 0.06899 | 0.91859 | −0.00611 | 1.04E−06 | 8.29E−06 | [decreased] |
| SGB15452 | 0.02834 | 0.02608 | 0.92022 | −0.00226 | 1.08E−05 | 7.18E−05 | [decreased] |
| SGB7985 | 0.00387 | 0.00173 | 0.44749 | −0.00214 | 1.73E−05 | 1.04E−04 | [decreased] |
| SGB14837 | 0.00228 | 0.00154 | 0.67649 | −0.00074 | 5.24E−05 | 2.74E−04 | [decreased] |
| SGB15271 | 0.02012 | 0.01677 | 0.83336 | −0.00335 | 9.27E−05 | 4.28E−04 | [decreased] |
| SGB5184 | 0.00312 | 0.00227 | 0.72759 | −0.00085 | 1.63E−04 | 7.25E−04 | [decreased] |
| SGB4573_{—}group | 0.00645 | 0.00462 | 0.71628 | −0.00183 | 2.51E−04 | 1.00E−03 | [decreased] |
| SGB4791 | 0.00286 | 0.00173 | 0.60567 | −0.00113 | 3.86E−04 | 1.48E−03 | [decreased] |
| SGB4837_{—}group | 0.12811 | 0.12102 | 0.94470 | −0.00708 | 3.95E−04 | 1.48E−03 | [decreased] |
| SGB4703 | 0.00175 | 0.00121 | 0.69440 | −0.00053 | 6.19E−04 | 2.18E−03 | [decreased] |
| SGB4037_{—}group | 0.00315 | 0.00208 | 0.66053 | −0.00107 | 1.09E−03 | 3.45E−03 | [decreased] |
| SGB4753 | 0.00515 | 0.00373 | 0.72378 | −0.00142 | 1.99E−03 | 5.96E−03 | [decreased] |
| SGB10068 | 0.02226 | 0.01884 | 0.84615 | −0.00343 | 2.85E−03 | 7.78E−03 | [decreased] |
| SGB4721 | 0.01562 | 0.01404 | 0.89910 | −0.00158 | 6.31E−03 | 1.68E−02 | [decreased] |
| SGB14874 | 0.00740 | 0.00440 | 0.59425 | −0.00300 | 6.54E−03 | 1.71E−02 | [decreased] |
| SGB7253 | 0.00053 | 0.00031 | 0.59094 | −0.00022 | 9.20E−03 | 2.12E−02 | [decreased] |
| SGB4035 | 0.00068 | 0.00036 | 0.53162 | −0.00032 | 9.18E−03 | 2.12E−02 | [decreased] |
| SGB4746 | 0.00253 | 0.00203 | 0.80250 | −0.00050 | 1.19E−02 | 2.70E−02 | [decreased] |
| SGB4572 | 0.00096 | 0.00055 | 0.57217 | −0.00041 | 1.48E−02 | 3.22E−02 | [decreased] |
| SGB8163 | 0.00040 | 0.00020 | 0.50317 | −0.00020 | 1.73E−02 | 3.71E−02 | [decreased] |
| SGB4630_{—}group | 0.00282 | 0.00229 | 0.81219 | −0.00053 | 2.66E−02 | 5.15E−02 | [decreased] |
| SGB4588_{—}group | 0.00451 | 0.00313 | 0.69388 | −0.00138 | 2.82E−02 | 5.37E−02 | [decreased] |
| SGB4862 | 0.00609 | 0.00444 | 0.72930 | −0.00165 | 4.27E−02 | 7.54E−02 | [decreased] |
| SGB6744 | 0.00328 | 0.00300 | 0.91587 | −0.00028 | 8.13E−02 | 1.35E−01 | [decreased] |
| SGB4742 | 0.00087 | 0.00066 | 0.76536 | −0.00020 | 8.87E−02 | 1.46E−01 | [decreased] |
| SGB4758_{—}group | 0.00449 | 0.00390 | 0.86753 | −0.00059 | 1.00E−01 | 1.60E−01 | [decreased] |
| SGB4761 | 0.00227 | 0.00256 | 1.13010 | 0.00029 | 1.27E−01 | 1.96E−01 | [increased] |
| SGB4699 | 0.00182 | 0.00156 | 0.85691 | −0.00026 | 1.76E−01 | 2.68E−01 | [decreased] |
| SGB4762 | 0.00153 | 0.00116 | 0.75794 | −0.00037 | 1.86E−01 | 2.76E−01 | [decreased] |
| SGB15158 | 0.00621 | 0.00610 | 0.98286 | −0.00011 | 2.29E−01 | 3.27E−01 | [decreased] |
| SGB4529 | 0.00108 | 0.00086 | 0.79648 | −0.00022 | 2.35E−01 | 3.31E−01 | [decreased] |
| SGB14809 | 0.01323 | 0.01225 | 0.92551 | −0.00099 | 2.54E−01 | 3.54E−01 | [decreased] |
| SGB4797 | 0.00052 | 0.00030 | 0.58245 | −0.00022 | 2.60E−01 | 3.59E−01 | [decreased] |
| SGB5193 | 0.00160 | 0.00131 | 0.81735 | −0.00029 | 2.75E−01 | 3.71E−01 | [decreased] |
| SGB4583 | 0.00136 | 0.00106 | 0.78175 | −0.00030 | 2.95E−01 | 3.90E−01 | [decreased] |
| SGB4861 | 0.00045 | 0.00026 | 0.57452 | −0.00019 | 3.33E−01 | 4.33E−01 | [decreased] |
| SGB1814 | 0.12783 | 0.12588 | 0.98477 | −0.00195 | 3.36E−01 | 4.33E−01 | [decreased] |
| SGB8028_{—}group | 0.00069 | 0.00053 | 0.76611 | −0.00016 | 4.69E−01 | 5.73E−01 | [decreased] |
| SGB8255_{—}group | 0.00006 | 0.00011 | 1.93861 | 0.00005 | 4.80E−01 | 5.73E−01 | [increased] |
| SGB71883 | 0.00017 | 0.00020 | 1.17225 | 0.00003 | 4.77E−01 | 5.73E−01 | [increased] |
| SGB4785 | 0.00043 | 0.00026 | 0.60487 | −0.00017 | 4.77E−01 | 5.73E−01 | [decrease] |
| SGB4041 | 0.00232 | 0.00272 | 1.16872 | 0.00039 | 4.74E−01 | 5.73E−01 | [increased] |
| SGB79883 | 0.00017 | 0.00020 | 1.17460 | 0.00003 | 5.23E−01 | 6.02E−01 | [increased] |
| SGB29328 | 0.00057 | 0.00053 | 0.94659 | −0.00003 | 5.38E−01 | 6.09E−01 | [decreased] |
| SGB4763 | 0.00066 | 0.00076 | 1.15491 | 0.00010 | 5.96E−01 | 6.62E−01 | [increased] |
| SGB4760 | 0.00122 | 0.00130 | 1.06376 | 0.00008 | 6.18E−01 | 6.80E−01 | [increased] |
| SGB4988 | 0.00304 | 0.00341 | 1.12182 | 0.00037 | 7.15E−01 | 7.78E−01 | [increased] |
| SGB4786 | 0.00191 | 0.00168 | 0.88170 | −0.00023 | 7.20E−01 | 7.78E−01 | [decreased] |
| SGB6769 | 0.00058 | 0.00062 | 1.07255 | 0.00004 | 7.67E−01 | 8.22E−01 | [increased] |
| SGB4724 | 0.00110 | 0.00117 | 1.06095 | 0.00007 | 8.99E−01 | 9.38E−01 | [increased] |
| SGB4688 | 0.00073 | 0.00080 | 1.08873 | 0.00006 | 9.51E−01 | 9.76E−01 | [increased] |
| SGB4798 | 0.00232 | 0.00238 | 1.02470 | 0.00006 | 9.72E−01 | 9.80E−01 | [increased] |
| SGB4617 | 0.00280 | 0.00298 | 1.06724 | 0.00019 | 9.70E−01 | 9.80E−01 | [increased] |
| SGB4794_{—}group | 0.00064 | 0.00067 | 1.05663 | 0.00004 | 1.00E+00 | 1.00E+00 | [increased] |

TABLE 4
Good Bugs extracted only with health markers (no diet markers) from the first group of 440 participants
Normalised relative abundances of the 60 good bugs extracted only with health markers at the first test
(Initial Abundance) and at retest time (Retest Abundance). Also shown are the Fold Change, the p-values
relative to the Wilcoxon signed rank test (p-value) and the p-values adjusted for False Discovery Rate
(Adjusted p-value). The field Type of Change reports whether after the diet intervention the relative
abundance increased or decreased. The table is sorted according to the Adjusted p-value. Italics
indicates the microbes for which the normalized relative abundance change was not considered significant.

| | Initial | Retest | Fold | Delta | | Adjusted | Type of |
|---|---|---|---|---|---|---|---|
| SGB | Abundance | Abundance | Change | Change | p-value | p-value | Change |
| SGB4964 | 0.04689 | 0.07045 | 1.50261 | 0.02357 | 1.23E−24 | 1.48E−22 | [increased] |
| SGB4782 | 0.02312 | 0.03313 | 1.43302 | 0.01001 | 6.29E−22 | 3.77E−20 | [increased] |
| SGB14899 | 0.01213 | 0.00884 | 0.72892 | −0.00329 | 2.24E−12 | 6.71E−11 | [decreased] |
| SGB15053_group | 0.05702 | 0.06836 | 1.19892 | 0.01134 | 1.51E−10 | 2.60E−09 | [increased] |
| SGB15265_group | 0.04415 | 0.05845 | 1.32393 | 0.01430 | 6.60E−10 | 9.90E−09 | [increased] |
| SGB4706 | 0.02008 | 0.02263 | 1.12673 | 0.00255 | 9.50E−08 | 9.50E−07 | [increased] |
| SGB4953 | 0.01208 | 0.01690 | 1.39925 | 0.00482 | 3.23E−07 | 2.98E−06 | [increased] |
| SGB15368 | 0.09352 | 0.10651 | 1.13893 | 0.01299 | 7.62E−06 | 6.53E−05 | [increased] |
| SGB2290 | 0.04599 | 0.05066 | 1.10163 | 0.00467 | 1.81E−05 | 1.36E−04 | [increased] |
| SGB47656 | 0.00924 | 0.01132 | 1.22517 | 0.00208 | 5.65E−05 | 3.99E−04 | [increased] |
| SGB54300 | 0.00337 | 0.00472 | 1.39885 | 0.00135 | 7.16E−05 | 4.77E−04 | [increased] |
| SGB5087 | 0.02865 | 0.03414 | 1.19141 | 0.00548 | 8.16E−05 | 5.16E−04 | [increased] |
| SGB15410 | 0.00948 | 0.01092 | 1.15162 | 0.00144 | 2.31E−04 | 1.21E−03 | [increased] |
| SGB4781 | 0.02757 | 0.02959 | 1.07321 | 0.00202 | 7.03E−04 | 3.01E−03 | [increased] |
| SGB4778 | 0.01243 | 0.01346 | 1.08292 | 0.00103 | 1.01E−03 | 4.19E−03 | [increased] |
| SGB4643 | 0.01352 | 0.01631 | 1.20604 | 0.00279 | 1.50E−03 | 5.82E−03 | [increased] |
| SGB6749 | 0.00355 | 0.00461 | 1.29893 | 0.00106 | 2.72E−03 | 9.89E−03 | [increased] |
| SGB13981 | 0.00663 | 0.00768 | 1.15842 | 0.00105 | 3.12E−03 | 1.07E−02 | [increased] |
| SGB4882 | 0.01616 | 0.01967 | 1.21739 | 0.00351 | 6.92E−03 | 2.20E−02 | [increased] |
| SGB8601 | 0.01986 | 0.02429 | 1.22319 | 0.00443 | 6.98E−03 | 2.20E−02 | [increased] |
| SGB15249 | 0.04772 | 0.05064 | 1.06123 | 0.00292 | 7.50E−03 | 2.31E−02 | [increased] |
| SGB4198_{—}group | 0.04744 | 0.04375 | 0.92223 | −0.00369 | 1.23E−02 | 3.42E−02 | [decreased] |
| SGB4963 | 0.00562 | 0.00484 | 0.86230 | −0.00077 | 1.88E−02 | 4.91E−02 | [decreased] |
| SGB6276 | 0.01563 | 0.01806 | 1.15576 | 0.00243 | 2.23E−02 | 5.70E−02 | [increased] |
| SGB15317 | 0.05164 | 0.04905 | 0.94986 | −0.00259 | 2.49E−02 | 6.10E−02 | [decreased] |
| SGB6367 | 0.00772 | 0.00875 | 1.13422 | 0.00104 | 2.47E−02 | 6.10E−02 | [increased] |
| SGB14954 | 0.00992 | 0.00851 | 0.85830 | −0.00141 | 3.19E−02 | 7.22E−02 | [decreased] |
| SGB14253 | 0.01404 | 0.01503 | 1.07082 | 0.00099 | 3.38E−02 | 7.50E−02 | [increased] |
| SGB15106 | 0.05964 | 0.06358 | 1.06600 | 0.00394 | 3.86E−02 | 8.42E−02 | [increased] |
| SGB4665 | 0.00563 | 0.00639 | 1.13538 | 0.00076 | 4.20E−02 | 9.00E−02 | [increased] |
| SGB4893 | 0.00753 | 0.00905 | 1.20194 | 0.00152 | 4.82E−02 | 9.98E−02 | [increased] |
| SGB4644 | 0.03980 | 0.03676 | 0.92359 | −0.00304 | 4.92E−02 | 1.00E−01 | [decreased] |
| SGB49188 | 0.00320 | 0.00276 | 0.85987 | −0.00045 | 5.96E−02 | 1.19E−01 | [decreased] |
| SGB15229 | 0.01847 | 0.01958 | 1.06002 | 0.00111 | 9.62E−02 | 1.78E−01 | [increased] |
| SGB15236 | 0.04411 | 0.04473 | 1.01403 | 0.00062 | 1.27E−01 | 2.25E−01 | [increased] |
| SGB15131 | 0.00613 | 0.00756 | 1.23411 | 0.00144 | 1.41E−01 | 2.46E−01 | [increased] |
| SGB4638 | 0.00600 | 0.00516 | 0.86065 | −0.00084 | 1.86E−01 | 3.10E−01 | [decreased] |
| SGB14042 | 0.00884 | 0.00956 | 1.08131 | 0.00072 | 2.10E−01 | 3.41E−01 | [increased] |
| SGB6340 | 0.01590 | 0.01514 | 0.95202 | −0.00076 | 2.16E−01 | 3.46E−01 | [decreased] |
| SGB4777 | 0.01750 | 0.01789 | 1.02234 | 0.00039 | 2.70E−01 | 4.04E−01 | [increased] |
| SGB13982 | 0.00972 | 0.01023 | 1.05338 | 0.00052 | 2.79E−01 | 4.14E−01 | [increased] |
| SGB13979 | 0.00783 | 0.00839 | 1.07171 | 0.00056 | 2.87E−01 | 4.20E−01 | [increased] |
| SGB3952 | 0.00465 | 0.00432 | 0.92917 | −0.00033 | 3.44E−01 | 4.75E−01 | [decreased] |
| SGB6174_{—}group | 0.01623 | 0.01547 | 0.95331 | −0.00076 | 3.54E−01 | 4.83E−01 | [decreased] |
| SGB15031 | 0.03859 | 0.03999 | 1.03646 | 0.00141 | 3.83E−01 | 5.17E−01 | [increased] |
| SGB4803 | 0.00254 | 0.00247 | 0.97214 | −0.00007 | 4.48E−01 | 5.97E−01 | [decreased] |
| SGB79840 | 0.00334 | 0.00347 | 1.03700 | 0.00012 | 4.82E−01 | 6.02E−01 | [increased] |
| SGB7258 | 0.00100 | 0.00091 | 0.91305 | −0.00009 | 4.98E−01 | 6.16E−01 | [decreased] |
| SGB15123 | 0.00583 | 0.00540 | 0.92689 | −0.00043 | 5.10E−01 | 6.25E−01 | [decreased] |
| SGB4191 | 0.02997 | 0.02915 | 0.97261 | −0.00082 | 5.27E−01 | 6.26E−01 | [decreased] |
| SGB4894 | 0.01494 | 0.01633 | 1.09299 | 0.00139 | 5.24E−01 | 6.26E−01 | [increased] |
| SGB4805 | 0.01411 | 0.01468 | 1.04044 | 0.00057 | 5.61E−01 | 6.53E−01 | [increased] |
| SGB14252 | 0.01124 | 0.01131 | 1.00598 | 0.00007 | 5.80E−01 | 6.69E−01 | [increased] |
| SGB14311 | 0.02995 | 0.03076 | 1.02718 | 0.00081 | 5.90E−01 | 6.75E−01 | [increased] |
| SGB14114 | 0.01620 | 0.01688 | 1.04201 | 0.00068 | 6.60E−01 | 7.41E−01 | [increased] |
| SGB14306 | 0.03693 | 0.03729 | 1.00964 | 0.00036 | 8.17E−01 | 8.91E−01 | [increased] |
| SGB14921 | 0.01638 | 0.01649 | 1.00675 | 0.00011 | 8.49E−01 | 9.10E−01 | [increased] |
| SGB14179 | 0.00546 | 0.00552 | 1.01003 | 0.00005 | 8.61E−01 | 9.14E−01 | [increased] |
| SGB15225 | 0.01667 | 0.01683 | 1.00984 | 0.00016 | 8.96E−01 | 9.38E−01 | [increased] |
| SGB15234 | 0.03842 | 0.03788 | 0.98589 | −0.00054 | 9.90E−01 | 9.98E−01 | [decreased] |

TABLE 5
Bad Bugs extracted only with health markers (no diet markers) from the first
group of 440 participants - changes in the first group of 440 participants
Normalized relative abundances of the 60 bad bugs extracted only with health markers at the first test
(Initial Abundance) and at retest time (Retest Abundance). Also shown are the Fold Change, the p-
values relative to the Wilcoxon signed rank test (Wilcoxon p-value) and the p-values adjusted for False
Discovery Rate (Adjusted p-value). The field Type of Change reports whether after the diet intervention the
relative abundance increased or decreased. The table is sorted according to the Adjusted p-value. Italics
indicates the microbes for which the normalized relative abundance change was not considered significant.

| | Initial | Retest | Fold | Delta | | Adjusted | Type of |
|---|---|---|---|---|---|---|---|
| SGB | Abundance | Abundance | Change | Change | p-value | p-value | Change |
| SGB4608 | 0.02952 | 0.01888 | 0.63975 | −0.01063 | 1.50E−16 | 6.02E−15 | [decreased] |
| SGB15078 | 0.03215 | 0.02687 | 0.83589 | −0.00528 | 1.12E−10 | 2.23E−09 | [decreased] |
| SGB15132 | 0.01559 | 0.01153 | 0.73948 | −0.00406 | 1.09E−10 | 2.23E−09 | [decreased] |
| SGB4606 | 0.00367 | 0.00215 | 0.58389 | −0.00153 | 1.23E−08 | 1.64E−07 | [decreased] |
| SGB4584 | 0.01084 | 0.00657 | 0.60576 | −0.00428 | 3.28E−08 | 3.93E−07 | [decreased] |
| SGB4826_{—}group | 0.05771 | 0.05059 | 0.87655 | −0.00712 | 7.24E−08 | 7.90E−07 | [decreased] |
| SGB15452 | 0.02834 | 0.02608 | 0.92022 | −0.00226 | 1.08E−05 | 8.61E−05 | [decreased] |
| SGB15271 | 0.02012 | 0.01677 | 0.83336 | −0.00335 | 9.27E−05 | 5.56E−04 | [decreased] |
| SGB19850_{—}group | 0.00063 | 0.00022 | 0.35617 | −0.00041 | 1.52E−04 | 8.71E−04 | [decreased] |
| SGB5184 | 0.00312 | 0.00227 | 0.72759 | −0.00085 | 1.63E−04 | 8.89E−04 | [decreased] |
| SGB4573_{—}group | 0.00645 | 0.00462 | 0.71628 | −0.00183 | 2.51E−04 | 1.25E−03 | [decreased] |
| SGB4791 | 0.00286 | 0.00173 | 0.60567 | −0.00113 | 3.86E−04 | 1.82E−03 | [decreased] |
| SGB4837_{—}group | 0.12811 | 0.12102 | 0.94470 | −0.00708 | 3.95E−04 | 1.82E−03 | [decreased] |
| SGB4703 | 0.00175 | 0.00121 | 0.69440 | −0.00053 | 6.19E−04 | 2.75E−03 | [decreased] |
| SGB4037_{—}group | 0.00315 | 0.00208 | 0.66053 | −0.00107 | 1.09E−03 | 4.38E−03 | [decreased] |
| SGB4753 | 0.00515 | 0.00373 | 0.72378 | −0.00142 | 1.99E−03 | 7.45E−03 | [decreased] |
| SGB10068 | 0.02226 | 0.01884 | 0.84615 | −0.00343 | 2.85E−03 | 1.01E−02 | [decreased] |
| SGB14874 | 0.00740 | 0.00440 | 0.59425 | −0.00300 | 6.54E−03 | 2.18E−02 | [decreased] |
| SGB53821 | 0.00089 | 0.00059 | 0.66235 | −0.00030 | 7.73E−03 | 2.32E−02 | [decreased] |
| SGB4035 | 0.00068 | 0.00036 | 0.53162 | −0.00032 | 9.18E−03 | 2.69E−02 | [decreased] |
| SGB4746 | 0.00253 | 0.00203 | 0.80250 | −0.00050 | 1.19E−02 | 3.40E−02 | [decreased] |
| SGB4572 | 0.00096 | 0.00055 | 0.57217 | −0.00041 | 1.48E−02 | 4.03E−02 | [decreased] |
| SGB8163 | 0.00040 | 0.00020 | 0.50317 | −0.00020 | 1.73E−02 | 4.62E−02 | [decreased] |
| SGB4630_{—}group | 0.00282 | 0.00229 | 0.81219 | −0.00053 | 2.66E−02 | 6.38E−02 | [decreased] |
| SGB4588_{—}group | 0.00451 | 0.00313 | 0.69388 | −0.00138 | 2.82E−02 | 6.51E−02 | [decreased] |
| SGB17137 | 0.00014 | 0.00004 | 0.25203 | −0.00011 | 2.81E−02 | 6.51E−02 | [decreased] |
| SGB4862 | 0.00609 | 0.00444 | 0.72930 | −0.00165 | 4.27E−02 | 9.00E−02 | [decreased] |
| SGB6744 | 0.00328 | 0.00300 | 0.91587 | −0.00028 | 8.13E−02 | 1.60E−01 | [decreased] |
| SGB79823 | 0.00181 | 0.00153 | 0.84612 | −0.00028 | 8.64E−02 | 1.67E−01 | [decreased] |
| SGB4742 | 0.00087 | 0.00066 | 0.76536 | −0.00020 | 8.87E−02 | 1.69E−01 | [decreased] |
| SGB4447 | 0.01234 | 0.01385 | 1.12245 | 0.00151 | 9.42E−02 | 1.77E−01 | [increased] |
| SGB4758_{—}group | 0.00449 | 0.00390 | 0.86753 | −0.00059 | 1.00E−01 | 1.82E−01 | [decreased] |
| SGB4761 | 0.00227 | 0.00256 | 1.13010 | 0.00029 | 1.27E−01 | 2.25E−01 | [increased] |
| SGB4699 | 0.00182 | 0.00156 | 0.85691 | −0.00026 | 1.76E−01 | 3.02E−01 | [decreased] |
| SGB4762 | 0.00153 | 0.00116 | 0.75794 | −0.00037 | 1.86E−01 | 3.10E−01 | [decreased] |
| SGB7984 | 0.00141 | 0.00085 | 0.60145 | −0.00056 | 1.89E−01 | 3.11E−01 | [decreased] |
| SGB15158 | 0.00621 | 0.00610 | 0.98286 | −0.00011 | 2.29E−01 | 3.62E−01 | [decreased] |
| SGB4529 | 0.00108 | 0.00086 | 0.79648 | −0.00022 | 2.35E−01 | 3.66E−01 | [decreased] |
| SGB14809 | 0.01323 | 0.01225 | 0.92551 | −0.00099 | 2.54E−01 | 3.90E−01 | [decreased] |
| SGB4797 | 0.00052 | 0.00030 | 0.58245 | −0.00022 | 2.60E−01 | 3.96E−01 | [decreased] |
| SGB4583 | 0.00136 | 0.00106 | 0.78175 | −0.00030 | 2.95E−01 | 4.27E−01 | [decreased] |
| SGB10115 | 0.00278 | 0.00439 | 1.58077 | 0.00161 | 3.22E−01 | 4.60E−01 | [increased] |
| SGB4861 | 0.00045 | 0.00026 | 0.57452 | −0.00019 | 3.33E−01 | 4.69E−01 | [decreased] |
| SGB1814 | 0.12783 | 0.12588 | 0.98477 | −0.00195 | 3.36E−01 | 4.69E−01 | [decreased] |
| SGB8028_{—}group | 0.00069 | 0.00053 | 0.76611 | −0.00016 | 4.69E−01 | 6.02E−01 | [decreased] |
| SGB4041 | 0.00232 | 0.00272 | 1.16872 | 0.00039 | 4.74E−01 | 6.02E−01 | [increased] |
| SGB4785 | 0.00043 | 0.00026 | 0.60487 | −0.00017 | 4.77E−01 | 6.02E−01 | [decreased] |
| SGB71883 | 0.00017 | 0.00020 | 1.17225 | 0.00003 | 4.77E−01 | 6.02E−01 | [increased] |
| SGB8255_{—}group | 0.00006 | 0.00011 | 1.93861 | 0.00005 | 4.80E−01 | 6.02E−01 | [increased] |
| SGB79883 | 0.00017 | 0.00020 | 1.17460 | 0.00003 | 5.23E−01 | 6.26E−01 | [increased] |
| SGB29328 | 0.00057 | 0.00053 | 0.94659 | −0.00003 | 5.38E−01 | 6.33E−01 | [decreased] |
| SGB4760 | 0.00122 | 0.00130 | 1.06376 | 0.00008 | 6.18E−01 | 6.99E−01 | [increased] |
| SGB4988 | 0.00304 | 0.00341 | 1.12182 | 0.00037 | 7.15E−01 | 7.93E−01 | [increased] |
| SGB4786 | 0.00191 | 0.00168 | 0.88170 | −0.00023 | 7.20E−01 | 7.93E−01 | [decreased] |
| SGB1836_{—}group | 0.14643 | 0.14827 | 1.01253 | 0.00183 | 8.27E−01 | 8.94E−01 | [increased] |
| SGB4724 | 0.00110 | 0.00117 | 1.06095 | 0.00007 | 8.99E−01 | 9.38E−01 | [increased] |
| SGB4688 | 0.00073 | 0.00080 | 1.08873 | 0.00006 | 9.51E−01 | 9.84E−01 | [increased] |
| SGB4798 | 0.00232 | 0.00238 | 1.02470 | 0.00006 | 9.72E−01 | 9.89E−01 | [increased] |
| SGB4617 | 0.00280 | 0.00298 | 1.06724 | 0.00019 | 9.70E−01 | 9.89E−01 | [increased] |
| SGB4794_{—}group | 0.00064 | 0.00067 | 1.05663 | 0.00004 | 1.00E+00 | 1.00E+00 | [increased] |

TABLE 6
Changes in the second group of 1124 participants
All SGBs with a significant change between pre and post-intervention in the second
group of 1124 participants (Wilcoxon rank-sum test, Bonferroni correcter p-values <
0.01). Changes in relative abundance and prevalence are reported. The fields “Health
rank” and “Diet rank” report those ranks for SGBs present in Table 1A.

| | Mean | Mean | Log2 ratio | | | | |
|---|---|---|---|---|---|---|---|
| | relative | relative | of post- | | | | |
| | abundance | abundance | pre mean | Prevalence | Prevalence | | |
| | pre- | post- | relative | pre- | post- | Health | Diet |
| SGB_ID | intervention | intervention | abundance | intervention | intervention | rank | rank |
| SGB13981 | 0.02830 | 0.04379 | 0.62966 | 61.83 | 66.28 | 0.15359 | 0.22002 |
| SGB13995 | 0.00835 | 0.01656 | 0.98781 | 9.61 | 10.77 | | |
| SGB14125 | 0.00540 | 0.00217 | −1.31476 | 23.31 | 13.97 | 0.63048 | 0.63440 |
| SGB14127 | 0.00289 | 0.00084 | −1.78471 | 44.84 | 16.73 | 0.52101 | 0.89270 |
| SGB14128 | 0.00876 | 0.00453 | −0.95018 | 33.19 | 28.02 | 0.29839 | 0.42665 |
| SGB14142 | 0.00044 | 0.00014 | −1.61507 | 8.63 | 3.29 | 0.71137 | 0.87651 |
| SGB14177 | 0.00994 | 0.00686 | −0.53388 | 25.71 | 16.73 | 0.33375 | 0.40690 |
| SGB14179 | 0.00518 | 0.00369 | −0.48938 | 41.46 | 34.07 | 0.07117 | 0.29014 |
| SGB14250 | 0.02124 | 0.01318 | −0.68840 | 39.23 | 35.77 | 0.26758 | 0.22981 |
| SGB14259 | 0.00385 | 0.00197 | −0.96622 | 10.68 | 5.60 | 0.63067 | 0.56722 |
| SGB14262 | 0.00136 | 0.00051 | −1.40443 | 15.21 | 6.49 | 0.39667 | 0.52128 |
| SGB14263 | 0.03414 | 0.02194 | −0.63780 | 34.79 | 31.41 | | |
| SGB14397 | 0.00787 | 0.00513 | −0.61642 | 12.99 | 12.01 | | |
| SGB14546_group | 0.46789 | 0.35533 | −0.39702 | 89.32 | 89.32 | 0.88488 | 0.84324 |
| SGB14749 | 0.00504 | 0.00272 | −0.89261 | 5.52 | 4.09 | | |
| SGB14809 | 0.02286 | 0.01952 | −0.22748 | 37.81 | 32.74 | 0.97745 | 0.87237 |
| SGB14837 | 0.00375 | 0.00166 | −1.17456 | 43.24 | 24.91 | 0.85756 | 0.86524 |
| SGB14838 | 0.00054 | 0.00017 | −1.66492 | 11.03 | 3.83 | 0.36298 | 0.87849 |
| SGB14844 | 0.03118 | 0.01628 | −0.93790 | 36.74 | 28.56 | 0.51506 | 0.74680 |
| SGB14853 | 0.03646 | 0.01211 | −1.58997 | 67.70 | 49.82 | 0.70551 | 0.92778 |
| SGB14861 | 0.29381 | 0.21087 | −0.47854 | 91.19 | 91.10 | 0.26452 | 0.72218 |
| SGB14890 | 0.00121 | 0.00081 | −0.57909 | 12.72 | 7.12 | 0.82233 | 0.76740 |
| SGB14891 | 0.00343 | 0.00187 | −0.87263 | 31.14 | 18.42 | 0.61174 | 0.59551 |
| SGB14894 | 0.01170 | 0.00565 | −1.05115 | 58.36 | 39.41 | 0.44115 | 0.81531 |
| SGB14898 | 0.00352 | 0.00106 | −1.73786 | 10.68 | 6.67 | 0.70586 | 0.75568 |
| SGB14899 | 0.03170 | 0.01297 | −1.28965 | 74.47 | 53.11 | 0.17381 | 0.75893 |
| SGB14906 | 0.02089 | 0.01464 | −0.51284 | 32.92 | 27.67 | 0.55077 | 0.46613 |
| SGB14909 | 0.03202 | 0.02244 | −0.51264 | 25.18 | 21.35 | 0.66775 | 0.76782 |
| SGB14912 | 0.01581 | 0.00904 | −0.80625 | 24.47 | 18.68 | 0.41339 | 0.44394 |
| SGB14921 | 0.03099 | 0.02486 | −0.31762 | 75.53 | 74.82 | 0.12060 | 0.28506 |
| SGB14932 | 0.01870 | 0.01277 | −0.55031 | 48.84 | 42.79 | 0.38798 | 0.63602 |
| SGB14941 | 0.00438 | 0.00271 | −0.69287 | 34.61 | 26.69 | 0.37726 | 0.49344 |
| SGB14951 | 0.00210 | 0.00065 | −1.68541 | 11.92 | 6.41 | 0.49105 | 0.54206 |
| SGB14952 | 0.00212 | 0.00069 | −1.61279 | 10.68 | 5.60 | 0.49009 | 0.69974 |
| SGB14958 | 0.01091 | 0.00579 | −0.91452 | 17.88 | 13.70 | 0.62092 | 0.65270 |
| SGB14963 | 0.00436 | 0.00257 | −0.76313 | 22.06 | 13.97 | 0.79926 | 0.85623 |
| SGB14966 | 0.01299 | 0.00790 | −0.71717 | 65.66 | 57.74 | 0.44307 | 0.27769 |
| SGB14975 | 0.00901 | 0.00624 | −0.53003 | 39.15 | 34.79 | 0.44419 | 0.55940 |
| SGB14980_group | 0.04624 | 0.06817 | 0.55988 | 22.15 | 22.15 | | |
| SGB14987 | 0.00180 | 0.00402 | 1.15903 | 16.10 | 20.82 | 0.74710 | 0.51924 |
| SGB14991 | 0.21218 | 0.31027 | 0.54819 | 55.78 | 54.54 | 0.32438 | 0.31065 |
| SGB14993_group | 0.31414 | 0.43919 | 0.48342 | 97.60 | 98.04 | 0.45146 | 0.12727 |
| SGB14995 | 0.00415 | 0.00277 | −0.58484 | 38.70 | 29.27 | 0.83301 | 0.84854 |
| SGB14999 | 0.01781 | 0.01397 | −0.35065 | 54.80 | 50.36 | 0.80495 | 0.54885 |
| SGB15031 | 0.35602 | 0.28380 | −0.32709 | 72.51 | 72.69 | 0.15023 | 0.42827 |
| SGB15045 | 0.00458 | 0.00270 | −0.76173 | 22.86 | 16.99 | 0.45927 | 0.40008 |
| SGB15051 | 0.09014 | 0.12599 | 0.48298 | 50.62 | 52.31 | 0.21791 | 0.22919 |
| SGB15053_group | 0.63416 | 1.00039 | 0.65764 | 87.10 | 87.81 | 0.09860 | 0.17996 |
| SGB15065 | 0.03550 | 0.04646 | 0.38832 | 39.86 | 41.28 | 0.25561 | 0.17700 |
| SGB15067 | 0.02187 | 0.01581 | −0.46762 | 66.73 | 57.56 | 0.43302 | 0.45189 |
| SGB15073 | 0.04003 | 0.02569 | −0.64005 | 65.04 | 58.54 | 0.55999 | 0.37222 |
| SGB15076 | 0.01035 | 0.00756 | −0.45333 | 19.48 | 12.10 | 0.77132 | 0.73382 |
| SGB15078 | 0.20455 | 0.15851 | −0.36787 | 95.11 | 90.12 | 0.89839 | 0.90447 |
| SGB15081 | 0.02205 | 0.01590 | −0.47173 | 56.49 | 50.71 | 0.40618 | 0.23764 |
| SGB15087 | 0.01192 | 0.00550 | −1.11647 | 16.46 | 12.19 | 0.45724 | 0.41920 |
| SGB15089 | 0.07409 | 0.04969 | −0.57636 | 80.16 | 73.13 | 0.38915 | 0.57715 |
| SGB15093 | 0.26983 | 0.20391 | −0.40413 | 56.67 | 55.60 | 0.28937 | 0.35860 |
| SGB15124 | 0.02601 | 0.02062 | −0.33519 | 84.34 | 77.40 | 0.74468 | 0.83067 |
| SGB15132 | 0.07584 | 0.04720 | −0.68406 | 72.69 | 58.36 | 0.98122 | 0.90076 |
| SGB15140 | 0.00499 | 0.00419 | −0.25292 | 33.45 | 30.07 | 0.39244 | 0.45510 |
| SGB15149 | 0.01199 | 0.00931 | −0.36497 | 14.77 | 11.03 | 0.80693 | 0.64107 |
| SGB15154 | 0.17908 | 0.14150 | −0.33979 | 96.98 | 96.00 | 0.56368 | 0.54930 |
| SGB15156_group | 0.00778 | 0.00386 | −1.00958 | 14.41 | 9.61 | 0.70985 | 0.71003 |
| SGB15158 | 0.02343 | 0.01565 | −0.58164 | 20.55 | 17.08 | 0.94161 | 0.87802 |
| SGB15164 | 0.04936 | 0.04195 | −0.23482 | 75.71 | 71.53 | 0.28671 | 0.64250 |
| SGB15180 | 0.37381 | 0.50411 | 0.43144 | 56.05 | 56.85 | 0.20880 | 0.16489 |
| SGB15196 | 0.05699 | 0.08471 | 0.57192 | 27.67 | 28.29 | | |
| SGB15198 | 0.18712 | 0.28803 | 0.62229 | 29.98 | 30.07 | | |
| SGB15201 | 0.02930 | 0.00926 | −1.66203 | 15.57 | 10.68 | 0.30548 | 0.65334 |
| SGB15203 | 0.04481 | 0.02876 | −0.63954 | 39.77 | 33.10 | 0.30556 | 0.23852 |
| SGB15216 | 0.19936 | 0.16055 | −0.31232 | 91.46 | 90.66 | 0.31727 | 0.46799 |
| SGB15224 | 0.17339 | 0.13580 | −0.35254 | 66.01 | 65.75 | 0.25047 | 0.33171 |
| SGB15249 | 0.60804 | 0.67474 | 0.15017 | 83.90 | 84.52 | 0.01295 | 0.21809 |
| SGB15265_group | 0.44843 | 0.70790 | 0.65868 | 70.37 | 71.35 | 0.17739 | 0.28924 |
| SGB15271 | 0.08635 | 0.04960 | −0.79997 | 95.55 | 86.03 | 0.90009 | 0.89290 |
| SGB15272 | 0.00775 | 0.00344 | −1.17213 | 23.58 | 13.26 | 0.67121 | 0.72943 |
| SGB15273 | 0.00574 | 0.00370 | −0.63119 | 33.36 | 17.97 | 0.56841 | 0.72302 |
| SGB15278 | 0.00454 | 0.00175 | −1.37379 | 14.23 | 8.36 | 0.52785 | 0.41325 |
| SGB15286 | 0.96340 | 0.82870 | −0.21728 | 94.31 | 94.66 | 0.49463 | 0.60366 |
| SGB15318_group | 0.99515 | 0.82169 | −0.27633 | 93.68 | 93.77 | 0.46856 | 0.47856 |
| SGB15350 | 0.03866 | 0.02537 | −0.60769 | 63.70 | 56.94 | 0.65891 | 0.51735 |
| SGB15368 | 2.15478 | 2.94275 | 0.44962 | 76.87 | 76.60 | 0.10061 | 0.23491 |
| SGB15377 | 0.04710 | 0.06152 | 0.38539 | 24.29 | 25.27 | | |
| SGB15385 | 0.03486 | 0.04406 | 0.33805 | 46.98 | 48.49 | 0.29887 | 0.29595 |
| SGB15395 | 0.03241 | 0.01998 | −0.69791 | 44.75 | 37.63 | 0.22050 | 0.56572 |
| SGB15452 | 0.17155 | 0.15077 | −0.18625 | 88.70 | 88.17 | 0.89858 | 0.83942 |
| SGB17241 | 0.02690 | 0.01345 | −1.00029 | 11.92 | 8.81 | | |
| SGB17278 | 0.07239 | 0.11041 | 0.60894 | 34.07 | 43.77 | 0.60140 | 0.14099 |
| SGB1790 | 0.32163 | 0.28907 | −0.15396 | 95.46 | 95.82 | 0.35057 | 0.78297 |
| SGB1812 | 0.52461 | 0.41882 | −0.32491 | 41.55 | 39.86 | 0.62439 | 0.46036 |
| SGB1829 | 0.45090 | 0.33318 | −0.43650 | 32.30 | 29.36 | 0.59289 | 0.49488 |
| SGB1844 | 0.54246 | 0.70241 | 0.37281 | 61.21 | 61.12 | 0.50839 | 0.33457 |
| SGB1855_group | 0.17756 | 0.16248 | −0.12809 | 42.70 | 39.15 | 0.83040 | 0.71057 |
| SGB1858 | 0.02726 | 0.04388 | 0.68677 | 38.17 | 39.06 | 0.38225 | 0.34213 |
| SGB1860 | 0.17086 | 0.14358 | −0.25097 | 25.98 | 23.84 | 0.65815 | 0.61674 |
| SGB1949 | 0.48340 | 0.37123 | −0.38092 | 73.84 | 73.13 | 0.60178 | 0.86732 |
| SGB1957 | 0.19535 | 0.33329 | 0.77069 | 41.46 | 40.48 | 0.69338 | 0.29523 |
| SGB19850_group | 0.00078 | 0.00022 | −1.82498 | 15.48 | 5.25 | 0.89309 | 0.56934 |
| SGB2135 | 0.23898 | 0.43015 | 0.84796 | 9.70 | 8.54 | | |
| SGB2286 | 0.28726 | 0.24304 | −0.24116 | 41.46 | 39.77 | 0.59962 | 0.33318 |
| SGB2290 | 0.55307 | 0.72587 | 0.39227 | 87.99 | 89.23 | 0.16936 | 0.31361 |
| SGB2295 | 0.77071 | 0.97540 | 0.33980 | 84.52 | 84.79 | 0.23847 | 0.38328 |
| SGB2296 | 0.08233 | 0.11703 | 0.50743 | 43.51 | 42.08 | 0.37703 | 0.48447 |
| SGB2301 | 0.56031 | 0.73836 | 0.39809 | 81.23 | 80.87 | 0.41860 | 0.68356 |
| SGB2303 | 1.28676 | 1.56298 | 0.28056 | 87.10 | 87.81 | 0.48890 | 0.62047 |
| SGB25416 | 0.00286 | 0.00190 | −0.58797 | 58.54 | 44.31 | 0.19393 | 0.65673 |
| SGB25497 | 0.05462 | 0.10116 | 0.88930 | 39.06 | 41.64 | 0.19225 | 0.22377 |
| SGB28999 | 0.00052 | 0.00026 | −0.97534 | 5.96 | 1.51 | | |
| SGB29302 | 0.00034 | 0.00017 | −1.02213 | 13.52 | 5.52 | 0.34465 | 0.70104 |
| SGB29305 | 0.00073 | 0.00037 | −0.98786 | 20.82 | 11.12 | 0.58074 | 0.63961 |
| SGB29339 | 0.00122 | 0.00095 | −0.36481 | 22.60 | 17.70 | 0.52358 | 0.75227 |
| SGB3573 | 0.00171 | 0.00074 | −1.21203 | 15.12 | 8.72 | 0.30320 | 0.56169 |
| SGB3574 | 0.01319 | 0.00651 | −1.01923 | 60.41 | 51.07 | 0.54514 | 0.84655 |
| SGB3813 | 0.00201 | 0.00068 | −1.55965 | 20.11 | 9.34 | 0.34588 | 0.42258 |
| SGB3952 | 0.01215 | 0.00685 | −0.82687 | 50.53 | 45.02 | 0.08883 | 0.37533 |
| SGB3957 | 0.00438 | 0.00548 | 0.32301 | 46.00 | 54.98 | 0.51872 | 0.63065 |
| SGB3983 | 0.01124 | 0.00778 | −0.53064 | 14.68 | 13.70 | | |
| SGB3986 | 0.00100 | 0.00038 | −1.39125 | 8.63 | 4.98 | | |
| SGB3988 | 0.01383 | 0.00524 | −1.39997 | 48.67 | 32.74 | 0.29699 | 0.52489 |
| SGB3989 | 0.02319 | 0.00998 | −1.21588 | 32.92 | 27.85 | 0.44355 | 0.30965 |
| SGB3991 | 0.00118 | 0.00101 | −0.23095 | 7.38 | 2.58 | 0.68047 | 0.66263 |
| SGB3993 | 0.00174 | 0.00063 | −1.46498 | 7.30 | 3.83 | 0.68224 | 0.64763 |
| SGB3996 | 0.01076 | 0.00604 | −0.83280 | 25.27 | 17.79 | 0.37920 | 0.50822 |
| SGB4027 | 0.00620 | 0.00390 | −0.67013 | 16.28 | 11.74 | 0.39242 | 0.24616 |
| SGB4029 | 0.01691 | 0.00836 | −1.01672 | 26.87 | 24.38 | 0.41726 | 0.68616 |
| SGB4030 | 0.00145 | 0.00044 | −1.71777 | 13.97 | 4.72 | 0.67851 | 0.67685 |
| SGB4044 | 0.00262 | 0.00147 | −0.83318 | 18.42 | 10.94 | 0.86052 | 0.68509 |
| SGB4045 | 0.00748 | 0.00393 | −0.92910 | 35.23 | 26.96 | 0.49781 | 0.49780 |
| SGB4046 | 0.00675 | 0.00359 | −0.90886 | 43.68 | 29.72 | 0.82932 | 0.72047 |
| SGB4062 | 0.00185 | 0.00059 | −1.64229 | 14.50 | 8.63 | | |
| SGB4063 | 0.00291 | 0.00124 | −1.23845 | 20.37 | 13.88 | 0.45494 | 0.62551 |
| SGB4166 | 0.07707 | 0.15469 | 1.00511 | 27.22 | 27.85 | 0.53655 | 0.53449 |
| SGB4198_group | 0.48597 | 0.41837 | −0.21610 | 64.50 | 54.27 | 0.14354 | 0.58523 |
| SGB4247 | 0.17448 | 0.29069 | 0.73641 | 18.33 | 21.53 | | |
| SGB4262 | 0.63717 | 0.48066 | −0.40667 | 81.23 | 79.89 | 0.31291 | 0.44000 |
| SGB4269 | 0.37727 | 0.59094 | 0.64742 | 98.04 | 98.58 | 0.47325 | 0.20958 |
| SGB4285_group | 0.82558 | 0.47104 | −0.80957 | 68.68 | 64.32 | 0.58126 | 0.62731 |
| SGB4348 | 0.13457 | 0.06095 | −1.14269 | 25.44 | 20.91 | 0.75728 | 0.51951 |
| SGB4367 | 0.15687 | 0.08351 | −0.90961 | 35.85 | 33.01 | 0.56938 | 0.52969 |
| SGB4436 | 0.00303 | 0.00172 | −0.81185 | 23.67 | 16.81 | 0.44585 | 0.41628 |
| SGB4438 | 0.00147 | 0.00074 | −0.98629 | 13.35 | 8.90 | 0.48774 | 0.61445 |
| SGB4552_group | 0.03100 | 0.02687 | −0.20613 | 92.26 | 91.37 | 0.71219 | 0.32914 |
| SGB4563_group | 0.21229 | 0.14323 | −0.56770 | 88.52 | 87.63 | 0.73120 | 0.79290 |
| SGB4575 | 0.11266 | 0.09710 | −0.21443 | 97.78 | 98.67 | 0.76271 | 0.49931 |
| SGB4581 | 0.26208 | 0.22368 | −0.22858 | 88.70 | 88.26 | 0.58431 | 0.64875 |
| SGB4584 | 0.04254 | 0.01478 | −1.52464 | 22.86 | 13.70 | 0.98107 | 0.93219 |
| SGB4595 | 0.00133 | 0.00066 | −1.00308 | 27.58 | 17.08 | 0.67558 | 0.52407 |
| SGB4606 | 0.00579 | 0.00293 | −0.98236 | 20.91 | 13.97 | 0.98061 | 0.87432 |
| SGB4608 | 0.09784 | 0.05471 | −0.83872 | 60.68 | 46.71 | 0.99077 | 0.90382 |
| SGB4628 | 0.00245 | 0.00053 | −2.20566 | 8.27 | 4.54 | 0.39402 | 0.62647 |
| SGB4643 | 0.05950 | 0.09179 | 0.62537 | 45.91 | 49.38 | 0.07249 | 0.14152 |
| SGB4651 | 0.01821 | 0.03387 | 0.89491 | 28.65 | 29.36 | 0.40389 | 0.28397 |
| SGB4654 | 0.10523 | 0.17868 | 0.76386 | 29.98 | 31.05 | 0.19578 | 0.14378 |
| SGB4658 | 0.06234 | 0.09976 | 0.67824 | 28.56 | 32.03 | 0.24065 | 0.16983 |
| SGB4659 | 0.11830 | 0.18331 | 0.63186 | 49.64 | 53.11 | 0.34981 | 0.25558 |
| SGB4664 | 0.02736 | 0.04981 | 0.86451 | 16.28 | 21.71 | 0.25405 | 0.06562 |
| SGB4665 | 0.00620 | 0.01126 | 0.86019 | 24.56 | 29.80 | 0.16585 | 0.13764 |
| SGB4687 | 0.00299 | 0.00179 | −0.74245 | 24.56 | 19.40 | 0.33201 | 0.36953 |
| SGB4704 | 0.01272 | 0.02582 | 1.02126 | 11.83 | 12.81 | | |
| SGB4705 | 0.22244 | 0.18692 | −0.25099 | 92.62 | 93.15 | 0.69142 | 0.56855 |
| SGB4706 | 0.08499 | 0.09986 | 0.23270 | 89.32 | 90.39 | 0.10943 | 0.08436 |
| SGB4711 | 0.07875 | 0.08695 | 0.14290 | 88.08 | 89.59 | 0.25439 | 0.20220 |
| SGB4714 | 0.10468 | 0.12580 | 0.26517 | 83.90 | 86.12 | 0.27136 | 0.12781 |
| SGB4721 | 0.05892 | 0.04743 | −0.31312 | 63.97 | 58.72 | 0.85684 | 0.84121 |
| SGB4746 | 0.01322 | 0.01036 | −0.35130 | 10.94 | 8.45 | 0.98485 | 0.80934 |
| SGB4749 | 0.17731 | 0.14453 | −0.29490 | 91.64 | 91.19 | 0.80421 | 0.42808 |
| SGB4750 | 0.04536 | 0.03489 | −0.37871 | 90.48 | 89.68 | 0.71116 | 0.59891 |
| SGB4753 | 0.01796 | 0.00763 | −1.23494 | 13.70 | 9.16 | 0.99668 | 0.92772 |
| SGB4758_group | 0.00808 | 0.00589 | −0.45695 | 29.72 | 23.75 | 0.97753 | 0.89060 |
| SGB4765 | 0.00261 | 0.00715 | 1.45313 | 10.77 | 13.97 | 0.58059 | 0.26984 |
| SGB47656 | 0.01263 | 0.01924 | 0.60734 | 41.19 | 43.24 | 0.17999 | 0.18889 |
| SGB4770 | 0.01074 | 0.00604 | −0.82967 | 67.62 | 55.87 | 0.18992 | 0.31973 |
| SGB4772 | 0.00242 | 0.00135 | −0.84341 | 47.51 | 35.94 | 0.25693 | 0.28455 |
| SGB4774 | 0.00075 | 0.00027 | −1.48558 | 7.47 | 3.29 | 0.67089 | 0.61731 |
| SGB4778 | 0.01928 | 0.01522 | −0.34073 | 83.63 | 85.32 | 0.14045 | 0.10557 |
| SGB4780 | 0.05138 | 0.06266 | 0.28631 | 91.19 | 92.08 | 0.22571 | 0.20021 |
| SGB4782 | 0.04309 | 0.08222 | 0.93225 | 80.25 | 83.54 | 0.10281 | 0.12736 |
| SGB48024 | 0.02669 | 0.03448 | 0.36974 | 32.21 | 34.34 | 0.33498 | 0.23747 |
| SGB4810 | 0.21990 | 0.28706 | 0.38454 | 63.61 | 67.26 | 0.18853 | 0.12946 |
| SGB4816 | 0.11808 | 0.17147 | 0.53819 | 72.60 | 74.47 | 0.26564 | 0.06210 |
| SGB4820 | 0.26321 | 0.34081 | 0.37276 | 99.02 | 99.73 | 0.62433 | 0.55576 |
| SGB4826_group | 0.34611 | 0.28854 | −0.26249 | 89.86 | 86.57 | 0.91549 | 0.83956 |
| SGB4831_group | 0.03385 | 0.04435 | 0.38954 | 73.93 | 76.25 | 0.24824 | 0.42402 |
| SGB4837_group | 1.18137 | 1.05341 | −0.16539 | 99.73 | 99.82 | 0.96774 | 0.78072 |
| SGB4862 | 0.01221 | 0.00497 | −1.29623 | 11.03 | 7.83 | 0.98588 | 0.89260 |
| SGB4868 | 0.17220 | 0.13430 | −0.35864 | 79.36 | 78.11 | 0.43216 | 0.66742 |
| SGB4871_group | 0.19327 | 0.16330 | −0.24306 | 81.85 | 81.94 | 0.51636 | 0.28917 |
| SGB4882 | 0.08142 | 0.14083 | 0.79050 | 46.00 | 55.16 | 0.13224 | 0.15154 |
| SGB4894 | 0.09242 | 0.13030 | 0.49545 | 54.36 | 55.60 | 0.07226 | 0.19683 |
| SGB4906 | 0.02152 | 0.04486 | 1.05978 | 31.85 | 39.06 | 0.25437 | 0.21569 |
| SGB4914 | 0.85704 | 1.02558 | 0.25901 | 94.57 | 94.57 | 0.27733 | 0.23157 |
| SGB4933_group | 1.35137 | 0.92482 | −0.54718 | 94.84 | 93.24 | 0.88790 | 0.63459 |
| SGB4936 | 0.19336 | 0.30666 | 0.66534 | 94.57 | 94.93 | 0.28229 | 0.13806 |
| SGB4940 | 0.25604 | 0.14520 | −0.81831 | 88.26 | 82.56 | 0.72595 | 0.62357 |
| SGB4948 | 0.00872 | 0.01910 | 1.13177 | 6.23 | 9.70 | | |
| SGB4953 | 0.04209 | 0.09935 | 1.23895 | 45.37 | 55.78 | 0.13203 | 0.22868 |
| SGB4957 | 0.02531 | 0.03973 | 0.65011 | 41.64 | 44.40 | 0.19498 | 0.20421 |
| SGB4959 | 0.06648 | 0.05143 | −0.37021 | 79.36 | 76.78 | 0.53790 | 0.56183 |
| SGB4964 | 0.14563 | 0.36731 | 1.33475 | 82.74 | 91.01 | 0.03551 | 0.09527 |
| SGB4966 | 0.00753 | 0.01095 | 0.53990 | 38.61 | 44.48 | 0.22442 | 0.22360 |
| SGB4987 | 0.00895 | 0.00465 | −0.94452 | 18.77 | 12.46 | 0.80732 | 0.72394 |
| SGB4990 | 0.00227 | 0.00100 | −1.18622 | 11.21 | 7.12 | 0.59722 | 0.59990 |
| SGB4993 | 0.34855 | 0.20741 | −0.74892 | 94.57 | 93.77 | 0.21794 | 0.51285 |
| SGB5045 | 0.04790 | 0.03062 | −0.64564 | 69.04 | 60.68 | 0.60255 | 0.53646 |
| SGB5051 | 0.00553 | 0.00208 | −1.41083 | 16.73 | 11.21 | 0.72479 | 0.49085 |
| SGB5065 | 0.38417 | 0.19225 | −0.99874 | 17.88 | 15.93 | | |
| SGB5075_group | 0.46584 | 0.38727 | −0.26652 | 65.48 | 59.43 | 0.60293 | 0.57089 |
| SGB5082_group | 0.64040 | 0.50894 | −0.33148 | 93.42 | 93.51 | 0.27636 | 0.11462 |
| SGB5090_group | 0.38171 | 0.43451 | 0.18691 | 95.46 | 96.62 | 0.34253 | 0.17676 |
| SGB5180 | 0.01178 | 0.00721 | −0.70882 | 57.38 | 48.75 | 0.49881 | 0.42245 |
| SGB5184 | 0.00724 | 0.00400 | −0.85333 | 13.97 | 10.59 | 0.91435 | 0.89389 |
| SGB5190 | 0.14867 | 0.11498 | −0.37073 | 73.58 | 72.24 | 0.54760 | 0.27200 |
| SGB5290 | 0.00104 | 0.00057 | −0.85887 | 8.10 | 4.00 | | |
| SGB53497 | 0.00395 | 0.00250 | −0.65958 | 37.99 | 25.71 | 0.38007 | 0.73860 |
| SGB53515 | 0.00299 | 0.00199 | −0.58865 | 47.51 | 36.48 | 0.43386 | 0.46903 |
| SGB53517 | 0.01532 | 0.01231 | −0.31635 | 49.11 | 41.90 | 0.50726 | 0.67893 |
| SGB53821 | 0.00362 | 0.00182 | −0.99223 | 9.16 | 6.41 | 0.89842 | 0.82834 |
| SGB54300 | 0.01207 | 0.01825 | 0.59692 | 30.43 | 34.61 | 0.16384 | 0.39723 |
| SGB54347 | 0.00359 | 0.00182 | −0.98101 | 24.11 | 14.32 | 0.51933 | 0.72904 |
| SGB5785 | 0.13936 | 0.11530 | −0.27340 | 14.59 | 11.92 | | |
| SGB5792 | 0.39212 | 0.29332 | −0.41885 | 50.89 | 46.89 | 0.70708 | 0.61859 |
| SGB5803 | 0.17056 | 0.12938 | −0.39868 | 15.48 | 14.23 | 0.63832 | 0.46033 |
| SGB59869 | 0.00089 | 0.00047 | −0.92062 | 19.57 | 12.63 | 0.58592 | 0.62772 |
| SGB6153 | 0.00208 | 0.00078 | −1.41806 | 6.85 | 2.40 | 0.87063 | 0.71038 |
| SGB6276 | 0.14501 | 0.08738 | −0.73069 | 55.16 | 56.14 | 0.07948 | 0.27967 |
| SGB6305 | 0.03065 | 0.01617 | −0.92217 | 26.25 | 23.58 | 0.38788 | 0.43855 |
| SGB6308 | 0.19275 | 0.10549 | −0.86955 | 24.56 | 25.53 | | |
| SGB6317 | 0.12311 | 0.05240 | −1.23232 | 30.96 | 26.96 | 0.22377 | 0.46727 |
| SGB63278 | 0.00150 | 0.00084 | −0.82961 | 21.35 | 17.44 | | |
| SGB63325 | 0.00007 | 0.00001 | −2.53487 | 7.47 | 1.69 | | |
| SGB63326 | 0.00025 | 0.00010 | −1.28205 | 8.81 | 2.94 | 0.65878 | 0.87531 |
| SGB63327 | 0.00134 | 0.00040 | −1.75156 | 29.72 | 10.14 | 0.54542 | 0.89646 |
| SGB63342 | 0.00089 | 0.00036 | −1.30432 | 22.42 | 13.52 | 0.39919 | 0.53574 |
| SGB63343 | 0.00008 | 0.00002 | −2.04493 | 4.36 | 1.33 | 0.60211 | 0.69693 |
| SGB63353 | 0.00066 | 0.00020 | −1.73662 | 11.03 | 4.09 | 0.53874 | 0.63985 |
| SGB63369 | 0.00111 | 0.00047 | −1.24966 | 23.04 | 14.86 | 0.57423 | 0.66557 |
| SGB6340 | 0.10023 | 0.06233 | −0.68531 | 45.02 | 47.33 | 0.01448 | 0.09446 |
| SGB6360 | 0.00456 | 0.01032 | 1.17972 | 5.52 | 9.52 | | |
| SGB6465 | 0.05429 | 0.02711 | −1.00181 | 14.23 | 12.19 | | |
| SGB6473 | 0.07089 | 0.03136 | −1.17674 | 29.27 | 27.22 | | |
| SGB66170 | 0.00563 | 0.00283 | −0.99275 | 27.58 | 19.84 | 0.30425 | 0.67962 |
| SGB6747 | 0.00438 | 0.00155 | −1.49905 | 24.38 | 13.70 | 0.56473 | 0.79401 |
| SGB6754 | 0.11537 | 0.07097 | −0.70111 | 86.39 | 81.23 | 0.70732 | 0.24065 |
| SGB6767 | 0.00081 | 0.00010 | −3.06392 | 6.49 | 1.96 | 0.80983 | 0.77551 |
| SGB6796_group | 0.14805 | 0.10515 | −0.49367 | 34.07 | 32.03 | 0.65626 | 0.58571 |
| SGB6939 | 0.00764 | 0.00464 | −0.71855 | 22.33 | 15.39 | 0.80289 | 0.64389 |
| SGB6952 | 0.00817 | 0.00433 | −0.91573 | 21.26 | 13.61 | 0.58050 | 0.50345 |
| SGB6962_group | 0.06880 | 0.04423 | −0.63734 | 8.63 | 6.76 | 0.69051 | 0.79612 |
| SGB714_group | 0.07662 | 0.10746 | 0.48810 | 38.79 | 40.66 | 0.25638 | 0.46599 |
| SGB71759 | 0.01226 | 0.01729 | 0.49602 | 68.68 | 74.64 | 0.20708 | 0.16377 |
| SGB72336 | 0.00063 | 0.00026 | −1.27618 | 19.48 | 6.58 | 0.23951 | 0.60878 |
| SGB7253 | 0.00017 | 0.00006 | −1.46054 | 6.58 | 1.87 | 0.83757 | 0.84090 |
| SGB7258 | 0.00190 | 0.00149 | −0.35325 | 57.83 | 49.38 | 0.13209 | 0.11582 |
| SGB7259 | 0.00047 | 0.00019 | −1.28024 | 10.05 | 4.36 | 0.37219 | 0.62545 |
| SGB7263 | 0.00156 | 0.00069 | −1.17805 | 33.99 | 16.73 | 0.74876 | 0.83894 |
| SGB72916 | 0.01225 | 0.01435 | 0.22802 | 42.17 | 42.97 | 0.28450 | 0.29836 |
| SGB79798 | 0.00103 | 0.00078 | −0.41314 | 28.38 | 17.26 | 0.30667 | 0.57432 |
| SGB79833 | 0.00166 | 0.00108 | −0.61999 | 28.83 | 21.98 | 0.34637 | 0.27327 |
| SGB8002 | 0.03455 | 0.07005 | 1.01970 | 51.33 | 60.14 | 0.78487 | 0.54552 |
| SGB80143 | 0.00208 | 0.00120 | −0.78877 | 23.04 | 18.68 | 0.36942 | 0.14444 |
| SGB8071 | 0.00819 | 0.00626 | −0.38752 | 31.32 | 22.60 | 0.67925 | 0.77169 |
| SGB82503 | 0.00093 | 0.00034 | −1.44643 | 30.34 | 12.10 | 0.46808 | 0.74084 |
| SGB9203 | 0.06317 | 0.08106 | 0.35971 | 48.49 | 49.20 | 0.52849 | 0.39535 |
| SGB9205 | 0.05421 | 0.08370 | 0.62661 | 30.43 | 30.96 | 0.26485 | 0.44622 |
| SGB9226 | 0.87382 | 1.16288 | 0.41231 | 58.45 | 60.59 | 0.43836 | 0.37239 |
| SGB9283 | 0.38498 | 0.44730 | 0.21647 | 57.21 | 56.23 | 0.75081 | 0.59965 |
| SGB9286 | 0.24963 | 0.21243 | −0.23284 | 32.92 | 31.14 | 0.72025 | 0.50376 |
| SGB9391 | 0.02003 | 0.02963 | 0.56491 | 35.77 | 41.10 | 0.32317 | 0.21905 |

### IX. Closing Paragraphs

As will be understood by one of ordinary skill in the art, each embodiment disclosed herein can comprise, consist essentially of, or consist of its particular stated element, step, ingredient, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment. A material effect, in this context, is an alteration in the correlation between the presence, absence, or abundance of a microbe with a selected biological condition, or an alteration in a microbiome in a subject.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, other written text, and web site content throughout this specification (referenced materials herein). Each of the referenced materials are individually incorporated herein by reference in their entirety for their referenced teaching(s), as of the filing date of the first application in the priority chain in which the specific reference was included. For instance, with regard to chemical compounds, nucleic acid, amino acids sequences, and species-level genome bins (SGBs) referenced herein that are available in a public database, the information in the database entry is incorporated herein by reference as of the date of an application in the priority chain in which the database identifier for that compound or sequence or microbe was first included in the text.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 11th Edition or a dictionary known to those of ordinary skill in the art, such as the Oxford Dictionary of Biochemistry and Molecular Biology, 2^{nd} Edition (Ed. Anthony Smith, Oxford University Press, Oxford, 2006), and/or A Dictionary of Chemistry, 8^{th} Edition (Ed. J. Law & R. Rennie, Oxford University Press, 2020).

What is claimed is:

1. A method comprising:
receiving first test data from a remote device, the test data representing quantities of microbes present in a microbiome associated with an individual at a first time;
accessing a ranked list of indicator microbes;
determining, based at least in part on the first test data and the list of indicator microbes, a first microbiome score representing a quality of the microbiome associated with the individual at the first time; and
sending the microbiome score to a storage location accessible by a device associated with the individual.

2. The method of claim 1, further comprising:
using one or more weighted values associated with types of microbes in determining the microbiome score.

3. The method of claim 1 or 2, further comprising:
receiving first diet data from a remote device, the diet data representing the diet of the individual at the first time; and
using the first diet data at least in part in determining the microbiome score.

4. The method of any one of claims 1 to 3, further comprising:
generating, based at least in part on the first microbiome score and the first test data, a first recommended action, the first recommended action to increase a relative quantity of pro-health associated indicator microbes or decrease a relative quantity of poor health associated indicator microbes in the microbiome associated with the individual.

5. The method of claim 4, wherein the first recommended action is a change in diet of the individual.

6. The method of claim 4, wherein the first recommended action is a consumption of prebiotics or probiotics by the individual.

7. The method of any of one of claims 1 to 6, further comprising:
receiving second test data from the remote device, the second test data representing quantities of microbes present in the microbiome associated with the individual at a second time, the second time subsequent to the first time;
determining, based at least in part on the second test data and the list of indicator microbes, a second microbiome score representing a quality of the microbiome associated with the individual at the second time; and
sending the second microbiome score to the storage location accessible by the device associated with the individual.

8. The method of claim 7, further comprising:
receiving tracking data associated with the first recommended action from the device associated with the individual; and
wherein the second microbiome score's improvement compared against the first microbiome score is correlated to determining, based at least in part on the tracking data, that the individual has followed the recommended action for a predetermined period for time or until a target associated with the first recommended action is achieved.

9. The method of any one of claims 2 to 8, further comprising:
determining the one or more weighted values based at least in part on health and/or diet data and microbiome data associated with a plurality of individuals.

10. The method of any one of claims 2 to 9, further comprising:
determining the one or more weighted values based at least in part on microbiome data associated with a plurality of individuals at two or more times per individual.

11. The method of any one of claims 2 to 10, wherein:
determining the one or more weighted values further comprises:
training one or more machine learned models using microbiome data associated with a plurality of individuals; and
receiving, from the one or more machine learned models, at least one weighted value associated with a type of microbe.

12. The method of any one of claims 1 to 11, further comprising:
determining the first microbiome score further comprises:
inputting the first test data into one or more machine learned models trained using microbiome data associated with a plurality of individuals; and
receiving, as an output from the one or more machine learned models, the first microbiome score.

13. The method of any one of claims 7 to 12, further comprising:
generating a graphical representation of changes in the microbiome associated with the individual over a period of time including the first time and the second time; and
causing the graphical representation to be presented on a display of the device associated with the individual.

14. The method of any one of claims 1 to 13, wherein the quantity of microbes is expressed as a percentage of the microbiome.

15. The method of any one of claims 1 to 14, wherein at least ten of the indicator microbes (identified by its species-level genome bin (SGB) designation) are selected from:
SGB15249, SGB6340, SGB4964, SGB14252, SGB15229, SGB6174_group, SGB15317, SGB14179, SGB15225, SGB4894, SGB4643, SGB4963, SGB79840, SGB4893, SGB6276, SGB3952, SGB4638, SGB15236, SGB4191, SGB15053_group, SGB15368, SGB4782, SGB14042, SGB4706, SGB4644, SGB49188, SGB4781, SGB4777, SGB14921, SGB15234, SGB8601, SGB5087, SGB14311, SGB4953, SGB7258, SGB4882, SGB6367, SGB15106, SGB4778, SGB15131, SGB4198_group, SGB15031, SGB13981, SGB15123, SGB54300, SGB4665, SGB13979, SGB15410, SGB2290, SGB14954, SGB14306, SGB4805, SGB14899, SGB4803, SGB13982, SGB15265_group, SGB14114, SGB47656, SGB6749, SGB14253, SGB15346, SGB4810, SGB4770, SGB14043, SGB4886, SGB25497, SGB4815_group, SGB25416, SGB4957, SGB4654, SGB15373, SGB15254, SGB6571, SGB15323, SGB71759, SGB4648, SGB15180, SGB15413, SGB49168, SGB14960, SGB4133, SGB15051, SGB4993, SGB15395, SGB15145, SGB5111, SGB6317, SGB4966, SGB4780, SGB14198, SGB63101, SGB4779, SGB15233, SGB4769, SGB2295, SGB72336, SGB4658, SGB14770, SGB6148, SGB25493, SGB4831_group, SGB14965, SGB15224, SGB4938, SGB15402, SGB15291, SGB9333, SGB4664, SGB4906, SGB4711, SGB15065, SGB714_group, SGB4772, SGB3958, SGB4629, SGB14048, SGB15052, SGB14861, SGB9205, SGB4280, SGB4829, SGB4816, SGB2317, SGB15411, SGB5117, SGB14250, SGB14924, SGB4767, SGB6376, SGB4714, SGB4691, SGB14341, SGB15244, SGB5082_group, SGB4910, SGB4914, SGB8599, SGB4936, SGB15374, SGB72916, SGB4909, SGB15390, SGB15164, SGB15093, SGB13983, SGB5042, SGB4771, SGB15356, SGB72479, SGB4557, SGB3988, SGB15041, SGB14128, SGB15385, SGB6750, SGB4184, SGB3573, SGB66170, SGB15201, SGB15203, SGB79798, SGB15382, SGB4652, SGB9346, SGB14969, SGB4262, SGB4394, SGB61601, SGB15216, SGB14027, SGB4674, SGB14937, SGB15090, SGB9391, SGB15383, SGB29347, SGB14991, SGB14940, SGB4809, SGB6141, SGB4687, SGB63163, SGB14177, SGB4832, SGB15160, SGB48024, SGB6179, SGB4768, SGB5090_group, SGB29302, SGB9712_group, SGB3813, SGB79833, SGB4659, SGB4328, SGB4776, SGB1790, SGB14313, SGB5043, SGB15127, SGB15049, SGB42321, SGB15403, SGB15115, SGB4905, SGB14838, SGB15012, SGB9202, SGB80143, SGB3992, SGB7259, SGB4546, SGB14974, SGB13976, SGB15342, SGB2296, SGB14941, SGB3996, SGB53497, SGB15470, SGB14020, SGB1858, SGB14851, SGB6305, SGB14932, SGB15089, SGB1862, SGB15401, SGB4027, SGB15140, SGB2325, SGB14317, SGB4628, SGB4669, SGB15299, SGB6478, SGB14262, SGB63342, SGB4960, SGB63333, SGB15316_group, SGB4651, SGB1965, SGB15081, SGB59819, SGB2326, SGB14912, SGB14322_group, SGB3940, SGB4029, SGB2301, SGB63167, SGB14797_group, SGB5200, SGB17347, SGB4868, SGB15067, SGB53515, SGB15075, SGB4421, SGB5121, SGB9226, SGB2318, SGB14894, SGB4817, SGB14966, SGB3989, SGB15370, SGB14975, SGB4436, SGB14839, SGB14993_group, SGB15322, SGB9387, SGB3959, SGB6362, SGB4063, SGB14773_group, SGB29334, SGB14151, SGB15087, SGB14022, SGB14972, SGB15045, SGB4712, SGB15389, SGB82503, SGB15318_group, SGB1857, SGB4828_group, SGB4788_group, SGB79822, SGB4269, SGB14923, SGB2328, SGB1784, SGB14137, SGB15204, SGB7256, SGB15295_group, SGB14182, SGB29342, SGB4438, SGB14824_group, SGB2303, SGB9262, SGB14952, SGB14951, SGB15459, SGB6358, SGB14929, SGB15286, SGB5060, SGB15332_group, SGB15126, SGB72433_group, SGB4045, SGB1626, SGB5180, SGB4867, SGB4825, SGB4925, SGB53517, SGB1844, SGB14844, SGB4871_group, SGB14050, SGB25547, SGB1815, SGB3957, SGB54347, SGB6140, SGB14127, SGB29339, SGB1832, SGB9342_group, SGB15278, SGB9203, SGB1962, SGB5076, SGB4808, SGB15119, SGB3962, SGB14892, SGB4775, SGB4166, SGB7144, SGB4959, SGB63353, SGB14953, SGB6139, SGB16971, SGB15300, SGB3574, SGB47850, SGB63327, SGB4181, SGB15506, SGB5190, SGB14181, SGB1846, SGB15068, SGB4537, SGB14906, SGB9347, SGB1786, SGB7265, SGB4571, SGB29321, SGB4531, SGB15073, SGB14933, SGB15154, SGB6747, SGB15273, SGB4367, SGB15091, SGB3965, SGB63369, SGB9224, SGB3964, SGB6952, SGB4765, SGB29305, SGB4285_group, SGB5089, SGB4581, SGB59869, SGB4727, SGB25431, SGB2299, SGB1829, SGB4990, SGB1891_group, SGB2286, SGB17278, SGB1949, SGB63343, SGB5045, SGB5075_group, SGB8007_group, SGB29375, SGB6847, SGB1798, SGB4670, SGB4582_group, SGB4290, SGB14891, SGB1785, SGB15209, SGB14150, SGB33551, SGB14958, SGB14807, SGB4820, SGB1812, SGB4716, SGB4425_group, SGB9340, SGB14779, SGB14125, SGB14259, SGB4784, SGB15260, SGB14741, SGB4577_group, SGB71281, SGB14307, SGB5803, SGB58519, SGB5077, SGB15125, SGB15143, SGB4116, SGB4677, SGB15467_group, SGB7202, SGB6178, SGB6796_group, SGB6783_group, SGB1860, SGB4059, SGB63326, SGB9272_group, SGB15350, SGB14334, SGB1941, SGB16986, SGB14909, SGB1963, SGB4774, SGB14143, SGB15272, SGB29380, SGB4811_group, SGB4595, SGB4834, SGB4030, SGB8071, SGB2311, SGB3991, SGB4722, SGB17244, SGB3993, SGB4553, SGB6956, SGB1699, SGB6962_group, SGB4705, SGB1957, SGB4532, SGB4327_group, SGB59559, SGB4594, SGB5765_group, SGB17169, SGB15120, SGB1948, SGB14853, SGB14898, SGB48424, SGB5792, SGB6754, SGB1934, SGB14854, SGB6768, SGB15156_group, SGB4750, SGB14142, SGB17153_group, SGB4552_group, SGB4951, SGB4303, SGB9286, SGB5051, SGB17237, SGB4940, SGB4597, SGB4563_group, SGB1867, SGB47515, SGB59562, SGB8059_group, SGB4991, SGB4540_group, SGB14862, SGB4422, SGB15124, SGB14987, SGB7263, SGB9283, SGB4348, SGB14895, SGB17154, SGB17167, SGB4626, SGB5843, SGB4575, SGB4613, SGB15076, SGB17130, SGB15904, SGB4080, SGB6846, SGB5825_group, SGB14808, SGB4874, SGB9228, SGB6320, SGB9260, SGB8002, SGB6936, SGB14962, SGB8053, SGB4701, SGB5197, SGB4036, SGB4744, SGB1877, SGB29313, SGB14845, SGB14963, SGB8056, SGB6939, SGB17256, SGB4749, SGB3961, SGB7142, SGB14999, SGB4747, SGB15149, SGB4987, SGB6767, SGB17248, SGB4725, SGB59576, SGB1903_group, SGB5183, SGB49059, SGB4121, SGB25538, SGB3970, SGB3969, SGB14890, SGB1830_group, SGB7967, SGB17168, SGB8047, SGB4046, SGB1855_group, SGB3922, SGB14995, SGB7253, SGB48013, SGB4741, SGB4671, SGB15878, SGB6769, SGB14180, SGB29433, SGB1871, SGB5182, SGB5736, SGB6771, SGB4721, SGB14837, SGB4044, SGB8095, SGB17152, SGB1861, SGB66069, SGB4031, SGB6153, SGB7264, SGB15121, SGB25437, SGB14546_group, SGB4933_group, SGB4763, SGB5193, SGB7985, SGB4699, SGB19850_group, SGB17137, SGB4785, SGB15078, SGB53821, SGB15452, SGB15271, SGB4724, SGB8255_group, SGB79823, SGB8028_group, SGB4573_group, SGB14874, SGB4988, SGB5184, SGB4786, SGB4826_group, SGB4041, SGB7984, SGB4761, SGB4447, SGB6744, SGB1836_group, SGB1814, SGB4630_group, SGB8163, SGB4617, SGB4588_group, SGB4742, SGB4572, SGB10115, SGB15158, SGB29328, SGB4791, SGB4688, SGB10068, SGB71883, SGB4760, SGB4037_group, SGB4529, SGB4837_group, SGB79883, SGB4762, SGB4797, SGB14809, SGB4758_group, SGB4703, SGB4606, SGB4584, SGB15132, SGB4746, SGB4862, SGB4798, SGB4861, SGB4608, SGB4035, SGB4794_group, SGB4753, SGB4583.

16. The method of claim 15, wherein the microbes are ranked based at least in part on a diet rank or health rank.

17. The method of claim 15, wherein at least ten of the selected microbes are ranked in the same order relative to each other as per their diet ranks as reflected in Table 1A.

18. The method of claim 15, wherein at least ten of the selected microbes are ranked in the same order relative to each other as per their health ranks as reflected in Table 1A.

19. The method of any one of claims 1 to 18, wherein the microbiome is a gut microbiome.

20. A computer program product comprising coded instructions that, when run on a computer, implement a method as claimed in any one of claims 1 to 19.

21. A system comprising:
one or more processors; and
one or more non-transitory computer readable media storing instructions executable by the one or more processors, wherein the instruction, when executed, causes the one or more processors to perform operations comprising:
receiving one or more weighted values from one or more first machine learned models trained on microbiome data associated with a plurality of individuals, each of the one or more weighted values representing at least in part a pro-health or poor health impact of a microbe or a plurality of microbes;
receiving first test data representing a presence of and quantities of microbes present in a microbiome associated with an individual at a first time;
inputting the test data into one or more second machine learned models which use at least in part the one or more weighted values;
receiving as a first output of the one or more second machine learned models a first microbiome score representing a quality of the microbiome associated with the individual at the first time; and
sending the microbiome score to storage location accessible by a device associated with the individual.

22. The system of claim 21, wherein the operations further comprise:
inputting the test data into one or more third machine learned models trained at least in part on observed changes in microbiome data for a plurality of test subjects over time; and
receiving as an output of the one or more third machine learned models a recommended action, the recommended action intended to increase a relative quantity of pro-health indicator microbes or decrease a relative quantity of poor health indicator microbes in the microbiome associated with the individual.

23. The system of claim 21 or 22, wherein the operations further comprise:
receiving second test data representing quantities of microbes present in the microbiome associated with the individual at a second time, the second time subsequent to the first time;
inputting the second test data into the one or more second machine learned models and receiving as a second output of the one or more second machine learned models a second microbiome score representing a quality of the microbiome associated with the individual at the second time; and
sending the second microbiome score to the storage location accessible by the device associated with the individual.

24. The system of any one of claims 21 to 23, wherein the operations further comprise:
inputting the test data into one or more fourth machine learned models trained at least in part on observed changes in microbiome data for a plurality of test subjects over time; and
receiving as an output of the one or more fourth machine learned models, a plurality of predicted microbiome scores, each of the plurality of predicted microbiome scores representing a quality of the microbiome of the individual at a future time in response to the individual performing a corresponding recommended action.

25. The system of any one of claims 21 to 24, wherein a negative weighted value represents a poor health associated indicator microbe and a positive weighted value represents a pro-health associated indicator microbe.

26. The system of any one of claims 21 to 25, wherein inputting the test data into one or more second machine learned models further comprises:
selecting specific microbes from the test data; and
inputting the quantities of the specific microbes into the one or more second machine learned models.

27. The system of any one of claims 21 to 26, wherein the operations further comprise:
receiving user data associated with the individual, the user data representing at least one of a health of the individual, a demographic associated with the individual, a diet of the individual, or a geographic region associated with the individual.

28. The system of any one of claims 21 to 27, wherein at least ten of the microbes (identified by its species-level genome bin (SGB) designation) are selected from: SGB15249, SGB6340, SGB4964, SGB14252, SGB15229, SGB6174_group, SGB15317, SGB14179, SGB15225, SGB4894, SGB4643, SGB4963, SGB79840, SGB4893, SGB6276, SGB3952, SGB4638, SGB15236, SGB4191, SGB15053_group, SGB15368, SGB4782, SGB14042, SGB4706, SGB4644, SGB49188, SGB4781, SGB4777, SGB14921, SGB15234, SGB8601, SGB5087, SGB14311, SGB4953, SGB7258, SGB4882, SGB6367, SGB15106, SGB4778, SGB15131, SGB4198_group, SGB15031, SGB13981, SGB15123, SGB54300, SGB4665, SGB13979, SGB15410, SGB2290, SGB14954, SGB14306, SGB4805, SGB14899, SGB4803, SGB13982, SGB15265_group, SGB14114, SGB47656, SGB6749, SGB14253, SGB15346, SGB4810, SGB4770, SGB14043, SGB4886, SGB25497, SGB4815_group, SGB25416, SGB4957, SGB4654, SGB15373, SGB15254, SGB6571, SGB15323, SGB71759, SGB4648, SGB15180, SGB15413, SGB49168, SGB14960, SGB4133, SGB15051, SGB4993, SGB15395, SGB15145, SGB5111, SGB6317, SGB4966, SGB4780, SGB14198, SGB63101, SGB4779, SGB15233, SGB4769, SGB2295, SGB72336, SGB4658, SGB14770, SGB6148, SGB25493, SGB4831_group, SGB14965, SGB15224, SGB4938, SGB15402, SGB15291, SGB9333, SGB4664, SGB4906, SGB4711, SGB15065, SGB714_group, SGB4772, SGB3958, SGB4629, SGB14048, SGB15052, SGB14861, SGB9205, SGB4280, SGB4829, SGB4816, SGB2317, SGB15411, SGB5117, SGB14250, SGB14924, SGB4767, SGB6376, SGB4714, SGB4691, SGB14341, SGB15244, SGB5082_group, SGB4910, SGB4914, SGB8599, SGB4936, SGB15374, SGB72916, SGB4909, SGB15390, SGB15164, SGB15093, SGB13983, SGB5042, SGB4771, SGB15356, SGB72479, SGB4557, SGB3988, SGB15041, SGB14128, SGB15385, SGB6750, SGB4184, SGB3573, SGB66170, SGB15201, SGB15203, SGB79798, SGB15382, SGB4652, SGB9346, SGB14969, SGB4262, SGB4394, SGB61601, SGB15216, SGB14027, SGB4674, SGB14937, SGB15090, SGB9391, SGB15383, SGB29347, SGB14991, SGB14940, SGB4809, SGB6141, SGB4687, SGB63163, SGB14177, SGB4832, SGB15160, SGB48024, SGB6179, SGB4768, SGB5090_group, SGB29302, SGB9712_group, SGB3813, SGB79833, SGB4659, SGB4328, SGB4776, SGB1790, SGB14313, SGB5043, SGB15127, SGB15049, SGB42321, SGB15403, SGB15115, SGB4905, SGB14838, SGB15012, SGB9202, SGB80143, SGB3992, SGB7259, SGB4546, SGB14974, SGB13976, SGB15342, SGB2296, SGB14941, SGB3996, SGB53497, SGB15470, SGB14020, SGB1858, SGB14851, SGB6305, SGB14932, SGB15089, SGB1862, SGB15401, SGB4027, SGB15140, SGB2325, SGB14317, SGB4628, SGB4669, SGB15299, SGB6478, SGB14262, SGB63342, SGB4960, SGB63333, SGB15316_group, SGB4651, SGB1965, SGB15081, SGB59819, SGB2326, SGB14912, SGB14322_group, SGB3940, SGB4029, SGB2301, SGB63167, SGB14797_group, SGB5200, SGB17347, SGB4868, SGB15067, SGB53515, SGB15075, SGB4421, SGB5121, SGB9226, SGB2318, SGB14894, SGB4817, SGB14966, SGB3989, SGB15370, SGB14975, SGB4436, SGB14839, SGB14993_group, SGB15322, SGB9387, SGB3959, SGB6362, SGB4063, SGB14773_group, SGB29334, SGB14151, SGB15087, SGB14022, SGB14972, SGB15045, SGB4712, SGB15389, SGB82503, SGB15318_group, SGB1857, SGB4828_group, SGB4788_group, SGB79822, SGB4269, SGB14923, SGB2328, SGB1784, SGB14137, SGB15204, SGB7256, SGB15295_group, SGB14182, SGB29342, SGB4438, SGB14824_group, SGB2303, SGB9262, SGB14952, SGB14951, SGB15459, SGB6358, SGB14929, SGB15286, SGB5060, SGB15332_group, SGB15126, SGB72433_group, SGB4045, SGB1626, SGB5180, SGB4867, SGB4825, SGB4925, SGB53517, SGB1844, SGB14844, SGB4871_group, SGB14050, SGB25547, SGB1815, SGB3957, SGB54347, SGB6140, SGB14127, SGB29339, SGB1832, SGB9342_group, SGB15278, SGB9203, SGB1962, SGB5076, SGB4808, SGB15119, SGB3962, SGB14892, SGB4775, SGB4166, SGB7144, SGB4959, SGB63353, SGB14953, SGB6139, SGB16971, SGB15300, SGB3574, SGB47850, SGB63327, SGB4181, SGB15506, SGB5190, SGB14181, SGB1846, SGB15068, SGB4537, SGB14906, SGB9347, SGB1786, SGB7265, SGB4571, SGB29321, SGB4531, SGB15073, SGB14933, SGB15154, SGB6747, SGB15273, SGB4367, SGB15091, SGB3965, SGB63369, SGB9224, SGB3964, SGB6952, SGB4765, SGB29305, SGB4285_group, SGB5089, SGB4581, SGB59869, SGB4727, SGB25431, SGB2299, SGB1829, SGB4990, SGB1891_group, SGB2286, SGB17278, SGB1949, SGB63343, SGB5045, SGB5075_group, SGB8007_group, SGB29375, SGB6847, SGB1798, SGB4670, SGB4582_group, SGB4290, SGB14891, SGB1785, SGB15209, SGB14150, SGB33551, SGB14958, SGB14807, SGB4820, SGB1812, SGB4716, SGB4425_group, SGB9340, SGB14779, SGB14125, SGB14259, SGB4784, SGB15260, SGB14741, SGB4577_group, SGB71281, SGB14307, SGB5803, SGB58519, SGB5077, SGB15125, SGB15143, SGB4116, SGB4677, SGB15467_group, SGB7202, SGB6178, SGB6796_group, SGB6783_group, SGB1860, SGB4059, SGB63326, SGB9272_group, SGB15350, SGB14334, SGB1941, SGB16986, SGB14909, SGB1963, SGB4774, SGB14143, SGB15272, SGB29380, SGB4811_group, SGB4595, SGB4834, SGB4030, SGB8071, SGB2311, SGB3991, SGB4722, SGB17244, SGB3993, SGB4553, SGB6956, SGB1699, SGB6962_group, SGB4705, SGB1957, SGB4532, SGB4327_group, SGB59559, SGB4594, SGB5765_group, SGB17169, SGB15120, SGB1948, SGB14853, SGB14898, SGB48424, SGB5792, SGB6754, SGB1934, SGB14854, SGB6768, SGB15156_group, SGB4750, SGB14142, SGB17153_group, SGB4552_group, SGB4951, SGB4303, SGB9286, SGB5051, SGB17237, SGB4940, SGB4597, SGB4563_group, SGB1867, SGB47515, SGB59562, SGB8059_group, SGB4991, SGB4540_group, SGB14862, SGB4422, SGB15124, SGB14987, SGB7263, SGB9283, SGB4348, SGB14895, SGB17154, SGB17167, SGB4626, SGB5843, SGB4575, SGB4613, SGB15076, SGB17130, SGB15904, SGB4080, SGB6846, SGB5825_group, SGB14808, SGB4874, SGB9228, SGB6320, SGB9260, SGB8002, SGB6936, SGB14962, SGB8053, SGB4701, SGB5197, SGB4036, SGB4744, SGB1877, SGB29313, SGB14845, SGB14963, SGB8056, SGB6939, SGB17256, SGB4749, SGB3961, SGB7142, SGB14999, SGB4747, SGB15149, SGB4987, SGB6767, SGB17248, SGB4725, SGB59576, SGB1903_group, SGB5183, SGB49059, SGB4121, SGB25538, SGB3970, SGB3969, SGB14890, SGB1830_group, SGB7967, SGB17168, SGB8047, SGB4046, SGB1855_group, SGB3922, SGB14995, SGB7253, SGB48013, SGB4741, SGB4671, SGB15878, SGB6769, SGB14180, SGB29433, SGB1871, SGB5182, SGB5736, SGB6771, SGB4721, SGB14837, SGB4044, SGB8095, SGB17152, SGB1861, SGB66069, SGB4031, SGB6153, SGB7264, SGB15121, SGB25437, SGB14546_group, SGB4933_group, SGB4763, SGB5193, SGB7985, SGB4699, SGB19850_group, SGB17137, SGB4785, SGB15078, SGB53821, SGB15452, SGB15271, SGB4724, SGB8255_group, SGB79823, SGB8028_group, SGB4573_group, SGB14874, SGB4988, SGB5184, SGB4786, SGB4826_group, SGB4041, SGB7984, SGB4761, SGB4447, SGB6744, SGB1836_group, SGB1814, SGB4630_group, SGB8163, SGB4617, SGB4588_group, SGB4742, SGB4572, SGB10115, SGB15158, SGB29328, SGB4791, SGB4688, SGB10068, SGB71883, SGB4760, SGB4037_group, SGB4529, SGB4837_group, SGB79883, SGB4762, SGB4797, SGB14809, SGB4758_group, SGB4703, SGB4606, SGB4584, SGB15132, SGB4746, SGB4862, SGB4798, SGB4861, SGB4608, SGB4035, SGB4794_group, SGB4753, SGB4583.

29. The system of claim 28, wherein the microbes are ranked based at least in part on a diet rank or health rank.

30. The system of claim 28, wherein at least ten of the selected microbes are ranked in the same order relative to each other as per their diet ranks as reflected in Table 1A.

31. The system of claim 28, wherein at least ten of the selected microbes are ranked in the same order relative to each other as per their health ranks as reflected in Table 1A.

32. The system of any one of claims 22 to 31, wherein the microbiome data is gut microbiome data.

33. The system of any one of claims 22 to 32, wherein the quantity of microbes is expressed as a percentage of the microbiome.

34. A method comprising:
receiving first test data from a remote device, the first test data representing quantities of microbes present in one or more first microbiome samples;
receiving second test data from a remote device, the second test data representing quantities of microbes present in one or more second microbiome samples;
accessing a list of indicator microbes and their associations with pro-health versus poor health;
determining which indicator microbes increase or decrease between the first and second microbiome samples;
determining whether the increasing indicator microbes have stronger pro-health associations or stronger poor health associations compared to the decreasing indicator microbes, representing respectively an increase or decrease in the quality of the microbiome between the first and second microbiomes samples; and
sending the comparison to a storage location.

35. The method of claim 34, wherein the microbiome is a gut microbiome.

36. The method of claim 34 or 35, wherein the quantity of microbes is expressed as a percentage of the microbiome.

37. The method of any one of claims 34 to 36, wherein at least ten of the microbes (identified by its species-level genome bin (SGB) designation) are selected from: SGB15249, SGB6340, SGB4964, SGB14252, SGB15229, SGB6174_group, SGB15317, SGB14179, SGB15225, SGB4894, SGB4643, SGB4963, SGB79840, SGB4893, SGB6276, SGB3952, SGB4638, SGB15236, SGB4191, SGB15053_group, SGB15368, SGB4782, SGB14042, SGB4706, SGB4644, SGB49188, SGB4781, SGB4777, SGB14921, SGB15234, SGB8601, SGB5087, SGB14311, SGB4953, SGB7258, SGB4882, SGB6367, SGB15106, SGB4778, SGB15131, SGB4198_group, SGB15031, SGB13981, SGB15123, SGB54300, SGB4665, SGB13979, SGB15410, SGB2290, SGB14954, SGB14306, SGB4805, SGB14899, SGB4803, SGB13982, SGB15265_group, SGB14114, SGB47656, SGB6749, SGB14253, SGB15346, SGB4810, SGB4770, SGB14043, SGB4886, SGB25497, SGB4815_group, SGB25416, SGB4957, SGB4654, SGB15373, SGB15254, SGB6571, SGB15323, SGB71759, SGB4648, SGB15180, SGB15413, SGB49168, SGB14960, SGB4133, SGB15051, SGB4993, SGB15395, SGB15145, SGB5111, SGB6317, SGB4966, SGB4780, SGB14198, SGB63101, SGB4779, SGB15233, SGB4769, SGB2295, SGB72336, SGB4658, SGB14770, SGB6148, SGB25493, SGB4831_group, SGB14965, SGB15224, SGB4938, SGB15402, SGB15291, SGB9333, SGB4664, SGB4906, SGB4711, SGB15065, SGB714_group, SGB4772, SGB3958, SGB4629, SGB14048, SGB15052, SGB14861, SGB9205, SGB4280, SGB4829, SGB4816, SGB2317, SGB15411, SGB5117, SGB14250, SGB14924, SGB4767, SGB6376, SGB4714, SGB4691, SGB14341, SGB15244, SGB5082_group, SGB4910, SGB4914, SGB8599, SGB4936, SGB15374, SGB72916, SGB4909, SGB15390, SGB15164, SGB15093, SGB13983, SGB5042, SGB4771, SGB15356, SGB72479, SGB4557, SGB3988, SGB15041, SGB14128, SGB15385, SGB6750, SGB4184, SGB3573, SGB66170, SGB15201, SGB15203, SGB79798, SGB15382, SGB4652, SGB9346, SGB14969, SGB4262, SGB4394, SGB61601, SGB15216, SGB14027, SGB4674, SGB14937, SGB15090, SGB9391, SGB15383, SGB29347, SGB14991, SGB14940, SGB4809, SGB6141, SGB4687, SGB63163, SGB14177, SGB4832, SGB15160, SGB48024, SGB6179, SGB4768, SGB5090_group, SGB29302, SGB9712_group, SGB3813, SGB79833, SGB4659, SGB4328, SGB4776, SGB1790, SGB14313, SGB5043, SGB15127, SGB15049, SGB42321, SGB15403, SGB15115, SGB4905, SGB14838, SGB15012, SGB9202, SGB80143, SGB3992, SGB7259, SGB4546, SGB14974, SGB13976, SGB15342, SGB2296, SGB14941, SGB3996, SGB53497, SGB15470, SGB14020, SGB1858, SGB14851, SGB6305, SGB14932, SGB15089, SGB1862, SGB15401, SGB4027, SGB15140, SGB2325, SGB14317, SGB4628, SGB4669, SGB15299, SGB6478, SGB14262, SGB63342, SGB4960, SGB63333, SGB15316_group, SGB4651, SGB1965, SGB15081, SGB59819, SGB2326, SGB14912, SGB14322_group, SGB3940, SGB4029, SGB2301, SGB63167, SGB14797_group, SGB5200, SGB17347, SGB4868, SGB15067, SGB53515, SGB15075, SGB4421, SGB5121, SGB9226, SGB2318, SGB14894, SGB4817, SGB14966, SGB3989, SGB15370, SGB14975, SGB4436, SGB14839, SGB14993_group, SGB15322, SGB9387, SGB3959, SGB6362, SGB4063, SGB14773_group, SGB29334, SGB14151, SGB15087, SGB14022, SGB14972, SGB15045, SGB4712, SGB15389, SGB82503, SGB15318_group, SGB1857, SGB4828_group, SGB4788_group, SGB79822, SGB4269, SGB14923, SGB2328, SGB1784, SGB14137, SGB15204, SGB7256, SGB15295_group, SGB14182, SGB29342, SGB4438, SGB14824_group, SGB2303, SGB9262, SGB14952, SGB14951, SGB15459, SGB6358, SGB14929, SGB15286, SGB5060, SGB15332_group, SGB15126, SGB72433_group, SGB4045, SGB1626, SGB5180, SGB4867, SGB4825, SGB4925, SGB53517, SGB1844, SGB14844, SGB4871_group, SGB14050, SGB25547, SGB1815, SGB3957, SGB54347, SGB6140, SGB14127, SGB29339, SGB1832, SGB9342_group, SGB15278, SGB9203, SGB1962, SGB5076, SGB4808, SGB15119, SGB3962, SGB14892, SGB4775, SGB4166, SGB7144, SGB4959, SGB63353, SGB14953, SGB6139, SGB16971, SGB15300, SGB3574, SGB47850, SGB63327, SGB4181, SGB15506, SGB5190, SGB14181, SGB1846, SGB15068, SGB4537, SGB14906, SGB9347, SGB1786, SGB7265, SGB4571, SGB29321, SGB4531, SGB15073, SGB14933, SGB15154, SGB6747, SGB15273, SGB4367, SGB15091, SGB3965, SGB63369, SGB9224, SGB3964, SGB6952, SGB4765, SGB29305, SGB4285_group, SGB5089, SGB4581, SGB59869, SGB4727, SGB25431, SGB2299, SGB1829, SGB4990, SGB1891_group, SGB2286, SGB17278, SGB1949, SGB63343, SGB5045, SGB5075_group, SGB8007_group, SGB29375, SGB6847, SGB1798, SGB4670, SGB4582_group, SGB4290, SGB14891, SGB1785, SGB15209, SGB14150, SGB33551, SGB14958, SGB14807, SGB4820, SGB1812, SGB4716, SGB4425_group, SGB9340, SGB14779, SGB14125, SGB14259, SGB4784, SGB15260, SGB14741, SGB4577_group, SGB71281, SGB14307, SGB5803, SGB58519, SGB5077, SGB15125, SGB15143, SGB4116, SGB4677, SGB15467_group, SGB7202, SGB6178, SGB6796_group, SGB6783_group, SGB1860, SGB4059, SGB63326, SGB9272_group, SGB15350, SGB14334, SGB1941, SGB16986, SGB14909, SGB1963, SGB4774, SGB14143, SGB15272, SGB29380, SGB4811_group, SGB4595, SGB4834, SGB4030, SGB8071, SGB2311, SGB3991, SGB4722, SGB17244, SGB3993, SGB4553, SGB6956, SGB1699, SGB6962_group, SGB4705, SGB1957, SGB4532, SGB4327_group, SGB59559, SGB4594, SGB5765_group, SGB17169, SGB15120, SGB1948, SGB14853, SGB14898, SGB48424, SGB5792, SGB6754, SGB1934, SGB14854, SGB6768, SGB15156_group, SGB4750, SGB14142, SGB17153_group, SGB4552_group, SGB4951, SGB4303, SGB9286, SGB5051, SGB17237, SGB4940, SGB4597, SGB4563_group, SGB1867, SGB47515, SGB59562, SGB8059_group, SGB4991, SGB4540_group, SGB14862, SGB4422, SGB15124, SGB14987, SGB7263, SGB9283, SGB4348, SGB14895, SGB17154, SGB17167, SGB4626, SGB5843, SGB4575, SGB4613, SGB15076, SGB17130, SGB15904, SGB4080, SGB6846, SGB5825_group, SGB14808, SGB4874, SGB9228, SGB6320, SGB9260, SGB8002, SGB6936, SGB14962, SGB8053, SGB4701, SGB5197, SGB4036, SGB4744, SGB1877, SGB29313, SGB14845, SGB14963, SGB8056, SGB6939, SGB17256, SGB4749, SGB3961, SGB7142, SGB14999, SGB4747, SGB15149, SGB4987, SGB6767, SGB17248, SGB4725, SGB59576, SGB1903_group, SGB5183, SGB49059, SGB4121, SGB25538, SGB3970, SGB3969, SGB14890, SGB1830_group, SGB7967, SGB17168, SGB8047, SGB4046, SGB1855_group, SGB3922, SGB14995, SGB7253, SGB48013, SGB4741, SGB4671, SGB15878, SGB6769, SGB14180, SGB29433, SGB1871, SGB5182, SGB5736, SGB6771, SGB4721, SGB14837, SGB4044, SGB8095, SGB17152, SGB1861, SGB66069, SGB4031, SGB6153, SGB7264, SGB15121, SGB25437, SGB14546_group, SGB4933_group, SGB4763, SGB5193, SGB7985, SGB4699, SGB19850_group, SGB17137, SGB4785, SGB15078, SGB53821, SGB15452, SGB15271, SGB4724, SGB8255_group, SGB79823, SGB8028_group, SGB4573_group, SGB14874, SGB4988, SGB5184, SGB4786, SGB4826_group, SGB4041, SGB7984, SGB4761, SGB4447, SGB6744, SGB1836_group, SGB1814, SGB4630_group, SGB8163, SGB4617, SGB4588_group, SGB4742, SGB4572, SGB10115, SGB15158, SGB29328, SGB4791, SGB4688, SGB10068, SGB71883, SGB4760, SGB4037_group, SGB4529, SGB4837_group, SGB79883, SGB4762, SGB4797, SGB14809, SGB4758_group, SGB4703, SGB4606, SGB4584, SGB15132, SGB4746, SGB4862, SGB4798, SGB4861, SGB4608, SGB4035, SGB4794_group, SGB4753, SGB4583.

38. The method of claim 37, wherein the microbes are ranked based at least in part on a diet rank or health rank.

39. The method of claim 37, wherein at least ten of the selected microbes are ranked in the same order relative to each other as per their diet ranks as reflected in Table 1A.

40. The method of claim 37, wherein at least ten of the selected microbes are ranked in the same order relative to each other as per their health ranks as reflected in Table 1A.

41. A method comprising:
receiving first test data from a remote device, the test data representing quantities of microbes present in a microbiome associated with an individual at a first time;
accessing a list of pro-health indicator microbes and/or poor health indicator microbes;
determining, based at least in part on the first test data and the list of pro-health and/or poor health indicator microbes, a first microbiome score representing a quality of the microbiome associated with the individual at the first time; and
sending the microbiome score to a storage location accessible by a device associated with the individual.

42. The method of claim 41, further comprising:
using one or more weighted values associated with types of microbes in determining the microbiome score.

43. The method of claim 41 or 42, further comprising:
receiving first diet data from a remote device, the diet data representing the diet of the individual at the first time; and
using the first diet data at least in part in determining the microbiome score.

44. The method of any one of claims 41 to 43, further comprising:
generating, based at least in part on the first microbiome score and the first test data, a first recommended action, the first recommended action to increase a relative quantity of pro-health indicator microbes or decrease a relative quantity of poor health indicator microbes in the microbiome associated with the individual.

45. The method of claim 44, wherein the first recommended action is a change in diet of the individual.

46. The method of claim 44, wherein the first recommended action is a consumption of prebiotics or probiotics by the individual.

47. The method of any one of claims 41 to 46, further comprising:
receiving second test data from the remote device, the second test data representing quantities of microbes present in the microbiome associated with the individual at a second time, the second time subsequent to the first time;
determining, based at least in part on the second test data and the list of pro-health indicator microbes and/or poor health indicator microbes, a second microbiome score representing a quality of the microbiome associated with the individual at the second time; and
sending the second microbiome score to the storage location accessible by the device associated with the individual.

48. The method of claim 47, further comprising:
receiving tracking data associated with the first recommended action from the device associated with the individual;
wherein the second microbiome score's improvement compared against the first microbiome score is correlated to determining, based at least in part on the tracking data, that the individual has followed the recommended action for a predetermined period for time or until a target associated with the first recommended action is achieved.

49. The method of any one of claims 42 to 48, further comprising:
determining the one or more weighted values based at least in part on health data, and/or diet data, and/or microbiome data associated with a plurality of individuals.

50. The method of any one of claims 42 to 49, further comprising:
determining the one or more weighted values based at least in part on microbiome data associated with a plurality of individuals at two or more times per individual.

51. The method of any one of claims 42 to 50, wherein:
determining the one or more weighted values further comprises:
training one or more machine learned models using microbiome data associated with a plurality of individuals; and
receiving, from the one or more machine learned models, at least one weighted value associated with a type of microbe.

52. The method of any one of claims 41 to 51, further comprising:
determining the first microbiome score further comprises:
inputting the first test data into one or more machine learned models trained using microbiome data associated with a plurality of individuals; and
receiving, as an output from the one or more machine learned models, the first microbiome score.

53. The method of any one of claims 47 to 52, further comprising:
generating a graphical representation of changes in the microbiome associated with the individual over a period of time including the first time and the second time; and
causing the graphical representation to be presented on a display of the device associated with the individual.

54. The method of any one of claims 41 to 53, wherein the quantity of microbes is expressed as a percentage of the microbiome.

55. The method of any one of claims 41 to 54, wherein at least one of the pro-health indicator microbes (identified by its species-level genome bin (SGB) designation) is selected from the group consisting of SGB15249, SGB6340, SGB4964, SGB14252, SGB15229, SGB6174 group, SGB15317, SGB14179, SGB15225, SGB4894, SGB4643, SGB4963, SGB79840, SGB4893, SGB6276, SGB3952, SGB4638, SGB15236, SGB4191, SGB15053 group, SGB15368, SGB4782, SGB14042, SGB4706, SGB4644, SGB49188, SGB4781, SGB4777, SGB14921, SGB15234, SGB8601, SGB5087, SGB14311, SGB4953, SGB7258, SGB4882, SGB6367, SGB15106, SGB4778, SGB15131, SGB4198 group, SGB15031, SGB13981, SGB15123, SGB54300, SGB4665, SGB13979, SGB15410, SGB2290, SGB14954, SGB14306, SGB4805, SGB14899, SGB4803, SGB13982, SGB15265 group, SGB14114, SGB47656, SGB6749, SGB14253, SGB15346, SGB4810, SGB4770, SGB25497, SGB4957, SGB4654, SGB15373, SGB15254, SGB15323, SGB71759, SGB15180, SGB49168, SGB15051, SGB15145, SGB4966, SGB4780, SGB15291, SGB4816, and SGB4714; and/or
wherein at least one of the poor health indicator microbe (identified by its SGB designation) is selected from the group consisting of SGB7253, SGB6769, SGB4721, SGB14837, SGB14546 group, SGB4763, SGB5193, SGB7985, SGB4699, SGB19850 group, SGB17137, SGB4785, SGB15078, SGB53821, SGB15452, SGB15271, SGB4724, SGB8255 group, SGB79823, SGB8028 group, SGB4573 group, SGB14874, SGB4988, SGB5184, SGB4786, SGB4826 group, SGB4041, SGB7984, SGB4761, SGB4447, SGB6744, SGB1836 group, SGB1814, SGB4630 group, SGB8163, SGB4617, SGB4588 group, SGB4742, SGB4572, SGB10115, SGB15158, SGB29328, SGB4791, SGB4688, SGB10068, SGB71883, SGB4760, SGB4037 group, SGB4529, SGB4837 group, SGB79883, SGB4762, SGB4797, SGB14809, SGB4758 group, SGB4703, SGB4606, SGB4584, SGB15132, SGB4746, SGB4862, SGB4798, SGB4861, SGB4608, SGB4035, SGB4794 group, SGB4753, and SGB4583.

56. The method of any one of claims 41 to 55, wherein the microbiome is a gut microbiome.

57. A computer program product comprising coded instructions that, when run on a computer, implement a method as claimed in any one of claims 41 to 56.

58. A system comprising:
one or more processors; and
one or more non-transitory computer readable media storing instructions executable by the one or more processors, wherein the instruction, when executed, causes the one or more processors to perform operations comprising:
receiving one or more weighted values from one or more first machine learned models trained on microbiome data associated with a plurality of individuals, each of the one or more weighted values representing at least in part a pro-health or poor health impact of a microbe or a plurality of microbes;
receiving first test data representing a presence of and quantities of microbes present in a microbiome associated with an individual at a first time;
inputting the test data into one or more second machine learned models which use at least in part the one or more weighted values;
receiving as a first output of the one or more second machine learned models a first microbiome score representing a quality of the microbiome associated with the individual at the first time; and
sending the microbiome score to storage location accessible by a device associated with the individual.

59. The system of claim 58, wherein the operations further comprise:
inputting the test data into one or more third machine learned models trained at least in part on observed changes in microbiome data for a plurality of test subjects over time; and
receiving as an output of the one or more third machine learned models a recommended action, the recommended action intended to increase a relative quantity of pro-health indicator microbes or decrease a relative quantity of poor health indicator microbes in the microbiome associated with the individual.

60. The system of claim 58 or 59, wherein the operations further comprise:
receiving second test data representing quantities of microbes present in the microbiome associated with the individual at a second time, the second time subsequent to the first time;
inputting the second test data into the one or more second machine learned models and receiving as a second output of the one or more second machine learned models a second microbiome score representing a quality of the microbiome associated with the individual at the second time; and
sending the second microbiome score to the storage location accessible by the device associated with the individual.

61. The system of any one of claims 58 to 60, wherein the operations further comprise:
inputting the test data into one or more fourth machine learned models trained at least in part on observed changes in microbiome data for a plurality of test subjects over time; and
receiving as an output of the one or more fourth machine learned models, a plurality of predicted microbiome scores, each of the plurality of predicted microbiome scores representing a quality of the microbiome of the individual at a future time in response to the individual performing a corresponding recommended action.

62. The system of any one of claims 58 to 61, wherein a negative weighted value represents a poor health indicator microbe and a positive weighted value represents a pro-health indicator microbe.

63. The system of any one of claims 58 to 62, wherein inputting the test data into one or more second machine learned models further comprises:
selecting specific microbes from the test data; and
inputting the quantities of the specific microbes into the one or more second machine learned models.

64. The system of any one of claims 58 to 63, wherein the operations further comprise:
receiving user data associated with the individual, the user data representing at least one of a health of the individual, a demographic associated with the individual, a diet of the individual, or a geographic region associated with the individual.

65. The system of any one of claims 58 to 64, wherein at least one of the pro-health indicator microbe (identified by its species-level genome bin (SGB) designation) is selected from the group consisting of SGB15249, SGB6340, SGB4964, SGB14252, SGB15229, SGB6174 group, SGB15317, SGB14179, SGB15225, SGB4894, SGB4643, SGB4963, SGB79840, SGB4893, SGB6276, SGB3952, SGB4638, SGB15236, SGB4191, SGB15053 group, SGB15368, SGB4782, SGB14042, SGB4706, SGB4644, SGB49188, SGB4781, SGB4777, SGB14921, SGB15234, SGB8601, SGB5087, SGB14311, SGB4953, SGB7258, SGB4882, SGB6367, SGB15106, SGB4778, SGB15131, SGB4198 group, SGB15031, SGB13981, SGB15123, SGB54300, SGB4665, SGB13979, SGB15410, SGB2290, SGB14954, SGB14306, SGB4805, SGB14899, SGB4803, SGB13982, SGB15265 group, SGB14114, SGB47656, SGB6749, SGB14253, SGB15346, SGB4810, SGB4770, SGB25497, SGB4957, SGB4654, SGB15373, SGB15254, SGB15323, SGB71759, SGB15180, SGB49168, SGB15051, SGB15145, SGB4966, SGB4780, SGB15291, SGB4816, and SGB4714; and/or
wherein at least one of the poor health indicator microbe (identified by its SGB designation) is selected from the group consisting of SGB7253, SGB6769, SGB4721, SGB14837, SGB14546 group, SGB4763, SGB5193, SGB7985, SGB4699, SGB19850 group, SGB17137, SGB4785, SGB15078, SGB53821, SGB15452, SGB15271, SGB4724, SGB8255 group, SGB79823, SGB8028 group, SGB4573 group, SGB14874, SGB4988, SGB5184, SGB4786, SGB4826 group, SGB4041, SGB7984, SGB4761, SGB4447, SGB6744, SGB1836 group, SGB1814, SGB4630 group, SGB8163, SGB4617, SGB4588 group, SGB4742, SGB4572, SGB10115, SGB15158, SGB29328, SGB4791, SGB4688, SGB10068, SGB71883, SGB4760, SGB4037 group, SGB4529, SGB4837 group, SGB79883, SGB4762, SGB4797, SGB14809, SGB4758 group, SGB4703, SGB4606, SGB4584, SGB15132, SGB4746, SGB4862, SGB4798, SGB4861, SGB4608, SGB4035, SGB4794 group, SGB4753, and SGB4583.

66. The system of any one of claims 58 to 65, wherein the microbiome data is gut microbiome data.

67. The system of any one of claims 58 to 66, wherein the quantity of microbes is expressed as a percentage of the microbiome.

68. A method of using a group of microbes to determine a health condition in a human subject comprising:
obtaining a biological sample containing a microbiome from the human subject;
extracting DNA from microorganisms in the microbiome of the sample;
identifying, from the extracted DNA, a microbial composition of the sample;
analyzing the microbial composition to determine presence, absence, or relative abundance of at least one pro-health indicator microbes and/or at least one poor health indicator microbes in the microbiome of the sample; and
determining the health condition of the human subject based on the presence, absence, or relative abundance of the pro-health and/or poor health indicator microbes detected in the sample;
wherein the at least one of the pro-health indicator microbe (identified by its species-level genome bin (SGB) designation) is selected from the group consisting of SGB15249, SGB6340, SGB4964, SGB14252, SGB15229, SGB6174 group, SGB15317, SGB14179, SGB15225, SGB4894, SGB4643, SGB4963, SGB79840, SGB4893, SGB6276, SGB3952, SGB4638, SGB15236, SGB4191, SGB15053 group, SGB15368, SGB4782, SGB14042, SGB4706, SGB4644, SGB49188, SGB4781, SGB4777, SGB14921, SGB15234, SGB8601, SGB5087, SGB14311, SGB4953, SGB7258, SGB4882, SGB6367, SGB15106, SGB4778, SGB15131, SGB4198 group, SGB15031, SGB13981, SGB15123, SGB54300, SGB4665, SGB13979, SGB15410, SGB2290, SGB14954, SGB14306, SGB4805, SGB14899, SGB4803, SGB13982, SGB15265 group, SGB14114, SGB47656, SGB6749, SGB14253, SGB15346, SGB4810, SGB4770, SGB25497, SGB4957, SGB4654, SGB15373, SGB15254, SGB15323, SGB71759, SGB15180, SGB49168, SGB15051, SGB15145, SGB4966, SGB4780, SGB15291, SGB4816, and SGB4714; and
wherein at least one of the poor health indicator microbe (identified by its species-level genome bin (SGB) designation) is selected from the group consisting of SGB7253, SGB6769, SGB4721, SGB14837, SGB14546 group, SGB4763, SGB5193, SGB7985, SGB4699, SGB19850 group, SGB17137, SGB4785, SGB15078, SGB53821, SGB15452, SGB15271, SGB4724, SGB8255 group, SGB79823, SGB8028 group, SGB4573 group, SGB14874, SGB4988, SGB5184, SGB4786, SGB4826 group, SGB4041, SGB7984, SGB4761, SGB4447, SGB6744, SGB1836 group, SGB1814, SGB4630 group, SGB8163, SGB4617, SGB4588 group, SGB4742, SGB4572, SGB10115, SGB15158, SGB29328, SGB4791, SGB4688, SGB10068, SGB71883, SGB4760, SGB4037 group, SGB4529, SGB4837 group, SGB79883, SGB4762, SGB4797, SGB14809, SGB4758 group, SGB4703, SGB4606, SGB4584, SGB15132, SGB4746, SGB4862, SGB4798, SGB4861, SGB4608, SGB4035, SGB4794 group, SGB4753, and SGB4583.

69. The method of claim 68, further comprising:
determining a microbiome score for the human subject, wherein the microbiome score is calculated using weighted values for each microbe in the sample.

70. The method of claim 69, wherein determining the weighted values comprises:
training one or more machine learned models using microbiome data associated with a plurality of individuals; and
receiving, from the one or more machine learned models, at least one weighted value associated with a type of microbe.

71. The method of claim 69 or 70, wherein the microbiome score represents a quality of the microbiome associated with the human subject.

72. The method of any one of claims 69 to 71, wherein the weighted value comprises at least in part a responsiveness of a microbe to a change in diet.

73. The method of any one of claims 68 to 72, comprising:
identifying in the biological sample at least 10, at least 20, at least 30, at least 40, at least 50, at least 60, at least 70, at least 80, at least 90, at least 100, at least 125, at least 150, at least 175, at least 200, or more than 200 different microbes in the biological sample; and
determining the health condition of the human subject based on presence, absence, and/or absolute or relative abundance of the identified microbes in the biological sample.

74. The method of any one of claims 68 to 73, wherein the group of microbes comprises:
at least three pro-health indicator microbes;
at least five pro-health indicator microbes;
at least ten pro-health indicator microbes; or
more than 10 listed pro-health indicator microbes.

75. The method of any one of claims 68 to 74, wherein the group of microbes comprises:
at least three poor health indicator microbes;
at least five poor health indicator microbes;
at least ten poor health indicator microbes; or
more than 10 listed poor health indicator microbes.

76. The method of any one of claims 68 to 75, wherein the group of microbes comprises: the microbes listed in Table 1A, or another subset of pro-health/good microbes described herein.

77. The method of any one of claims 68 to 75, wherein the group of microbes comprises: the microbes listed in Table 1B, or another subset of poor health/bad microbes described herein.

78. The method of any one of claims 68 to 77, wherein the health condition comprises at least one of: overall good health, overall poor health, obesity, BMI, diabetes risk, cardiometabolic risk, cardiovascular disease risk, or postprandial response to food intake.

79. The method of any one of claims 68 to 78, wherein the identifying comprises one or more of: sequencing one or more nucleic acids of a pro-health or poor health microbe and hybridizing a nucleic acid probe to a nucleic acid of a pro-health or poor health microbe.

80. The method of claim 79, wherein the identifying comprises shotgun metagenomics.

81. The method of any one of claims 68 to 80, wherein the biological sample comprises a stool sample.

82. A method of predicting a health condition in a human subject, comprising:
determining presence, absence, or relative abundance of at least three pro-health indicator microbes in a microbiome of the subject;
determining presence, absence, or relative abundance of at least three poor health indicator microbes in a microbiome of the subject; and
predicting the health condition of the subject, based on the presence, absence, or relative abundance of the pro-health and/or poor health indicator microbes in the microbiome of the subject;
wherein at least one of the pro-health indicator microbes is selected from the group consisting of GOOD BUGS listed in Table 1A, or a subset of pro-health/good microbes described herein; and
wherein at least one of the poor health indicator microbes is selected from the group consisting of BAD BUGS listed in Table 1B, or a subset of poor-health/bad microbes described herein.

83. The method of claim 82, wherein:
the health condition comprises at least one of obesity, increased cardiometabolic risk, increased diabetes risk, or overall poor health; and the health condition is predicted by the presence and/or abundance of more poor health indicator microbes than pro-health indicator microbes; and/or
the health condition comprises at least one of overall good health or absence of obesity, reduced cardiometabolic risk, or reduced diabetes risk; and the health condition is predicted by the presence and/or abundance of more pro-health indicator microbes than poor health indicator microbes.

84. The method of claim 83, wherein the health condition is determined via one or more health metrics, the health metric selected from blood pressure, weight, fasting glucose, and cholesterol levels.

85. A method to predict overall good or poor general health in a non-diseased human subject, comprising:
obtaining a microbiome sample from the human subject;
isolating a nucleic acid fraction from the microbiome sample;
detecting, within the nucleic acid fraction, presence, absence, or relative abundance of at least one unique marker sequence indicative of:
a pro-health indicator microbe selected from the group consisting of GOOD BUGS in Table 1A or a subset of pro-health/good microbes described herein; or
a poor health indicator microbes selected from the group consisting of BAD BUGS in Table 1B, or a subset of poor-health/bad microbes described herein; and at least one of
predicting the human subject has overall good general health if the pro-health indicator microbes outnumber or are relatively more abundant than the poor-health indicator microbes; or
predicting the human subject has overall poor general health if the poor health indicator microbes outnumber or are relatively more abundant than the pro-health indicator microbes.

86. The method of claim 85, further comprising providing to the human subject a dietary recommendation based on the presence, absence, or relative abundance of one or more poor health indicator microbes and/or one or more pro-health indicator microbes.

87. An assay, comprising:
subjecting nucleic acid extracted from a test sample of a human subject to a genotyping assay that detects at least one of GOOD BUGS in Table 1A, the test sample comprising microbiota from a gut of the subject;
determining a relative abundance of the at least one of the GOOD BUGS that is below a predetermined abundance; and
selecting, when the relative abundance is below the predetermined abundance, a treatment regimen that comprises at least one of:
(i) modifying microbiota of the gut of the subject using at least one of a prebiotic, probiotic, or pharmaceutical, or
(ii) altering a diet of the human subject.

88. An assay, comprising:
subjecting nucleic acid extracted from a test sample of a human subject to a genotyping assay that detects at least one of BAD BUGS in Table 1B, the test sample comprising microbiota from a gut of the subject;
determining a relative abundance of the at least one the BAD BUGS that is above a predetermined abundance; and
selecting, when the relative abundance is above the predetermined abundance, a treatment regimen that comprises at least one of:
(i) modifying microbiota of the gut of the subject using at least one of a prebiotic, probiotic, or pharmaceutical, or
(ii) altering a diet of the human subject.

89. A method of diagnosing a human subject as having a healthy diet, comprising detecting in a microbiome sample from the subject a presence of one or more GOOD BUG(s) shown herein to have statistically significant increase using health metrics or both health metrics and diet metrics, and/or an absence of one or more GOOD BUG(s) shown herein to have statistically significant increase using health metrics or both health metrics and diet metrics.

90. A method of diagnosing a human subject as having an unhealthy diet, comprising detecting in a microbiome sample from the subject a presence of one or more BAD BUG(s) shown herein to have statistically significant decrease using health metrics or both health metrics and diet metrics and/or an absence of one or more BAD BUG(s) shown herein to have statistically significant decrease using health metrics or both health metrics and diet metrics.

91. A microbial signature for good health, comprising presence or relatively high abundance of at least three microbes selected from the group consisting of GOOD BUGS listed in Table 1A, or a subset of pro-health/good microbes described herein, and/or absence or relatively low abundance of at least three microbes selected from the group consisting of BAD BUGS listed in Table 1B, or a subset of poor-health/bad microbes described herein.

92. A microbial signature for poor health, comprising absence or relatively low abundance of at least three microbes selected from the group consisting of GOOD BUGS listed in Table 1A, or a subset of pro-health/good microbes described herein, and/or presence or relatively high abundance of at least three microbes selected from the group consisting of BAD BUGS listed in Table 1B, or a subset of poor-health/bad microbes described herein.

93. The microbial signature of claim 91 or 92, wherein the signature comprises:
at least three pro-health indicator microbes;
at least five pro-health indicator microbes;
at least ten pro-health indicator microbes; or
more than 10 listed pro-health indicator microbes.

94. The microbial signature of any one of claims 91 to 93, wherein the group of microbes comprises one or more of:
pro-health indicator microbes showing a statistically significant change in frequency and selected using both health metrics and diet markers, selected from the group consisting of: SGB4964, SGB4782, SGB15053_group, SGB15265_group, SGB15180, SGB4706, SGB4714, SGB4953, SGB4816, SGB15368, SGB25497, SGB4810, SGB15346, SGB47656, SGB5087, SGB4770, SGB15410, SGB4654, SGB4781, SGB15373, SGB4778, SGB4643, SGB4780, SGB15051, SGB6749, SGB8601, SGB4882, SGB15249, SGB71759, SGB15323, SGB15291, SGB4963, SGB6276, SGB6367, SGB15317, SGB14253, SGB15106, SGB4966, SGB4665, SGB4893, SGB4644, SGB15254, SGB15229, SGB4957, and SGB15236; or
pro-health indicator microbes showing a statistically significant increase and selected using both health metrics and diet markers, selected from the group consisting of: SGB4964, SGB4782, SGB15053_group, SGB15265_group, SGB15180, SGB4706, SGB4714, SGB4953, SGB4816, SGB15368, SGB25497, SGB4810, SGB47656, SGB5087, SGB15410, SGB4654, SGB4781, SGB15373, SGB4778, SGB4643, SGB4780, SGB15051, SGB6749, SGB8601, SGB4882, SGB15249, SGB71759, SGB15291, SGB6276, SGB6367, SGB14253, SGB15106, SGB4966, SGB4665, SGB4893, SGB15254, SGB15229, SGB4957, and SGB15236; or
pro-health indicator microbes showing a statistically significant decrease and selected using both health metrics and diet markers, selected from the group consisting of: SGB15346, SGB4770, SGB15323, SGB4963, SGB15317, and SGB4644; or
pro-health indicator microbes showing a statistically significant change in frequency and selected using only health metrics, selected from the group consisting of: SGB4964, SGB4782, SGB14899, SGB15053_group, SGB15265_group, SGB4706, SGB4953, SGB15368, SGB2290, SGB47656, SGB54300, SGB5087, SGB15410, SGB4781, SGB4778, SGB4643, SGB6749, SGB13981, SGB4882, SGB8601, SGB15249, SGB4198_group, SGB4963, SGB6276, SGB15317, SGB6367, SGB14954, SGB14253, SGB15106, SGB4665, SGB4893, SGB4644, SGB49188, and SGB15229; or
pro-health indicator microbes showing a statistically significant increase and selected using only health metrics, selected from the group consisting of: SGB4964, SGB4782, SGB15053_group, SGB15265_group, SGB4706, SGB4953, SGB15368, SGB2290, SGB47656, SGB54300, SGB5087, SGB15410, SGB4781, SGB4778, SGB4643, SGB6749, SGB13981, SGB4882, SGB8601, SGB15249, SGB6276, SGB6367, SGB14253, SGB15106, SGB4665, SGB4893, and SGB15229; or
pro-health indicator microbes showing statistically significant decrease and selected using only health metrics, selected from the group consisting of: SGB14899, SGB4198_group, SGB4963, SGB15317, SGB14954, SGB4644, and SGB49188.

95. The microbial signature of any one of claims 93 to 94, wherein the group of microbes comprises:
at least three poor health indicator microbes;
at least five poor health indicator microbes;
at least ten poor health indicator microbes; or
more than 10 listed poor health indicator microbes.

96. The microbial signature of any one of claims 93 to 95, wherein the group of microbes comprises one or more of:
poor health indicator microbes showing a statistically significant change in frequency and selected using both health metrics and diet markers, selected from the group consisting of: SGB4608, SGB15132, SGB15078, SGB4606, SGB4584, SGB4826_group, SGB14546_group, SGB15452, SGB7985, SGB14837, SGB15271, SGB5184, SGB4573_group, SGB4791, SGB4837_group, SGB4703, SGB4037_group, SGB4753, SGB10068, SGB4721, SGB14874, SGB7253, SGB4035, SGB4746, SGB4572, SGB8163, SGB4630_group, SGB4588_group, SGB4862, SGB6744, SGB4742, SGB4758_group, and SGB4761; or
poor health indicator microbe showing statistically significant increase and selected using both health metrics and diet markers: SGB4761; or
poor health indicator microbes showing a statistically significant decrease and showing statistically significant changes, selected using both health metrics and diet markers, selected from the group consisting of: SGB4608, SGB15132, SGB15078, SGB4606, SGB4584, SGB4826_group, SGB14546_group, SGB15452, SGB7985, SGB14837, SGB15271, SGB5184, SGB4573_group, SGB4791, SGB4837_group, SGB4703, SGB4037_group, SGB4753, SGB10068, SGB4721, SGB14874, SGB7253, SGB4035, SGB4746, SGB4572, SGB8163, SGB4630_group, SGB4588_group, SGB4862, SGB6744, SGB4742, and SGB4758_group; or
poor health indicator microbes showing a statistically significant changes and selected using only health metrics, selected from the group consisting of: SGB4608, SGB15078, SGB15132, SGB4606, SGB4584, SGB4826_group, SGB15452, SGB15271, SGB19850_group, SGB5184, SGB4573_group, SGB4791, SGB4837_group, SGB4703, SGB4037_group, SGB4753, SGB10068, SGB14874, SGB53821, SGB4035, SGB4746, SGB4572, SGB8163, SGB4630_group, SGB4588_group, SGB17137, SGB4862, SGB6744, SGB79823, SGB4742, SGB4447, and SGB4758_group; or
poor health indicator microbes showing statistically significant increase and selected using only health metrics: SGB4447; or
poor health indicator microbes showing a statistically significant decrease and selected using only health metrics, selected from the group consisting of: SGB4608, SGB15078, SGB15132, SGB4606, SGB4584, SGB4826_group, SGB15452, SGB15271, SGB19850_group, SGB5184, SGB4573_group, SGB4791, SGB4837_group, SGB4703, SGB4037_group, SGB4753, SGB10068, SGB14874, SGB53821, SGB4035, SGB4746, SGB4572, SGB8163, SGB4630_group, SGB4588_group, SGB17137, SGB4862, SGB6744, SGB79823, SGB4742, and SGB4758_group.

97. Use of the microbial signature of any one of claims 93 to 96, to guide treatment decisions for a human subject.

98. The use of claim 97, wherein the treatment decision comprises selecting one or more of: modifying overall diet, increasing intake of at least one specified food or supplement, decreasing intake of at least one specified food or supplement, administration of a probiotic composition, administration of a prebiotic composition, or administration of an antibiotic compound.

99. A method for targeting a microbiome of a human subject to promote health, comprising:
detecting in a microbiome sample from the human subject the presence, absence, or relative abundance of one or more pro-health indicator microbes selected from the group consisting of GOOD BUGS listed in Table 1A, or another subset of pro-health/good microbes described herein; and administering to the human a composition that increases growth or survival of the pro-health indicator microbe(s); and/or
detecting in a microbiome sample from the human subject a presence, absence, or relative abundance of one or more poor health indicator microbe selected from the group consisting of BAD BUGS listed in Table 1B, or another subset of poor-health/bad microbes described herein; and administering to the human a composition that decreases growth or survival of the poor health indicator microbe(s).

100. The method of claim 99, comprising detecting:
at least three pro-health indicator microbes;
at least five pro-health indicator microbes;
at least ten pro-health indicator microbes; or
more than 10 listed pro-health indicator microbes.

101. The method of claim 99 or claim 100, wherein the pro-health indicator microbes comprise one or more of:
pro-health indicator microbes showing a statistically significant change in frequency and selected using both health metrics and diet markers, selected from the group consisting of: SGB4964, SGB4782, SGB15053_group, SGB15265_group, SGB15180, SGB4706, SGB4714, SGB4953, SGB4816, SGB15368, SGB25497, SGB4810, SGB15346, SGB47656, SGB5087, SGB4770, SGB15410, SGB4654, SGB4781, SGB15373, SGB4778, SGB4643, SGB4780, SGB15051, SGB6749, SGB8601, SGB4882, SGB15249, SGB71759, SGB15323, SGB15291, SGB4963, SGB6276, SGB6367, SGB15317, SGB14253, SGB15106, SGB4966, SGB4665, SGB4893, SGB4644, SGB15254, SGB15229, SGB4957, and SGB15236; or
pro-health indicator microbes showing a statistically significant increase and selected using both health metrics and diet markers, selected from the group consisting of: SGB4964, SGB4782, SGB15053_group, SGB15265_group, SGB15180, SGB4706, SGB4714, SGB4953, SGB4816, SGB15368, SGB25497, SGB4810, SGB47656, SGB5087, SGB15410, SGB4654, SGB4781, SGB15373, SGB4778, SGB4643, SGB4780, SGB15051, SGB6749, SGB8601, SGB4882, SGB15249, SGB71759, SGB15291, SGB6276, SGB6367, SGB14253, SGB15106, SGB4966, SGB4665, SGB4893, SGB15254, SGB15229, SGB4957, and SGB15236; or
pro-health indicator microbes showing a statistically significant decrease and selected using both health metrics and diet markers, selected from the group consisting of: SGB15346, SGB4770, SGB15323, SGB4963, SGB15317, and SGB4644; or
pro-health indicator microbes showing a statistically significant change in frequency and selected using only health metrics, selected from the group consisting of: SGB4964, SGB4782, SGB14899, SGB15053_group, SGB15265_group, SGB4706, SGB4953, SGB15368, SGB2290, SGB47656, SGB54300, SGB5087, SGB15410, SGB4781, SGB4778, SGB4643, SGB6749, SGB13981, SGB4882, SGB8601, SGB15249, SGB4198_group, SGB4963, SGB6276, SGB15317, SGB6367, SGB14954, SGB14253, SGB15106, SGB4665, SGB4893, SGB4644, SGB49188, and SGB15229; or
pro-health indicator microbes showing a statistically significant increase and selected using only health metrics, selected from the group consisting of: SGB4964, SGB4782, SGB15053_group, SGB15265_group, SGB4706, SGB4953, SGB15368, SGB2290, SGB47656, SGB54300, SGB5087, SGB15410, SGB4781, SGB4778, SGB4643, SGB6749, SGB13981, SGB4882, SGB8601, SGB15249, SGB6276, SGB6367, SGB14253, SGB15106, SGB4665, SGB4893, and SGB15229; or
pro-health indicator microbes showing statistically significant decrease and selected using only health metrics, selected from the group consisting of: SGB14899, SGB4198_group, SGB4963, SGB15317, SGB14954, SGB4644, and SGB4918.

102. The method of any one of claims 99 to 101, comprising detecting:
at least three poor health indicator microbes;
at least five poor health indicator microbes;
at least ten poor health indicator microbes; or
more than 10 listed poor health indicator microbes.

103. The method of any one of claims 99 to 102, wherein the poor health indicator microbes comprise one or more of:
poor health indicator microbes showing a statistically significant change in frequency and selected using both health metrics and diet markers, selected from the group consisting of: SGB4608, SGB15132, SGB15078, SGB4606, SGB4584, SGB4826_group, SGB14546_group, SGB15452, SGB7985, SGB14837, SGB15271, SGB5184, SGB4573_group, SGB4791, SGB4837_group, SGB4703, SGB4037 group, SGB4753, SGB10068, SGB4721, SGB14874, SGB7253, SGB4035, SGB4746, SGB4572, SGB8163, SGB4630_group, SGB4588_group, SGB4862, SGB6744, SGB4742, SGB4758_group, and SGB4761; or
poor health indicator microbe showing statistically significant increase and selected using both health metrics and diet markers: SGB4761; or
poor health indicator microbes showing a statistically significant decrease and showing statistically significant changes, selected using both health metrics and diet markers, selected from the group consisting of: SGB4608, SGB15132, SGB15078, SGB4606, SGB4584, SGB4826_group, SGB14546_group, SGB15452, SGB7985, SGB14837, SGB15271, SGB5184, SGB4573_group, SGB4791, SGB4837_group, SGB4703, SGB4037_group, SGB4753, SGB10068, SGB4721, SGB14874, SGB7253, SGB4035, SGB4746, SGB4572, SGB8163, SGB4630_group, SGB4588_group, SGB4862, SGB6744, SGB4742, and SGB4758_group; or
poor health indicator microbes showing a statistically significant changes and selected using only health metrics, selected from the group consisting of: SGB4608, SGB15078, SGB15132, SGB4606, SGB4584, SGB4826_group, SGB15452, SGB15271, SGB19850_group, SGB5184, SGB4573_group, SGB4791, SGB4837_group, SGB4703, SGB4037_group, SGB4753, SGB10068, SGB14874, SGB53821, SGB4035, SGB4746, SGB4572, SGB8163, SGB4630_group, SGB4588_group, SGB17137, SGB4862, SGB6744, SGB79823, SGB4742, SGB4447, and SGB4758_group; or
poor health indicator microbes showing statistically significant increase and selected using only health metrics: SGB4447; or
poor health indicator microbes showing a statistically significant decrease and selected using only health metrics, selected from the group consisting of: SGB4608, SGB15078, SGB15132, SGB4606, SGB4584, SGB4826_group, SGB15452, SGB15271, SGB19850_group, SGB5184, SGB4573_group, SGB4791, SGB4837_group, SGB4703, SGB4037_group, SGB4753, SGB10068, SGB14874, SGB53821, SGB4035, SGB4746, SGB4572, SGB8163, SGB4630_group, SGB4588_group, SGB17137, SGB4862, SGB6744, SGB79823, SGB4742, and SGB4758_group.

104. A system to assay a biological condition in a subject, comprising:
a nucleic acid sample isolation device, which is adapted to isolate a nucleic acid sample from the subject;
a sequencing device, which is connected to the nucleic acid sample isolation device and adapted to sequence the nucleic acid sample, thereby obtaining a sequencing result; and
an alignment device, which is connected to the sequencing device and adapted to align the sequencing result against sequence from one or more of microbes in order to determine presence, absence, or relative abundance of the microbe(s) based on the alignment result, wherein the microbes comprise one or more of:
pro-health indicator microbes selected from the group consisting of GOOD BUGS listed in Table 1A, or another subset of pro-health/good microbes described herein; and/or
poor health indicator microbes selected from the group consisting of BAD BUGS listed in Table 1B, or another subset of poor-health/bad microbes described herein.

105. A method, comprising:
preparing a food guidance program for a subject;
communicating the food guidance program to the subject;
wherein, when the subject follows the food guidance program:
presence or relative abundance of at least one pro-health indicator microbe in a microbiome of the subject is increased, wherein the at least one pro-health indicator microbes are selected from the group consisting of GOOD BUGS listed in Table 1A, or another subset of pro-health/good microbes described herein; or
presence or relative abundance of at least one poor health indicator microbe in the microbiome of the subject is decreased, wherein the at least one poor health indicator microbe selected from the group consisting of BAD BUGS listed in Table 1B, or another subset of poor-health/bad microbes described herein;
or both.

106. Use of a food guidance program to improve a gut microbiome of a subject, wherein the improvement to the gut microbiome comprises:
increasing presence or relative abundance of at least one pro-health indicator microbe selected from the group consisting of GOOD BUGS listed in Table 1A, or another subset of pro-health/good microbes described herein in the microbiome of the subject;
decreasing presence or relative abundance of at least one poor health indicator microbe selected from the group consisting of BAD BUGS listed in Table 1B, or another subset of poor-health/bad microbes described herein in the microbiome of the subject; or
both.

107. The use of claim 106, wherein the food guidance program is prepared at least in part based on:
presence, absence, or relative abundance of at least one pro-health indicator microbe selected from the group consisting of GOOD BUGS listed in Table 1A, or another subset of pro-health/good microbes described herein in the microbiome of the subject; and/or
presence, absence, or relative abundance of at least one poor health indicator microbe selected from the group consisting of BAD BUGS listed in Table 1B, or another subset of poor-health/bad microbes described herein in the microbiome of the subject.

108. A method, comprising:
preparing a food guidance program for a subject;
communicating the food guidance program to the subject;
wherein, when the subject follows the food guidance program:
presence or relative abundance of at least one pro-health indicator microbe selected from the group consisting of GOOD BUGS listed in Table 1A, or another subset of pro-health/good microbes described herein in a microbiome of the subject is increased;
presence or relative abundance of at least one poor health indicator microbe selected from the group consisting of BAD BUGS listed in Table 1B, or another subset of poor-health/bad microbes described herein in the microbiome of the subject is decreased;
or both.

109. A method, comprising:
detecting in a microbiome sample from a human subject the presence, absence, or relative abundance of one or more pro-health indicator microbes selected from the group consisting of GOOD BUGS listed in Table 1A, or another subset of pro-health/good microbes described herein; and
modifying dietary intake of the human using a food guidance program that increases growth or survival of one or more of the pro-health indicator microbe(s);
and/or
detecting in a microbiome sample from the human subject the presence, absence, or relative abundance of one or more poor health indicator microbe selected from the group consisting of BAD BUGS listed in Table 1B, or another subset of poor-health/bad microbes described herein; and
modifying dietary intake of the human using a food guidance program that decreases growth or survival of one or more of the poor health indicator microbe(s).

110. The method of any one of claim 105, 108, or 109, or the use of any one of claim 106 or 107, wherein the food guidance program is personalized for the subject.

111. A method of altering abundance of at least one microbial species selected from the group consisting of GOOD BUGS listed in Table 1A and BAD BUGS listed in Table 1B in a gut microbiome of a subject, comprising:
providing the subject with a personalized food guidance program, which personalized food guidance program is developed based at least in part on a food score personalized for the subject, and
modifying food intake of the subject based on the personalized food guidance program, thereby altering the abundance of the at least one microbial species in the gut microbiome of the subject.

112. The method of claim 111, wherein altering abundance comprises:
increasing presence or relative abundance of at least one pro-health indicator microbe selected from the group consisting of GOOD BUGS listed in Table 1A, or another subset of pro-health/good microbes described herein in the microbiome of the subject;
decreasing presence or relative abundance of at least one poor health indicator microbe selected from the group consisting of BAD BUGS listed in Table 1B, or another subset of poor-health/bad microbes described herein in the microbiome of the subject; or
both.

113. A method of improving a gut microbiome profile of a subject, comprising:
assaying the gut microbiome of the subject, thereby producing a first microbiome signature of the subject;
developing a personalized food guidance program for the subject, which personalized food guidance program will improve the gut microbiome of the subject when the subject follows the program;
communicating the personalized food guidance program to the subject;
assaying the gut microbiome of the subject at a time the after the subject begins to follow the personalized food guidance program, thereby producing a second microbiome signature of the subject;
comparing the first microbiome signature of the subject to the second microbiome signature of the subject,
wherein the first and second microbiome signatures comprise the presence, absence, or relative abundance of one or more pro-health indicator microbes selected from the group consisting of GOOD BUGS listed in Table 1A and/or the presence, absence, or relative abundance of one or more poor health indicator microbes selected from the group consisting of BAD BUGS listed in Table 1B; and
wherein an increase in the presence or relative abundance of the one or more pro-health indicator microbes and/or a decrease in the presence or relative abundance of the one or more poor health indicator microbes from the first signature to the second signature constitutes an improved gut microbiome profile of the subject.

114. The use or the method of claim 113, or the method of any one of claims 111 to 113, wherein the personalized food guidance program is based at least based in part on one or more of:
a non-microbial biomarker of the subject;
a nutritional response of the subject;
medical history of the subject;
a health condition of the subject;
a health goal of the subject;
predicted hunger of the subject;
predicted response to food consumption of the subject;
glucose response of the subject;
fat response of the subject;
microbiome data of the subject;
data about the subject's overall health; and/or
potential health risks for the subject.

115. The use or the method of any one of claims 105 to 114, wherein the pro-health indicator microbe comprises GOOD BUG(S) selected from a subset of pro-health/good microbes described herein.

116. The use or the method of any one of claims 105 to 114, wherein the poor health indicator microbe comprises one or more of BAD BUG(S) selected from a subset of poor-health/bad microbes described herein.

117. The use or the method of any one of claims 105 to 116, wherein the food guidance program is developed based at least in part based on or in reference to a database comprising one or more of:
correlations between food consumed and microbiome members for thousands of individual subjects; and/or
a plurality of foods designated as “to be promoted in a diet”, wherein this designation indicates the food tends to support a healthy condition in the subject, or tends to not support an unhealthy condition in the subject, or tends to move a subject incorporating the food in their diet more toward a healthy state; and/or
a plurality of foods designated as “to be demoted in a diet”, wherein this designation indicates the food tends to not support a healthy condition in the subject, or tends to support an unhealthy condition in the subject, or tends to move a subject incorporating the food in their diet more toward a less healthy state.
